SEQUENCE LISTING (17 sequences)

SEQ ID NO: 1 - DNA, 72 bp, Artificial sequence
  synthetic construct
gggagacataaaccctggcgcgctcgcggcccggcactcttctggtccccacagactcagagagaagccacc

SEQ ID NO: 2 - DNA, 72 bp, Artificial sequence
  synthetic construct
gggagacataaaccctggcgcgctcgcgggccggcactcttctggtccccacagactcagagagaagccacc

SEQ ID NO: 3 - DNA, 43 bp, Artificial sequence
  Synthetic construct
gggagactcttctggtccccacagactcagagagaacgccacc

SEQ ID NO: 4 - DNA, 511 bp, Artificial sequence
  Synthetic construct
gttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatggccacaacc

SEQ ID NO: 5 - DNA, 143 bp, Artificial sequence
  Synthetic construct
aaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagca

SEQ ID NO: 6 - DNA, 47 bp, Artificial sequence
  Synthetic construct
gggagacaagagagaaaagaagagcaagaagaaatataagagccacc

SEQ ID NO: 7 - DNA, 66 bp, Artificial sequence
  Synthetic construct
gggagacccaagctggctagcgtttaaacttaagcttggcaatccggtactgttggtaaagccacc

SEQ ID NO: 8 - DNA, 291 bp, Artificial sequence
  Synthetic construct
gggagacccaagctggctagcgtttaaacttaagctttcctttccgggccggctgggcgcgccgaagcgcctgcgccttggctgctggtcggttgctgggtaaccgcgtcagggagttggattctatcctgcaagggcacggggacccacaacgacggctgtccctaaagaaccgttgcgactggtaactgaagtggaagagagtccagatttcttgtgtgtggtcaaggagacggacaaactttttgtcttcagacgagggagcgttttgtaggctctccaggggttgag

SEQ ID NO: 9 - DNA, 186 bp, Artificial sequence
  Synthetic construct
ggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccattgtcttc

SEQ ID NO: 10 - DNA, 114 bp, Artificial sequence
  Synthetic construct
tcagtagggtcatgaaggtttttcttttcctgagaaaacaacacgtattgttttctcaggttttgctttttggcctttttctagcttaaaaaaaaaaaaagcaaaattgtcttc

SEQ ID NO: 11 - DNA, 112 bp, Artificial sequence
  Synthetic construct
tcagtagggttgtaaaggtttttcttttcctgagaaaacaaccttttgttttctcaggttttgctttttggcctttccctagctttaaaaaaaaaaaagcaaaattgtcttc

SEQ ID NO: 12 - DNA, 68 bp, Artificial sequence
  Synthetic construct
gaagtggcggttcggccggaggttccatcgtatccaaaaggctcttttcagagccacccattgtcttc

SEQ ID NO: 13 - DNA, 239 bp, Artificial sequence
  Synthetic construct
ggggctggcctcagtctctgtcccatcgcttgaatacagtactcctagggcttgaccctggtacccagcccagccttagcacccagcatgtgaccccactcctgatcaggtcccagcatcttcccttcttgttctgttccttaaggtcccagcaccttaccccaggacttggtcttcaaccaccattacccctctaactttgcacaaataaacctgtgtagaaacccaccccaaaaaaa

SEQ ID NO: 14 - DNA, 66 bp, Artificial sequence
  Synthetic construct
gggagacccaagctggctagcgtttaaacttcagcttggcaatccggtactgttggtaaagccacc

SEQ ID NO: 15 - DNA, 2100 bp, Artificial sequence
  Synthetic construct
atgatcccagcaagcgccaaggcaccacacaagcagccccacaagcagagcatcagcatcggcaggggcacaaggaagagggacgaggacagcggaaccgaagtgggagagggaacagacgagtgggcacagagcaaggcaaccgtgcgcccacccgaccagctggagctgacagacgccgagctgaaggaggagttcaccaggatcctgacagccaacaacccacacgccccccagaacatcgtgcgctacagcttcaaggagggcacatacaagccaatcggcttcgtgaaccagctggccgtgcactacacccaagtgggcaacctgatccccaaggacagcgacgagggccggagacagcactacagggacgagctggtggcaggaagccaggagagcgtgaaagtgatcagcgagaccggcaacctggaggaggacgaggagccaaaggagctggagaccgagccaggaagccagacagacgtgcccgcagcaggagcagcagagaaggtgaccgaggaggagctgatgacacccaagcagccaaaggagcggaagctgaccaaccagttcaacttcagcgagagagccagccagacatacaacaacccagtgcgggacagagagtgccagaccgagccaccccccagaaccaacttcagcgccacagccaaccagtgggagatctacgacgcctacgtggaggagctggagaagcaggagaagaccaaggagaaggagaaggccaagacacccgtggccaagaagagcggcaagatggccatgcggaagctgaccagcatggagagccagacagacgacctgatcaagctgagccaggccgccaagatcatggagagaatggtgaaccagaacacctacgacgacatcgcccaggacttcaagtactacgacgacgcagcagacgagtacagggaccaagtgggcacactgctgcccctgtggaagttccagaacgacaaggccaagaggctgagcgtgaccgccctgtgctggaacccaaagtacagggacctgttcgcagtgggatacggaagctacgacttcatgaagcagagcagaggcatgctgctgctgtacagcctgaagaaccccagcttccccgagtacatgttcagcagcaacagcggcgtgatgtgcctggacatccacgtggaccacccctacctggtggccgtgggccactacgacggcaacgtggccatctacaacctgaagaagccccacagccagcccagcttctgcagcagcgccaagagcggcaagcacagcgaccccgtgtggcaggtgaagtggcagaaggacgacatggaccagaacctgaacttcttcagcgtgagcagcgacggcaggatcgtgagctggaccctggtgaagcgcaagctggtgcacatcgacgtgatcaagctgaaggtggagggcagcaccacagaggtgccagagggactgcagctgcacccagtgggatgcggcacagccttcgacttccacaaggagatcgactacatgttcctggtgggcaccgaggagggcaagatctacaagtgcagcaagagctacagcagccagttcctggacacatacgacgcccacaacatgagcgtggacaccgtgagctggaacccctaccacacaaaggtgttcatgagctgcagcagcgactggaccgtgaagatctgggaccacaccatcaagacacccatgttcatctacgacctgaacagcgccgtgggcgacgtggcatgggcaccatacagcagcacagtgttcgcagcagtgaccacagacggcaaggcacacatcttcgacctggccatcaacaagtacgaggccatctgcaaccagcccgtggccgccaagaagaacaggctgacccacgtgcagttcaacctgatccaccccatcatcatcgtgggcgacgaccggggccacatcatcagcctgaagctgagccccaacctgagaaagatgcccaaggagaagaagggacaggaggtgcagaagggaccagcagtggagatcgcaaagctggacaagctgctgaacctggtgcgcgaggtgaagatcaagacctga

SEQ ID NO: 16 - DNA, 2100 bp, Artificial sequence
  Synthetic construct
atgattcctgcttctgcgaaggctccccataaacagcctcataagcagagcatcagcataggcagaggaaccaggaagagagatgaagattcagggactgaagtgggagaaggcacagatgaatgggcccaatccaaagccacagttagaccccctgaccagctggagttgaccgatgcggagttaaaggaggagttcactcggattttgacagccaacaacccacacgcaccccagaacattgtcaggtacagcttcaaagaaggcacatataagcctattggctttgtgaaccaactggcagttcactacacccaggttgggaacctgatccccaaagactcagatgaaggacggcggcagcattaccgcgatgaattagtggcaggttctcaggagtctgtcaaggtgatttcagaaacaggaaacctcgaagaagacgaagagcccaaggagttagaaactgagcctgggagtcaaacagatgtgcctgcagctggggcagctgaaaaagtgactgaagaagaattgatgactcctaagcagcccaaggagagaaagctcactaaccagttcaacttcagtgagagggcctcacagacctacaacaaccctgtccgggatcgagaatgccagacggagcctcctcccaggacaaacttttcagccacagccaatcagtgggagatctatgatgcctatgtagaggaacttgagaagcaggaaaagaccaaagagaaggagaaggcaaagaccccagtggctaaaaaatcagggaagatggccatgaggaagctgacatctatggagtctcagactgatgatctcatcaaattgtcccaagctgctaagatcatggagcggatggtcaaccagaatacatatgatgacattgctcaagattttaagtactatgacgatgctgctgatgaataccgggaccaggtgggtaccctgctgccgctctggaagttccaaaatgacaaagccaagcgcctgtccgtcactgccctctgctggaatccaaagtacagggatctgtttgcagtgggatatggctcttatgacttcatgaagcagagccggggcatgctgctgctctacagcctgaagaaccccagcttccctgagtacatgttcagcagcaacagcggcgtcatgtgtctcgacatccacgtggaccacccctacctggtggcagtaggccactatgacggcaacgtggccatttacaacctcaagaagccccactcccagccctccttctgcagctcagccaagtctggcaagcactcagaccctgtgtggcaggtcaagtggcagaaggatgacatggaccaaaaccttaacttcttctctgtgtcatctgacggcaggattgtgtcttggactctcgtgaagagaaagctggttcacatagatgtcatcaagctgaaggtggaaggcagcaccacggaagttcctgaggggttgcagctgcacccagtgggttgtggcactgcctttgacttccacaaagagattgactacatgttcctagtgggcacagaggagggaaaaatctacaagtgctctaaatcctactccagccaattcctcgacacctatgacgcccacaacatgtcagtggacactgtgtcctggaacccataccacaccaaggtcttcatgtcctgcagctccgactggacagtgaagatctgggaccacaccatcaagaccccgatgttcatctatgacctgaactcagccgtgggtgatgtggcctgggcgccatactcttctactgtgttcgcagcagtcaccacagatgggaaggcccacatatttgacttagccatcaacaagtatgaggccatctgcaaccagcctgtggcggccaaaaagaacaggctcacccacgtgcagttcaatctcatccaccccatcatcattgtgggcgatgaccgtgggcacatcatcagcctcaagctctcacccaatttgcgcaagatgccaaaggaaaagaaggggcaggaggtgcagaagggtccagctgtggagattgcgaaactggacaaactgctgaacctggtgagggaagtgaaaatcaagacctga

SEQ ID NO: 17 - DNA, 2100 bp, Artificial sequence
  Synthetic construct
atgatcccagcaagcgccaaggcaccacacaagcagccccacaagcagagcatctccatcggcaggggcacaaggaagagggacgaggatagcggaaccgaagtgggagagggaacagacgagtgggcacagtccaaggcaaccgtgcgcccacctgaccagctggagctgacagatgccgagctgaaggaggagttcaccaggatcctgacagccaacaatccacacgccccccagaacatcgtgcgctactctttcaaggagggcacatataagccaatcggctttgtgaaccagctggccgtgcactatacccaagtgggcaatctgatccccaaggactccgatgagggccggagacagcactacagggacgagctggtggcaggatcccaggagtctgtgaaagtgatctctgagaccggcaatctggaggaggacgaggagccaaaggagctggagaccgagccaggaagccagacagatgtgcctgcagcaggagcagcagagaaggtgaccgaggaggagctgatgacacctaagcagccaaaggagcggaagctgaccaaccagttcaatttttccgagagagcctctcagacatacaacaatccagtgcgggacagagagtgccagaccgagccaccccctagaaccaacttttccgccacagccaatcagtgggagatctacgatgcctatgtggaggagctggagaagcaggagaagaccaaggagaaggagaaggccaagacacccgtggccaagaagtccggcaagatggccatgcggaagctgaccagcatggagtcccagacagacgatctgatcaagctgtctcaggccgccaagatcatggagagaatggtgaaccagaatacctatgacgatatcgcccaggacttcaagtactatgacgatgcagcagacgagtacagggatcaagtgggcacactgctgcctctgtggaagtttcagaacgataaggccaagaggctgagcgtgaccgccctgtgctggaatccaaagtacagggacctgttcgcagtgggatacggatcttatgacttcatgaagcagagcagaggcatgctgctgctgtattccctgaagaacccctctttccctgagtacatgtttagctccaattccggcgtgatgtgcctggacatccacgtggatcacccctacctggtggccgtgggccactatgacggcaacgtggccatctacaatctgaagaagcctcactctcagcccagcttctgttctagcgccaagagcggcaagcactccgatcccgtgtggcaggtgaagtggcagaaggacgatatggaccagaacctgaatttcttttccgtgtcctctgatggcaggatcgtgtcttggaccctggtgaagcgcaagctggtgcacatcgacgtgatcaagctgaaggtggagggcagcaccacagaggtgccagagggactgcagctgcacccagtgggatgcggcacagccttcgactttcacaaggagatcgattatatgttcctggtgggcaccgaggagggcaagatctacaagtgttctaagagctatagctcccagtttctggacacatatgatgcccacaacatgagcgtggataccgtgtcctggaatccttaccacacaaaggtgttcatgagctgctctagcgactggaccgtgaagatctgggatcacaccatcaagacacctatgtttatctatgacctgaactccgccgtgggcgatgtggcatgggcaccatactcctctacagtgttcgcagcagtgaccacagacggcaaggcacacatctttgatctggccatcaacaagtacgaggccatctgtaatcagcccgtggccgccaagaagaacaggctgacccacgtgcagttcaatctgatccaccctatcatcatcgtgggcgacgatcggggccacatcatctctctgaagctgagccccaacctgagaaagatgcctaaggagaagaagggacaggaggtgcagaagggaccagcagtggagatcgcaaagctggacaagctgctgaatctggtgcgcgaggtgaagatcaagacctga

(19) United States
(12) Patent Application Publication
(10) Pub. No.: US 2024/0398983 A1
(43) Pub. Date: Dec. 5, 2024

(54) POLYNUCLEOTIDE COMPOSITIONS, RELATED FORMULATIONS, AND METHODS OF USE THEREOF

(71) Applicant: ReCode Therapeutics, Inc., Menlo Park, CA (US)

(72) Inventors: Vladimir Kharitonov, Menlo Park, CA (US); Brandon Wustman, Menlo Park, CA (US); Jackson Eby, Menlo Park, CA (US); Rumpa Bhattacharjee, Menlo Park, CA (US); Daniella Ishimaru, Menlo Park, CA (US); Mirko Hennig, Menlo Park, CA (US)

(21) Appl. No.: 18/282,699

(22) PCT Filed: Mar. 22, 2022
(86) PCT No.: PCT/US2022/021437

§ 371 Date: Sep. 18, 2023

Related U.S. Application Data
(60) Provisional application No. 63/164,522, filed on Mar. 22, 2021.
(60) Provisional application No. 63/164,577, filed on Mar. 23, 2021.
(60) Provisional application No. 63/229,495, filed on Aug. 4, 2021.

(51) Int. Cl. A61K 48/00; A61K 9/00; A61K 9/127; A61K 9/51; A61K 38/17; A61K 47/24; A61P 11/00

(52) U.S. Cl. CPC A61K 48/005; A61K 9/0078; A61K 9/127; A61K 9/5123; A61K 38/1709; A61K 47/24; A61K 48/0075; A61P 11/00

(57) ABSTRACT

Compositions of polynucleotide(s) are disclosed. A polynucleotide may encode for a polypeptide, protein, or functional fragment thereof associated with primary ciliary dyskinesia (PCD), such as dynein axonemal intermediate chain 1 (DNAI1). Pharmaceutical compositions, kits, and methods for treating a disease or condition associated with cilia maintenance and function, and impaired function of the axoneme are also disclosed. The polynucleotide may be assembled with a lipid composition for delivery to an organ, such as the lung, of a subject. The lipid composition may comprise an ionizable cationic lipid. The polynucleotide can be expressed within cells of the organ of the subject.

### CROSS-REFERENCE

This application claims the benefit of is a U.S. national stage application under 35 U.S.C. 371 of International Patent Application No. PCT/US2022/021437 filed on Mar. 22, 2022, which claims priority to U.S. Provisional Patent Application No. 63/164,522, filed on Mar. 22, 2021, and U.S. Provisional Patent Application No. 63/164,577, filed on Mar. 23, 2021, U.S. Provisional Application Patent No. 63/229,495, filed Aug. 4, 2021, each of which is incorporated by reference herein in its entirety.

### BACKGROUND

Nucleic acids, such as messenger ribonucleic acid (mRNA) may be used by cells to express proteins and polypeptides. Some cells may be deficient in a certain protein or nucleic acid and result in disease states. A cell can also take up and translate an exogenous RNA, but many factors influence efficient uptake and translation. For instance, the immune system recognizes many exogenous RNAs as foreign and triggers a response that is aimed at inactivating the RNAs.

### SEQUENCE LISTING

The instant application contains a Sequence Listing, which has been submitted electronically in ASCII format and is hereby incorporated by reference in its entirety. Said ASCII copy, created on Jul. 1, 2024, is named 061529-738N01US_ST25.txt and is 13.4 KB in size.

### SUMMARY

Provided here are composition and methods for delivery of nucleic acids. Nucleic acids may be used as a therapeutic. In particular, mRNA may be delivered to a cell of a subject. Upon delivery of a nucleic acid to a cell, the nucleic acid may be used to synthesize a polypeptide. In the case of a cell or subject with a disease or disorder, the nucleic acid may be effective at acting as a therapeutic by increasing the expression of a polypeptide. In cases, where a disorder or disease is caused or correlated to aberrant expression or activity of polypeptide, the increased in expression of the polypeptide may be beneficial. However, the cells may have limited uptake of exogenous nucleic acids and the delivery of the nucleic acids may benefit from compositions that allow for increase uptake of a nucleic acid.

Additionally, therapeutic agents such as proteins and small molecule therapeutic agents could benefit from organ specific delivery. Many different types of compounds such as chemotherapeutic agents exhibit significant cytotoxicity. If these compounds could be better directed towards delivery to the desired organs, then fewer off target effects will be seen.

In an aspect, the present disclosure provides a pharmaceutical composition comprising a polynucleotide assembled with a lipid composition, wherein: the polynucleotide encodes a dynein axonemal intermediate chain 1 (DNAI1) protein; and the lipid composition comprises (i) an ionizable cationic lipid, and (ii) a selective organ targeting (SORT) lipid separate from the ionizable cationic lipid. In some embodiments, the lipid composition further comprises (iii) a phospholipid.

In some embodiments, the polynucleotide comprises a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15. In some embodiments, the nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence over at least 1,000 bases (e.g., nucleotide residues 1 to 1,000) of SEQ ID NO: 15. In some embodiments, the nucleic acid sequence has 100% sequence identity to a sequence over at least 1,000 bases (e.g., nucleotide residues 1 to 1,000) of SEQ ID NO: 15. In some embodiments, at least 90%, 95%, or 97% nucleotides replacing uridine within the polynucleotide are nucleotide analogues. In some embodiments, fewer than 15% of nucleotides within the polynucleotide are nucleotide analogues. In some embodiments, the polynucleotide comprises 1-methylpseudouridine. In some embodiments, the nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CAG, AGG, CGG, CGA, CGT, CGC, TCG, TCA, TCT, TCC, ACG, ACT, GTA, GTT, GTC, and TAT, as compared to a corresponding wild-type sequence selected from SEQ ID NO: 16. In some embodiments, the nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence selected from SEQ ID NO: 16. In some embodiments, the nucleic acid sequence comprises fewer codon types encoding an amino acid as compared to a corresponding wild-type sequence selected from SEQ ID NO: 16. In some embodiments, at least one type of an isoleucine-encoding codons in the corresponding wild-type sequence is substituted with a synonymous codon type in the nucleic acid sequence. In some embodiments, at least one type of a valine-encoding codons in the corresponding wild-type sequence is substituted with a synonymous codon type in the nucleic acid sequence. In some embodiments, at least one type of an alanine-encoding codons in the corresponding wild-type sequence is substituted with a synonymous codon type in the nucleic acid sequence. In some embodiments, at least one type of a glycine-encoding codons in the corresponding wild-type sequence is substituted with a synonymous codon type in the nucleic acid sequence. In some embodiments, at least one type of a proline-encoding codons in the corresponding wild-type sequence is substituted with a synonymous codon type in the nucleic acid sequence. In some embodiments, at least one type of a threonine-encoding codons in the corresponding wild-type sequence is substituted with a synonymous codon type in the nucleic acid sequence. In some embodiments, at least one type of a leucine-encoding codons in the corresponding wild-type sequence is substituted with a synonymous codon type in the nucleic acid sequence. In some embodiments, at least one type of an arginine-encoding codons in the corresponding wild-type sequence is substituted with a synonymous codon type in the nucleic acid sequence. In some embodiments, at least one type of a serine-encoding codons in the corresponding wild-type sequence is substituted with a synonymous codon type in the nucleic acid sequence.

In some embodiments, the pharmaceutical composition comprises an excipient. In some embodiments, polynucleotide is present in the pharmaceutical composition at a concentration of no more than 1 mg/mL. In some embodiments, polynucleotide is present in the pharmaceutical composition at a concentration of no more than 5 mg/mL. In some embodiments, a molar ratio of nitrogen in the lipid composition to phosphate in the polynucleotide (N/P ratio) is no more than about 20:1. In some embodiments, the N/P ratio is from about 5:1 to about 20:1. In some embodiments, a molar ratio of the polynucleotide to total lipids of the lipid composition is no more than about 1:1, 1:10, 1:50, or 1:100. In some embodiments, at least about 85% of the polynucleotide is encapsulated in particles of the lipid compositions. In some embodiments, the lipid composition comprises particles characterized by a (e.g., average) size of 100 nanometers (nm) or less. In some embodiments, the lipid composition comprises a plurality of particles characterized by a polydispersity index (PDI) of no more than about 0.2. In some embodiments, the lipid composition comprises a plurality of particles characterized by a negative zeta potential of −5, −4, or −3 millivolts (mV) or a lower negative number.

In some embodiments, the SORT lipid is present in an amount in the lipid composition to effect a (e.g., 1.1- or 10-fold) greater expression or activity of the polynucleotide in a (e.g., lung) cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect a (e.g., 1.1- or 10-fold) greater expression or activity of the polynucleotide in a (e.g., lung) cell compared to that achieved with a corresponding reference lipid composition that does not comprise the SORT lipid. In some embodiments, the cell is a ciliated cell. In some embodiments, the SORT lipid is present in an amount in the lipid composition to effect an expression or activity of the polynucleotide in a (e.g., 1.1- or 10-fold) greater plurality of (e.g., lung) cells compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect an expression or activity of the polynucleotide in a (e.g., 1.1- or 10-fold) greater plurality of (e.g., lung) cells compared to that achieved with a corresponding reference lipid composition that does not comprises the SORT lipid. In some embodiments, the plurality of cells are ciliated cells. In some embodiments, the lipid composition comprises the SORT lipid at a molar percentage from about 20% to about 65%. In some embodiments, the lipid composition comprises the ionizable cationic lipid at a molar percentage from about 5% to about 30%. In some embodiments, the lipid composition comprises the phospholipid at a molar percentage from about 8% to about 23%. In some embodiments, the phospholipid is not an ethylphosphocholine. In some embodiments, the lipid composition further comprises a steroid or steroid derivative (e.g., at a molar percentage from about 15% to about 46%). In some embodiments, the lipid composition further comprises a polymer-conjugated lipid (e.g., poly(ethylene glycol) (PEG)-conjugated lipid) (e.g., at a molar percentage from about 0.5% to about 10%). In some embodiments, the lipid composition has an apparent ionization constant (pKa) is of about 8 or higher (e.g., about 8 to about 13). In some embodiments, the SORT lipid comprises a permanently positively charged moiety (e.g., a quaternary ammonium ion). In some embodiments, the SORT lipid comprises a counterion. In some embodiments, the SORT lipid is a phosphocholine lipid. In some embodiments, the SORT lipid is an ethylphosphocholine, optionally selected from 1,2-dimyristoleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, and 1-palmitoyl-2-oleoyl-sn-glycero-3-ethylphosphocholine.

In some embodiments, the SORT lipid comprises a headgroup having a structural formula:

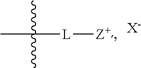

wherein L is a (e.g., biodegradable) linker; Z⁺ is positively charged moiety (e.g., a quatemary ammonium ion); and X⁻ is a counterion. In some embodiments, the SORT lipid has a structural formula:

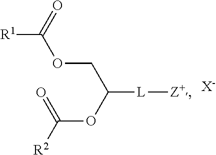

wherein R¹ and R² are each independently an optionally substituted C₆-C₂₄ alkyl, or an optionally substituted C₆-C₂₄ alkenyl. In some embodiments, the SORT lipid has a structural formula:

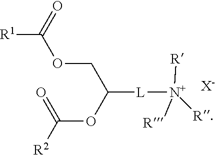

In some embodiments, L is

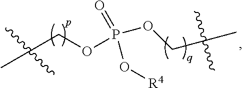

wherein: p and q are each independently 1, 2, or 3; and R⁴ is an optionally substituted C₁-C₆ alkyl. In some embodiments, the SORT lipid has a structural formula:

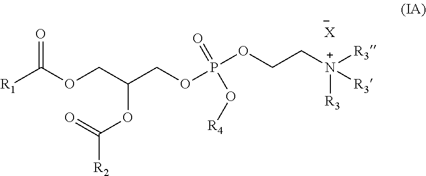

wherein: R₁ and R₂ are each independently alkyl_{(C8-C24)}, alkenyl_{(C8-C24)}, or a substituted version of either group; R₃, R₃′, and R₃″ are each independently alkyl_{(C≤6)} or substituted alkyl_{(C≤6)}; R₄ is alkyl_{(C≤6)} or substituted alkyl_{(C≤6)}; and X⁻ is a monovalent anion. In some embodiments, the SORT lipid has a structural formula:

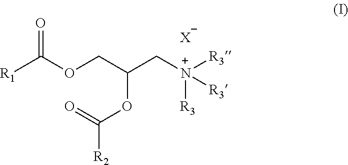

wherein: R₁ and R₂ are each independently alkyl_{(C8-C24)}, alkenyl_{(C8-C24)}, or a substituted version of either group, R₃, R₃′, and R₃″ are each independently alkyl_{(C≤6)} or substituted alkyl_{(C≤6)}; and X⁻ is a monovalent anion. In some embodiments, the SORT lipid has a structural formula:

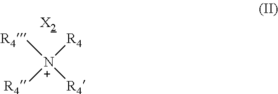

wherein: R₄ and R₄′ are each independently alkyl_{(C6-C24)}, alkenyl_{(C6-C24)}, or a substituted version of either group; R₄′ is alkyl_{(C≤24)}, alkenyl_{(C≤24)}, or a substituted version of either group; R₄′″ is alkyl_{(C1-C8)}, alkenyl_{(C2-C8)}, or a substituted version of either group; and X₂⁻ is a monovalent anion.

In some embodiments, the ionizable cationic lipid is a dendrimer or dendron having the formula:

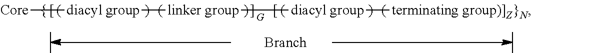

or a pharmaceutically acceptable salt thereof, wherein: (a) the core comprises a structural formula (X_{Core}):

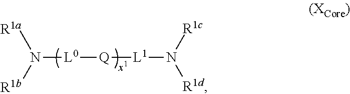

wherein: Q is independently at each occurrence a covalent bond, —O—, —S—, —NR²—, or —CR^{3a}R^{3b}—; R² is independently at each occurrence R^{1g} or —L²—NR^{1e}R^{1f}; R^{3a} and R^{3b} are each independently at each occurrence hydrogen or an optionally substituted (e.g., C₁-C₆, such as C₁-C₃) alkyl; R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl; L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or, alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and x¹ is 0, 1, 2, 3, 4, 5, or 6; and (b) each branch of the plurality (N) of branches independently comprises a structural formula (X_{Branch}):

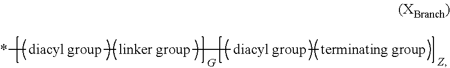

wherein: * indicates a point of attachment of the branch to the core; g is 1, 2, 3, or 4; Z=2^{(g-1)}; G=0, when g=1; or G=Σᵢ₌₀^{i=g-2}2ⁱ, when g≠1; (c) each diacyl group independently comprises a structural formula

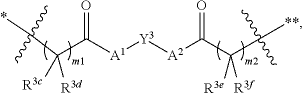

wherein: * indicates a point of attachment of the diacyl group at the proximal end thereof; ** indicates a point of attachment of the diacyl group at the distal end thereof; Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) alkenylene; A¹ and A² are each independently at each occurrence —O—, —S—, or —NR⁴—, wherein: R⁴ is hydrogen or optionally substituted (e.g., C₁-C₆) alkyl; m¹ and m² are each independently at each occurrence 1, 2, or 3; and R^{3c}, R^{3d}, R^{3e}, and R^{3f} are each independently at each occurrence hydrogen or an optionally substituted (e.g., C₁-C₈) alkyl; and (d) each linker group independently comprises a structural formula

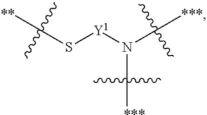

wherein: ** indicates a point of attachment of the linker to a proximal diacyl group;*** indicates a point of attachment of the linker to a distal diacyl group; and Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and (e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol. In some embodiments, x¹ is 0, 1, 2, or 3. In some embodiments, R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl

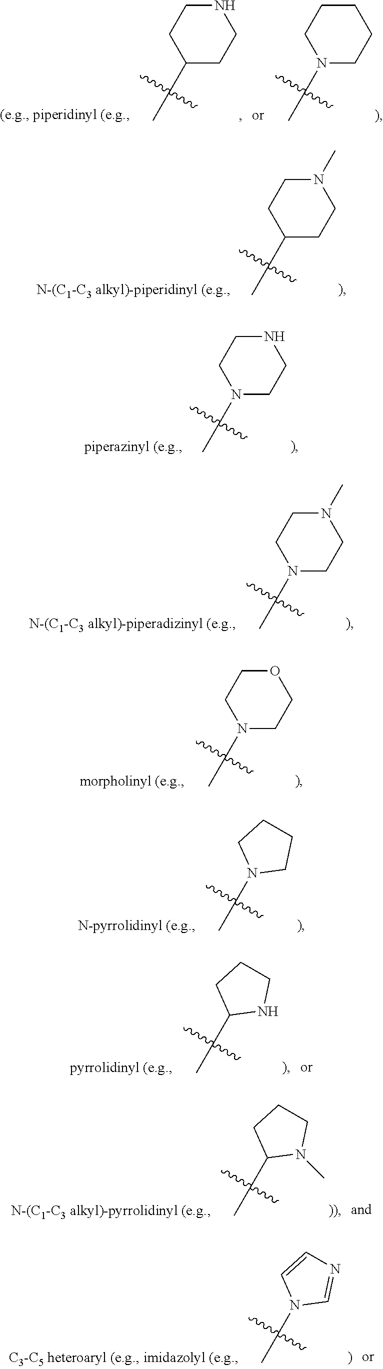

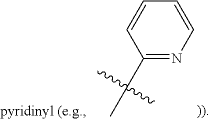 [SMILES: *c1ccccn1]

In some embodiments, R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH. In some embodiments, R³a and R^{3b} are each independently at each occurrence hydrogen.

In some embodiments, the plurality (N) of branches comprises at least 3 (e.g., at least 4, or at least 5) branches. In some embodiments, g=1; G=0; and Z=1. In some embodiments, each branch of the plurality of branches comprises a structural formula *diacyl group terminating group). In some embodiments, g=2; G=1; and Z=2. In some embodiments, each branch of the plurality of branches comprises a structural formula

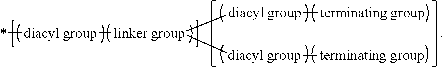

In some embodiments, g=3; G=3; and Z=4. In some embodiments, each branch of the plurality of branches comprises a structural formula

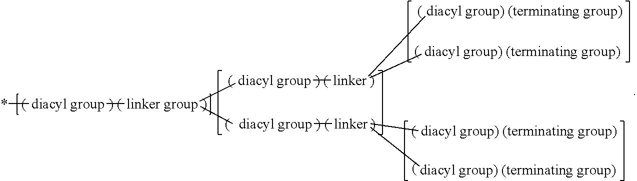

In some embodiments, g=4; G=7; and Z=8. In some embodiments, each branch of the plurality of branches comprises a structural formula

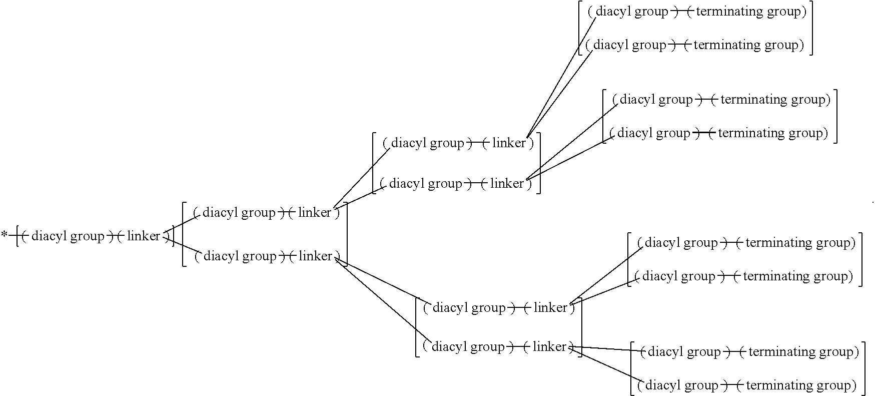

In some embodiments, the core comprises a structural formula

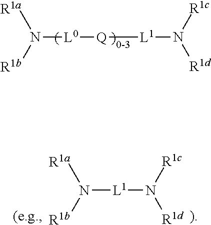

In some embodiments, the core comprises a structural formula:

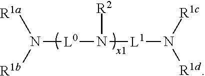

In some embodiments, the core comprises a structural formula:

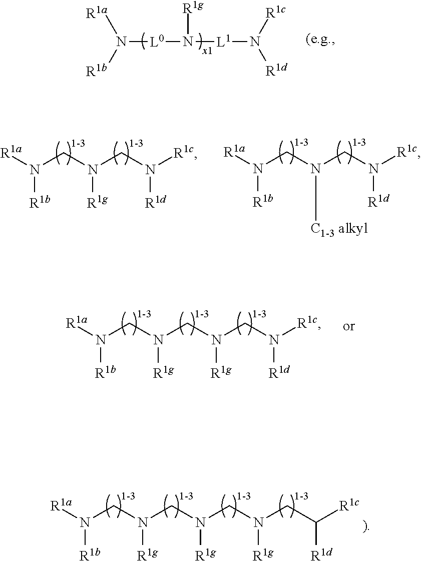

In some embodiments, the core comprises a structural formula:

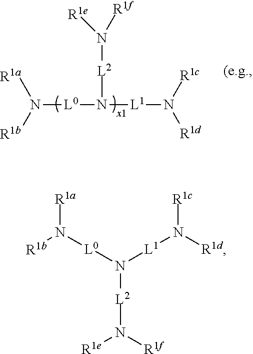

such as

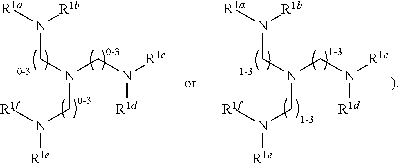

In some embodiments, the core comprises a structural formula:

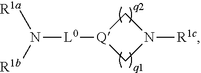

wherein Q′ is —NR²— or —CR^{3a}R^{3b}—; q¹ and q² are each independently 1 or 2 In some embodiments, the core comprises a structural formula:

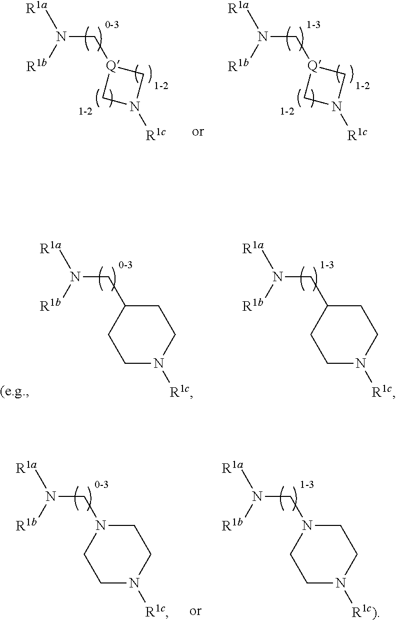

In some embodiments, the core comprises a structural

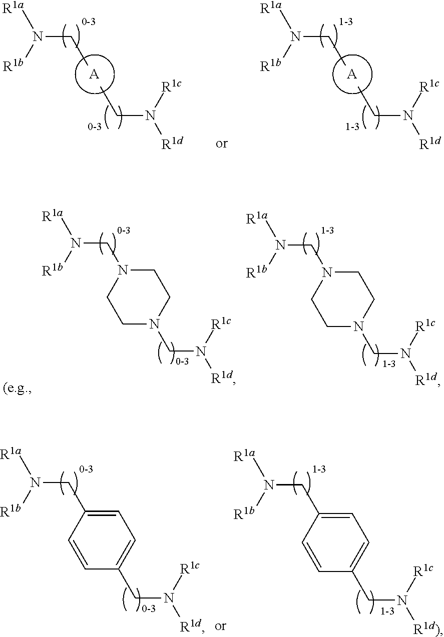

wherein ring A is an optionally substituted aryl or an optionally substituted (e.g., C₃-C₁₂, such as C₃-C₅) heteroaryl. In some embodiments, the core comprises has a structural formula

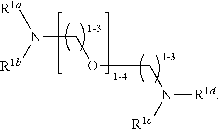

In some embodiments, the wherein the core comprises a structural formula selected from the group consisting of

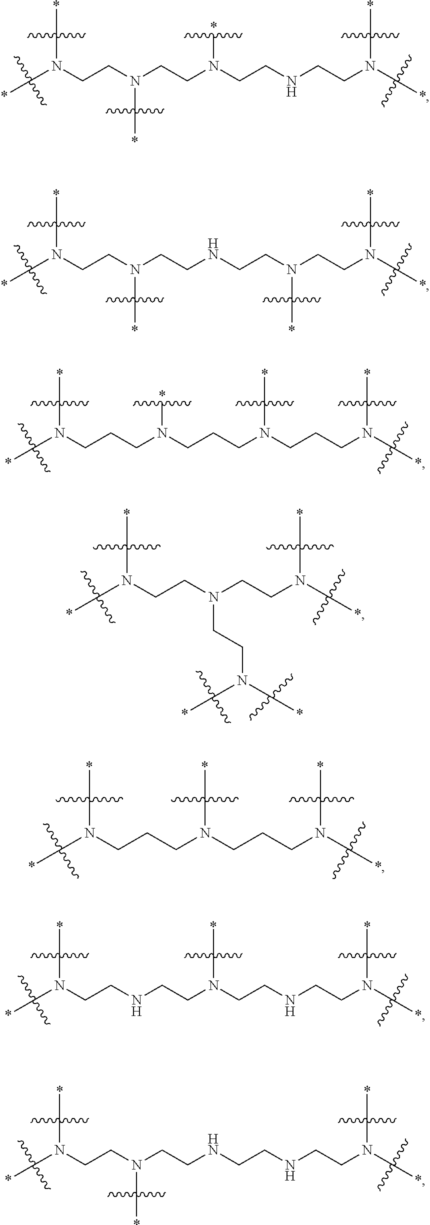

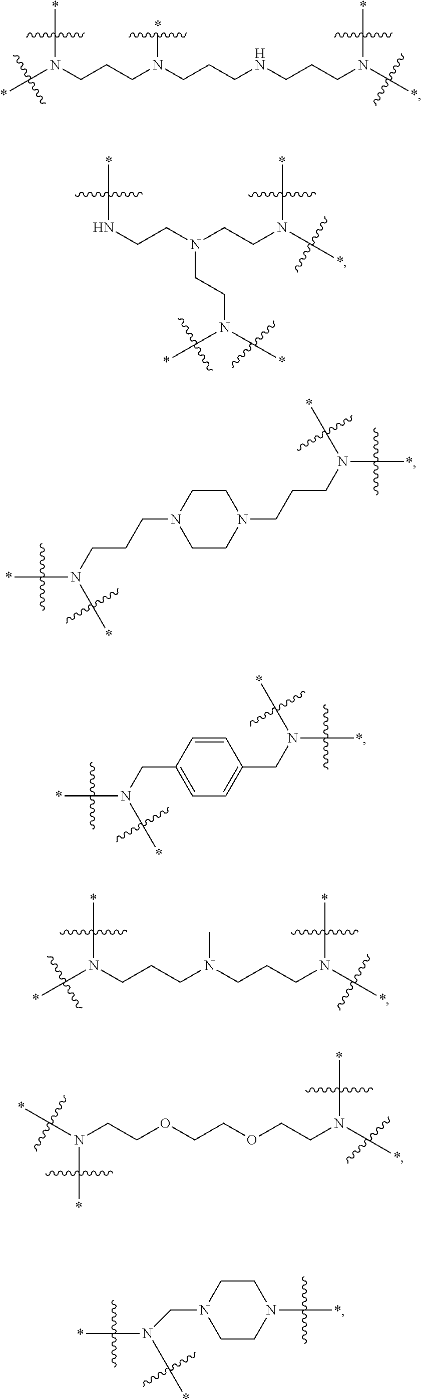

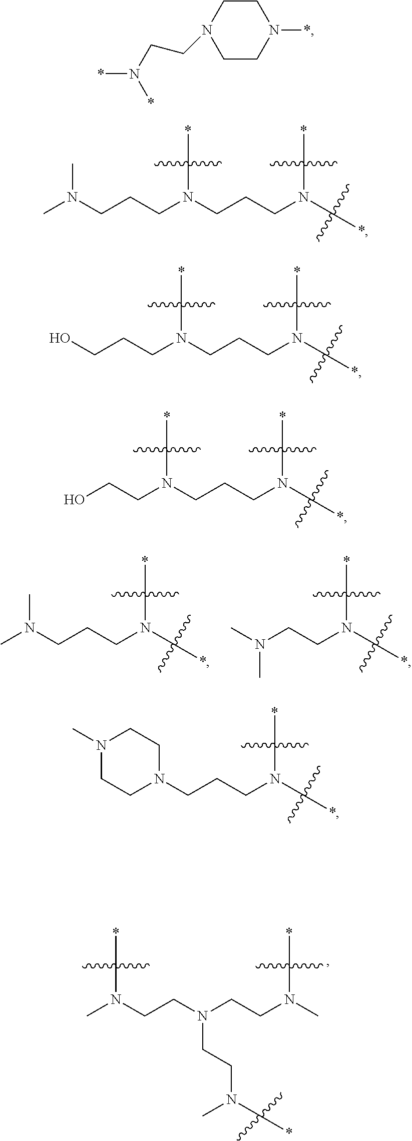

and pharmaceutically acceptable salts thereof, wherein * indicates a point of attachment of the core to a branch of the plurality of branches. In some embodiments, A¹ is —O— or —NH—. In some embodiments, A¹ is —O—. In some embodiments, A² is —O— or —NH—. In some embodiments, the A² is —O—. In some embodiments, Y³ is C₁-C₁₂ (e.g., C₁-C₆, such as C₁-C₃) alkylene. In some embodiments, wherein the diacyl group independently at each occurrence comprises a structural formula

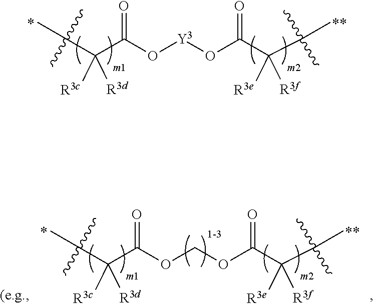

such as

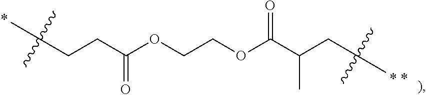 [SMILES: *CCC(=O)OCCOC(=O)C(C)C*]

optionally wherein R^{3c}, R^{3d}, R^{3e}, and R^{3f} are each independently at each occurrence hydrogen or C₁-C₃ alkyl. In some embodiments, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

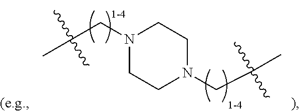

and [(C₁-C₄) alkylene]-phenylene-[(C₁-C₄) alkylene]

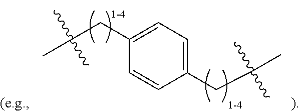

In some embodiments, L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-0)1. 4—(C₁-C₃ alkylene)). In some embodiments, L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-). In some embodiments, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

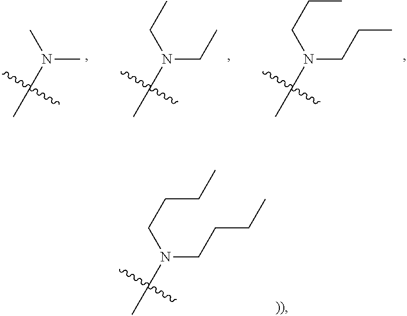

C₄-C₆ N-heterocycloalkyl (e.g., N-pyrrolidinyl

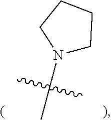 [SMILES: *N1CCCC1]

### N-piperidinyl

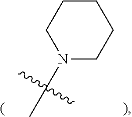 [SMILES: *N1CCCCC1]

### N-azepanyl,

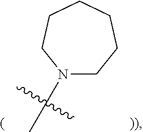 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

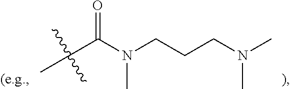 [SMILES: *C(=O)N(C)CCCN(C)C]

—C(O)N(C₁-C₃ alkyl)-(C₁-C₆ alkylene)-(C₄-C₆ N-heterocycloalkyl)

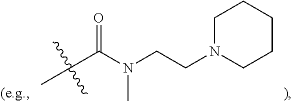 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

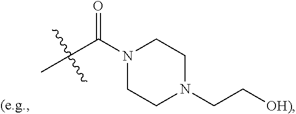

wherein the C₄-C₆ N-heterocycloalkyl moiety of any of the preceding substituents is optionally substituted with C₁-C₃ alkyl or C₁-C₃ hydroxyalkyl. In some embodiments, each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

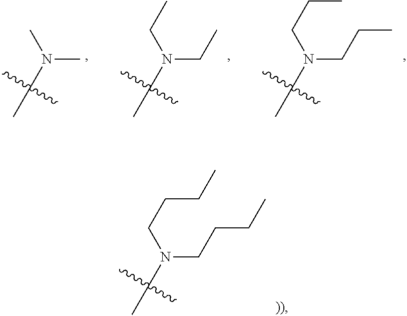

C₄-C₆ N-heterocycloalkyl (e.g., N-pyrrolidinyl

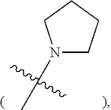 [SMILES: *N1CCCC1]

### N-piperidinyl

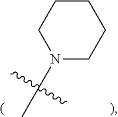 [SMILES: *N1CCCCC1]

### N-azepanyl

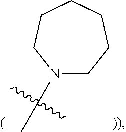 [SMILES: *N1CCCCCC1]

—OH, —C(O)OH, —C(O)N(C₁-C₃ alkyl)-(C₁-C₆ alkylene)-(C₁-C₁₂ alkylamino (e.g.,

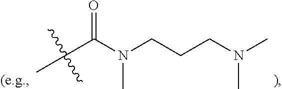 [SMILES: *C(=O)N(C)CCCN(C)C]

—C(O)N(C₁-C₃ alkyl)-(C₁-C₆ alkylene)-(C₄-C₆ N-heterocycloalkyl)

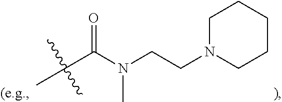 [SMILES: *C(=O)N(C)CCN1CCCCC1]

and —C(O)—(C₄-C₆ N-heterocycloalkyl)

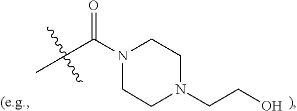 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl In some embodiments, each terminating group is independently C₁-C₁₈ (e.g., C₄-C₁₈) alkylthiol, wherein the alkyl moiety is optionally substituted with one substituent —OH. In some embodiments, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

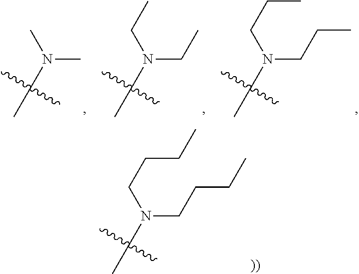

and C₄-C₆ N-heterocycloalkyl

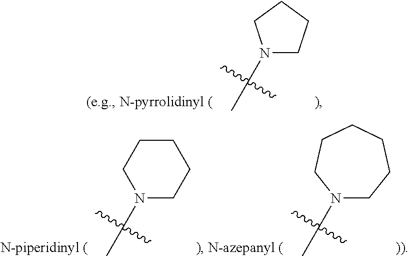 [SMILES: *N1CCCC1 ; *N1CCCCC1 ; *N1CCCCCC1]

In some embodiments, each terminating group is independently C₁-C₁₈ (e.g., C₄-C₁₈) alkenylthiol or C₁—C₁₈ (e.g., C₄-C₁₈) alkylthiol. In some embodiments, each terminating group is independently C₁-C₁₈ (e.g., C₄-C₁₈) alkylthiol.

In some embodiments, each terminating group is independently selected from the group consisting of

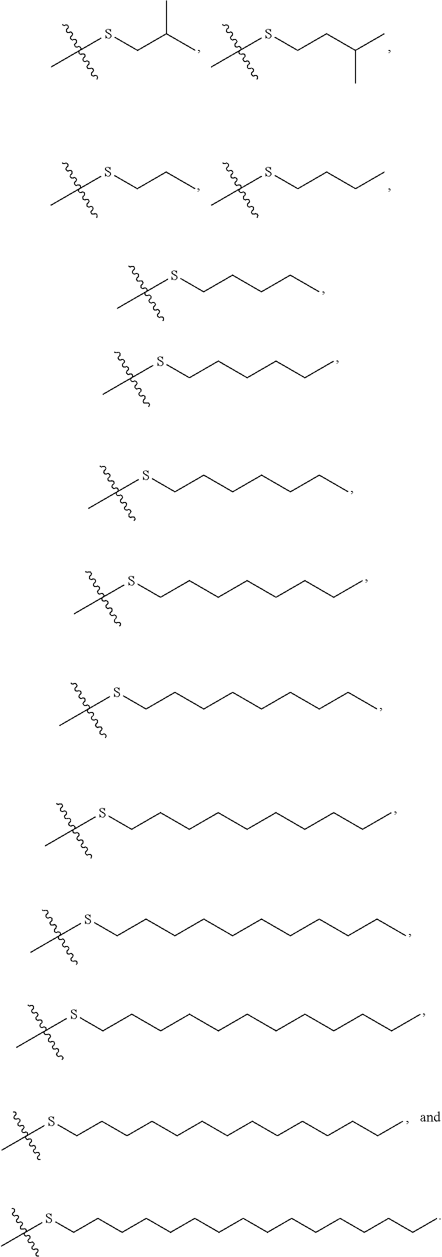

In some embodiments, the dendrimer or dendron is selected from the group consisting of

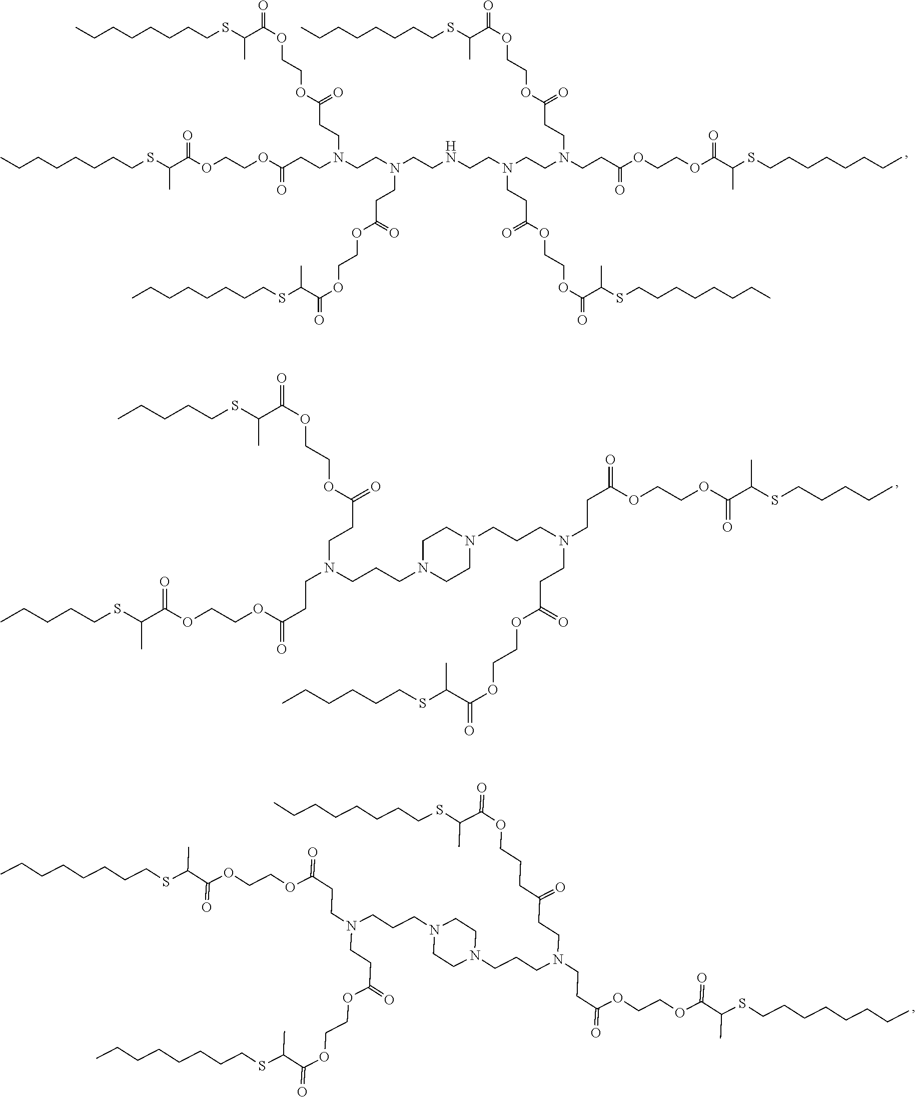

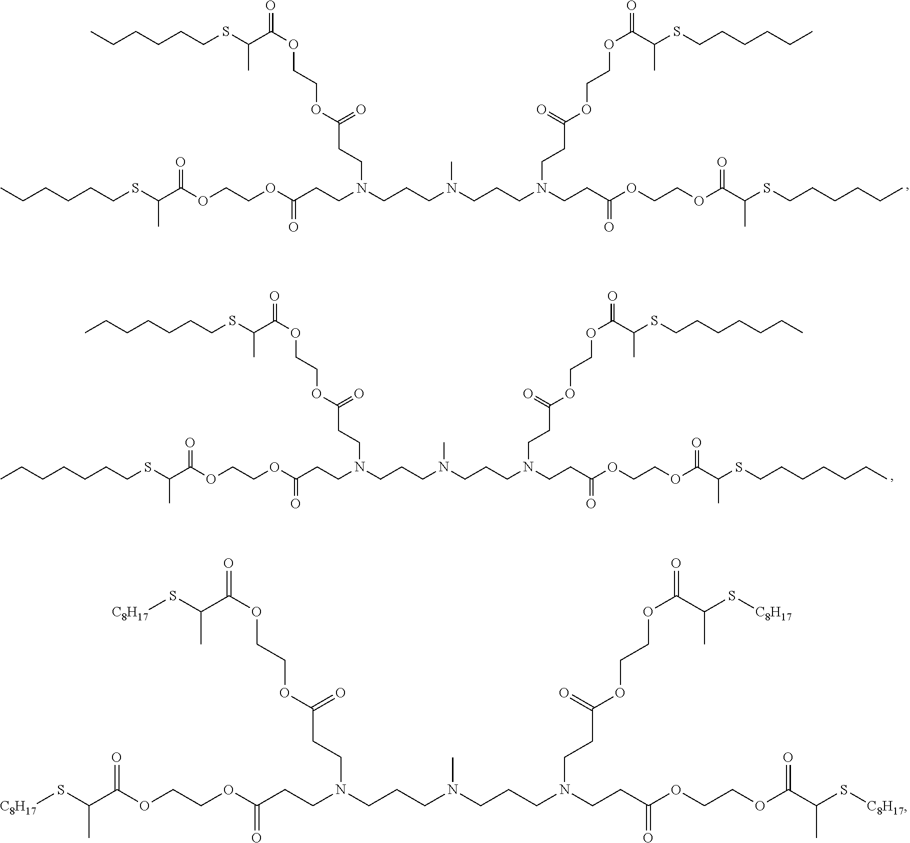

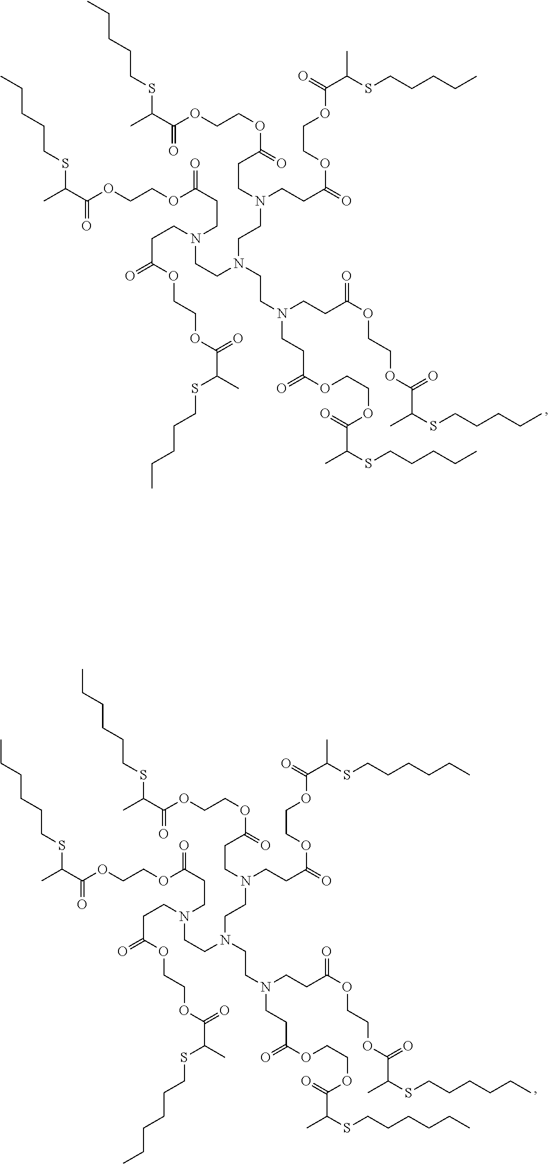 [SMILES: CCCCCSC(C)C(=O)OCCOC(=O)CCN(CCC(=O)OCCOC(=O)C(C)SCCCCC)CCN(CCN(CCC(=O)OCCOC(=O)C(C)SCCCCC)CCC(=O)OCCOC(=O)C(C)SCCCCC)CCN(CCC(=O)OCCOC(=O)C(C)SCCCCC)CCC(=O)OCCOC(=O)C(C)SCCCCC ; CCCCCCSC(C)C(=O)OCCOC(=O)CCN(CCC(=O)OCCOC(=O)C(C)SCCCCCC)CCN(CCN(CCC(=O)OCCOC(=O)C(C)SCCCCCC)CCC(=O)OCCOC(=O)C(C)SCCCCCC)CCN(CCC(=O)OCCOC(=O)C(C)SCCCCCC)CCC(=O)OCCOC(=O)C(C)SCCCCCC]

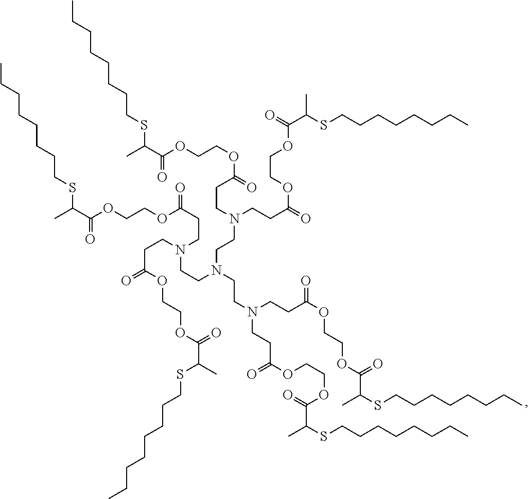 [SMILES: CCCCCCCCSC(C)C(=O)OCCOC(=O)CCN(CCC(=O)OCCOC(=O)C(C)SCCCCCCCC)CCN(CCN(CCC(=O)OCCOC(=O)C(C)SCCCCCCCC)CCC(=O)OCCOC(=O)C(C)SCCCCCCCC)CCN(CCC(=O)OCCOC(=O)C(C)SCCCCCCCC)CCC(=O)OCCOC(=O)C(C)SCCCCCCCC]

and pharmaceutically acceptable salts thereof.

In another aspect, the present disclosure provides a method for treating a subject having or suspected of having primary ciliary dyskinesia (PCD), comprising administering to the subject a pharmaceutical composition comprising a heterologous polynucleotide assembled with a lipid composition, which heterologous polynucleotide encodes a dynein axonemal intermediate chain 1 (DNAI1) protein, thereby resulting in a heterologous expression of the DNAI1 protein within cells of the subject, wherein the lipid composition comprises (i) an ionizable cationic lipid, and (ii) a selective organ targeting (SORT) lipid separate from the ionizable cationic lipid. In some embodiments, the lipid composition further comprises a phospholipid.

In some embodiments, the pharmaceutical formulation is formulated for inhalation. In some embodiments, the pharmaceutical composition is an (e.g., inhalable) aerosol composition. In some embodiments, the aerosol composition is generated by a nebulizer (e.g., at a nebulization rate from 0.2 milliliter (mL) per minute (mL/min) to 1 mL/min). In some embodiments, the aerosol composition has a (e.g., median, or average) droplet size from 1 micron (μm) to 10 μm. In some embodiments, the pharmaceutical composition is an aerosol composition. In some embodiments, the aerosol composition is generated by a nebulizer at a nebulization rate of no more than 70 mL/minute. In some embodiments, the aerosol composition is generated by a nebulizer at a nebulization rate of no more than 50 mL/minute. In some embodiments, the aerosol composition is generated by a nebulizer at a nebulization rate of no more than 30 mL/minute.

In some embodiments, the aerosol composition has an average droplet size from about to about 0.5 micron (μm) to about 10 μm. In some embodiments, the aerosol composition has an average droplet size from about to about 0.5 micron (μm) to about 10 μm. In some embodiments, the aerosol composition has an average droplet size from about to about 1 micron (μm) to about 10 μm. In some embodiments, the aerosol composition has an average droplet size from about to about 0.5 micron (μm) to about 5 μm. In some embodiments, the aerosol droplets are generated by a nebulizer at a nebulization rate of no more than 70 mL/minute. In some embodiments, the aerosol droplets have a mass median aerodynamic diameter (MMAD) from about 0.5 micron (μm) to about 10 μm. In some embodiments, the droplet size varies less than about 50% for a duration of about 24 hours under a storage condition. In some embodiments, droplets of said aerosol composition are characterized by a geometric standard deviation (GSD) of no more than about 3.

In some embodiments, the administrating comprises administering to a lung by nebulization. In some embodiments, the subject is determined to exhibit an aberrant expression or activity of DNAI1 gene or protein. In some embodiments, the subject is a human. In some embodiments, the cells are in a lung of the subject. In some embodiments, the cells are ciliated cells. In some embodiments, the cells are undifferentiated. In some embodiments, the cells are differentiated. In some embodiments, the ciliated cells are ciliated epithelial cells (e.g., ciliated airway epithelial cells). In some embodiments, the ciliated epithelial cells are undifferentiated. In some embodiments, the ciliated epithelial cells are differentiated.

In another aspect, the present disclosure provides an aerosol composition comprising a pharmaceutical composition described elsewhere herein.

In another aspect, the present disclosure provides a method for treating a subject having or suspected of having primary ciliary dyskinesia (PCD), comprising administering to the subject a pharmaceutical composition described elsewhere herein.

Provided herein, in some aspects, include a method for enhancing an expression or activity of dynein axonemal intermediate chain 1 (DNAI1) protein in a (e.g., lung) cell, the method comprising: contacting said (e.g., lung) cell with a composition comprising a synthetic polynucleotide assembled with a lipid composition, wherein said synthetic polynucleotide encodes a DNAI1 protein, wherein said lipid composition comprises an ionizable cationic lipid and a selective organ targeting (SORT) lipid separate from said ionizable cationic lipid, thereby providing a(n) (e.g., therapeutically) effective amount or activity of a functional variant (e.g., wild-type form) of DNAI1 protein in said (e.g., lung) cell.

In some embodiments, the method provides a(n) (e.g., therapeutically) effective amount or activity of said functional variant (e.g., wild-type form) of DNAI1 protein in said (e.g., lung) cell at least about 6 hours after said contacting. The contacting may be in vivo. The contacting may be ex vivo. The contacting may be in vitro.

In some embodiments, the method provides a(n) (e.g., therapeutically) effective amount or activity of said functional variant (e.g., wild-type form) of DNAI1 protein in at least about 5%, 10%, 15%, or 20% lung epithelial cells comprising said (e.g., lung) cell. In some embodiments, the method provides a(n) (e.g., therapeutically) effective amount or activity of said functional variant (e.g., wild-type form) of DNAI1 protein in at least about 2%, 5%, or 10% lung ciliated cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung secretory cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung club cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung goblet cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung basal cells comprising said (e.g., lung) cell.

In some embodiments, the (e.g., lung) cell is in a ciliary axoneme. In some embodiments, the (e.g., lung) cell is an airway epithelial cell (e.g., a bronchial epithelial cell). In some embodiments, the (e.g., lung) cell is a ciliated cell, a basal cell, a goblet cell, or a club cell. In some embodiments, the (e.g., lung) cell is a ciliated cell, a basal cell, or a club cell. In some embodiments, the (e.g., lung) cell exhibits a mutation in DNAI1 gene or transcript.

In some embodiments, the contacting comprises contacting a plurality of (e.g., lung) cells that comprises said (e.g., lung) cell. In some embodiments, the plurality of (e.g., lung) cells comprises ciliated cell(s), basal cell(s), goblet cell(s), club cell(s), or a combination thereof. In some embodiments, the plurality of (e.g., lung) cells comprises ciliated cell(s), basal cell(s), club cell(s), or a combination thereof. In some embodiments, mucus is present in said contacting.

In some embodiments, the contacting is repeated (e.g., at least about 2, 4, 6, 8, or 10 times). In some embodiments, the repeated contacting is at least once a week, at least twice a week, or at least three times a week. In some embodiments, at least one contacting steps of said repeated contacting is followed by a treatment holiday. In some embodiments, the repeated contacting is characterized by a duration of at least 1, 2, 3, 4, or 5 week(s). In some embodiments, mucus is present in one or more contacting steps of said repeated contacting.

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, or 7 days) after said contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The contacting may be ex vivo or in vitro.

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, 7, 8, 9, 10, 11, 12, 13, or 14 days) after a contacting of said repeated contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The repeated contacting(s) may be (e.g., partially) ex vivo or in vitro.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings (also “figure” and “Fig.” herein), of which:

FIG. 1 shows the chemical structures of lipids.

FIG. 2 shows the chemical structures of dendrimer or dendron lipids.

FIG. 3 shows a chart of cells type and expression levels of a delivered mRNA using different compositions of LNP.

FIG. 4 shows images using in vivo imaging of bioluminescence of a mouse after inhaled aerosol delivery of a Luc mRNA/LNP using multiple compositions of LNP.

FIG. 5 shows a chart regarding cell toxicity of various LNP compositions in human bronchial epithelial (hBE) cells.

FIG. 6 shows the stability and general characteristics of various LNP compositions.

FIG. 7 shows a chart of tissue specific radiance over time in a mouse of a LNP composition (“Lung-SORT”; 5A2-SC8 DOTAP).

FIG. 8 shows images of tissue specific radiance over time in a mouse of a LNP composition (“Lung-SORT”; 5A2-SC8 DOTAP).

FIG. 9A shows a workflow for a safety and tolerability study in humans using a composition described herein.

FIG. 9B illustrates an ex vivo model of ciliated epithelial cells (mouse tracheal epithelial cells or MTECs cultured at an air-liquid Interface (ALI) for testing the efficacy of rescue by the DNAI1 mRNA treatment described herein.

FIG. 9C illustrates that ciliary activity in KO mouse cells was rescued by the DNAI1 mRNA treatment, and the treatment effect remained stable for weeks after dosing was stopped.

FIG. 10A shows summaries of experiments performed to measure properties of lipid compositions described herein.

FIG. 10B shows outcomes of experiments performed to measure properties of lipid compositions described herein.

FIG. 10C illustrates positive lung labeling (red) for DNAI1 mRNA (left) and DNAI1 protein (right) in non-human primates using a lipid composition (e.g., comprising a SORT lipid) as described herein.

FIG. 10D illustrates that by replacing 100% of U's in the mRNA with modified nucleotide m1Ψ minimized cytokine response.

FIG. 10E illustrates data collected from experiments demonstrating functional restoration of cilia, tolerability, and selectivity of lipid compositions described herein.

FIG. 11A shows images of immunofluorescence of human DNAI1 knock-down cells treated with LNPs containing DNA1-HA mRNA. Well-differentiated human DNAI1 knock-down cells were treated with a single dose of a formulation of DNAI1-HA mRNA described herein and immunostained with anti-acetylated tubulin and anti-HA. Integration of newly-expressed DNAI1-HA into axoneme of cilia peaked between 48 to 72 hours after treatment. DNAI1-HA was detected in ciliary axoneme for more than 24 days after single administration. Repeated administration resulted in rescue of ciliary activity that remained for weeks after the dosing was stopped.

FIG. 11B illustrates that newly-made HA-tagged DNAI1 was rapidly incorporated into the cilia of human bronchial epithelial cells (hBEs). Well-differentiated human DNAI1 knock-down cells were treated (basal administration) with a single dose of LNP formulated DNAI1-HA (10 μg in 2 ml of media). Cells were immunostained with anti-acetylated tubulin and anti-HA 72 hours after dosing. More than 90% of ciliated cells was positive for DNAI1-HA.

FIG. 12 shows a multiplexed immunofluorescence panel of the respiratory epithelium of NHPs that may be used to distinguish cell types showing newly translated DNAI1 protein.

FIG. 13A shows cell tropism signatures of LNP formulations described herein.

FIG. 13B illustrates aerosol administration of formulated DNAI1 mRNA rescued ciliary activity in knock-down primary hBE ALI cultures. Well-differentiated human DNAI1—knock-down cells (hBEs) were treated 2 times per week with LNP-formulated DNAi1 (300 μg per Vitrocell nebulization) starting on day 25 post ALI (culture age). Last dose was administered on day 50 post ALI. Increased ciliary activity in treated DNAI1 knock-down cultures was first detected seven days after dosing was initiated. Rescued ciliary activity had normal beat frequency (9-17 Hz) and appeared synchronized.

FIGS. 14A-B illustrate cell tropism signatures of a lipid composition comprising 20% ionizable cationic lipid (e.g., DODAP) under certain concentrations and conditions.

FIG. 14C illustrates a cell tropism signature of a lipid composition comprising 20% ionizable cationic lipid (e.g., DODAP) under certain concentrations and conditions.

FIG. 14D illustrates a cell tropism signature of a lipid composition comprising 20% permanently cationic lipid (e.g., 14:0 EPC) under certain concentrations and conditions.

FIG. 14E illustrates a cell tropism signature of a lipid composition comprising 20% permanently cationic lipid (e.g., 14:0 TAP) under certain concentrations and conditions.

FIG. 15 illustrates cell type related DNAI1 protein expression in target cells of the respiratory epithelium of NHPs.

FIG. 16A illustrates cell type related DNAI1 protein expression in target cells of the respiratory epithelium of NHPs.

FIG. 16B shows cell type related DNAI1-HA protein expression in target cells of the respiratory epithelium of NHPs (left panel, lung; right panel bronchi).

FIG. 17 illustrates a study of biodistribution, potency, and tolerability of LNP formulations described herein.

FIG. 18A illustrates the aerosol concentration administered to the NHPs.

FIG. 18B illustrates exemplary measurements of the doses delivered to the NHPs.

FIG. 18C illustrates characterization of the aerosol composition droplet (MMAD: mass median aerodynamic diameter; GSDL: geometric standard deviation). The droplet characterization results were within recommended range of the Organization for Economic Co-operation and Development (OECD) guidance 433 for inhalation toxicity studies with an MMAD≤4 μm and a GSD between 1.0 and 3.0.

FIGS. 19A-C illustrate measurement of LNP lipid (stemmed from aerosol droplet) in lung in both low dose and high dose NHP group (FIG. 19A: ionizable lipid in lung; FIG. 19B: DMG-PEG in lung; and FIG. 19C: SORT lipid).

FIG. 20A illustrates DNAI1-HA protein expression in the lung by Western blotting.

FIG. 20B illustrates DNAI1-HA protein expression in the lung by ELISA.

FIG. 21A illustrates clinical chemistry measurements for AST, ALT, and ALP. No significant changes of AST, ALT, or ALP were observed following treatment with a lipid composition described herein.

FIG. 21B illustrates the hematology counts of white blood cells and neutrophils. Some increase in neutrophils was observed in the post-treatment measurements of both vehicle and RTX0052 groups.

FIG. 21C illustrates BAL cell differentials. For cytokine and complement analysis, cytokines levels were measured in NHP serum and BAL. Analytes measured included IFN-α2a, IFN-γ, IL-1β, IL-4, IL-6, IL-10, IL-17A, IP-10, MCP-1, and TNFα. All cytokine levels were in the same range as normal reported elves.

FIG. 21D illustrates exemplary measurements of cytokine in serum.

FIG. 21E illustrates exemplary measurements of cytokine in BAL.

FIG. 21F illustrates exemplary complement measures of C3a and sC5b-9 measurements in plasma and serum respectively.

FIG. 21G illustrates exemplary complement measures of C3a and sC5b-9 measurements in BAL.

FIG. 22A illustrates the aerosol concentration administered to the rats.

FIG. 22B illustrates exemplary measurements aerosol homogeneity across three stages.

FIG. 22C illustrates the amount of doses delivered to the rats.

FIG. 22D illustrates characterization of the aerosol composition droplet (MMAD: mass median aerodynamic diameter; GSDL: geometric standard deviation).

FIGS. 23A-C illustrate measurement of LNP lipid (stemmed from aerosol droplet) in lung in low dose, mid dose, and high dose rat group (FIG. 23A: ionizable lipid in lung; FIG. 23B: DMG-PEG in lung; and FIG. 23C: SORT lipid).

FIG. 24A illustrates DNAI1-HA protein expression in the rat lung by Western blotting. Six out of ten lung samples I the 1.2 mg/kg, 6 hour group were positive for DNAI1-HA.

FIG. 24B illustrates DNAI1-HA protein expression in the rat lung by ELISA.

FIG. 25A illustrates clinical chemistry measurements for AST, ALT, and ALP in the treated rats.

FIG. 25B illustrates the hematology counts of white blood cells and neutrophils in the treated rats.

FIG. 25C illustrates BAL cell differentials in the treated rats.

FIG. 25D illustrates exemplary measurements of alpha-2-macroglobulin in the treated rats.

FIG. 26A illustrates the information relating to the four groups of mice to be repeatedly treated with nebulization of LNP/DNAI1-HA mRNA.

FIG. 26B illustrates the protocol for the dosing, imaging, and necropsy of the repeatedly dosed mice.

FIG. 27A-B illustrate whole body in vivo imaging (IVIS) of the repeatedly dosed mice. Animals, B6 Albino, male, about 7 weeks of age, naïve, were administered 4.0 mg of LNP-formulated DNAI1-HA/Luciferase by nebulization in 2 hours at 66.6 μL/min with Zero grade dry air flow at 2 L/min. 4 hour post-dosing, two mice were administered 2 mL of luciferin (30 mg/mL) by nebulization and imaged on IVIS within 1-15 min post-luciferin administration. Pseudo coloring was applied on the same scale for all images. Lung signal was plotted in graph of FIG. 27A. Whole body signal is plotted in the graph of FIG. 27B.

FIG. 27C illustrates histopathology results of the repeatedly dosed mice.

FIG. 27D illustrates qPCR results showing the relative abundance of DNAI1-HA mRNA. After the last imaging of the last dose (dose 8), 2 mice per group were perfused.

FIG. 27E illustrates Western blotting showing the protein expression of DNAI1-HA.

FIG. 28A illustrates delivery of 0.4 mg/kg of LNP-formulated DNAI1 mRNA by inhalation. NHPs were intubated, ventilated, and dosed for fewer than 30 minutes.

FIG. 28B illustrates LNP formulation aerosol characteristics. Aerosol particle size ranges for all three formulations were appropriate for deposition in the conducting airways.

FIG. 28C illustrates biodistribution of DNAI1-HA mRNA in the targeted cells.

FIG. 28D illustrates DNAI1-HA mRNA ISH results by H-Score. ISH results demonstrated high levels of DNAI1-HA mRNA were delivered to lung cells with lower levels in the bronchi and trachea.

FIGS. 29A-D illustrate delivery of high levels of DNAI1-HA mRNA to the lung without exposure to liver, spleen, or blood. Digital PCR was used to measure DNAI1-HA mRNA levels in whole blood, lung, liver, and spleen tissue following a single 0.4 mg/kg administration. High levels of DNAI1-HA mRNA were detected in all three lung regions sampled at 6 hours post-exposure with RTX0051 and RTX0052. No DNAI1-HA mRNA was detected above background in spleen (6 hours, FIG. 29B), liver (6 hours, FIG. 29C), or whole blood (30 minutes or 60 minutes, FIG. 29D).

FIG. 30A illustrates multiplex immunofluorescent (IF) images for epithelial cell types.

FIG. 30B illustrates multiplex IF analysis demonstrating expression of DNAI1-HA protein in target cells in lung.

FIG. 30C illustrates multiplex IF analysis demonstrating expression of DNAI1-HA protein in target cells in lung with RTX0051.

FIG. 30D illustrates multiplex IF analysis demonstrating expression of DNAI1-HA protein in target cells in bronchi.

FIG. 30E illustrates multiplex IF analysis demonstrating expression of DNAI1-HA protein in target cells in bronchi with RTX0051.

FIG. 30F illustrates multiplex IF analysis demonstrating expression of DNAI1-HA protein in target cells in trachea.

FIGS. 31A-E illustrate BAL cytokine and complement results.

FIGS. 32A-E illustrate plasma cytokine results.

FIG. 33 illustrates transient increase in neutrophils observed in BAL and blood at six hours post-exposure.

FIG. 34 illustrates selected clinical chemistry results. Small increases were observed for AST, LDH, and creatine kinase in individual animals after treatment.

FIG. 35 illustrates summary of tolerability as determined by clinical observations and organ weights for the single dose inhalation study.

FIG. 36A illustrates a diagram of the aerosol delivery system. The amount of aerosolized drug delivered past the endotracheal tube was estimated using the test setup shown on the left. Pre-weighed glass fiber and MCE filters were attached directly at the exit of the endotracheal tube. Multiple collections were performed before, during and after treatment of the animals. The glass filters were dried and quantified using both gravimetric analyses. The MCE filters were analyzed for amount of mRNA using a RiboGreen assay.

FIG. 36B illustrates the results of aerosol particle size measurements. Particle sizes for test article exposure were measured for deposition in the conducting airways (branching generations 0-15 in humans).

FIG. 37 illustrates DBAI1-HA mRNA dose present in the NHP. A dashed black horizontal line represents the targeted presented dose of 0.1 mg/kg. Open yellow circles show filter collections before and after dosing (GF, n=2) using RTX0001/DNAI1-HA mRNA. Open yellow squares show filter collections before and after dosing (GF, n=2) using RTX0004/DNAI1-HA mRNA. Similar results obtained for mRNA using MCE filters and a RiboGreen assay.

FIG. 38A illustrates DNAI1-HA mRNA ISH results for lung tissue. Data from assay qualification: 1 of 4 samples per animal analyzed. DNAI1-HA mRNA detected in all animals.

FIG. 38B illustrates that a significant fraction of lung cells contained DNAI1-HA mRNA after treatment with RTX0001 as measured by ISH and the bin scoring.

FIG. 38C illustrates the imaging of the lung tissue used for the ISH analysis.

FIG. 39A illustrates that the delivery of high levels of DNAI1-HA to the lung did not lead to similar deliver to liver or spleen. Digital PCR was used to measure DNAI1-HA mRNA levels in whole blood, lung, liver, and spleen tissue following a single 0.1 mg/kg administration. High levels of DNAI1-HA mRNA were detected in all three lung regions sampled at 6 hour post-exposure with RTX0001. In spleen and liver, DNAI1-HA mRNA was only measured at or below the LLOQ of the assay.

FIG. 39B illustrates the positive staining of DNAI1-HA tagged protein in NHPs. For RTX0001, DNAI1-HA was detected six hours or 24 hours after administration. Regions with higher mRNA levels correlated with regions showing highest levels of DNAI1-HA protein. DNAI1-HA mRNA was present in all eight treated animals. No signal detected in vehicle treated animals. mRNA levels were highest at six hours and lower at 24 hours.

FIG. 39C illustrates multiplex IF panel for key epithelial cell types. 10 NHP FFPE lung tissue blocks (1 from each animal) were used for mIF assay qualification. Two slides from each block were stained in duplicates. The cell counts of single marker positive cells, double positive cells with DNAI1 expression, and DNAI1 MFI in double positive cells were reported.

FIG. 40A illustrates multiplex IF panel results for NHP lung samples. DNAI1-HA was expressed in cells of the respiratory epithelium. Percentage of DNAI1-HA positive cell was calculated by combining cell counts from 1 examined lung section per animal. DNAI1-HA expression was detected in lung samples from NHPs treated with RTX0001. DNAI1-HA expression was co-localized with markers for epithelial cells, including the club, basal and ciliated cells (club and basal cells are precursors for ciliated cells). No staining detected was in lung samples from NHPs treated with RTX0004.

FIG. 40B illustrates multiplex IF analysis of expression of DNAI1-HA protein in target cell in the lung. Single dose of 0.1 mg/kg of RTX0001/DNAI1-HA mRNA was administered via inhalation. Lung sections were collected from two NHPs at six hours and 24 hours after dosing. Percentage of DNAI1-HA positive cell was calculated by combining cell counts from all 4 examined lung sections for an individual animal. Total number of cells counted per animal was about 690,000 to 1,100,000. Shown are the individual data points for each treated animal and the mean±std. dev. for each group (N=2).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “disease,” as used herein, generally refers to an abnormal physiological condition that affects part or all of a subject, such as an illness (e.g., primary ciliary dyskinesia) or another abnormality that causes defects in the action of cilia in, for example, the lining the respiratory tract (lower and upper, sinuses, Eustachian tube, middle ear), in a variety of lung cells, in the fallopian tube, or flagella of sperm cells.

The term “polynucleotide” or “nucleic acid” as used herein generally refers to a polymeric form of nucleotides of any length, either ribonucleotides or deoxyribonucleotides, that comprise purine and pyrimidine bases, purine and pyrimidine analogues, chemically or biochemically modified, natural or non-natural, or derivatized nucleotide bases. Polynucleotides include sequences of deoxyribonucleic acid (DNA), ribonucleic acid (RNA), or DNA copies of ribonucleic acid (cDNA), all of which can be recombinantly produced, artificially synthesized, or isolated and purified from natural sources. The polynucleotides and nucleic acids may exist as single-stranded or double-stranded. The backbone of the polynucleotide can comprise sugars and phosphate groups, as may typically be found in RNA or DNA, or analogues or substituted sugar or phosphate groups. A polynucleotide may comprise naturally occurring or non-naturally occurring nucleotides, such as methylated nucleotides and nucleotide analogues (or analogs).

The term “polyribonucleotide,” as used herein, generally refers to polynucleotide polymers that comprise ribonucleic acids. The term also refers to polynucleotide polymers that comprise chemically modified ribonucleotides. A polyribonucleotide can be formed of D-ribose sugars, which can be found in nature.

The term “polypeptides,” as used herein, generally refers to polymer chains comprised of amino acid residue monomers which are joined together through amide bonds (peptide bonds). A polypeptide can be a chain of at least three amino acids, a protein, a recombinant protein, an antigen, an epitope, an enzyme, a receptor, or a structure analogue or combinations thereof. As used herein, the abbreviations for the L-enantiomeric amino acids that form a polypeptide are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). X or Xaa can indicate any amino acid.

The term “engineered,” as used herein, generally refers to polynucleotides, vectors, and nucleic acid constructs that have been genetically designed and manipulated to provide a polynucleotide intracellularly. An engineered polynucleotide can be partially or fully synthesized in vitro. An engineered polynucleotide can also be cloned. An engineered polyribonucleotide can contain one or more base or sugar analogues, such as ribonucleotides not naturally-found in messenger RNAs. An engineered polyribonucleotide can contain nucleotide analogues that exist in transfer RNAs (tRNAs), ribosomal RNAs (rRNAs), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA.

### Chemical Definitions

When used in the context of a chemical group: “hydrogen” means —H; “hydroxy” means —OH; “oxo” means═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O—or a deprotonated form thereof; “mercapto” means —SH; and “thio” means=S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, for example, the formula

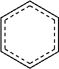

includes

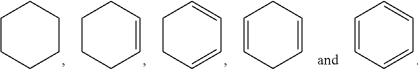

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

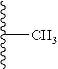

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

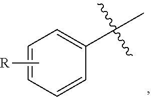

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

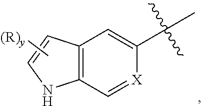

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C n” defines the maximum number (n) of carbon atoms that can be in the group/class, with the minimum number as small as possible for the group/class in question, e.g. it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃ (tert-butyl, t-butyl, t-Bu or ^{t}Bu), and —CH₂C(CH₃)₃(neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂—(methylene), —CH₂CH₂—, —CH₂C(CH₃)₂CH₂—, and —CH₂CH₂CH₂—are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C₁. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

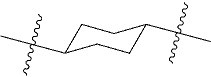 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)2, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂—are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

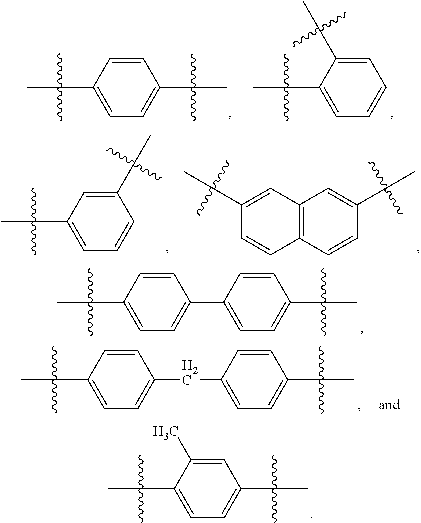

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl , —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl , —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

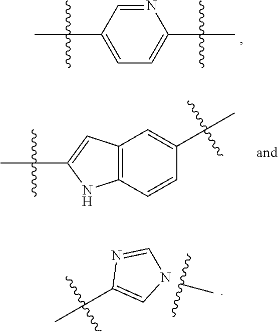

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl , —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to a divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

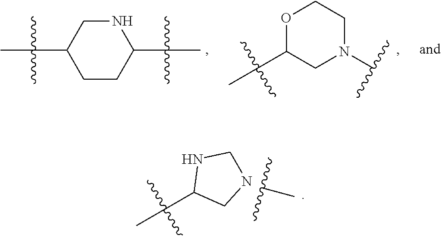

When these terms are used with the “substituted” modifier one or more hydrogen atom has been independently replaced by —OH, —F, —Cl , —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl , —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), —OC(CH₃)₃(tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl , —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl , —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally, the term “about,” as used herein when referring to a measurable value such as an amount of weight, time, dose, etc. is meant to encompass in one example variations of ±20% or ±10%, in another example ±5%, in another example ±T %, and in yet another example ±0.1% from the specified amount, as such variations are appropriate to perform the disclosed method.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e., an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Examples of lipid compositions include suspensions, emulsions, and vesicular compositions.

For example, as used herein, “RTX0001” refers to an example lipid composition tested herein. RTX0001 is a 5-component lipid nanoparticle composition comprising about 19.05% 4A3-SC7 (ionizable cationic lipid), about 20% DODAP (SORT lipid), about 19.05% DOPE, about 38.9% cholesterol, and about 3.81% DMG-PEG (PEG conjugated lipid), wherein each lipid component is defined as mol % of the total lipid composition.

As another example, as used herein, “RTX0004” refers to an example lipid composition tested herein. RTX0004 is a 4-component lipid nanoparticle composition comprising about 23.81% 5A2-SC8 (ionizable cationic lipid), about 23.81% DOPE, about 47.62% cholesterol, and about 4.76% DMG-PEG (PEG conjugated lipid), wherein each lipid component is defined as mol % of the total lipid composition.

As another example, as used herein, “RTX0051” refers to an example lipid composition tested herein. RTX0051 is a 5-component lipid nanoparticle composition comprising about 19.05% 4A3-SC7 (ionizable cationic lipid), about 20% 14:0 EPC (SORT lipid), about 19.05% DOPE, about 38.9% cholesterol, and about 3.81% DMG-PEG (PEG conjugated lipid), wherein each lipid component is defined as mol % of the total lipid composition.

As yet another example, as used herein, “RTX0052” refers to an example lipid composition tested herein. RTX0052 is a 5-component lipid nanoparticle composition comprising about 19.05% 4A3-SC7 (ionizable cationic lipid), about 20% 14:0 TAP (SORT lipid), about 19.05% DOPE, about 38.9% cholesterol, and about 3.81% DMG-PEG (PEG conjugated lipid), wherein each lipid component is defined as mol % of the total lipid composition.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, -[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer or dendron, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “molar percentage” or “molar %” as used herein in connection with lipid composition(s) generally refers to the molar proportion of that component lipid relative to compared to all lipids formulated or present in the lipid composition.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### Primary Ciliary Dyskinesia (PCD)

The present disclosure provides, in some embodiments, compositions and methods for the treatment of conditions associated with cilia maintenance and function, with nucleic acids encoding a protein or protein fragment(s). Numerous eukaryotic cells carry appendages, which are often referred to as cilia or flagella, whose inner core comprises a cytoskeletal structure called the axoneme. The axoneme can function as the skeleton of cellular cytoskeletal structures, both giving support to the structure and, in some instances, causing it to bend. Usually, the internal structure of the axoneme is common to both cilia and flagella. Cilia are often found in the linings of the airway, the reproductive system, and other organs and tissues. Flagella are tail-like structures that, similarly to cilia, can propel cells forward, such as sperm cells.

Without properly functioning cilia in the airway, bacteria can remain in the respiratory tract and cause infection. In the respiratory tract, cilia move back and forth in a coordinated way to move mucus towards the throat. This movement of mucus helps to eliminate fluid, bacteria, and particles from the lungs. Many infants afflicted with cilia and flagella malfunction experience breathing problems at birth, which suggests that cilia play an important role in clearing fetal fluid from the lungs. Beginning in early childhood, subjects afflicted with cilia malfunction can develop frequent respiratory tract infections.

Primary ciliary dyskinesia is a condition characterized by chronic respiratory tract infections, abnormally positioned internal organs, and the inability to have children (infertility). The signs and symptoms of this condition are caused by abnormal cilia and flagella. Subjects afflicted with primary ciliary dyskinesia often have year-round nasal congestion and a chronic cough. Chronic respiratory tract infections can result in a condition called bronchiectasis, which damages the passages, called bronchi, leading from the windpipe to the lungs and can cause life-threatening breathing problems.

The methods, constructs, and compositions of this disclosure provide a method to treat primary ciliary dyskinesia (PCD), also known as immotile ciliary syndrome or Kartagener syndrome. PCD is typically considered to be a rare, ciliopathic, autosomal recessive genetic disorder that often causes defects in the action of cilia lining the respiratory tract (lower and upper, sinuses, Eustachian tube, middle ear) and fallopian tube, as well as in the flagella of sperm cells.

Some individuals with primary ciliary dyskinesia have abnormally placed organs within their chest and abdomen. These abnormalities arise early in embryonic development when the differences between the left and right sides of the body are established. About 50 percent of people with primary ciliary dyskinesia have a mirror-image reversal of their internal organs (situs inversus totalis). For example, in these individuals the heart is on the right side of the body instead of on the left. When someone afflicted with primary ciliary dyskinesia has situs inversus totalis, they are often said to have Kartagener syndrome.

Approximately 12 percent of people with primary ciliary dyskinesia have a condition known as heterotaxy syndrome or situs ambiguus, which is characterized by abnormalities of the heart, liver, intestines, or spleen. These organs may be structurally abnormal or improperly positioned. In addition, affected individuals may lack a spleen (asplenia) or have multiple spleens (polysplenia). Heterotaxy syndrome results from problems establishing the left and right sides of the body during embryonic development. The severity of heterotaxy varies widely among affected individuals.

Primary ciliary dyskinesia can also lead to infertility. Vigorous movements of the flagella are can be needed to propel the sperm cells forward to the female egg cell. Because the sperm of subjects afflicted with primary ciliary dyskinesia does not move properly, males with primary ciliary dyskinesia are usually unable to father children. Infertility occurs in some affected females and it is usually associated with abnormal cilia in the fallopian tubes.

Another feature of primary ciliary dyskinesia is recurrent ear infections (otitis media), especially in young children. Otitis media can lead to permanent hearing loss if untreated. The ear infections are likely related to abnormal cilia within the inner ear.

Rarely, individuals with primary ciliary dyskinesia have an accumulation of fluid in the brain (hydrocephalus), likely due to abnormal cilia in the brain.

At least 21 mutations in the DNAI1 gene have been found to cause primary ciliary dyskinesia, which is a condition characterized by respiratory tract infections, abnormal organ placement, and an inability to have children (infertility). DNAI1 gene mutations result in an absent or abnormal intermediate chain 1. Without a normal version of this subunit, the ODAs cannot form properly and may be shortened or absent. As a result, cilia cannot produce the force needed to bend back and forth. Defective cilia lead to the features of primary ciliary dyskinesia. In some cases, the disclosure provides a nucleic acid that is engineered to replace or to supplement the function of the endogenous DNAI1 protein comprising the IVS1+2_3insT (219+3insT) mutation. In some cases, the disclosure provides a nucleic acid that is engineered to replace or to supplement the function of the endogenous DNAI1 protein comprising the A538T mutation, the second most common.

### Compositions

### Polynucleotides

Provided herein, in some embodiments, is a (e.g., pharmaceutical) composition that comprises a polynucleotide (e.g., comprising a particular sequence that encodes DNAI1). The polynucleotide may comprise a nucleic acid sequence having sequence identity to sequences listed elsewhere herein. The polynucleotide may comprise a nucleic acid sequence having sequence identity to a portion of sequences listed elsewhere herein. For example, the polynucleotide may comprise a nucleic acid sequence having sequence identity to SEQ ID: NO 15. The polynucleotide may comprise a nucleic acid sequence having at a least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence disclosed elsewhere herein. In some embodiments, the nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a fragment (e.g., over at least 500, 600, 700, 800, 900, or 1,000 bases) of SEQ ID NO: 15. In some embodiments, the nucleic acid sequence has 100% sequence identity to a sequence disclosed elsewhere herein. In some embodiments, the nucleic acid has 100% sequence identity to a fragment (e.g., over at least 500, 600, 700, 800, 900, or 1,000 bases) of SEQ ID NO: 15. The polynucleotide may comprise a nucleic acid sequence having at a least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence disclosed elsewhere herein. In some embodiments, the nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence over at least 1,000 bases (e.g., nucleotides 1 to 1,000) of SEQ ID NO: 15. In some embodiments, the nucleic acid sequence has 100% sequence identity to a sequence disclosed elsewhere herein. In some embodiments, the nucleic acid has 100% sequence identity to a sequence over at least 1,000 bases of SEQ ID NO: 15. Polynucleotides described elsewhere herein may be DNA or RNA. The sequences disclosed throughout the specification may have a uridine (U) substituted at any location that a thymidine (T) is present. The disclosure recognizes that a sequence disclosed herein of DNA may be used to generate a corresponding RNA sequence in which instances of thymidine have been replaced with uridine. As such the sequences described herein are not limited to thymidine containing sequence, and the corresponding uridine sequences are also contemplated herein.

The polynucleotide may comprise nucleotide analogues. In some embodiments, the nucleotide analogues replace uridines in a sequence. For example, a sequence using standard nucleotides (A, C, U, T, G) may comprises a uridine at a particular position in a sequence. A sequence may instead have a nucleotide analogue in place of the uridine. The nucleotide analogue may have structure that may still be recognized by the cellular translation machinery such that the polynucleotide comprising a nucleotide analogue may still be translated. The nucleotide analogue may be recognized as synonymous with a standard nucleotide. For example, the nucleotide analogue may be recognized as synonymous with uridine and the resulting translation product is generated as if the nucleotide analogue is a uridine. In some embodiments, at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% of nucleotides replacing uridine within the polynucleotide are nucleotide analogues. In some embodiments, fewer than 15% of nucleotides within the polynucleotide are nucleotide analogues. In some fewer than 30% of the nucleotides are nucleotide analogues. In other cases, fewer than 27.5%, fewer than 25%, fewer than 22.5%, fewer than 20%, fewer than 17.5%, fewer than 15%, fewer than 12.5%, fewer than 10%, fewer than 7.5%, fewer than 5%, or fewer than 2.5% of the nucleotides are nucleotide analogues.

In some embodiments, the nucleotide analogue is a purine or pyrimidine analogue. In some cases, a polyribonucleotide of the disclosure comprises a modified pyrimidine, such as a modified uridine. A nucleotide analogue may be a pseudouridine (Ψ). A nucleotide analogue may be a methylpseudouridine. A nucleotide analogue may be a 1-methylpseudouridine (m¹Ψ). In some embodiments, the polynucleotide comprises a 1-methylpseudouridine. In some cases a uridine analogue is selected from pseudouridine 1-methylpseudouridine, 2-thiouridine (s²U), 5-methyluridine (m⁵U), 5-methoxyuridine (mo⁵U), 4-thiouridine (s⁴U), 5-bromouridine (Br⁵U), 2′O-methyluridine (U2′m), 2′-amino-2′-deoxyuridine (U2′NH₂), 2′-azido-2′-deoxyuridine (U2′N₃), and 2′-fluoro-2′-deoxyuridine (U2′F).

A polyribonucleotide can have the same or a mixture of different nucleotide analogues or modified nucleotides. The nucleotide analogues or modified nucleotides can have structural changes that are naturally or not naturally occurring in messenger RNA. A mixture of various analogues or modified nucleotides can be used. For example, one or more analogues within a polynucleotide can have natural modifications, while another part has modifications that are not naturally found in mRNA. Additionally, some analogues or modified ribonucleotides can have a base modification, while other modified ribonucleotides have a sugar modification. In the same way, it is possible that all modifications are base modifications or all modifications are sugar modifications or any suitable mixture thereof.

A nucleotide analogue or modified nucleotide can be selected from the group comprising pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6- diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some cases, at least about 5% of the nucleic acid construct(s), a vector(s), engineered polyribonucleotide(s), or compositions includes non-naturally occurring (e.g., modified, analogues, or engineered) uridine, adenosine, guanine, or cytosine, such as the nucleotides described herein. In some cases, 100% of the modified nucleotides in the composition are either 1-methylpseudouridine or pseudouridine. In some cases, at least about 10%, 15%, 20%, 25%, 30%, 35%, 40%, 45%, 50%, 55%, 60%, 65%, 70%, 75%, 80%, 85%, 90%, 95% of the nucleic acid construct(s), a vector(s), engineered polyribonucleotide(s), or compositions includes non-naturally occurring uracil, adenine, guanine, or cytosine. In some cases, at most about 99%, 95%,90%,85%,80%,75%,70%,65%,60%,55%,50%,45%,40%,35%,30%,25%, 20%, 15%, 10%, 5%, 1% of the nucleic acid construct(s), a vector(s), engineered polyribonucleotide(s), or compositions includes non-naturally occurring uracil, adenine, guanine, or cytosine.

The polynucleotides may comprise an open reading frame (ORF) sequence. The ORF sequence may be characterized by a codon usage profile comprising: (1) a total number of codons, (2) a species number of codons (e.g., a total number of different codon types), (3) a number of each (unique) codon, and (4) a (usage) frequency of each codon among all synonymous codons (if present). The codon usage profile may be altered or compared to a corresponding wild type sequence. For example, the frequency or number of particular codons may be reduced or increased compared to a wild type sequence. The change in codon frequency of the polynucleotide may provide benefits over the wild type sequence. For example, the altered codon frequency may result in a less immunogenic polynucleotide. The polynucleotide with an altered codon frequency may result in a polynucleotide that is more quickly expressed or results in a greater amount of expression product. The polynucleotide with an altered codon frequency may have increase stability, such as increased half-life in sera, or may be less susceptible to hydrolysis or other reactions that may result in the degradation of the polynucleotide.

TABLE 1
Example ORF sequences

| | | SEQ |
|---|---|---|
| Construct | DNA sequence (from 5′ to 3′) | ID NO. |
| DNAII | ATGATCCCAGCAAGCGCCAAGGCACCACACAAGCAGCCCCACA | 14 |
| GeneScript | AGCAGAGCATCTCCATCGGCAGGGGCACAAGGAAGAGGGACGA | |
| Codon | GGATAGCGGAACCGAAGTGGGAGAGGGAACAGACGAGTGGGCA | |
| | CAGTCCAAGGCAACCGTGCGCCCACCTGACCAGCTGGAGCTGA | |
| | CAGATGCCGAGCTGAAGGAGGAGTTCACCAGGATCCTGACAGC | |
| | CAACAATCCACACGCCCCCCAGAACATCGTGCGCTACTCTTTC | |
| | AAGGAGGGCACATATAAGCCAATCGGCTTTGTGAACCAGCTGG | |
| | CCGTGCACTATACCCAAGTGGGCAATCTGATCCCCAAGGACTC | |
| | CGATGAGGGCCGGAGACAGCACTACAGGGACGAGCTGGTGGCA | |
| | GGATCCCAGGAGTCTGTGAAAGTGATCTCTGAGACCGGCAATC | |
| | TGGAGGAGGACGAGGAGCCAAAGGAGCTGGAGACCGAGCCAGG | |
| | AAGCCAGACAGATGTGCCTGCAGCAGGAGCAGCAGAGAAGGTG | |
| | ACCGAGGAGGAGCTGATGACACCTAAGCAGCCAAAGGAGCGGA | |
| | AGCTGACCAACCAGTTCAATTTTTCCGAGAGAGCCTCTCAGAC | |
| | ATACAACAATCCAGTGCGGGACAGAGAGTGCCAGACCGAGCCA | |
| | CCCCCTAGAACCAACTTTTCCGCCACAGCCAATCAGTGGGAGA | |
| | TCTACGATGCCTATGTGGAGGAGCTGGAGAAGCAGGAGAAGAC | |
| | CAAGGAGAAGGAGAAGGCCAAGACACCCGTGGCCAAGAAGTCC | |
| | GGCAAGATGGCCATGCGGAAGCTGACCAGCATGGAGTCCCAGA | |
| | CAGACGATCTGATCAAGCTGTCTCAGGCCGCCAAGATCATGGA | |
| | GAGAATGGTGAACCAGAATACCTATGACGATATCGCCCAGGAC | |
| | TTCAAGTACTATGACGATGCAGCAGACGAGTACAGGGATCAAG | |
| | TGGGCACACTGCTGCCTCTGTGGAAGTTTCAGAACGATAAGGC | |
| | CAAGAGGCTGAGCGTGACCGCCCTGTGCTGGAATCCAAAGTAC | |
| | AGGGACCTGTTCGCAGTGGGATACGGATCTTATGACTTCATGA | |
| | AGCAGAGCAGAGGCATGCTGCTGCTGTATTCCCTGAAGAACCC | |
| | CTCTTTCCCTGAGTACATGTTTAGCTCCAATTCCGGCGTGATG | |
| | TGCCTGGACATCCACGTGGATCACCCCTACCTGGTGGCCGTGG | |
| | GCCACTATGACGGCAACGTGGCCATCTACAATCTGAAGAAGCC | |
| | TCACTCTCAGCCCAGCTTCTGTTCTAGCGCCAAGAGCGGCAAG | |
| | CACTCCGATCCCGTGTGGCAGGTGAAGTGGCAGAAGGACGATA | |
| | TGGACCAGAACCTGAATTTCTTTTCCGTGTCCTCTGATGGCAG | |
| | GATCGTGTCTTGGACCCTGGTGAAGCGCAAGCTGGTGCACATC | |
| | GACGTGATCAAGCTGAAGGTGGAGGGCAGCACCACAGAGGTGC | |
| | CAGAGGGACTGCAGCTGCACCCAGTGGGATGCGGCACAGCCTT | |
| | CGACTTTCACAAGGAGATCGATTATATGTTCCTGGTGGGCACC | |
| | GAGGAGGGCAAGATCTACAAGTGTTCTAAGAGCTATAGCTCCC | |
| | AGTTTCTGGACACATATGATGCCCACAACATGAGCGTGGATAC | |
| | CGTGTCCTGGAATCCTTACCACACAAAGGTGTTCATGAGCTGC | |
| | TCTAGCGACTGGACCGTGAAGATCTGGGATCACACCATCAAGA | |
| | CACCTATGTTTATCTATGACCTGAACTCCGCCGTGGGCGATGT | |
| | GGCATGGGCACCATACTCCTCTACAGTGTTCGCAGCAGTGACC | |
| | ACAGACGGCAAGGCACACATCTTTGATCTGGCCATCAACAAGT | |
| | ACGAGGCCATCTGTAATCAGCCCGTGGCCGCCAAGAAGAACAG | |
| | GCTGACCCACGTGCAGTTCAATCTGATCCACCCTATCATCATC | |
| | GTGGGCGACGATCGGGGCCACATCATCTCTCTGAAGCTGAGCC | |
| | CCAACCTGAGAAAGATGCCTAAGGAGAAGAAGGGACAGGAGGT | |
| | GCAGAAGGGACCAGCAGTGGAGATCGCAAAGCTGGACAAGCTG | |
| | CTGAATCTGGTGCGCGAGGTGAAGATCAAGACCTGA | |
| DNAII | ATGATCCCAGCAAGCGCCAAGGCACCACACAAGCAGCCCCACA | 15 |
| Altered | AGCAGAGCATCAGCATCGGCAGGGGCACAAGGAAGAGGGACGA | |
| Nucleotide | GGACAGCGGAACCGAAGTGGGAGAGGGAACAGACGAGTGGGCA | |
| Usage 1 | CAGAGCAAGGCAACCGTGCGCCCACCCGACCAGCTGGAGCTGA | |
| | CAGACGCCGAGCTGAAGGAGGAGTTCACCAGGATCCTGACAGC | |
| | CAACAACCCACACGCCCCCCAGAACATCGTGCGCTACAGCTTC | |
| | AAGGAGGGCACATACAAGCCAATCGGCTTCGTGAACCAGCTGG | |
| | CCGTGCACTACACCCAAGTGGGCAACCTGATCCCCAAGGACAG | |
| | CGACGAGGGCCGGAGACAGCACTACAGGGACGAGCTGGTGGCA | |
| | GGAAGCCAGGAGAGCGTGAAAGTGATCAGCGAGACCGGCAACC | |
| | TGGAGGAGGACGAGGAGCCAAAGGAGCTGGAGACCGAGCCAGG | |
| | AAGCCAGACAGACGTGCCCGCAGCAGGAGCAGCAGAGAAGGTG | |
| | ACCGAGGAGGAGCTGATGACACCCAAGCAGCCAAAGGAGCGGA | |
| | AGCTGACCAACCAGTTCAACTTCAGCGAGAGAGCCAGCCAGAC | |
| | ATACAACAACCCAGTGCGGGACAGAGAGTGCCAGACCGAGCCA | |
| | CCCCCCAGAACCAACTTCAGCGCCACAGCCAACCAGTGGGAGA | |
| | TCTACGACGCCTACGTGGAGGAGCTGGAGAAGCAGGAGAAGAC | |
| | CAAGGAGAAGGAGAAGGCCAAGACACCCGTGGCCAAGAAGAGC | |
| | GGCAAGATGGCCATGCGGAAGCTGACCAGCATGGAGAGCCAGA | |
| | CAGACGACCTGATCAAGCTGAGCCAGGCCGCCAAGATCATGGA | |
| | GAGAATGGTGAACCAGAACACCTACGACGACATCGCCCAGGAC | |
| | TTCAAGTACTACGACGACGCAGCAGACGAGTACAGGGACCAAG | |
| | TGGGCACACTGCTGCCCCTGTGGAAGTTCCAGAACGACAAGGC | |
| | CAAGAGGCTGAGCGTGACCGCCCTGTGCTGGAACCCAAAGTAC | |
| | AGGGACCTGTTCGCAGTGGGATACGGAAGCTACGACTTCATGA | |
| | AGCAGAGCAGAGGCATGCTGCTGCTGTACAGCCTGAAGAACCC | |
| | CAGCTTCCCCGAGTACATGTTCAGCAGCAACAGCGGCGTGATG | |
| | TGCCTGGACATCCACGTGGACCACCCCTACCTGGTGGCCGTGG | |
| | GCCACTACGACGGCAACGTGGCCATCTACAACCTGAAGAAGCC | |
| | CCACAGCCAGCCCAGCTTCTGCAGCAGCGCCAAGAGCGGCAAG | |
| | CACAGCGACCCCGTGTGGCAGGTGAAGTGGCAGAAGGACGACA | |
| | TGGACCAGAACCTGAACTTCTTCAGCGTGAGCAGCGACGGCAG | |
| | GATCGTGAGCTGGACCCTGGTGAAGCGCAAGCTGGTGCACATC | |
| | GACGTGATCAAGCTGAAGGTGGAGGGCAGCACCACAGAGGTGC | |
| | CAGAGGGACTGCAGCTGCACCCAGTGGGATGCGGCACAGCCTT | |
| | CGACTTCCACAAGGAGATCGACTACATGTTCCTGGTGGGCACC | |
| | GAGGAGGGCAAGATCTACAAGTGCAGCAAGAGCTACAGCAGCC | |
| | AGTTCCTGGACACATACGACGCCCACAACATGAGCGTGGACAC | |
| | CGTGAGCTGGAACCCCTACCACACAAAGGTGTTCATGAGCTGC | |
| | AGCAGCGACTGGACCGTGAAGATCTGGGACCACACCATCAAGA | |
| | CACCCATGTTCATCTACGACCTGAACAGCGCCGTGGGCGACGT | |
| | GGCATGGGCACCATACAGCAGCACAGTGTTCGCAGCAGTGACC | |
| | ACAGACGGCAAGGCACACATCTTCGACCTGGCCATCAACAAGT | |
| | ACGAGGCCATCTGCAACCAGCCCGTGGCCGCCAAGAAGAACAG | |
| | GCTGACCCACGTGCAGTTCAACCTGATCCACCCCATCATCATC | |
| | GTGGGCGACGACCGGGGCCACATCATCAGCCTGAAGCTGAGCC | |
| | CCAACCTGAGAAAGATGCCCAAGGAGAAGAAGGGACAGGAGGT | |
| | GCAGAAGGGACCAGCAGTGGAGATCGCAAAGCTGGACAAGCTG | |
| | CTGAACCTGGTGCGCGAGGTGAAGATCAAGACCTGA | |
| Wild Type | ATGATTCCTGCTTCTGCGAAGGCTCCCCATAAACAGCCTCATA | 16 |
| DNAII | AGCAGAGCATCAGCATAGGCAGAGGAACCAGGAAGAGAGATGA | |
| | AGATTCAGGGACTGAAGTGGGAGAAGGCACAGATGAATGGGCC | |
| | CAATCCAAAGCCACAGTTAGACCCCCTGACCAGCTGGAGTTGA | |
| | CCGATGCGGAGTTAAAGGAGGAGTTCACTCGGATTTTGACAGC | |
| | CAACAACCCACACGCACCCCAGAACATTGTCAGGTACAGCTTC | |
| | AAAGAAGGCACATATAAGCCTATTGGCTTTGTGAACCAACTGG | |
| | CAGTTCACTACACCCAGGTTGGGAACCTGATCCCCAAAGACTC | |
| | AGATGAAGGACGGCGGCAGCATTACCGCGATGAATTAGTGGCA | |
| | GGTTCTCAGGAGTCTGTCAAGGTGATTTCAGAAACAGGAAACC | |
| | TCGAAGAAGACGAAGAGCCCAAGGAGTTAGAAACTGAGCCTGG | |
| | GAGTCAAACAGATGTGCCTGCAGCTGGGGCAGCTGAAAAAGTG | |
| | ACTGAAGAAGAATTGATGACTCCTAAGCAGCCCAAGGAGAGAA | |
| | AGCTCACTAACCAGTTCAACTTCAGTGAGAGGGCCTCACAGAC | |
| | CTACAACAACCCTGTCCGGGATCGAGAATGCCAGACGGAGCCT | |
| | CCTCCCAGGACAAACTTTTCAGCCACAGCCAATCAGTGGGAGA | |
| | TCTATGATGCCTATGTAGAGGAACTTGAGAAGCAGGAAAAGAC | |
| | CAAAGAGAAGGAGAAGGCAAAGACCCCAGTGGCTAAAAAATCA | |
| | GGGAAGATGGCCATGAGGAAGCTGACATCTATGGAGTCTCAGA | |
| | CTGATGATCTCATCAAATTGTCCCAAGCTGCTAAGATCATGGA | |
| | GCGGATGGTCAACCAGAATACATATGATGACATTGCTCAAGAT | |
| | TTTAAGTACTATGACGATGCTGCTGATGAATACCGGGACCAGG | |
| | TGGGTACCCTGCTGCCGCTCTGGAAGTTCCAAAATGACAAAGC | |
| | CAAGCGCCTGTCCGTCACTGCCCTCTGCTGGAATCCAAAGTAC | |
| | AGGGATCTGTTTGCAGTGGGATATGGCTCTTATGACTTCATGA | |
| | AGCAGAGCCGGGGCATGCTGCTGCTCTACAGCCTGAAGAACCC | |
| | CAGCTTCCCTGAGTACATGTTCAGCAGCAACAGCGGCGTCATG | |
| | TGTCTCGACATCCACGTGGACCACCCCTACCTGGTGGCAGTAG | |
| | GCCACTATGACGGCAACGTGGCCATTTACAACCTCAAGAAGCC | |
| | CCACTCCCAGCCCTCCTTCTGCAGCTCAGCCAAGTCTGGCAAG | |
| | CACTCAGACCCTGTGTGGCAGGTCAAGTGGCAGAAGGATGACA | |
| | TGGACCAAAACCTTAACTTCTTCTCTGTGTCATCTGACGGCAG | |
| | GATTGTGTCTTGGACTCTCGTGAAGAGAAAGCTGGTTCACATA | |
| | GATGTCATCAAGCTGAAGGTGGAAGGCAGCACCACGGAAGTTC | |
| | CTGAGGGGTTGCAGCTGCACCCAGTGGGTTGTGGCACTGCCTT | |
| | TGACTTCCACAAAGAGATTGACTACATGTTCCTAGTGGGCACA | |
| | GAGGAGGGAAAAATCTACAAGTGCTCTAAATCCTACTCCAGCC | |
| | AATTCCTCGACACCTATGACGCCCACAACATGTCAGTGGACAC | |
| | TGTGTCCTGGAACCCATACCACACCAAGGTCTTCATGTCCTGC | |
| | AGCTCCGACTGGACAGTGAAGATCTGGGACCACACCATCAAGA | |
| | CCCCGATGTTCATCTATGACCTGAACTCAGCCGTGGGTGATGT | |
| | GGCCTGGGCGCCATACTCTTCTACTGTGTTCGCAGCAGTCACC | |
| | ACAGATGGGAAGGCCCACATATTTGACTTAGCCATCAACAAGT | |
| | ATGAGGCCATCTGCAACCAGCCTGTGGCGGCCAAAAAGAACAG | |
| | GCTCACCCACGTGCAGTTCAATCTCATCCACCCCATCATCATT | |
| | GTGGGCGATGACCGTGGGCACATCATCAGCCTCAAGCTCTCAC | |
| | CCAATTTGCGCAAGATGCCAAAGGAAAAGAAGGGGCAGGAGGT | |
| | GCAGAAGGGTCCAGCTGTGGAGATTGCGAAACTGGACAAACTG | |
| | CTGAACCTGGTGAGGGAAGTGAAAATCAAGACCTGA | |

### Codon Optimizations

In some embodiments, the polynucleotide comprises an altered nucleotide usage as compared to a corresponding wild type sequence. The altered nucleotide usage may also be referred to as a “codon optimized” sequence or be generated by way of “codon optimization”.

Altered nucleotide usage schemes aiming to reduce the number of more reactive 5′-U(U/A)-3′ dinucleotides within codons as well as across codons of modified mRNAs partially alleviate limitations imposed by the inherent chemical instability of RNA. At the same time, lowering the U-content in RNA transcripts renders them less immunogenic. The present disclosure relates to RNA transcripts comprising altered open reading frames (ORF). For example, the codon optimized or altered nucleotide usage may comprise a substantial reduction of 5′-U(U/A)-3′ dinucleotides within protein coding regions leading to stabilized therapeutic mRNAs. The codon optimized polynucleotide may comprise a codon coding for a particular amino acid to be substituted or replaced of a with a synonymous codon. The codon optimized polynucleotide may encode a same or identical polypeptide as a corresponding wild type polynucleotide, with the polynucleotide comprising a different sequence of polynucleotide than the corresponding wild type. Multiple codons may encode for a same amino acid, however the qualities of a given codon are differ between even those that code for a same amino acid. Because multiple different codons may code for a same amino acid, a particular polynucleotide may encode for a same polypeptide and have advantageous features over another polynucleotide that codes for the same polypeptide. For example, a codon optimized polynucleotides may be transcribed faster, may comprise a higher stability (in vivo or in vitro), may result in increased expression yield or full length or functional polypeptides, or may result in an increase of soluble polypeptide and a decrease in polypeptide aggregates. Without being limited to a specific mechanism, the advantageous features of a codon optimized polynucleotides may be for example, a result of improved protein folding of the expressed product based on ribosomal interactions with the polynucleotides, or may be result of decreased hydrolysis of reactive bonds in solution. For example, the codon optimization may have altered or improved characteristics relating to ribosomal binding sites, Shine-Dalgarno sequences, or ribosomal or translational pausing. The advantageous features may be a result of decreased usage of “rare codons” which may have a lower concentration of cognate tRNAs, allowing for an improved translation reaction. The advantageous features may be a result of decreased usage of “rare codons” which may have a lower concentration of cognate tRNAs, allowing for an improved translation reaction. The advantageous features may be a result of decreasing degradation via enzymatic reaction. For example, hydrolysis of oligonucleotides suggests that the reactivity of the phosphodiester bond linking two ribonucleotides in single-stranded (ss)RNA depends on the nature of those nucleotides. At pH 8.5, dinucleotide cleavage susceptibility when embedded in ssRNA dodecamers may vary by an order of magnitude. Under near physiological conditions, hydrolysis of RNA usually involves an S_{N}2-type attack by the 2′-oxygen nucleophile on the adjacent phosphorus target center on the opposing side of the 5′-oxyanion leaving group, yielding two RNA fragments with 2′,3′-cyclic phosphate and 5′-hydroxyl termini. More reactive scissile phosphodiester bonds may include 5′-UpA-3′ (R₁=U₁, R₂=A) and 5′-CpA-3′ (R₁=C, R₂=A) because the backbone at these steps can most easily adopt the “in-line” conformation that is required for S_{N}2-type nucleophilic attack by the 2′—OH on the adjacent phosphodiester linkage. In addition, interferon-regulated dsRNA-activated antiviral pathways produce 2′-5′ oligoadenylates which bind to ankyrin repeats leading to activation of Rnase L endoribonuclease. Rnase L cleaves ssRNA efficiently at UA and UU dinucleotides. Lastly, U-rich sequences are potent activators of RNA sensors including Toll-like receptor 7 and 8 and RIG-I making global uridine content reduction a potentially attractive approach to reduce immunogenicity of therapeutic mRNAs.

In some embodiments, the nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CAG, AGG, CGG, CGA, CGT, CGC, TCG, TCA, TCT, TCC, ACG, ACT, GTA, GTT, GTC, and TAT, as compared to a corresponding wild-type sequence, e.g., SEQ ID NO: 16. In some embodiments, the nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence, e.g., SEQ ID NO: 16. In some embodiments, the nucleic acid sequence comprises fewer codon types encoding an amino acid as compared to a corresponding wild-type sequence, e.g., SEQ ID NO: 16.

In some cases, a codon coding for a particular amino acid in the polypeptide may be substituted or replaced with a synonymous codon. For example, a codon coding for leucine may be substituted for another codon coding for leucine. In this way, the resulting translation products may be identical with the polynucleotide differing in sequence. At least one type of an isoleucine-encoding codons in the corresponding wild-type sequence may be substituted with a synonymous codon type in the nucleic acid sequence. At least one type of a valine-encoding codons in the corresponding wild-type sequence may be substituted with a synonymous codon type in the nucleic acid sequence. At least one type of an alanine-encoding codons in the corresponding wild-type sequence may be substituted with a synonymous codon type in the nucleic acid sequence. At least one type of a glycine-encoding codons in the corresponding wild-type sequence may be substituted with a synonymous codon type in the nucleic acid sequence. At least one type of a proline-encoding codons in the corresponding wild-type sequence may be substituted with a synonymous codon type in the nucleic acid sequence. At least one type of a threonine-encoding codons in the corresponding wild-type sequence may be substituted with a synonymous codon type in the nucleic acid sequence. At least one type of a leucine-encoding codons in the corresponding wild-type sequence may be substituted with a synonymous codon type in the nucleic acid sequence. At least one type of an arginine-encoding codons in the corresponding wild-type sequence is substituted with a synonymous codon type in the nucleic acid sequence. At least one type of a serine-encoding codons in the corresponding wild-type sequence may be substituted with a synonymous codon type in the nucleic acid sequence.

In some aspects described herein, a particular codon of a particular amino acid comprises a percentage or amount of the total number of codons for that particular amino acid the polynucleotide. This may be referred to a “codon frequency”. For example, at least 50% of the total codons encoding a particular amino acid in the polynucleotide may be encoded by a first codon sequence. For example, at least 55% of the total codons encoding a particular amino acid in the polynucleotide may be encoded by a first codon sequence. At least 5%, 10%, 20%, 30%, 40%, 50%, 55%, 60%, 65%, 70%, 75%, 80%, 85%, 90%, 95%, or more of the total codons encoding a particular amino in the polynucleotide may be encoded by a first codon sequence. In some cases, no more than 5%, 10%, 20%, 30%, 40%, 50%, 55%, 60%, 65%, 70%, 75%, 80%, 85%, 90%, 95%, or less of the total codons encoding a particular amino in the polynucleotide are encoded by a first codon sequence. At least about 90% phenylalanine-encoding codons of the synthetic polynucleotide may be TTC (as opposed to TTT). At least about 60% cysteine-encoding codons of the synthetic polynucleotide may be TGC (as opposed to TGT). At least about 70% aspartic acid-encoding codons of the synthetic polynucleotide may be GAC (as opposed to GAT). At least about 50% glutamic acid-encoding codons of the synthetic polynucleotide may be GAG (as opposed to GAA). At least about 60% histidine-encoding codons of the synthetic polynucleotide may be CAC (as opposed to CAT). At least about 60% lysine-encoding codons of the synthetic polynucleotide may be AAG (as opposed to AAA). At least about 60% asparagine-encoding codons of the synthetic polynucleotide may be AAC (as opposed to AAT). At least about 70% glutamine-encoding codons of the synthetic polynucleotide may be CAG (as opposed to CAA). At least about 80% tyrosine-encoding codons of the synthetic polynucleotide may be TAC (as opposed to TAT). At least about 90% isoleucine-encoding codons of the synthetic polynucleotide may be ATC.

In some embodiments, a particular amino acid the polynucleotide may be encoded by a number of different codon sequences. For example, a particular amino acid in the polynucleotide may be encoded by no more than 2 different codon sequences. In some cases, the polynucleotide comprises no more than 2 types of isoleucine-encoding codons.

In some embodiments, a particular amino acid in the polynucleotide may be encoded by no more than 3 different codon sequences. The polynucleotide may comprise no more than 3 types of alanine (Ala)-encoding codons. The polynucleotide may comprise no more than 3 types of glycine (Gly)-encoding codons. The polynucleotide may comprise no more than 3 types of proline (Pro)-encoding codons. The polynucleotide may comprise no more than 3 types of threonine (Thr)-encoding codons.

In some embodiments, a particular amino acid in the polynucleotide may be encoded by no more than 4 different codon sequences. The polynucleotide may comprise no more than 4 types of arginine (Arg)-encoding codons. The polynucleotide may comprise no more than 4 types of serine (Ser)-encoding codons. In some embodiments, a particular amino acid in the polynucleotide may be encoded by no more than 5 different codon sequences. The polynucleotide may comprise no more than 5 types of arginine (Arg)-encoding codons. The polynucleotide may comprise no more than 5 types of serine (Ser)-encoding codons. In some embodiments, a particular amino acid in the polynucleotide may be encoded by no more than 6 different codon sequences. In some embodiments, a particular amino acid in the polynucleotide may be encoded by 1 or more different codon sequences. In some embodiments, a particular amino acid in the polynucleotide may be encoded by 2 or more different codon sequences. In some embodiments, a particular amino acid in the polynucleotide may be encoded by 3 or more different codon sequences. In some embodiments, a particular amino acid in the polynucleotide may be encoded by 4 or more different codon sequences. In some embodiments, a particular amino acid in the polynucleotide may be encoded by 5 or more different codon sequences. In some embodiments, a particular amino acid in the polynucleotide may be encoded by 6 or more different codon sequences.

In some cases, a frequency of a first codon sequence of a is higher, lower or the same as a frequency of a second codon sequence encoding for a particular amino acid in the polynucleotide. For example, a frequency of a first codon is higher than a frequency of second codon for a particular amino acid in the polynucleotide. The frequency of GCC codon may be higher than a frequency of GCT codon. The frequency of GCT codon may be lower than a frequency of GCA codon. The frequency of GCT codon may be higher than a frequency of GCA codon.

In some embodiments, the codon usage for alanine-encoding codons in the polynucleotide may have a particular parameter. For example, a frequency of GCG codon may be no more than about 10% or 5%. A frequency of GCA codon may be no more than about 20%. A frequency of GCT codon may be at least about 1%, 5%, 10%, 15%, 20%, or 25%. A frequency of GCT codon may be no more than about 30%, 25%, 20%, 15%, 10%, or 5%. A frequency of GCC codon may be at least about 60%, 70%, 80%, or 90%. A frequency of GCC codon is no more than about 95%, 90%, 85%, 80%, or 75%. The frequency of GCC codon may be higher than a frequency of GCT codon. The frequency of GCT codon may be lower than a frequency of GCA codon. The frequency of GCT codon may be higher than a frequency of GCA codon.

In some embodiments, the codon usage for glycine-encoding codons the polynucleotide may have a particular parameter. For example, a frequency of GGC codon may be lower than a frequency of GGA codon. For example, a frequency of GGC codon may be higher than a frequency of GGA codon. A frequency of GGG codon may be no more than about 10% or 5%. A frequency of GGG codon may be least about 1%. A frequency of GGA codon may be no more than about 30% or 20%. A frequency of GGA codon may be at least about 10% or 20%. A frequency of GGT codon may be more than about 10% or 5%. A frequency of GGC codon may be no more than about 90%, 80%, or 70%. A frequency of GGC codon may be least about 60%, 70%, or 80%.

In some embodiments, the codon usage for proline-encoding codons the polynucleotide may have a particular parameter. For example, a frequency of CCC codon may be lower than a frequency of CCT codon. A frequency of CCC codon may be higher than a frequency of CCT codon. A frequency of CCC codon may be lower than a frequency of CCA codon. A frequency of CCC codon may be higher than a frequency of CCA codon. A frequency of CCT codon may be lower than a frequency of CCA codon. A frequency of CCT codon may be higher than a frequency of CCA codon. A frequency of CCG codon may be no more than about 10% or 5%. Frequency of CCA codon may be no more than about 30%, 20%, or 10%. A frequency of CCA codon may be at least about 5%, 10%, 15%, 20%, or 25%. A frequency of CCT codon may be no more than about 60%, 50%, 40%, or 30%. A frequency of CCT codon may be at least about 20%, 30%, 40%, or 50%. A frequency of CCC codon may be no more than about 60%, 50%, or 40%. A frequency of CCC codon may be at least about 30%, 40%, 50%, 60%, or 70%.

In some embodiments, the codon usage for threonine-encoding codons the polynucleotide may have a particular parameter. For example, a frequency of ACA codon is higher than a frequency of ACT codon. A frequency of ACC codon may be higher than a frequency of ACT codon. A frequency of ACC codon may be lower than a frequency of ACA codon. A frequency of ACC codon may be higher than a frequency of ACA codon. A frequency of ACG codon may be no more than about 10% or 5%. A frequency of ACA codon may be no more than about 60%, 50%, 40%, or 30%. A frequency of ACA codon may be at least about 10%, 20%, 30%, 40%, or 50%. A frequency of ACT codon may be no more than about 10% or 5%. A frequency of ACC codon may be no more than about 90%, 80%, 70%, 60%, or 50%. A frequency of ACC codon is at least about 40%, 50%, 60%, 70%, or 80%.

In some embodiments, the codon usage for arginine-encoding codons the polynucleotide may have a particular parameter. For example, a frequency of AGA codon may be lower than a frequency of AGG codon. A frequency of AGA codon may be higher than a frequency of AGG codon. A frequency of AGA codon may be lower than a frequency of CGG codon. A frequency of AGA codon may be higher than a frequency of CGG codon. A frequency of CGG codon may be higher than a frequency of CGA codon. A frequency of CGG codon is higher than a frequency of CGC codon. A frequency of AGG codon may be no more than about 10%. A frequency of AGG codon may be less than about 10%. A frequency of AGA codon may be no more than about 70%, 60%, or 50%. A frequency of AGA codon may be at least about 40%, 50%, 60%, or 70%. A frequency of CGG codon may be no more than about 50%, 40%, or 30%. A frequency of CGG codon may be at least about 20%, 30%, or 40%. A frequency of CGA codon may be at least about 1%. A frequency of CGA codon may be no more than about 10% or 5%. A frequency of CGT codon may be no more about 10% or 5%. A frequency of CGC codon may be no more than about 20%, 10%, or 5%. A frequency of CGC codon may be at least about 1%, 2%, 3%, 4%, or 5%.

In some embodiments, the codon usage for serine-encoding codons the polynucleotide may have a particular parameter. For example, a frequency of AGC codon may be higher than a frequency of TCT codon. A frequency of TCT codon may be higher than a frequency of TCG codon. A frequency of TCT codon may be higher than a frequency of TCA codon. A frequency of TCT codon may be higher than a frequency of TCC codon. A frequency of AGT codon may be no more than about 10%. A frequency of AGT codon may be at least about 1%. A frequency of AGC codon may be no more about 95%, 90%, 85%, or 80%. A frequency of AGC codon may be at least about 70%, 80%, or 90%. A frequency of TCG codon may be no more than about 10% or 5%. A frequency of TCA codon may be no more than about 10% or 5%. A frequency of TCT codon may be no more than about 30%, 20%, or 10%. A frequency of TCT codon may be at least about 10%, or 20%. A frequency of TCC codon may be no more than about 10% or 5%.

### Untranslated Regions

In some instances, a polynucleotide, nucleic acid construct, vector, or composition of the disclosure comprises one or more nucleotide sequences that encode dynein axonemal intermediate chain 1 (DNAI1) protein or a variant thereof, and the sequences provide for heterologous or enhanced expression of the dynein axonemal intermediate chain 1 (DNAI1) protein or a variant thereof within cells of a subject. In some instances, the nucleic acid construct, vector, or composition also comprises a 5′ untranslated region (UTR) or 3′ UTR having at least 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to one set forth in SEQ ID NOs 1-14, SEQ ID NOs. 1-8 and 14, or SEQ ID NOs. 8-13. In some instances, the polynucleotide comprises a 5′ untranslated region (UTR) having at least 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID 14. In some embodiments, the nucleic acid sequence of the present disclosure comprises one or more (e.g., one or two) sequences set forth in SEQ ID NOs 1-8 and 14. In some embodiments, the nucleic acid sequence of the present disclosure comprises a sequence set forth in SEQ ID NOs 8-13. In some embodiments, the nucleic acid sequence of the present disclosure comprises the sequence set forth in SEQ ID NO 14.

TABLE 2
Example UTR sequences

| | | SEQ |
|---|---|---|
| UTR | DNA sequence (from 5′ to 3′) | ID NO. |
| α-globin 5′ | GGGAGACATAAACCCTGGCGCGCTCGCGGCCCGGCACTCTTC | 1 |
| UTR | TGGTCCCCACAGACTCAGAGAGAAGCCACC | |
| (HBA1) | | |
| α-globin 5′ | GGGAGACATAAACCCTGGCGCGCTCGCGGGCCGGCACTCTTC | 2 |
| UTR | TGGTCCCCACAGACTCAGAGAGAAGCCACC | |
| (HBA2) | | |
| α-globin 5′ | GGGAGACTCTTCTGGTCCCCACAGACTCAGAGAGAACGCCAC | 3 |
| UTR | C | |
| IRES of | GTTATTTTCCACCATATTGCCGTCTTTTGGCAATGTGAGGGC | 4 |
| EMCV 5′- | CCGGAAACCTGGCCCTGTCTTCTTGACGAGCATTCCTAGGGG | |
| UTR | TCTTTCCCCTCTCGCCAAAGGAATGCAAGGTCTGTTGAATGT | |
| | CGTGAAGGAAGCAGTTCCTCTGGAAGCTTCTTGAAGACAAAC | |
| | AACGTCTGTAGCGACCCTTTGCAGGCAGCGGAACCCCCCACC | |
| | TGGCGACAGGTGCCTCTGCGGCCAAAAGCCACGTGTATAAGA | |
| | TACACCTGCAAAGGCGGCACAACCCCAGTGCCACGTTGTGAG | |
| | TTGGATAGTTGTGGAAAGAGTCAAATGGCTCTCCTCAAGCGT | |
| | ATTCAACAAGGGGCTGAAGGATGCCCAGAAGGTACCCCATTG | |
| | TATGGGATCTGATCTGGGGCCTCGGTGCACATGCTTTACGTG | |
| | TGTTTAGTCGAGGTTAAAAAACGTCTAGGCCCCCCGAACCAC | |
| | GGGGACGTGGTTTTCCTTTGAAAAACACGATGATAATATGGC | |
| | CACAACC | |
| IRES of | AAATAACAAATCTCAACACAACATATACAAAACAAACGAATC | 5 |
| TEV 5′- | TCAAGCAATCAAGCATTCTACTTCTATTGCAGCAATTTAAAT | |
| UTR | CATTTCTTTTAAAGCAAAAGCAATTTTCTGAAAATTTTCACC | |
| | ATTTACGAACGATAGCA | |
| ssRNA1 | GGGAGACAAGAGAGAAAAGAAGAGCAAGAAGAAATATAAGAG | 6 |
| 5′UTR | CCACC | |
| ssRNA2 | GGGAGACCCAAGCTGGCTAGCGTTTAAACTTAAGCTTGGCAA | 7 |
| 5′UTR | TCCGGTACTGTTGGTAAAGCCACC | |
| ssRNA 3 + | GGGAGACCCAAGCTGGCTAGCGTTTAAACTTAAGCTTTCCTT | 8 |
| native 5′ | TCCGGGCCGGCTGGGCGCGCCGAAGCGCCTGCGCCTTGGCTG | |
| UTR | CTGGTCGGTTGCTGGGTAACCGCGTCAGGGAGTTGGATTCTA | |
| | TCCTGCAAGGGCACGGGGACCCACAACGACGGCTGTCCCTAA | |
| | AGAACCGTTGCGACTGGTAACTGAAGTGGAAGAGAGTCCAGA | |
| | TTTCTTGTGTGTGGTCAAGGAGACGGACAAACTTTTTGTCTT | |
| | CAGACGAGGGAGCGTTTTGTAGGCTCTCCAGGGGTTGAG | |
| TMV 3′- | GGATTGTGTCCGTAATCACACGTGGTGCGTACGATAACGCAT | 9 |
| UTR | AGTGTTTTTCCCTCCACTTAAATCGAAGGGTTGTGTCTTGGA | |
| | TCGCGCGGGTCAAATGTATATGGTTCATATACATCCGCAGGC | |
| | ACGTAATAAAGCGAGGGGTTCGAATCCCCCCGTTACCCCCGG | |
| | TAGGGGCCCATTGTCTTC | |
| MALATI | TCAGTAGGGTCATGAAGGTTTTTCTTTTCCTGAGAAAACAAC | 10 |
| 3′-UTR | ACGTATTGTTTTCTCAGGTTTTGCTTTTTGGCCTTTTTCTAG | |
| | CTTAAAAAAAAAAAAAGCAAAATTGTCTTC | |
| NEAT2 3′- | TCAGTAGGGTTGTAAAGGTTTTTCTTTTCCTGAGAAAACAAC | 11 |
| UTR | CTTTTGTTTTCTCAGGTTTTGCTTTTTGGCCTTTCCCTAGCT | |
| | TTAAAAAAAAAAAAGCAAAATTGTCTTC | |
| histone | GAAGTGGCGGTTCGGCCGGAGGTTCCATCGTATCCAAAAGGC | 12 |
| cluster 2, | TCTTTTCAGAGCCACCCATTGTCTTC | |
| H3c 3′-UTR | | |
| Native 3′ | GGGGCTGGCCTCAGTCTCTGTCCCATCGCTTGAATACAGTAC | 13 |
| UTR | TCCTAGGGCTTGACCCTGGTACCCAGCCCAGCCTTAGCACCC | |
| | AGCATGTGACCCCACTCCTGATCAGGTCCCAGCATCTTCCCT | |
| | TCTTGTTCTGTTCCTTAAGGTCCCAGCACCTTACCCCAGGAC | |
| | TTGGTCTTCAACCACCATTACCCCTCTAACTTTGCACAAATA | |
| | AACCTGTGTAGAAACCCACCCCAAAAAAA | |
| ssRNA2 | GGGAGACCCAAGCTGGCTAGCGTTTAAACTTCAGCTTGGCAA | 14 |
| 5′UTR | TCCGGTACTGTTGGTAAAGCCACC | |
| (A32C) | | |

In some embodiments, the polynucleotide is present in the (e.g., pharmaceutical) composition at a concentration of no more than 1 mg/mL. In some embodiments, the polynucleotide is present in the (e.g., pharmaceutical) composition at a concentration of no more than 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or less. In some embodiments, the polynucleotide is present in the (e.g., pharmaceutical) composition at a concentration of at least 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or more. In some embodiments, the polynucleotide is present in the (e.g., pharmaceutical) composition at a concentration of any one of the following values or within a range of any two of the following values: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or a range between any two of the foregoing values. In some embodiments, the polynucleotide is present in the (e.g., pharmaceutical) composition at a concentration from 0.5 mg/mL to 5 mg/mL. In some embodiments, the polynucleotide is present in the (e.g., pharmaceutical) composition at a concentration from 0.5 mg/mL to 1 mg/mL. In some embodiments, the polynucleotide is present in the (e.g., pharmaceutical) composition at a concentration from 2 mg/mL to 5 mg/mL.

### Lipid Formulations

The present disclosure provides a (e.g., pharmaceutical) composition comprising a polynucleotide assembled with a lipid composition, wherein the polynucleotide encodes a dynein axonemal intermediate chain 1 (DNAI1) protein; and wherein the lipid composition comprises a (e.g., ionizable) cationic lipid. The polynucleotide may be a polynucleotide as disclosed hereinabove or disclosed elsewhere herein. The polynucleotide may comprise a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15.

### LF92 Formulations

In an aspect, the lipid composition of the present disclosure comprises a cationic lipid having a structural formula (I′):

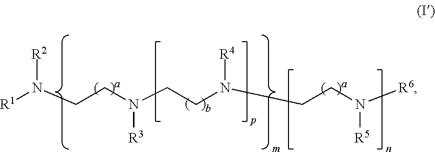

wherein:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (I′) may be protonated to provide a cationic lipid.

In some embodiments of the cationic lipid of formula (I′), a is 1. In some embodiments of the cationic lipid of formula (I′), b is 2. In some embodiments of the cationic lipid of formula (I′), m is 1. In some embodiments of the cationic lipid of formula (I′), n is 1. In some embodiments of the cationic lipid of formula (I′), R¹, R², R³, R⁴, R⁵, and R⁶ are each independently H or —CH₂CH(OH)R⁷. In some embodiments of the cationic lipid of formula (I′), R¹, R², R³, R⁴, R⁵, and R⁶ are each independently H or

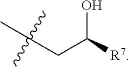

In some embodiments of the cationic lipid of formula (I′), R¹, R², R³, R⁴, R⁵, and R⁶ are each independently H or

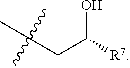

In some embodiments of the cationic lipid of formula (I′), R⁷ is C₃-C₁₈ alkyl (e.g., C₆-C₁₂ alkyl).

In some embodiments, the cationic lipid of formula (I′) is 13,16,20-tris(2-hydroxydodecyl)-13,16,20,23-tetraazapentatricontane-11,25-diol:

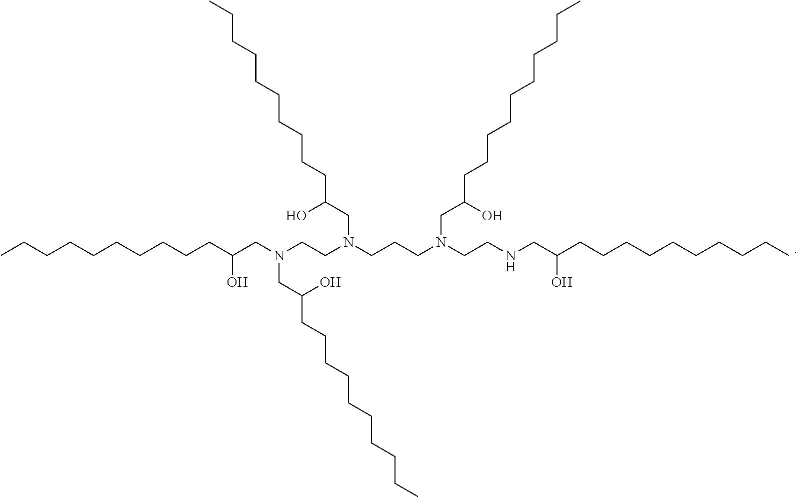 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid of formula (I′) is (11R,25R)-13,16,20-tris((R)-2-hydroxydodecyl)-13,16,20,23-tetraazapentatricontane-11,25-diol:

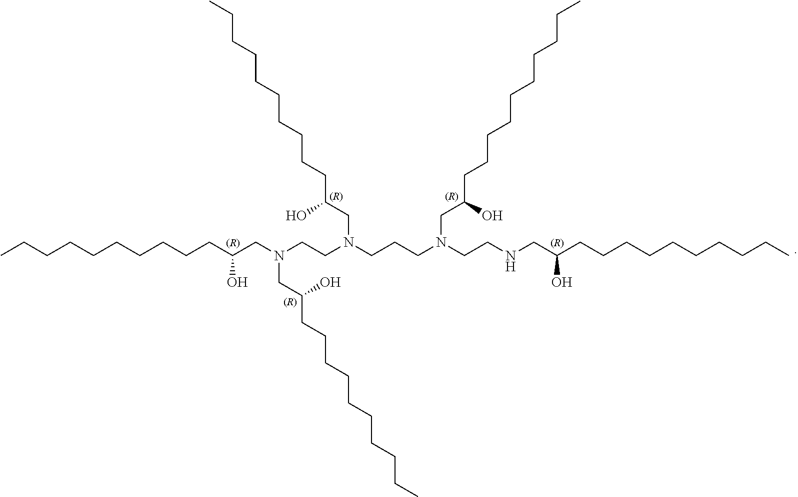

In some embodiments of the LF92 lipid composition, a lipid of the lipid composition can be in a particular amount or molar percentage. In some embodiments, the lipid composition comprises the cationic lipid of formula (I′) at a molar percentage of no more than 50% (e.g., no more than 45%). In some embodiments, the LF92 lipid composition further comprises a phospholipid. In some embodiments, the phospholipid is present in the LF92 lipid composition at a molar percentage of at least about 10%, 15%, 20%, or 25%. In some embodiments, the phospholipid is present in the LF92 lipid composition at a molar percentage of at most about 40%, 35%, or 30%. In some embodiments, the phospholipid is present in the LF92 lipid composition at a molar percentage of about 10%, 15%, 20%, 25%, 30%, 35%, or 40%, or any range between any two of the foregoing. In some embodiments, the phospholipid is present in the LF92 lipid composition at a molar percentage of 10% to 40%, or 20% to 40%. In some embodiments, lipid composition further comprises a steroid or steroid derivative. In some embodiments, the lipid composition further comprises a polymer-conjugated lipid (e.g., poly(ethylene glycol) (PEG)-conjugated lipid).

### SORT Formulations

In another aspect, the lipid composition of the present disclosure comprises (i) an ionizable cationic lipid, and (ii) a selective organ targeting (SORT) lipid separate from the ionizable cationic lipid. The lipid composition may further comprise a phospholipid. In some embodiments, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 5 to about 8. In some embodiments, the ionizable cationic lipid is a dendrimer or dendron. In some embodiments, the ionizable cationic lipid comprises at least two C₆-C₂₄ alkyl or alkenyl groups.

### Dendrimers or Dendrons of Formula (I)

In some embodiments, the ionizable cationic lipid comprises at least two C₈-C₂₄ alkyl groups. In some embodiments, the ionizable cationic lipid is a dendrimer or dendron further defined by the formula:

### Core-Repeating Unit-Terminating Group (I)

wherein the core is linked to the repeating unit by removing one or more hydrogen atoms from the core and replacing the atom with the repeating unit and wherein:
the core has the formula:

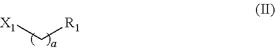

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof;
   R₁ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

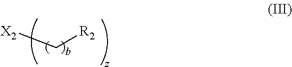

wherein:
   X₂ is N(R⁵)_{y};
   R⁵ is hydrogen, alkyl_{(C≤18)}, or substituted alkyl_{(C≤18)}; and
   y is 0, 1, or 2, provided that the sum of y and z is 3;
   R² is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3; or
the core has the formula:

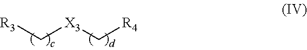

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))_{c}R_{d}

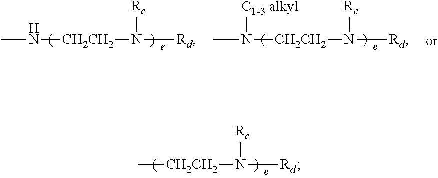

wherein:
   e and f are each independently 1, 2, or 3; provided that the sum of e and f is 3;
   R_{c}, R_{d}, and R_{f} are each independently hydrogen, alkyl_{(c≤6)}, or substituted alkyl_{(c≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
   the degradable diacyl group has the formula:

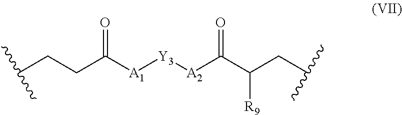

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted

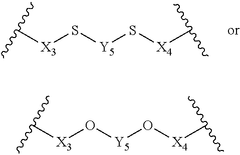

wherein:
   X₃ and X₄ are alkanediyl_{(C≤12)}, alkenediyl_{(C≤12)}, arenediyl_{(C≤12)}, or a substituted version of any of these groups;
   Y₅ is a covalent bond, alkanediyl_{(C≤12)}, alkenediyl_{(C≤12)}, arenediyl_{(C≤12)}, or a substituted version of any of these groups; and
   R₉ is alkyl_{(C≤8)} or substituted alkyl_{(C≤8)};
the linker group has the formula:

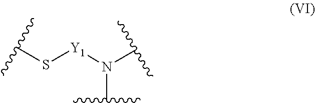

wherein:
   Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

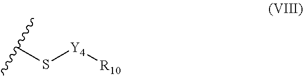

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, —Cl, —Br, —I, —SH, —OCH₃, —OCH₂CH₃, —SCH₃, or —OC(O)CH₃;
   R₁₀ is hydrogen, carboxy, hydroxy, or
   aryl_{(C≤12)}, alkylamino_{(C≤12)}, dialkylamino_{(C≤12)}, N-heterocycloalkyl_{(C<12)}, —C(O)N(R₁₁)-alkanediyl_{(C≤6)}-heterocycloalkyl_{(C≤12)}, —C(O)-alkyl-amino_{(C≤12)}, —C(O)-dialkylamino_{(C≤12)}, —C(O)—N-heterocycloalkyl_{(C≤12)}, wherein:
   R₁₁ is hydrogen, alkyl_{(C≤6)}, or substituted alkyl_{(C≤6)};
   wherein the final degradable diacyl in the chain is attached to a terminating group;
   n is 0, 1, 2, 3, 4, 5, or 6;
or a pharmaceutically acceptable salt thereof. In some embodiments, the terminating group is further defined by the formula:

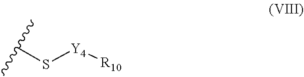

wherein:
   Y₄ is alkanediyl_{(C≤18)}; and
   R₁₀ is hydrogen. In some embodiments, A₁ and A₂ are each independently —O— or —NRₐ—.

In some embodiments, the core is further defined by the formula:

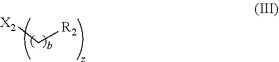

wherein:
   X₂ is N(R₅)_{y};
      R₅ is hydrogen or alkyl_{(C≤8)}, or substituted alkyl_{(C≤18)}; and
      y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3.

In some embodiments the core is further defined by the formula:

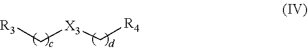

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))_{c}R_{d},

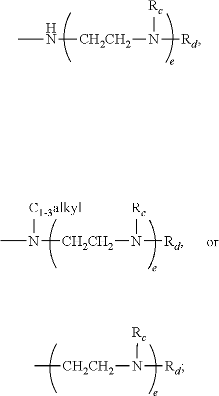

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6.

In some embodiments, the terminating group is represented by the formula:

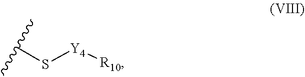

wherein:
Y₄ is alkanediyl_{(C≤18)}; and
R₁₀ is hydrogen.

In some embodiments, the core is further defined as:

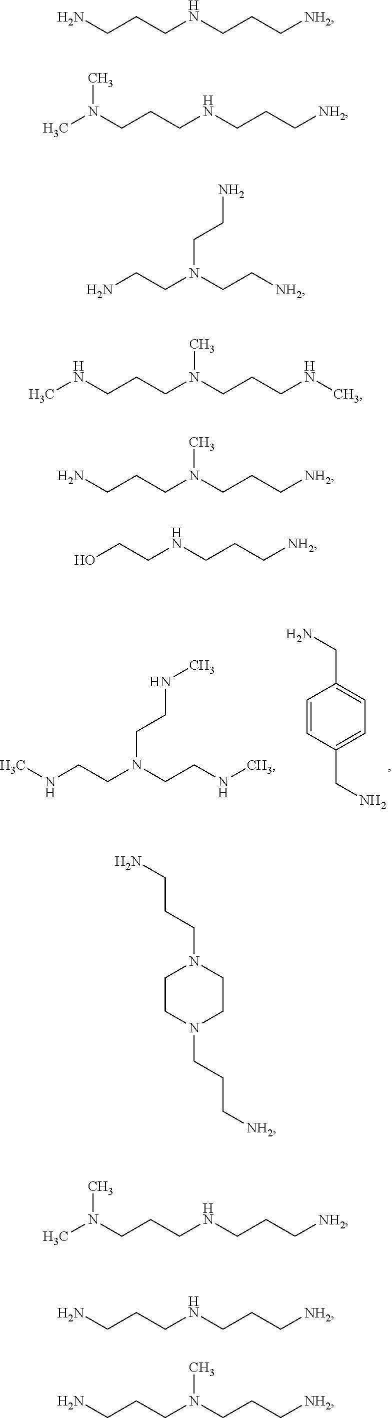

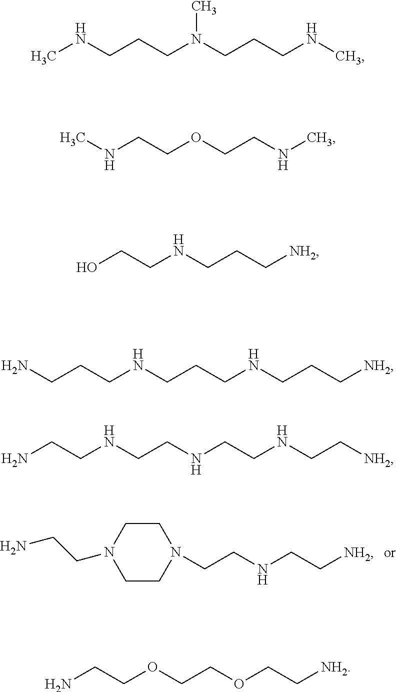

In some embodiments, the degradable diacyl is further defined as:

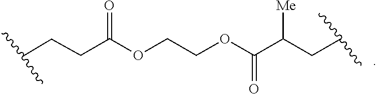 [SMILES: *CCC(=O)OCCOC(=O)C(C)C*]

In some embodiments, the linker is further defined as

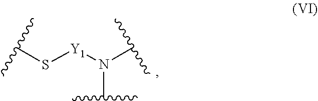

wherein Y₁ is alkanediyl_{(C≤8)} or substituted alkanediyl_{(C≤8)}.

In some embodiments, the dendrimer or dendron is further defined as:

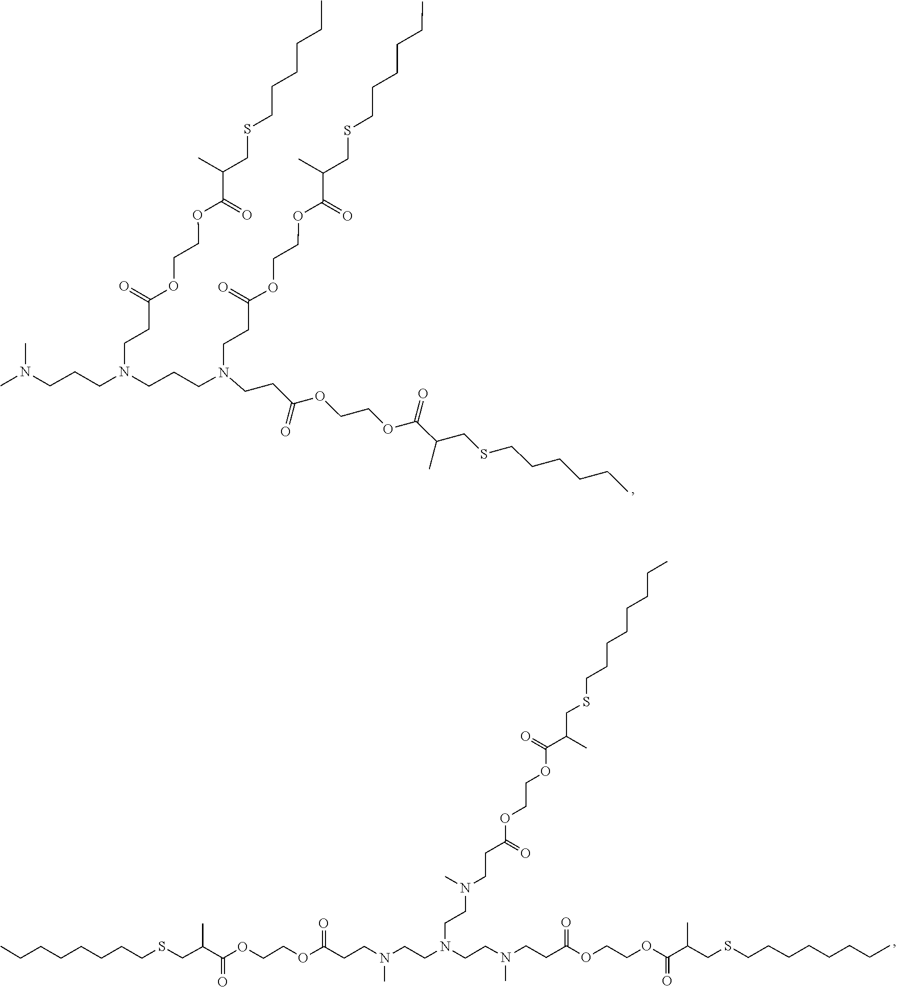

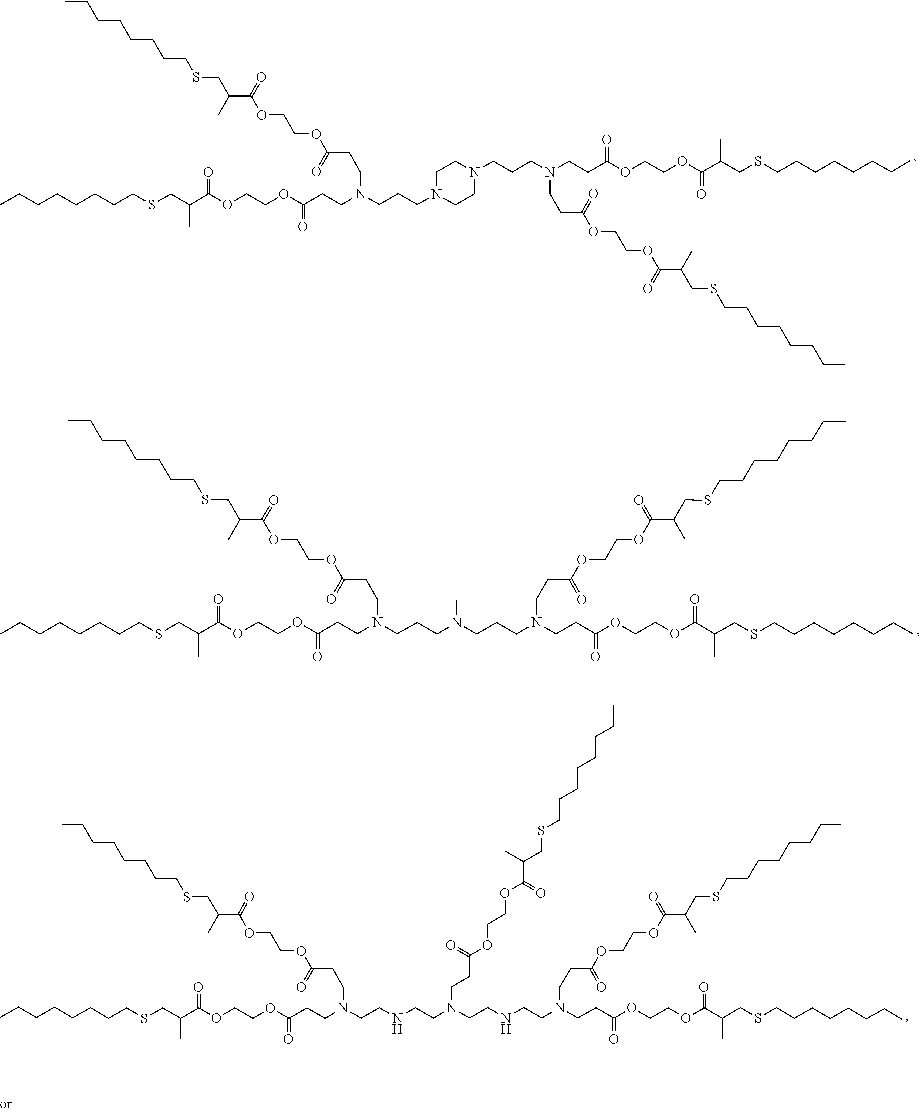

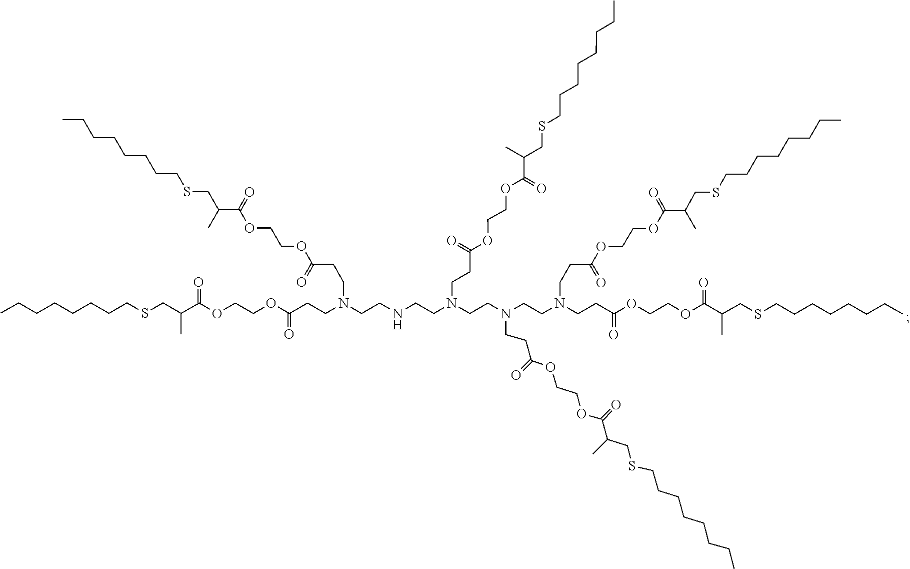 [SMILES: CCCCCCCCSCC(C)C(=O)OCCOC(=O)CCN(CCNCCN(CCC(=O)OCCOC(=O)C(C)CSCCCCCCCC)CCN(CCC(=O)OCCOC(=O)C(C)CSCCCCCCCC)CCN(CCC(=O)OCCOC(=O)C(C)CSCCCCCCCC)CCC(=O)OCCOC(=O)C(C)CSCCCCCCCC)CCC(=O)OCCOC(=O)C(C)CSCCCCCCCC]

or a pharmaceutically acceptable salt thereof

In some embodiments, an ionizable cationic lipid in the lipid composition comprises lipophilic and cationic components, wherein the cationic component is ionizable. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof

In some aspects of the present disclosure, composition containing compounds containing lipophilic and cationic components, wherein the cationic component is ionizable, are provided. In some embodiments, ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C₆-C₂₄ alkyl or alkenyl groups, but may have at least 1 or more that 6 tails. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvomic and Donald A. Tomalia in Chem. in Britain, 641-645, August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to describe compounds described herein which have a dendron like appearance (e.g., molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequential reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation.

Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers or dendrons requires a level of synthetic control achieved through series of stepwise reactions comprising building the dendrimer or dendron by each consecutive group. Dendrimer or dendron synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some aspects, the dendrimers or dendrons that may be used in the present compositions are assembled using the differential reactivity of the acrylate and methacrylate groups with amines and thiols. The dendrimers or dendrons may include secondary or tertiary amines and thioethers formed by the reaction of an acrylate group with a primary or secondary amine and a methacrylate with a mercapto group. Additionally, the repeating units of the dendrimers or dendrons may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer or dendron may be formed by removing one or more hydrogen atoms from this core. In some embodiments, these hydrogen atoms are on a heteroatom such as a nitrogen atom. In some embodiments, the terminating group is a lipophilic group such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—C(O)OH). In still other embodiments, the terminating group is an aliphatic or aromatic group containing one or more hydrogen bond donors such as a hydroxide group, an amide group, or an ester.

### Dendrimers or Dendrons of Formula (X)

In some embodiments, the ionizable cationic lipid is a dendrimer or dendron of the formula Core (Branch)N. In some embodiments, the ionizable cationic lipid is a dendrimer or dendron of the formula

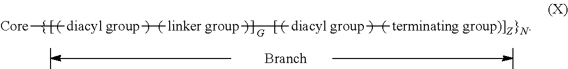

In some embodiments, the ionizable cationic lipid is a dendrimer or dendron of a generation (g) having a structural formula:

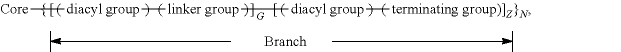

or a pharmaceutically acceptable salt thereof, wherein:
(a) the core comprises a structural formula (X_{Core}):

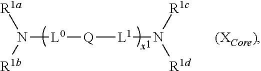

wherein:
   Q is independently at each occurrence a covalent bond, —O—, —S—, —NR²—, or —CR³aR³b—;
   R² is independently at each occurrence R^{1g} or —L²—NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

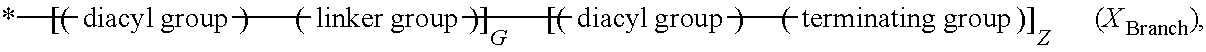

wherein:
   indicates a point of attachment of the branch to the core;
   g is 1, 2, 3, or 4;
   Z=2^{(g-1)};
   G=0, when g=1; or G=Σᵢ₌₀^{i=g-2}2ⁱ, when g≠1;
(c) each diacyl group independently comprises a structural formula

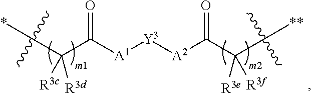

wherein
indicates a point of attachment of the diacyl group at the proximal end thereof;
indicates a point of attachment of the diacyl group at the distal end thereof;
Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR⁴—
   wherein:
   R₄ is hydrogen or optionally substituted (e.g., C₁-C₆) alkyl;
m¹ and m² are each independently at each occurrence 1, 2, or 3; and R^{3c}, R^{3d}, R^{3e}, and R^{3f} are each independently at each occurrence hydrogen or an optionally substituted (e.g., C₁-C₈) alkyl; and
(d) each linker group independently comprises a structural formula

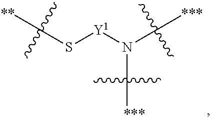

wherein:
   indicates a point of attachment of the linker to a proximal diacyl group;
   indicates a point of attachment of the linker to a distal diacyl group; and
   Y¹ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²—, or —CR^{3a}R^{3b}. In some embodiments of X_{Core} Q is independently at each occurrence a covalent bond. In some embodiments of X_{Core} Q is independently at each occurrence an —O—. In some embodiments of X_{Core} Q is independently at each occurrence a —S—. In some embodiments of X_{Core} Q is independently at each occurrence a —NR² and R² is independently at each occurrence R^{1g} or —L²—NR^{1e}R^{1f}. In some embodiments of X_{Core} Q is independently at each occurrence a —CR^{3a}R^{3b}R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1f}, R^{1e}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L¹ form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{Core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g., C₁-C₁₂, such as C₁-C₈ or C₁-C₆). In some embodiments of X_{Core}, L⁰, L¹, and L² are each independently at each occurrence can be a heteroalkylene (e.g., C₂-C₈ alkyleneoxide, such as oligo(ethyleneoxide)). In some embodiments of X_{Core}, L⁰, L¹, and L² are each independently at each occurrence can be a [alkylene]-[heterocycloalkyl]-[alkylene] [(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene] [(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

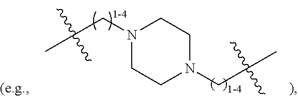

and [(C₁-C₄) alkylene]-phenylene-[(C₁-C₄) alkylene]

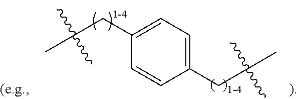

In some embodiments of X_{Core}, L⁰, L¹, and L² are each independently at each occurrence selected from C₁-C₆ alkylene (e.g., C₁-C₃ alkylene), —(C₁-C₃ alkylene-0)_{1.4}—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂—C₈) alkyleneoxide (e.g., —(C₁-C₃ alkylene-0)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments Of X_{Core}, x¹ is 0. In some embodiments Of X_{Core}, x¹ is 1. In some embodiments Of X_{Core} x¹ is 2. In some embodiments Of X_{Core}, x¹ is 0, 3. In some embodiments Of X_{Core} x¹ is 4. In some embodiments of X_{Core} x¹ is 5. In some embodiments Of X_{Core}, x¹ is 6.

In some embodiments Of X_{Core}, the core comprises a structural formula:

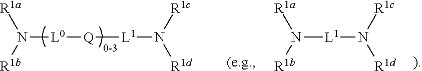

In some embodiments Of X_{Core}, the core comprises a structural formula:

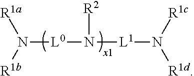

In some embodiments of X_{Core}, the core comprises a structural formula:

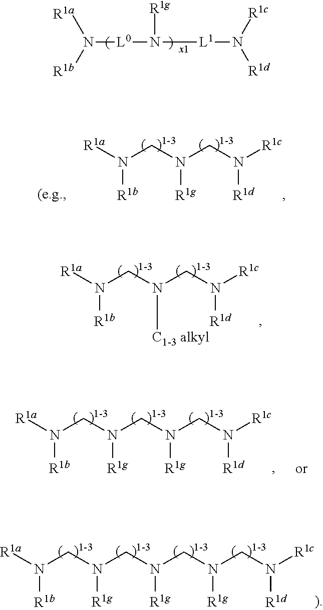

In some embodiments Of X_{Core}, the core comprises a structural formula

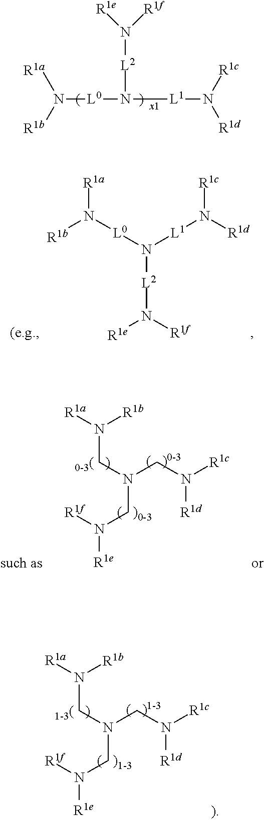

In some embodiments of X_{core} the core comprises a structural formula

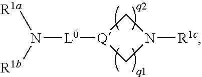

wherein Q′ is —NR²— or —CR^{3a}R^{3b}—; q¹ and q² are each independently 1 or 2. In some embodiments Of X_{Core}, the core
comprises a structural formula:

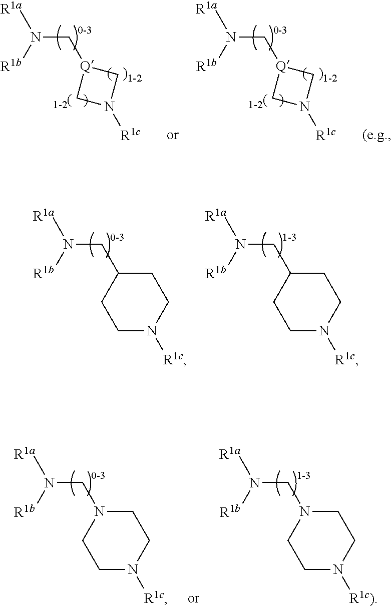

In some embodiments Of X_{Core}, the core comprises a structural formula

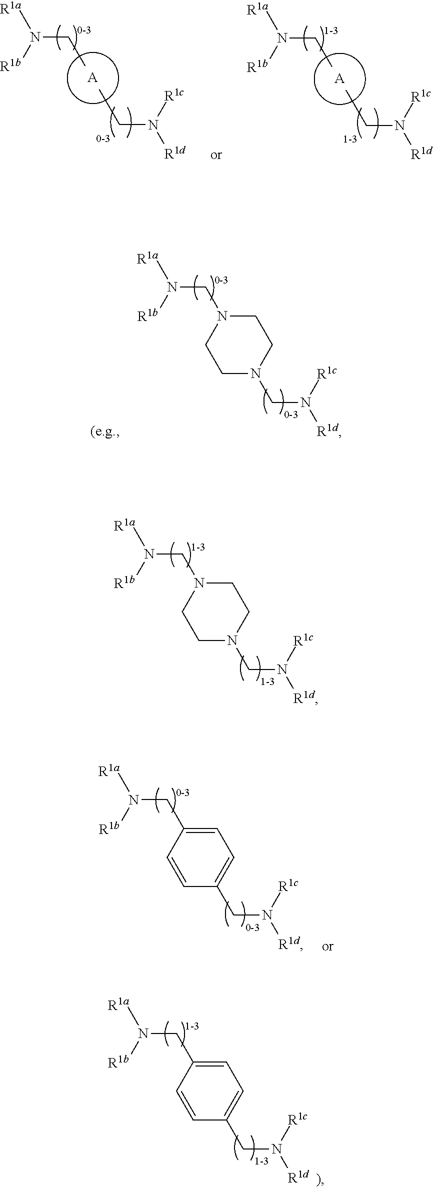

wherein ring A is an optionally substituted aryl or an optionally substituted (e.g., C₃-C₁₂, such as C₃-C₅) heteroaryl. In some embodiments of X_{Core}, the core comprises has a structural formula

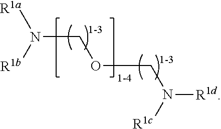

In some embodiments of X_{Core}, the core comprises a structural formula set forth in Table. 3 and pharmaceutically acceptable salts thereof, wherein * indicates a point of attachment of the core to a branch of the plurality of branches. In some embodiments, the example cores of Table. 3 are not limiting of the stereoisomers (i.e., enantiomers, diastereomers) listed.

TABLE 3
Example core structures

| ID # | Structure |
|---|---|
| 1A1 | 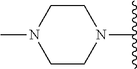 [SMILES: *N1CCN(C)CC1] |
| 1A2-1 | 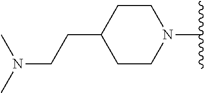 [SMILES: *N1CCC(CCN(C)C)CC1] |
| 1A2-2 | 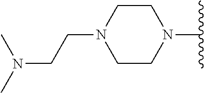 [SMILES: *N1CCN(CCN(C)C)CC1] |
| 1A3-1 | 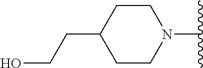 [SMILES: *N1CCC(CCO)CC1] |
| 1A3-2 | 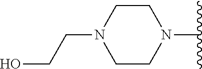 [SMILES: *N1CCN(CCO)CC1] |
| 1A4 | 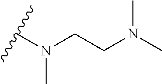 [SMILES: *N(C)CCN(C)C] |
| 1A5-1 | 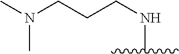 [SMILES: *NCCCN(C)C] |
| 1A5-2 | 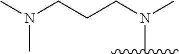 |
| 2A1-1 | 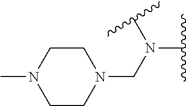 [SMILES: *N(*)CN1CCN(C)CC1] |
| 2A1-2 | 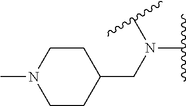 [SMILES: *N(*)CC1CCN(C)CC1] |
| 2A2-1 | 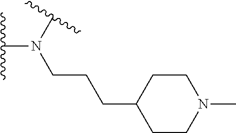 [SMILES: *N(*)CCCC1CCN(C)CC1] |
| 2A2-2 | 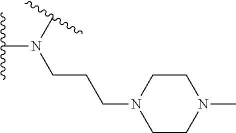 [SMILES: *N(*)CCCN1CCN(C)CC1] |
| 2A3 | 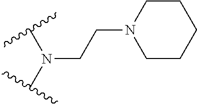 [SMILES: *N(*)CCN1CCCCC1] |
| 2A4 | 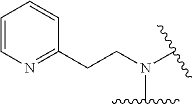 [SMILES: *N(*)CCc1ccccn1] |
| 2A5 | 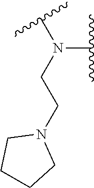 [SMILES: *N(*)CCN1CCCC1] |
| 2A6 | 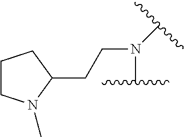 [SMILES: *N(*)CCC1CCCN1C] |
| 2A7 | 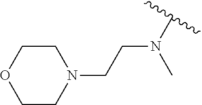 [SMILES: *N(C)CCN1CCOCC1] |
| 2A7-2 | 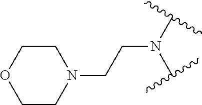 [SMILES: *N(*)CCN1CCOCC1] |
| 2A8 | 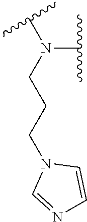 [SMILES: *N(*)CCCn1ccnc1] |
| 2A9 | 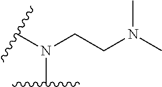 [SMILES: *N(*)CCN(C)C] |
| 2A9V | 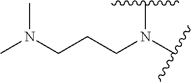 [SMILES: *N(*)CCCN(C)C] |
| 2A10 | 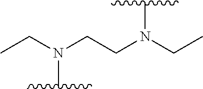 [SMILES: *N(CC)CCN(*)CC] |
| 2A11 | 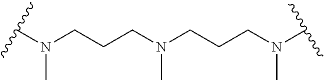 [SMILES: *N(C)CCCN(C)CCCN(*)C] |
| 2A12 | 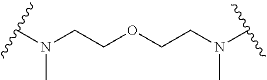 [SMILES: *N(C)CCOCCN(*)C] |
| 3A1 | 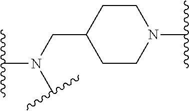 [SMILES: *N(*)CC1CCN(*)CC1] |
| 3A2 | 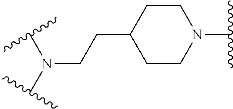 [SMILES: *N(*)CCC1CCN(*)CC1] |
| 3A3 | 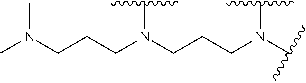 [SMILES: *N(*)CCCN(*)CCCN(C)C] |
| 3A4 | 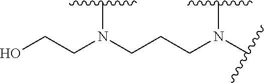 [SMILES: *N(*)CCCN(*)CCO] |
| 3A5 | 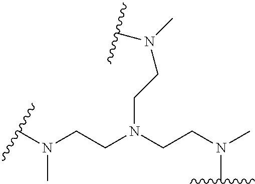 [SMILES: *N(C)CCN(CCN(*)C)CCN(*)C] |
| 3A6 | 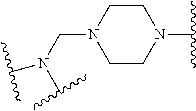 [SMILES: *N(*)CN1CCN(*)CC1] |
| 3A7 | 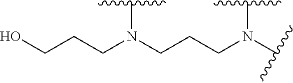 [SMILES: *N(*)CCCN(*)CCCO] |
| 4A1 | 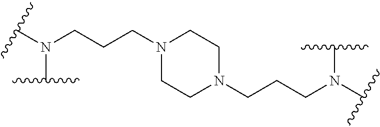 [SMILES: *N(*)CCCN1CCN(CCCN(*)*)CC1] |
| 4A2 | 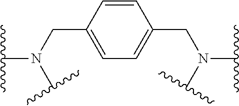 [SMILES: *N(*)Cc1ccc(CN(*)*)cc1] |
| 4A3 | 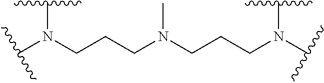 [SMILES: *N(*)CCCN(C)CCCN(*)*] |
| 4A4 | 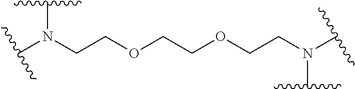 [SMILES: *N(*)CCOCCOCCN(*)*] |
| 5A1 | 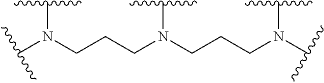 [SMILES: *N(*)CCCN(*)CCCN(*)*] |
| 5A2-1 (5-arm) | 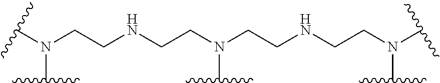 [SMILES: *N(*)CCNCCN(*)CCNCCN(*)*] |
| 5A2-2 (5-arm) | 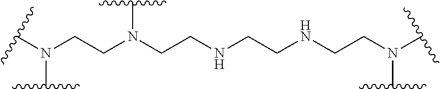 [SMILES: *N(*)CCNCCNCCN(*)CCN(*)*] |
| 5A2-3 (5-arm) | 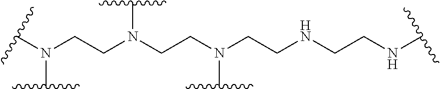 [SMILES: *NCCNCCN(*)CCN(*)CCN(*)*] |
| 5A2-4 (5-arm) | 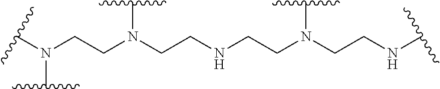 [SMILES: *NCCN(*)CCNCCN(*)CCN(*)*] |
| 5A3-1 (5-arm) | 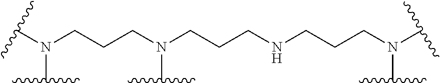 [SMILES: *N(*)CCCNCCCN(*)CCCN(*)*] |
| 5A4-1 (5-arm) | 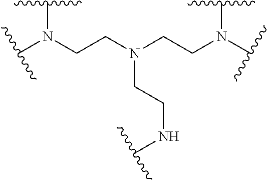 [SMILES: *NCCN(CCN(*)*)CCN(*)*] |
| 5A5 | 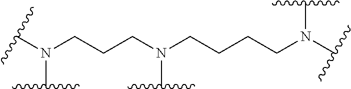 [SMILES: *N(*)CCCCN(*)CCCN(*)*] |
| 5A6 | 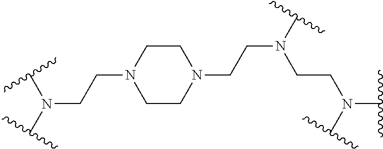 [SMILES: *N(*)CCN(*)CCN1CCN(CCN(*)*)CC1] |
| 5A2-4 (6-arm) | 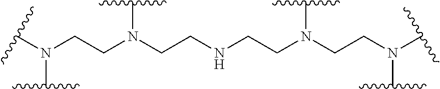 [SMILES: *N(*)CCN(*)CCNCCN(*)CCN(*)*] |
| 5A2-5 (6-arm) | 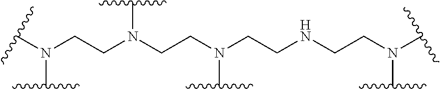 [SMILES: *N(*)CCNCCN(*)CCN(*)CCN(*)*] |
| 5A2-6 (6-arm) | 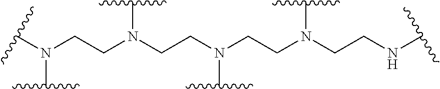 [SMILES: *NCCN(*)CCN(*)CCN(*)CCN(*)*] |
| 5A3-2 (6-arm) | 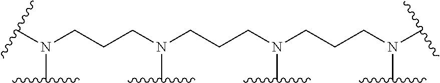 [SMILES: *N(*)CCCN(*)CCCN(*)CCCN(*)*] |
| 5A4-2 (6-arm) | 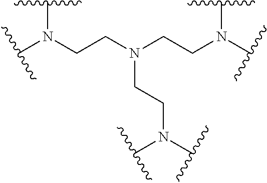 [SMILES: *N(*)CCN(CCN(*)*)CCN(*)*] |
| 6A4 | 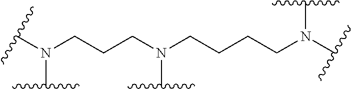 [SMILES: *N(*)CCCCN(*)CCCN(*)*] |
| 1H1 | 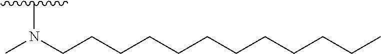 [SMILES: *N(C)CCCCCCCCCCCC] |
| 1H2 | 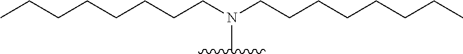 [SMILES: *N(CCCCCCCC)CCCCCCCC] |
| 1H3 | 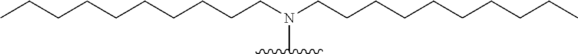 [SMILES: *N(CCCCCCCCCC)CCCCCCCCCC] |
| 2H1 | 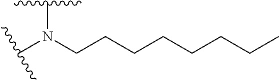 [SMILES: *N(*)CCCCCCCC] |
| 2H2 | 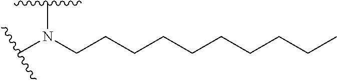 [SMILES: *N(*)CCCCCCCCCC] |
| 2H3 | 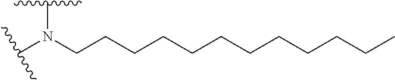 [SMILES: *N(*)CCCCCCCCCCCC] |
| 2H4 | 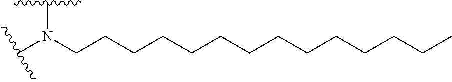 [SMILES: *N(*)CCCCCCCCCCCCCC] |
| 2H5 | 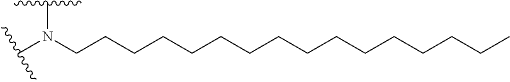 [SMILES: *N(*)CCCCCCCCCCCCCCCC] |
| 2H6 | 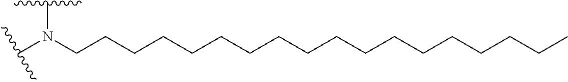 [SMILES: *N(*)CCCCCCCCCCCCCCCCCC] |

In some embodiments of X_{core}, the core comprises a structural formula selected from the group consisting of:

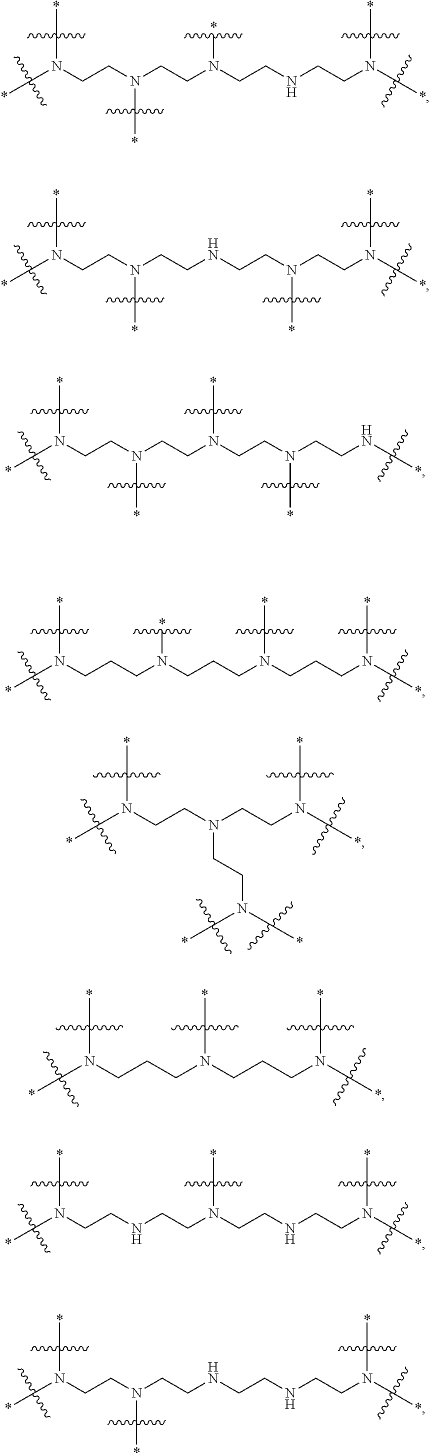

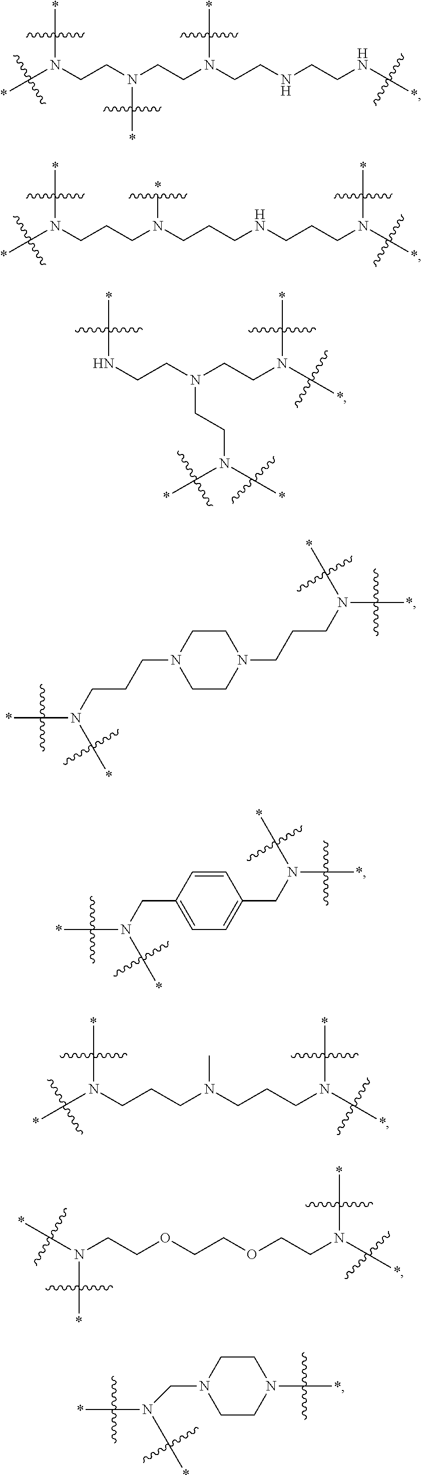

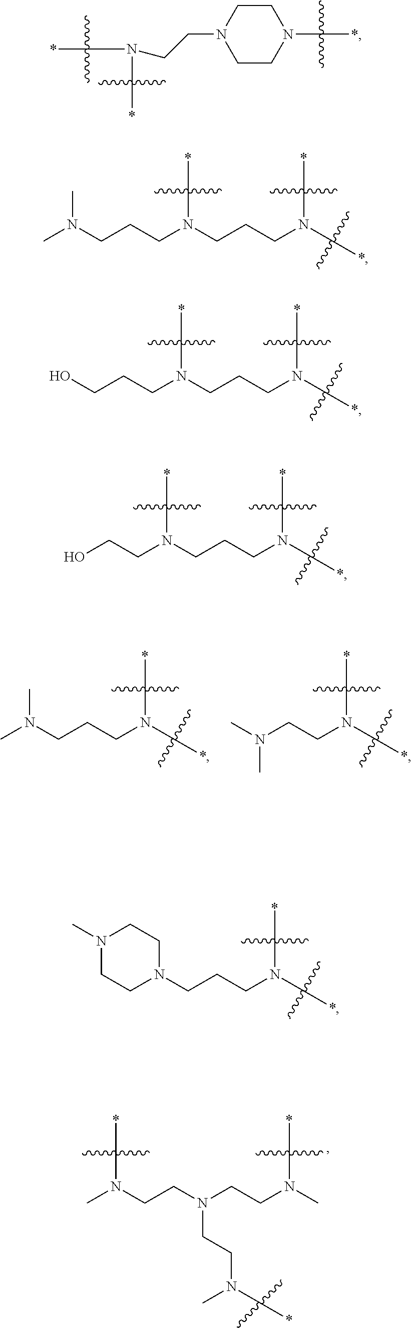

and pharmaceutically acceptable salts thereof, wherein * indicates a point of attachment of the core to a branch of the plurality of branches or H. In some embodiments, wherein * indicates a point of attachment of the core to a branch of the plurality of branches.

In some embodiments of X_{Core}, the core has the structure

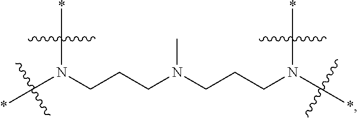

wherein * indicates a point of attachment of the core to a branch of the plurality of branches or H. In some embodiments, at least 2 branches are attached to the core. In some embodiments, at least 3 branches are attached to the core. In some embodiments, at least 4 branches are attached to the core.

In some embodiments of X_{Core}, the core has the structure

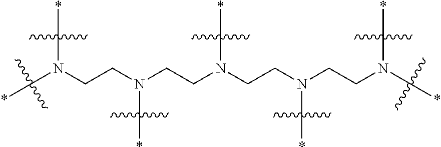

wherein * indicates a point of attachment of the core to a branch of the plurality of branches or H. In some embodiments, at least 4 branches are attached to the core. In some embodiments, at least 5 branches are attached to the core. In some embodiments, at least 6 branches are attached to the core.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of X_{Branch}, Z=2^{(g-1)} and when g=1, G=0. In some embodiments of X_{Branch}, Z=2^{(g-1)} and G=Σᵢ₌₀^{i=g-2} 2ⁱ, when g ≠1.

In some embodiments of X_{Branch}, g=1, G=0, Z=1, and each branch of the plurality of branches comprises a structural formula each branch of the plurality of branches comprises a structural formula *diacyl group terminating group).

In some embodiments of X_{Branch}, g=2, G=1, Z=2, and each branch of the plurality of branches comprises a structural formula

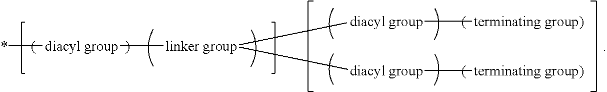

In some embodiments of X_{Branch}, g=3, G=3, Z=4, and each branch of the plurality of branches comprises a structural formula

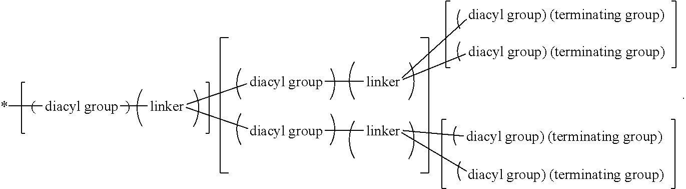

In some embodiments of X_{Branch}, g=4, G=7, Z=8, and each branch of the plurality of branches comprises a structural formula

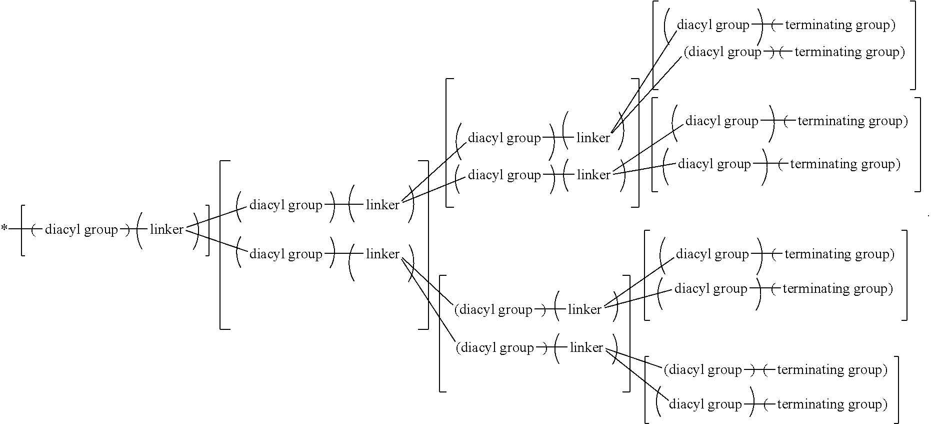

In some embodiments, the dendrimers or dendrons described herein with a generation (g)=1 has structure the structure:

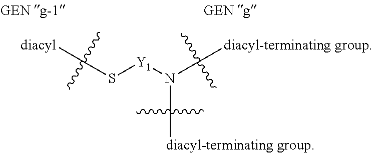

In some embodiments, the dendrimers or dendrons described herein with a generation (g)=1 has structure the structure:

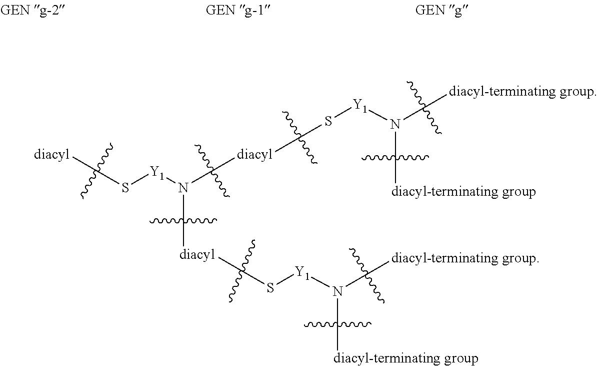

Example formulation of the dendrimers or dendrons described herein for generations 1 to 4 is shown in Table 4. The number of diacyl groups, linker groups, and terminating groups can be calculated based on g.

TABLE 4
Formulation of Dendrimer or dendron Groups Based on Generation (g)

| | g = 1 | g = 2 | g = 3 | g = 4 | |
|---|---|---|---|---|---|
| # of diacyl grp | 1 | 1 + 2 = 3 | 1 + 2 + 2² = 7 | 1 + 2 + 2² + 2³ = 15 | 1 + 2 + . . . + 2^{g-1} |
| # of linker grp | 0 | 1 | 1 + 2 | 1 + 2 + 2² | 1 + 2 + . . . + 2^{g-2} |
| # of terminating | 1 | 2 | 2² | 2³ | 2^{(g-1)} |
| grp | | | | | |

In some embodiments, the diacyl group independently comprises a structural formula

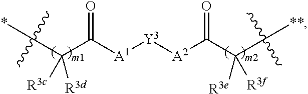

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g., C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}, A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR⁴—and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3b} R^{3d}, R^{3e}, and R^{3f} are each independently at each occurrence hydrogen or an optionally substituted alkyl. In some embodiments of the diacyl group of X_{Branch}, R^{3C}, R^{3a}, R^{3e}, and R^{3f} are each independently at each occurrence hydrogen. In some embodiments of the diacyl group of X_{Branch}, R^{3C}, R^{3a}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A¹ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

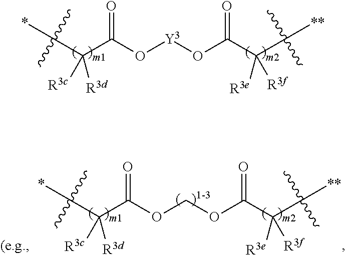

such as

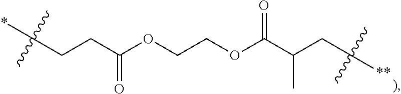

and optionally R^{3c}, R^{3d}R^{3c} and R^{3f} are each independently at each occurrence hydrogen or C₁-C₃ alkyl.

In some embodiments, linker group independently comprises a structural formula

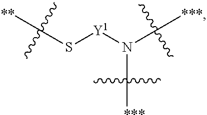

** indicates a point of attachment of the linker to a proximal diacyl group, and *** indicates a point of attachment of the linker to a distal diacyl group.

In some embodiments of the linker group Of X_{Branch} if present, Y₁ is independently at each occurrence an optionally substituted alkylene, an optionally substituted alkenylene, or an optionally substituted arenylene. In some embodiments of the linker group Of X_{Branch} if present, Y₁ is independently at each occurrence an optionally substituted alkylene (e.g., C₁-C₁₂). In some embodiments of the linker group of X_{Branch} if present, Y¹ is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

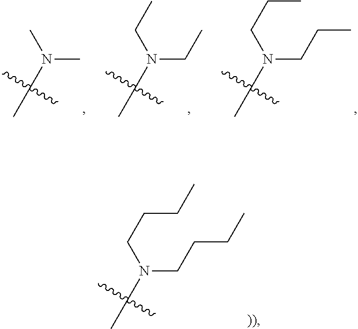

C₄-C₆ N-heterocycloalkyl (e.g., N-pyrrolidinyl

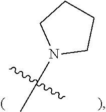 [SMILES: *N1CCCC1]

### N-piperidinyl

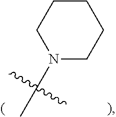 [SMILES: *N1CCCCC1]

### N-azepanyl

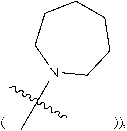 [SMILES: *N1CCCCCC1]

—OH, —C(O)OH, —C(O)N(C₁-C₃ alkyl)-(C₁-C₆ alkylene)-(C₁-C₁₂ alkylamino

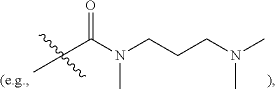 [SMILES: *C(=O)N(C)CCCN(C)C]

—C(O)N(C₁-C₃ alkyl)-(C₁-C₆ alkylene)-(C₄-C₆ N-heterocycloalkyl)

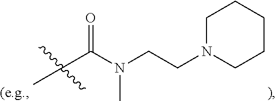 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

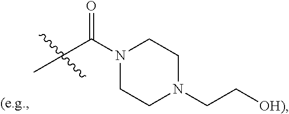

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent —OH. In some embodiments of the terminating group of X_{Branch}, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

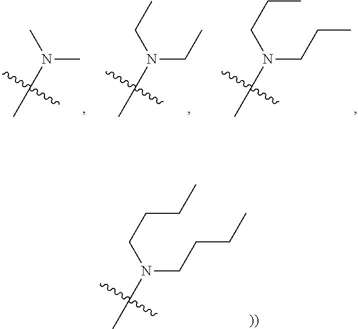

and C₄-C₆ N-heterocycloalkyl (e.g., N-pyrrolidinyl

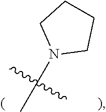 [SMILES: *N1CCCC1]

### N-piperidinyl

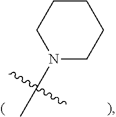 [SMILES: *N1CCCCC1]

### N-azepanyl

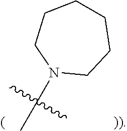 [SMILES: *N1CCCCCC1]

In some embodiments of the terminating group of X_{Branch}, each terminating group is independently C₁-₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table. 5. In some embodiments, the dendrimers or dendrons described herein can comprise a terminating group or pharmaceutically acceptable salt, or thereof selected in Table. 5. In some embodiments, the example terminating group of Table. 5 are not limiting of the stereoisomers (i.e., enantiomers, diastereomers) listed.

TABLE 5
Example terminating group/peripheries structures

| ID # | Structure |
|---|---|
| SC1 | 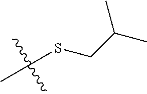 [SMILES: *SCC(C)C] |
| SC2 | 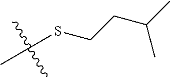 [SMILES: *SCCC(C)C] |
| SC3 | 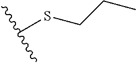 [SMILES: *SCCC] |
| SC4 | 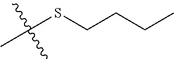 [SMILES: *SCCCC] |
| SC5 | 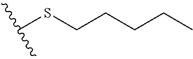 [SMILES: *SCCCCC] |
| SC6 | 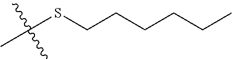 [SMILES: *SCCCCCC] |
| SC7 | 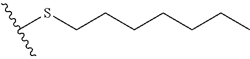 [SMILES: *SCCCCCCC] |
| SC8 | 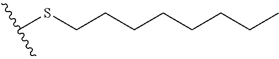 [SMILES: *SCCCCCCCC] |
| SC9 | 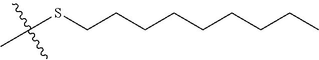 [SMILES: *SCCCCCCCCC] |
| SC10 | 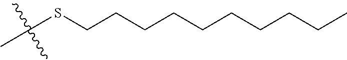 [SMILES: *SCCCCCCCCCC] |
| SC11 | 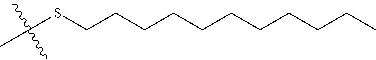 [SMILES: *SCCCCCCCCCCC] |
| SC12 | 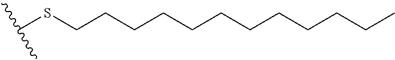 [SMILES: *SCCCCCCCCCCCC] |
| SC14 | 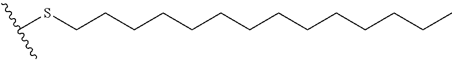 [SMILES: *SCCCCCCCCCCCCCC] |
| SC16 | 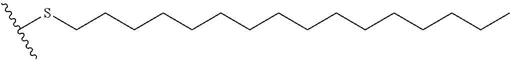 [SMILES: *SCCCCCCCCCCCCCCCC] |
| SC18 | 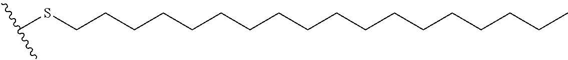 [SMILES: *SCCCCCCCCCCCCCCCCCC] |
| SC19 | 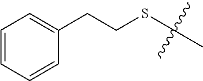 [SMILES: *SCCc1ccccc1] |
| SO1 | 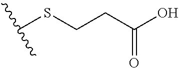 [SMILES: *SCCC(=O)O] |
| SO2 | 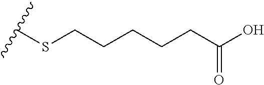 [SMILES: *SCCCCCC(=O)O] |
| SO3 | 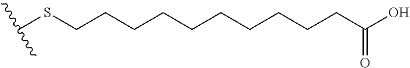 [SMILES: *SCCCCCCCCCCC(=O)O] |
| SO4 | 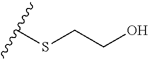 [SMILES: *SCCO] |
| SO5 | 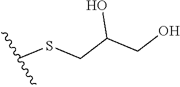 [SMILES: *SCC(O)CO] |
| SO6 | 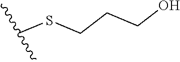 [SMILES: *SCCCO] |
| SO7 | 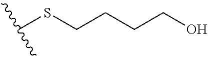 [SMILES: *SCCCCO] |
| SO8 | 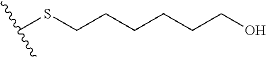 [SMILES: *SCCCCCCO] |
| SO9 | 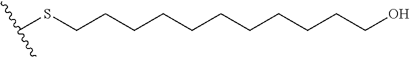 [SMILES: *SCCCCCCCCCCCO] |
| SN1 | 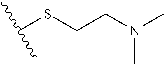 [SMILES: *SCCN(C)C] |
| SN2 | 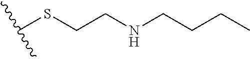 [SMILES: *SCCNCCCC] |
| SN3 | 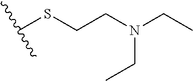 [SMILES: *SCCN(CC)CC] |
| SN4 | 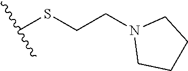 [SMILES: *SCCN1CCCC1] |
| SN5 | 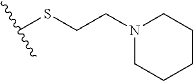 [SMILES: *SCCN1CCCCC1] |
| SN6 | 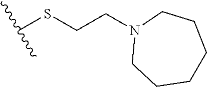 [SMILES: *SCCN1CCCCCC1] |
| SN7 | 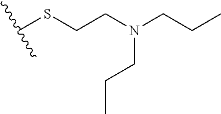 [SMILES: *SCCN(CCC)CCC] |
| SN8 | 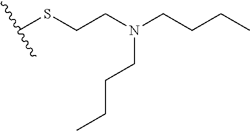 [SMILES: *SCCN(CCCC)CCCC] |
| SN9 | 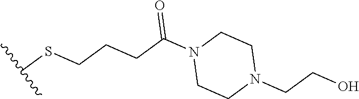 [SMILES: *SCCCC(=O)N1CCN(CCO)CC1] |
| SN10 | 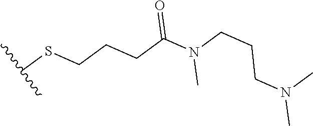 [SMILES: *SCCCC(=O)N(C)CCCN(C)C] |
| SN11 | 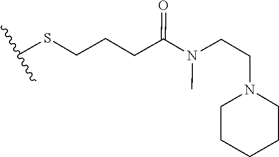 [SMILES: *SCCCC(=O)N(C)CCN1CCCCC1] |

In some embodiments, the dendrimer or dendron of Formula (X) is selected from those set forth in Table 6 and pharmaceutically acceptable salts thereof.

TABLE 6
Example ionizable cationic lipo-dendrimers or lipo-dendrons

| ID # | Structure |
|---|---|
| 2A2-SC14 | 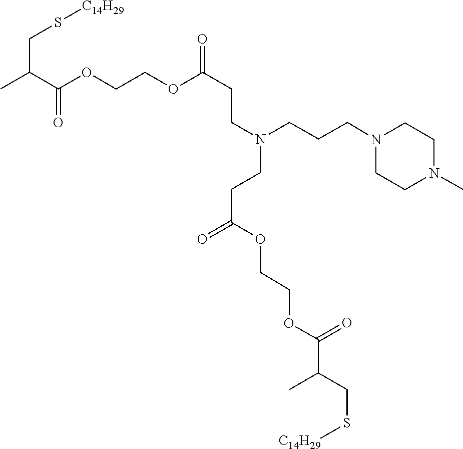 |
| 2A6-SC14 | 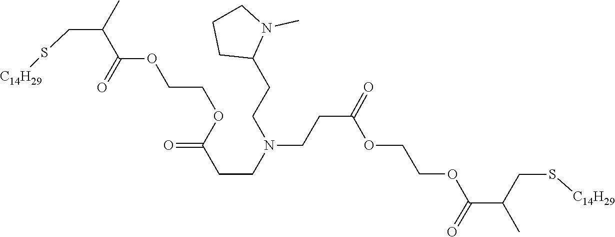 |
| 2A9-SC14 | 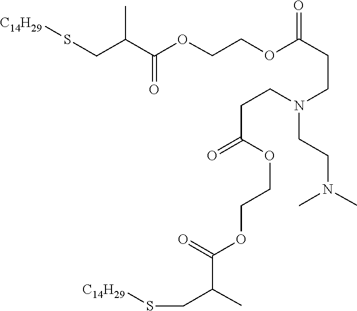 |
| 3A3-SC10 | 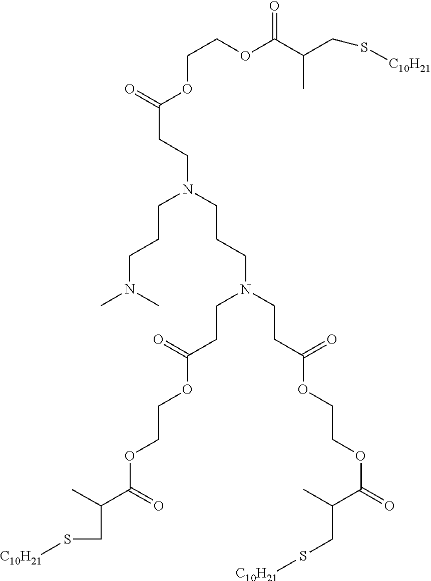 |
| 3A3-SC14 | 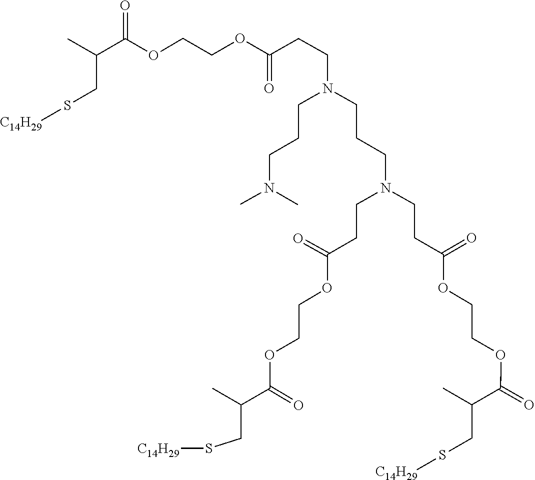 |
| 3A5-SC10 | 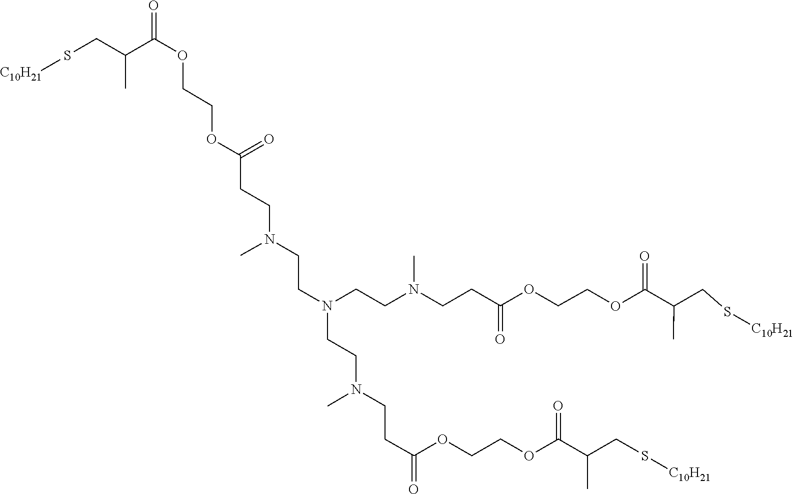 |
| 3A5-SC14 | 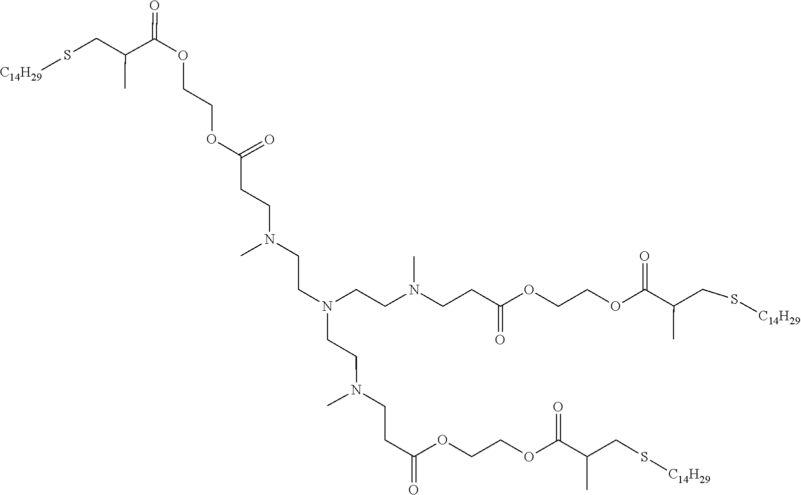 |
| 4A1-SC12 | 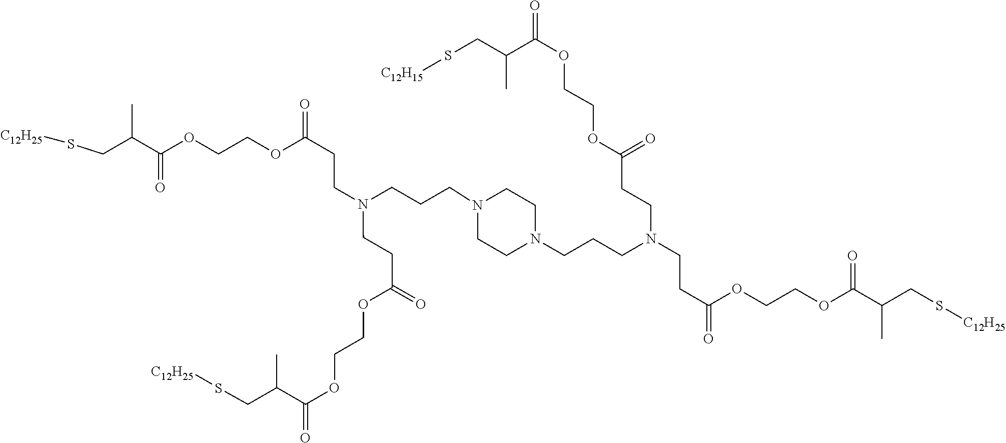 |
| 4A3-SC12 | 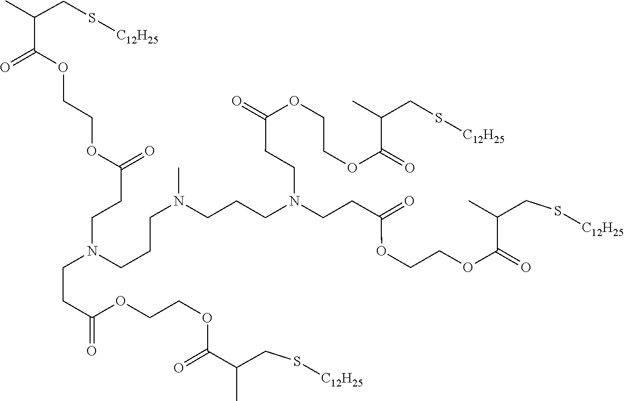 |
| 5A1-SC12 | 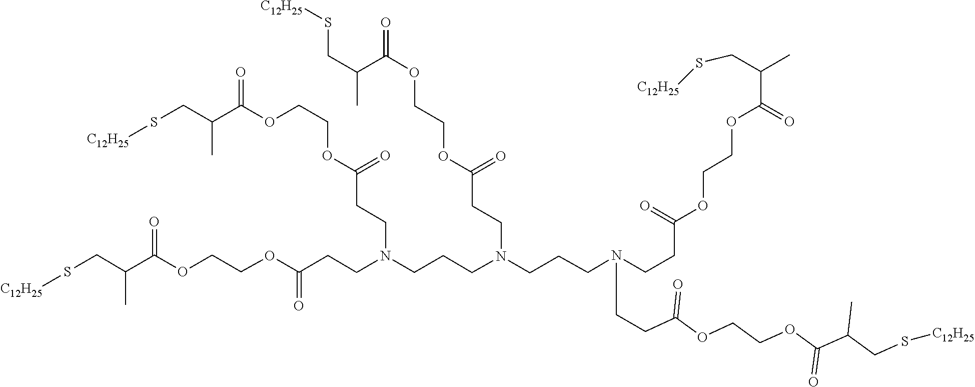 |
| 5A1-SC8 | 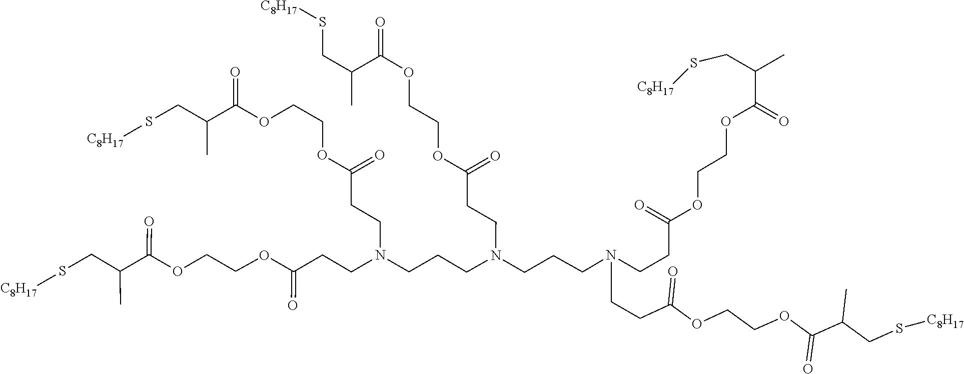 |
| 5A2-2-SC12 (5-arm) | 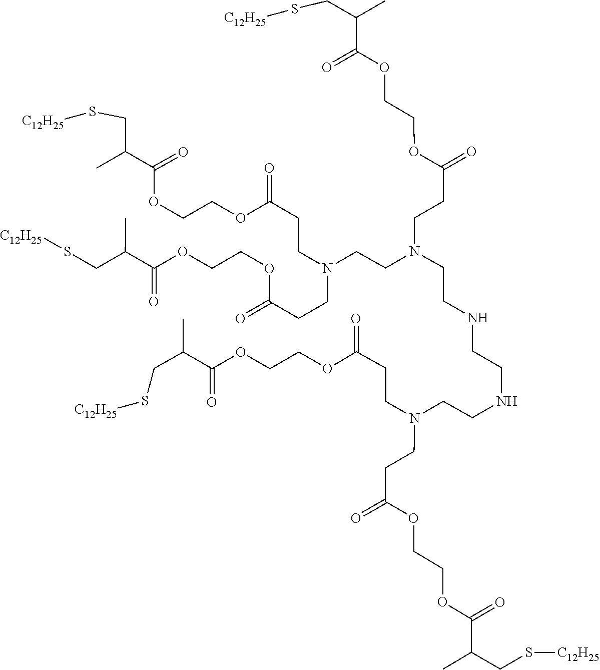 |
| 5A3-1-SC12 (5 arm) | 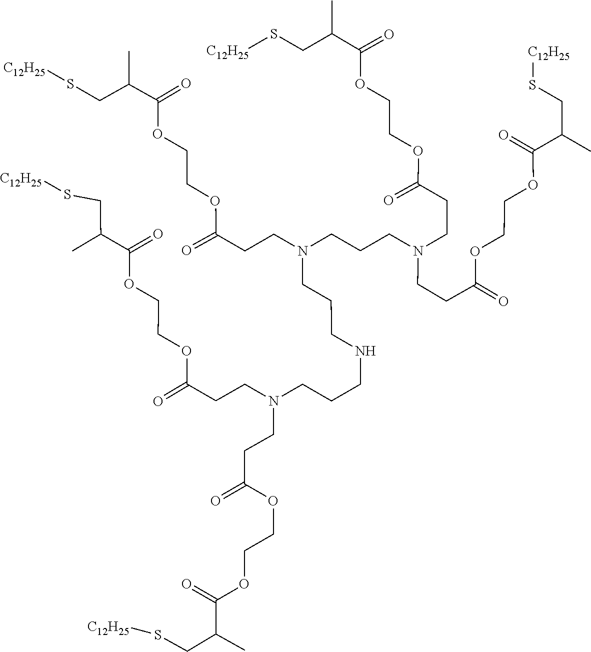 |
| 5A3-1-SC8 (5-arm) | 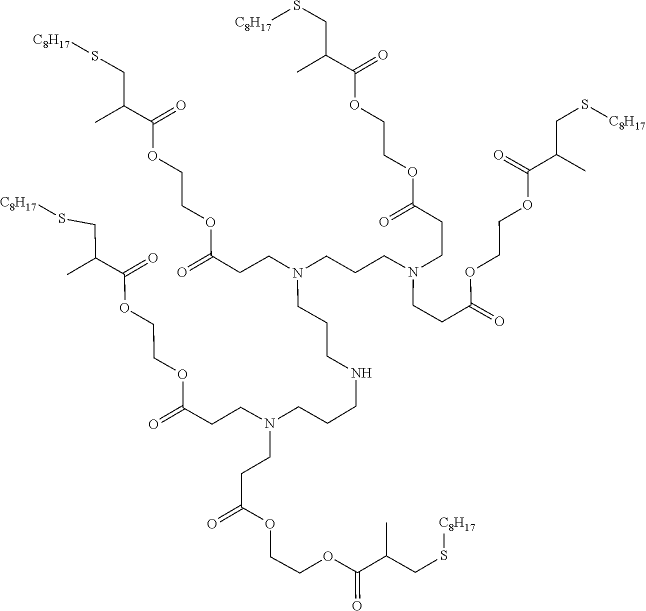 |
| 5A4-1-SC12 (5-arm) | 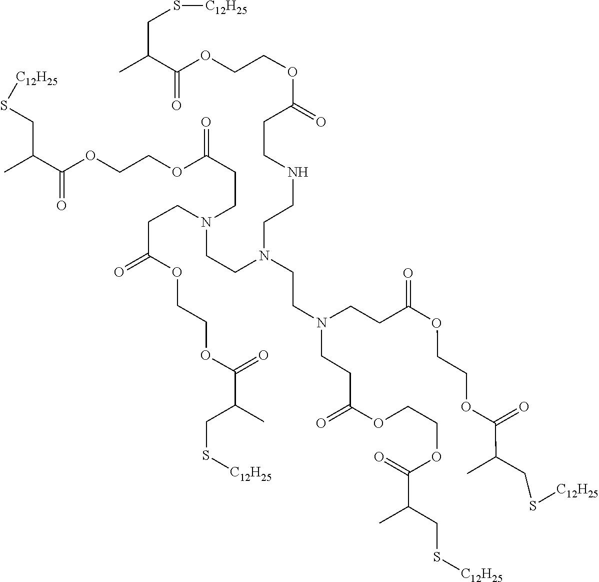 |
| 5A4-1-SC8 (5-arm) | 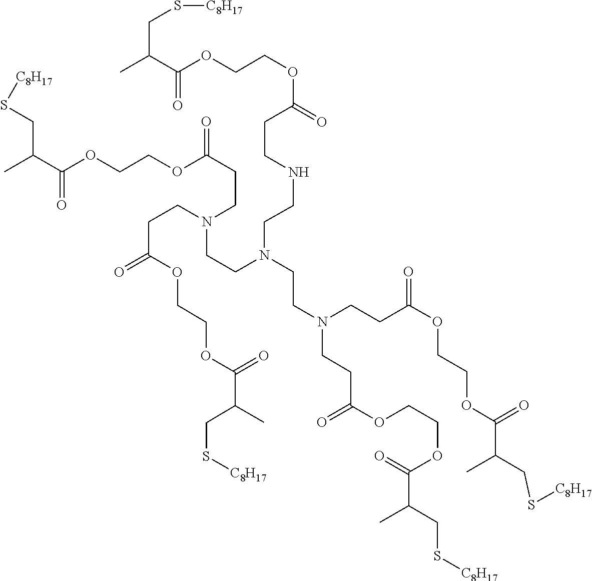 |
| 5A5-SC8 | 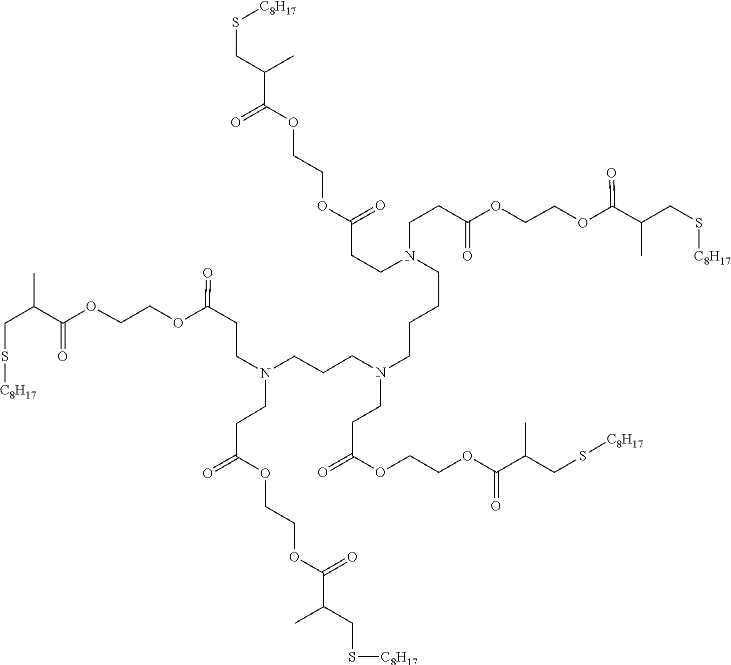 |
| 5A5-SC12 | 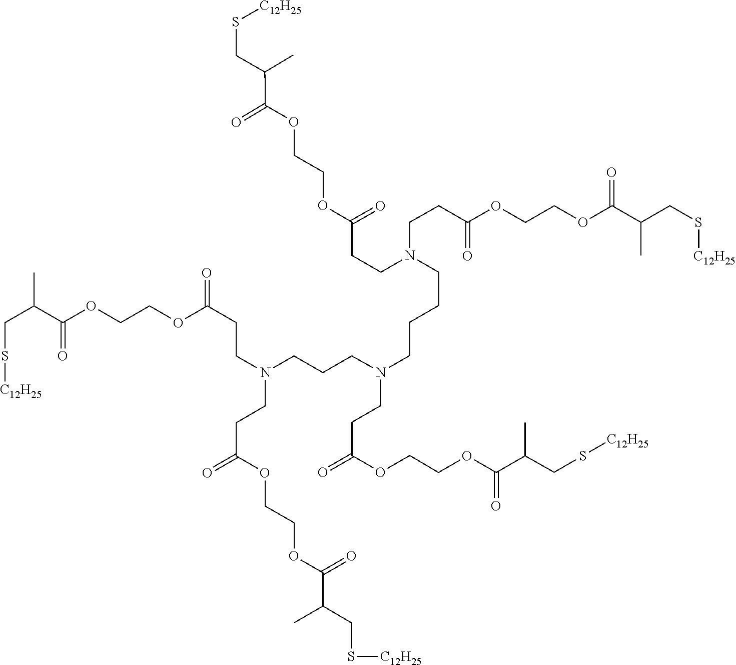 |
| 5A2-4-SC12 (6-arm) | 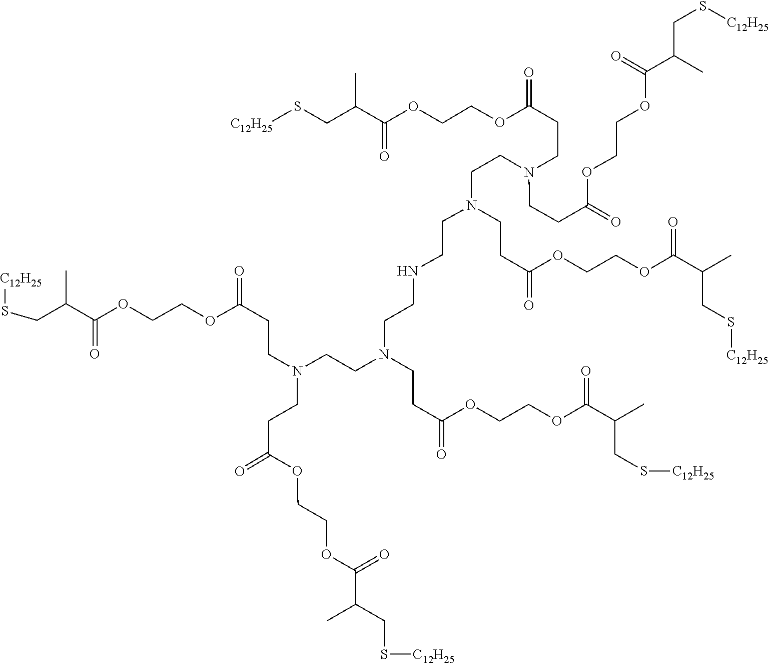 |
| 5A2-4-SC10 (6-arm) | 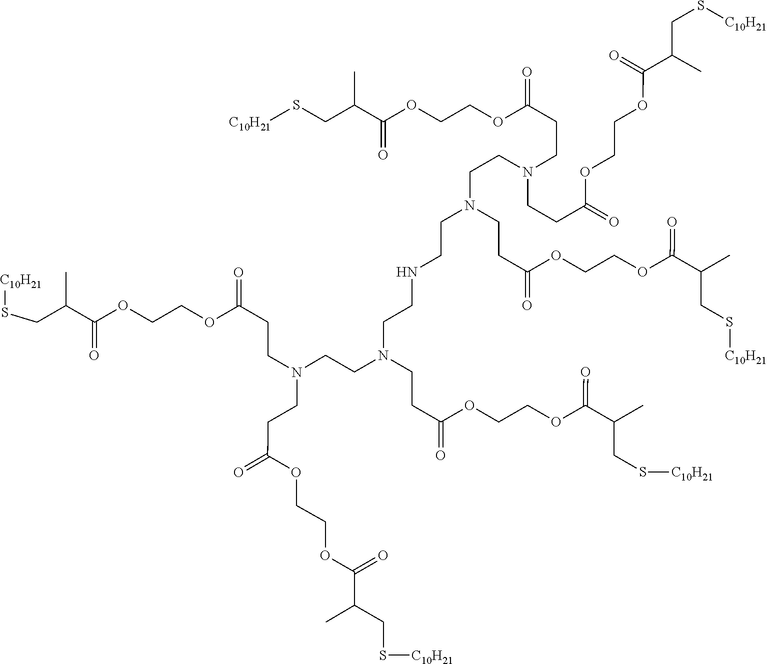 |
| 5A3-2-SC8 (6-arm) | 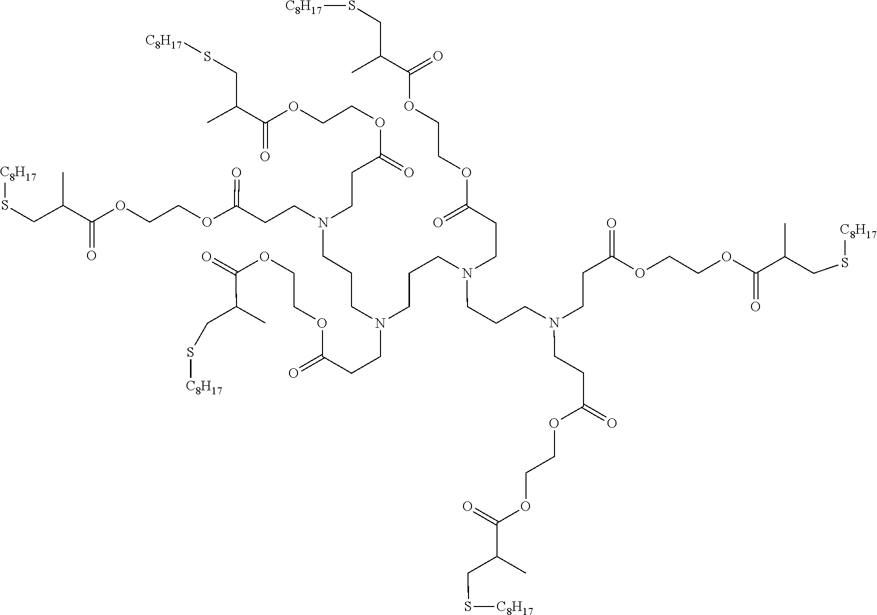 |
| 5A3-2-SC12 (6-arm) | 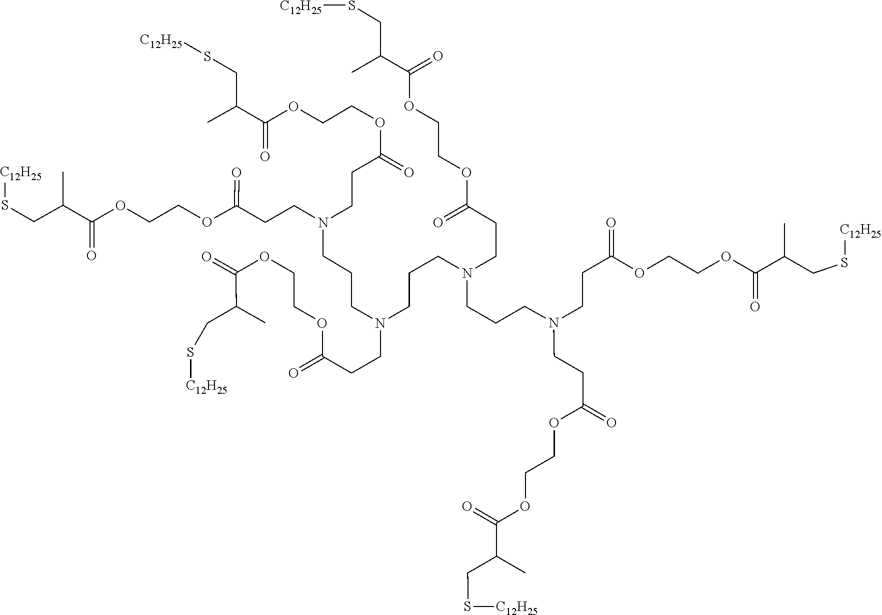 |
| 5A4-2-SC8 (6-arm) | 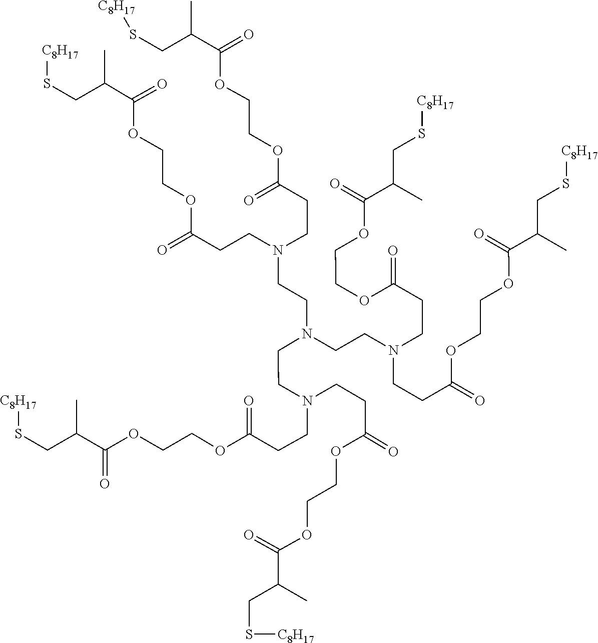 |
| 5A4-2-SC12 (6-arm) | 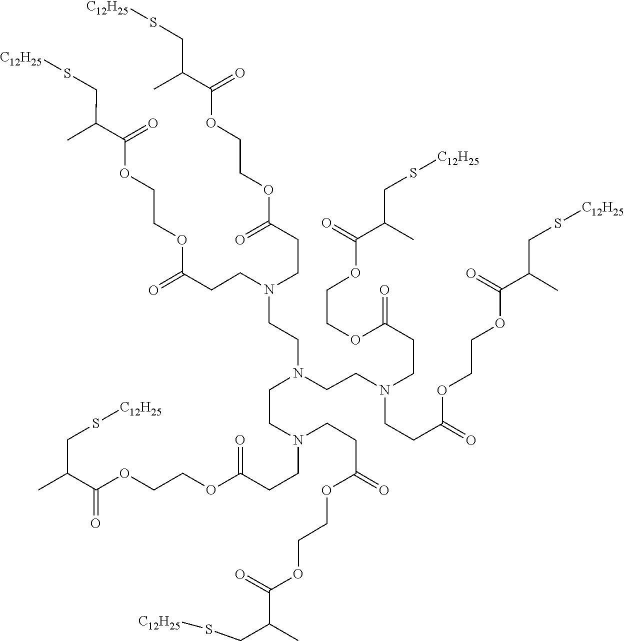 |
| 6A4-SC8 | 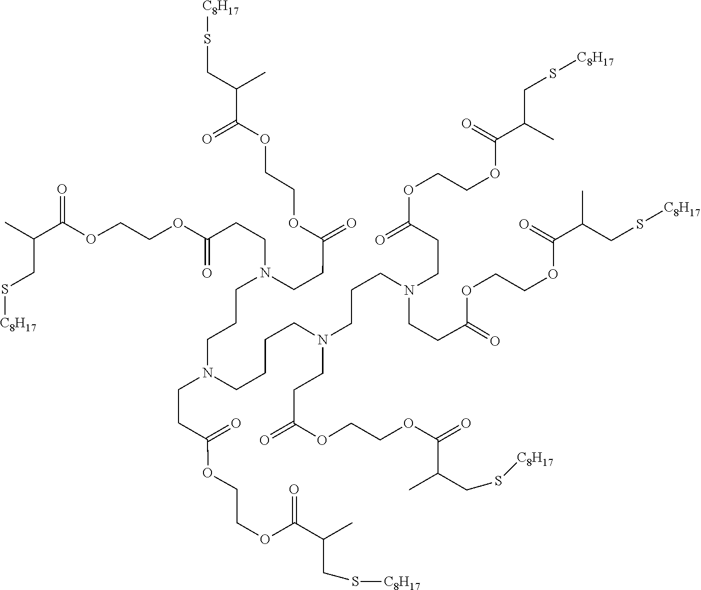 |
| 6A4-SC12 | 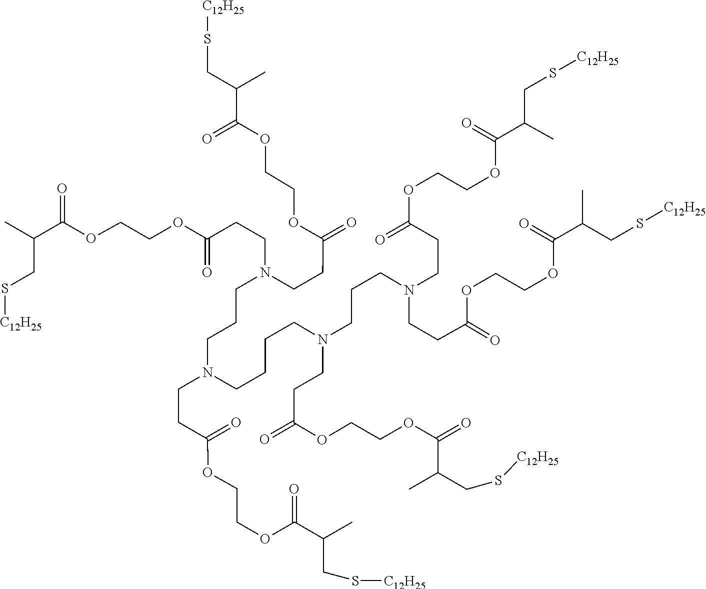 |
| 2A2-g2-SC12 | 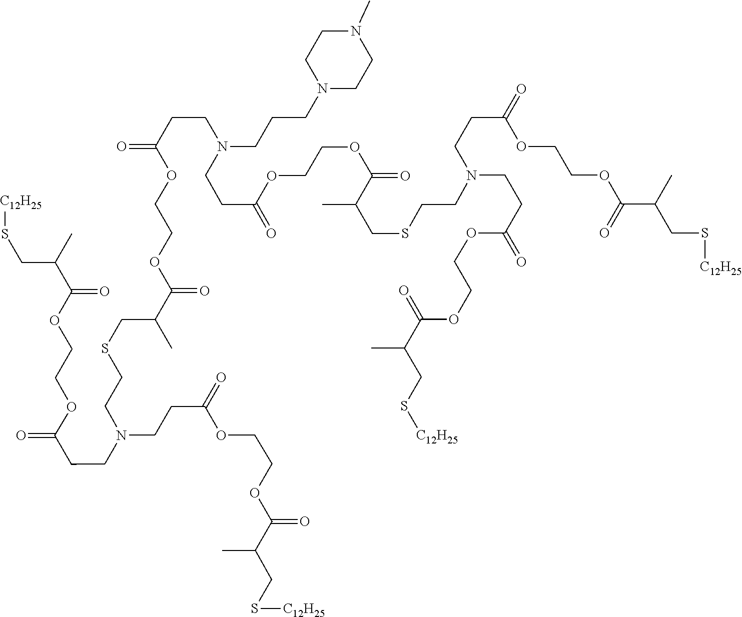 |
| 2A2-g2-SC8 | 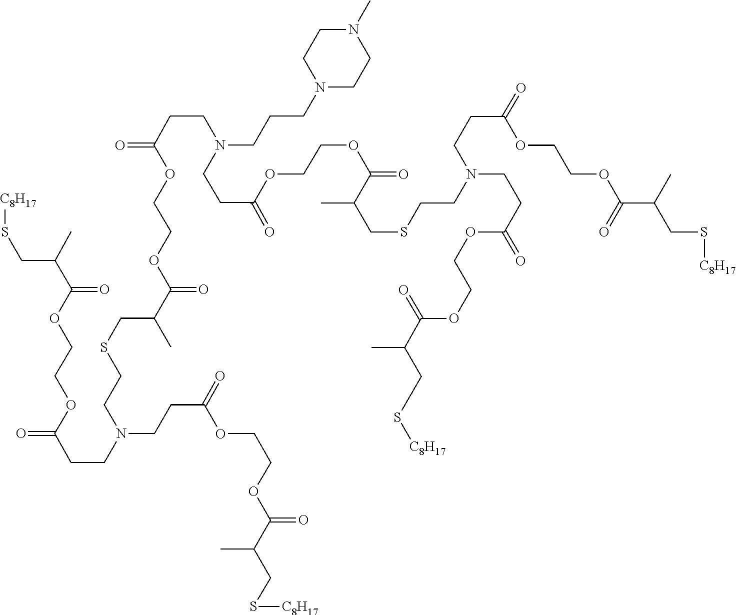 |
| 2A11-g2-SC12 | 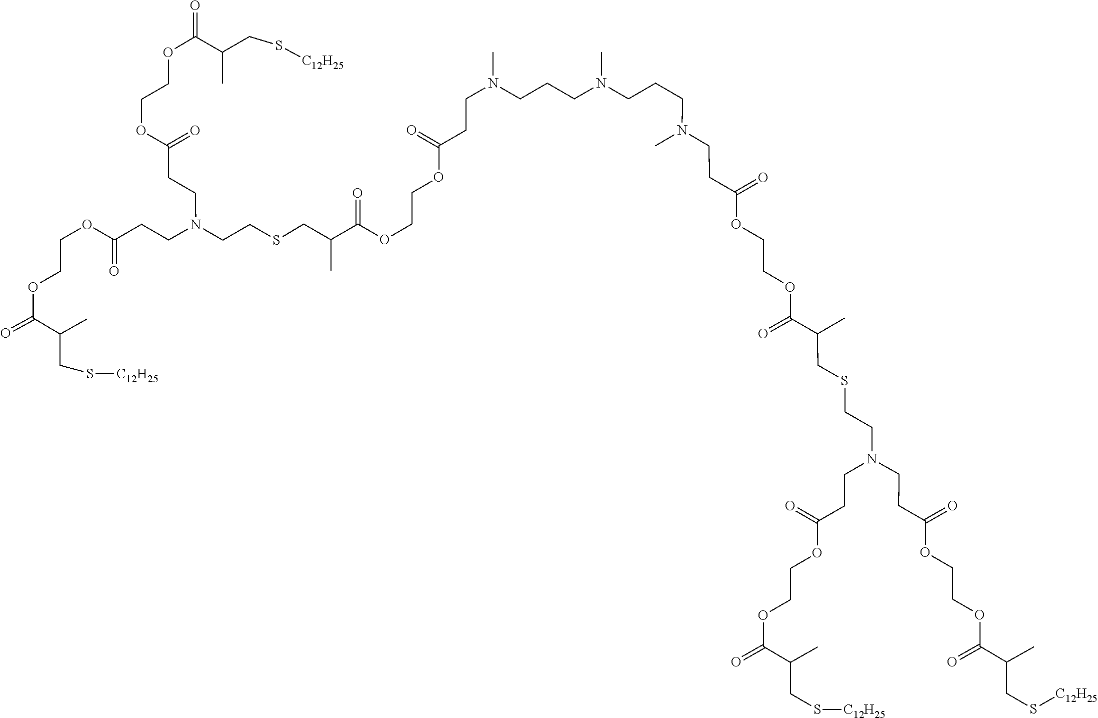 |
| 2A11-g2-SC8 | 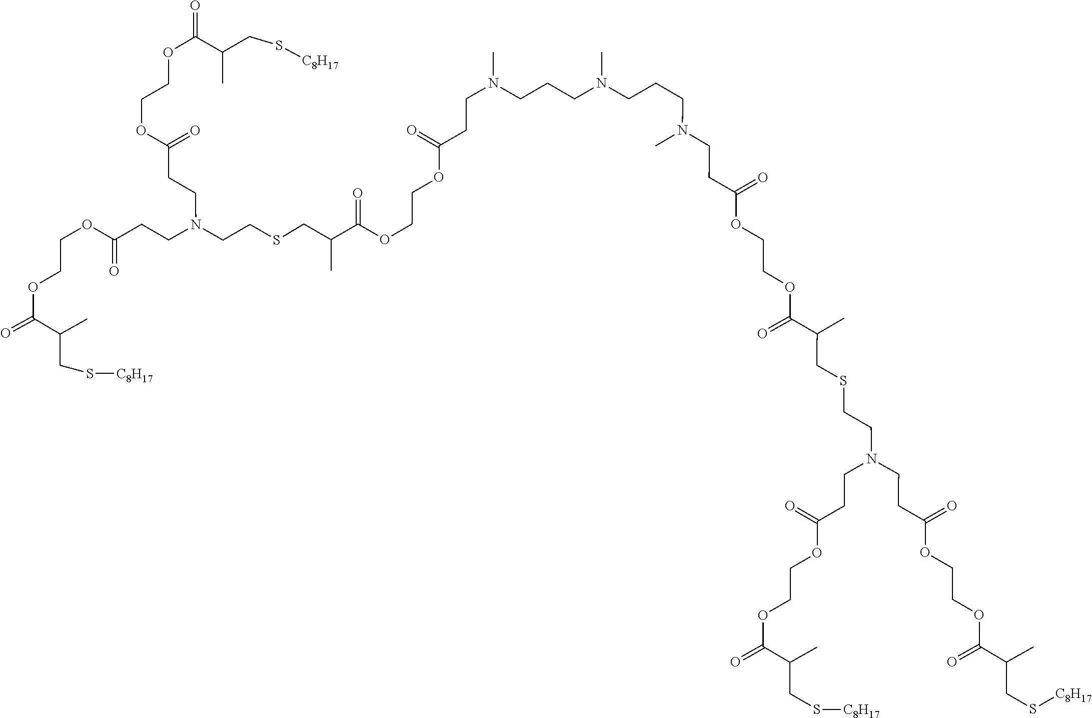 |
| 3A3-g2-SC12 | 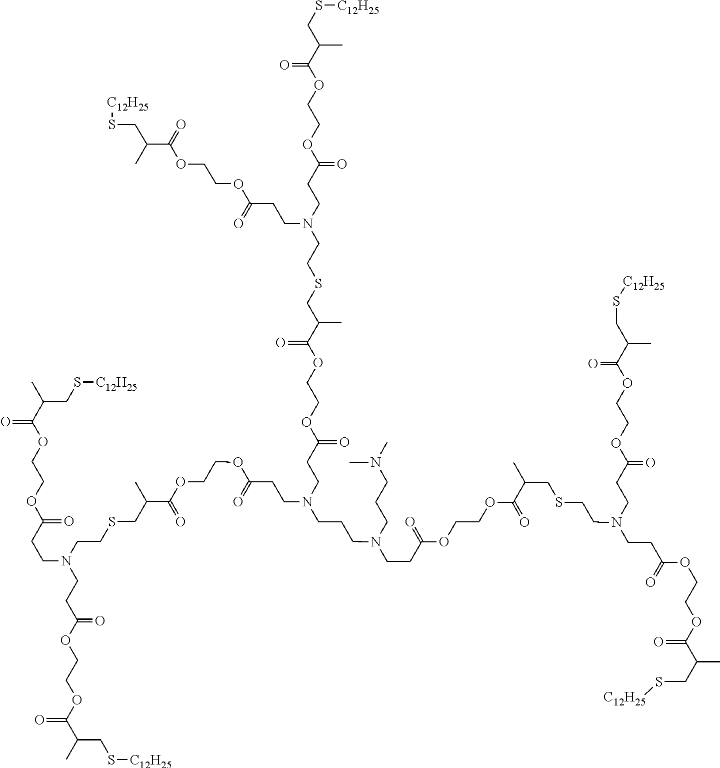 |
| 3A3-g2-SC8 | 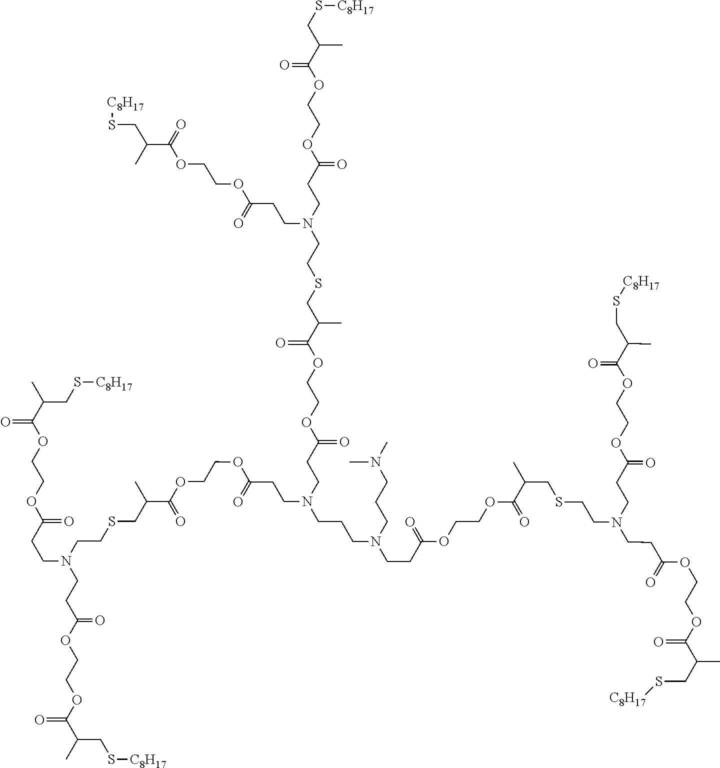 |
| 3A5-g2-SC12 | 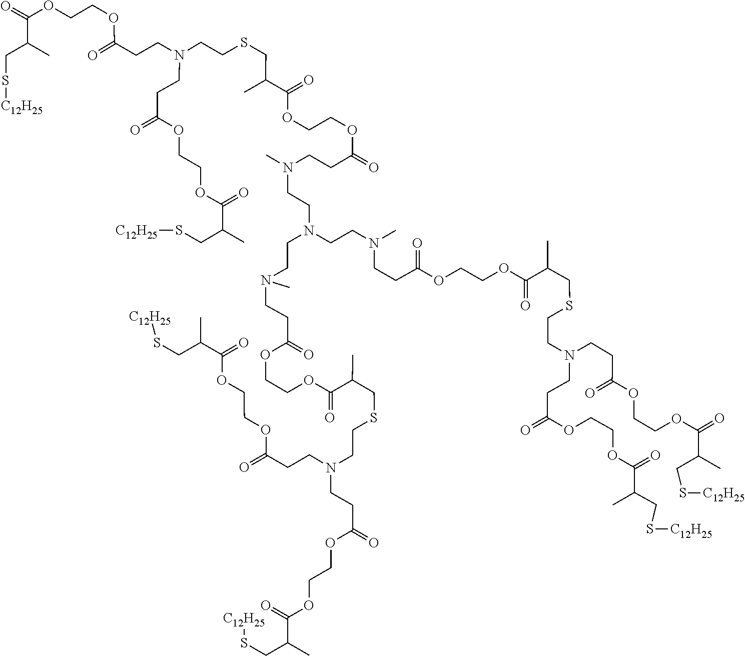 |
| 2A11-g3-SC12 | 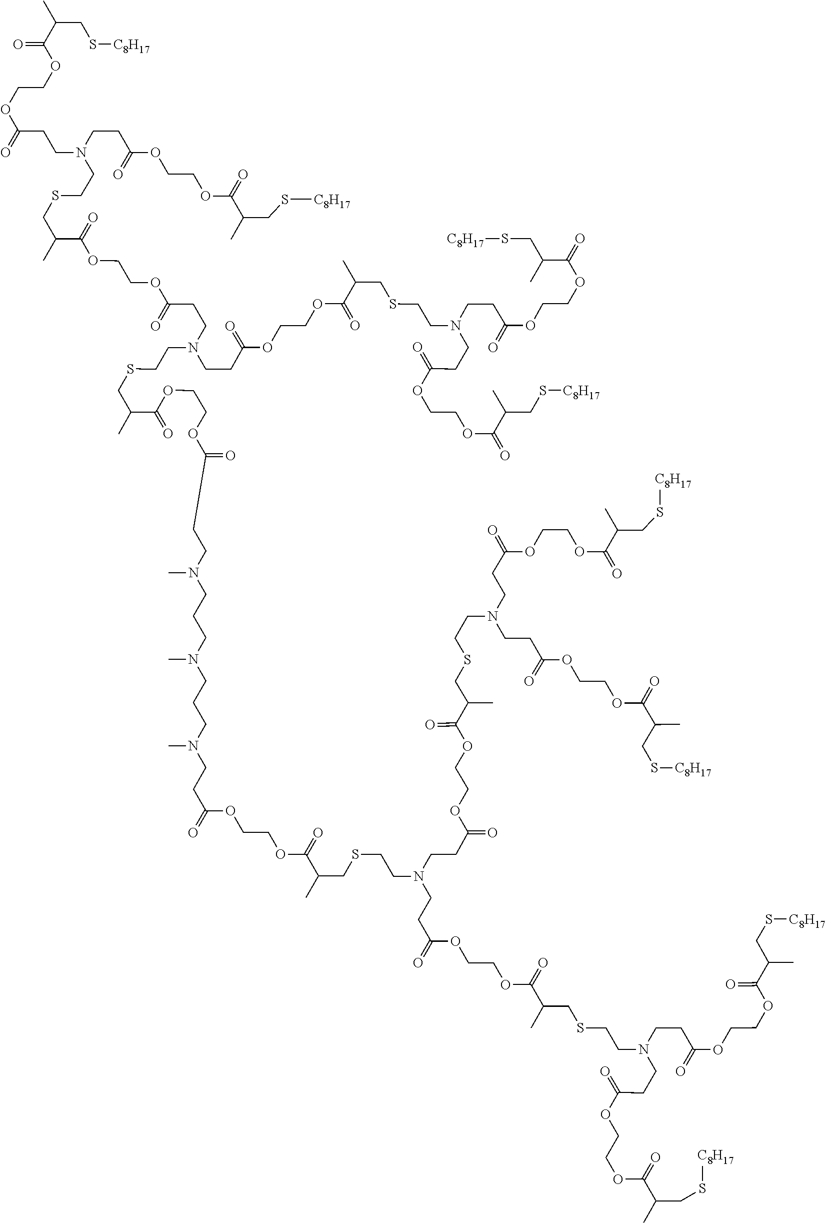 |
| 2A11-g3-SC8 | 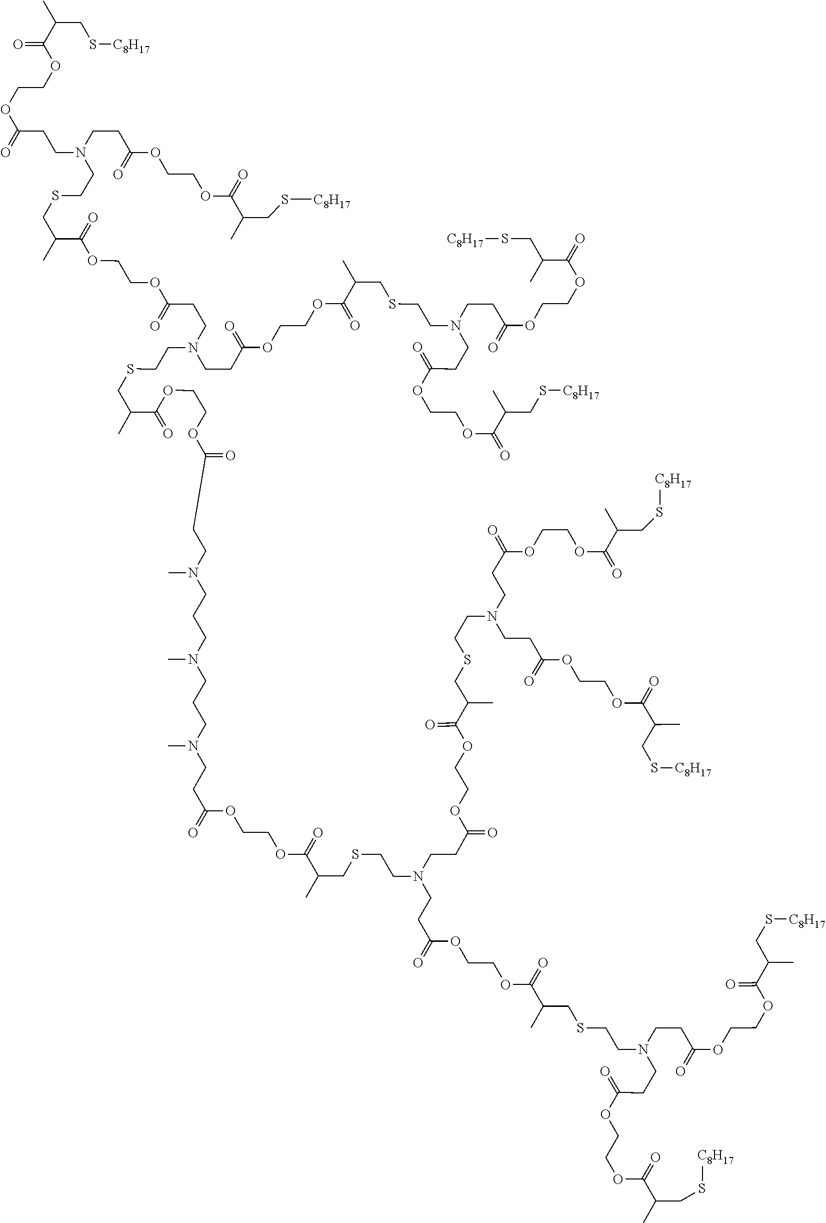 |
| 1A2-g4-SC12 | 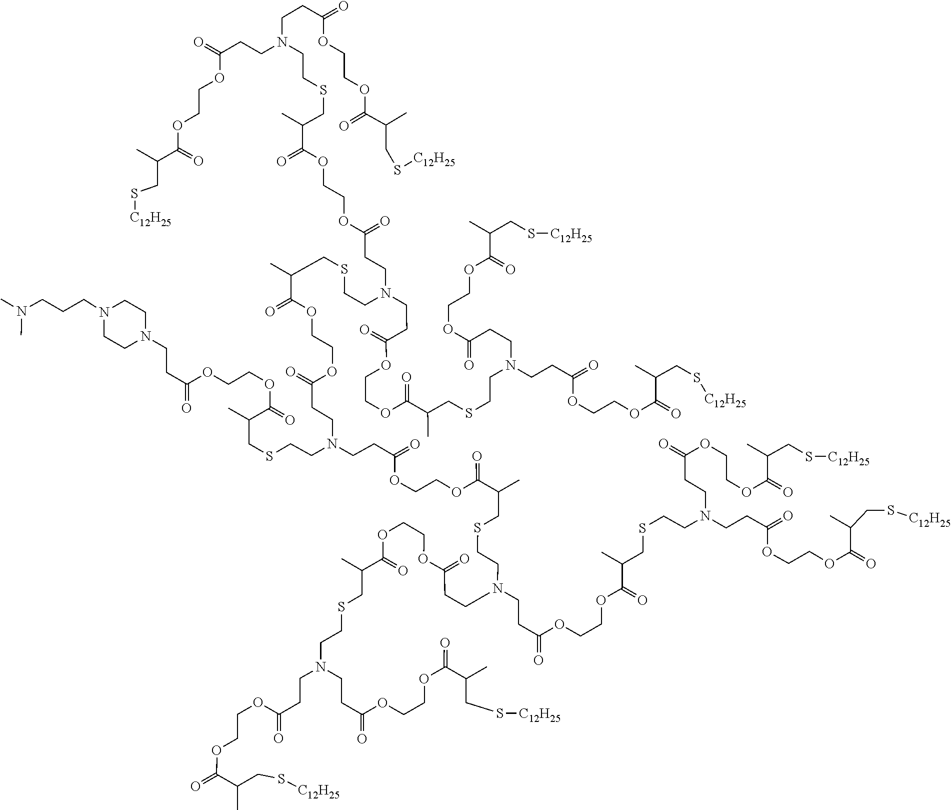 |
| 4A1-g2-SC12 | 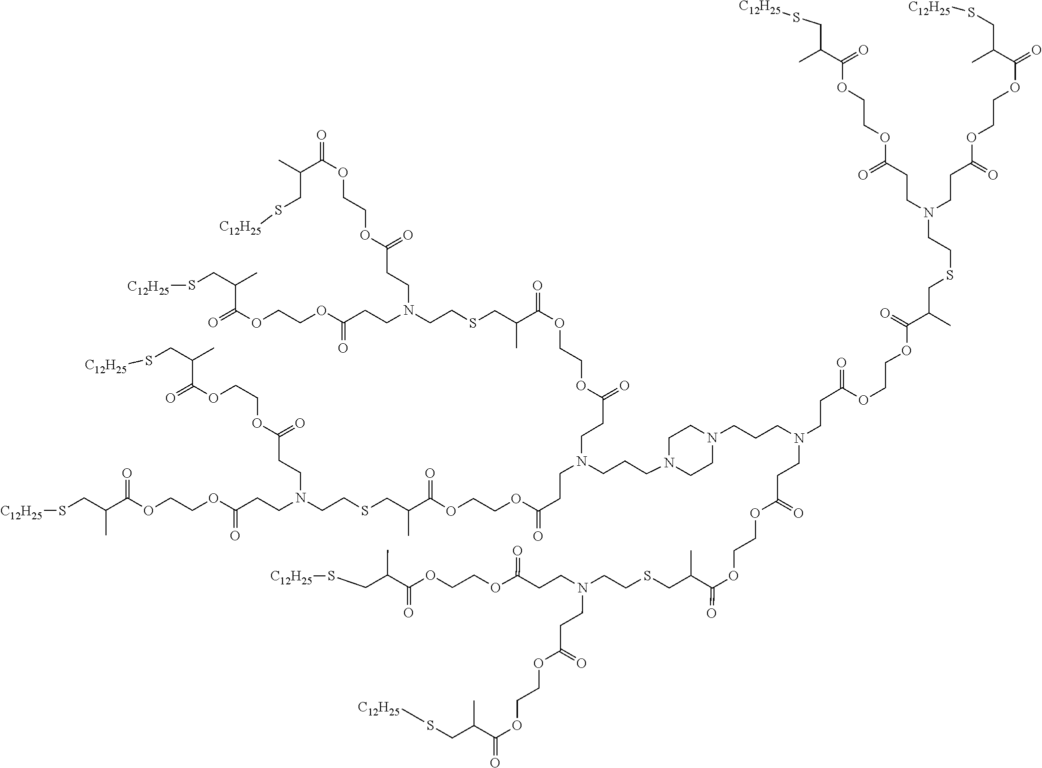 |
| 1A2-g4-SC8 | 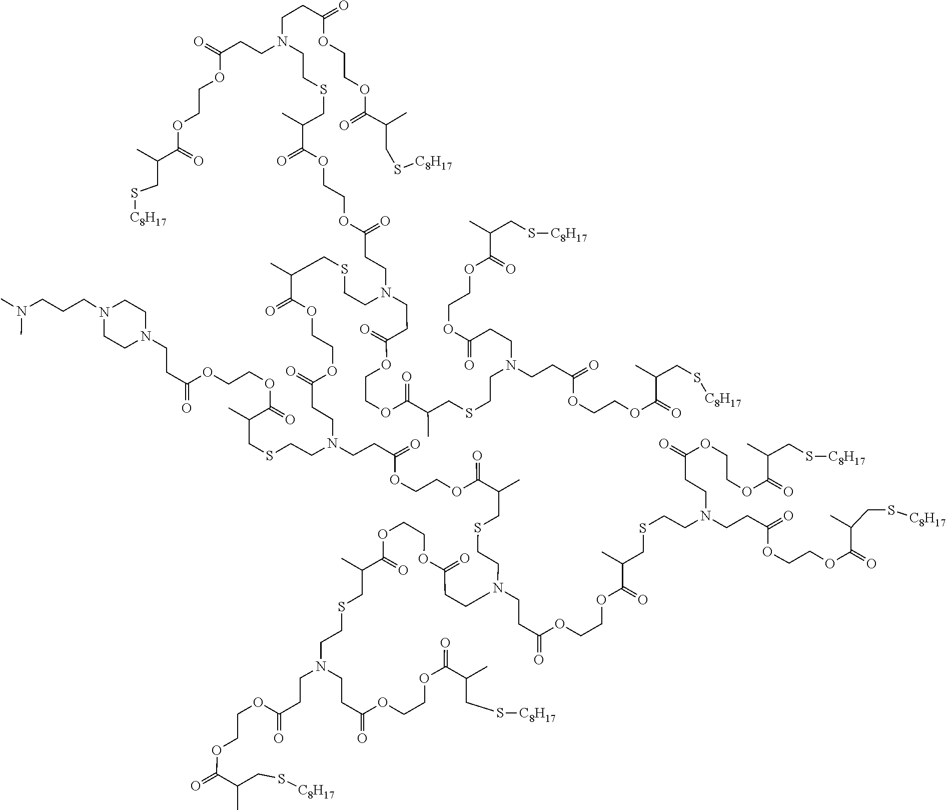 |
| 4A1-g2-SC8 | 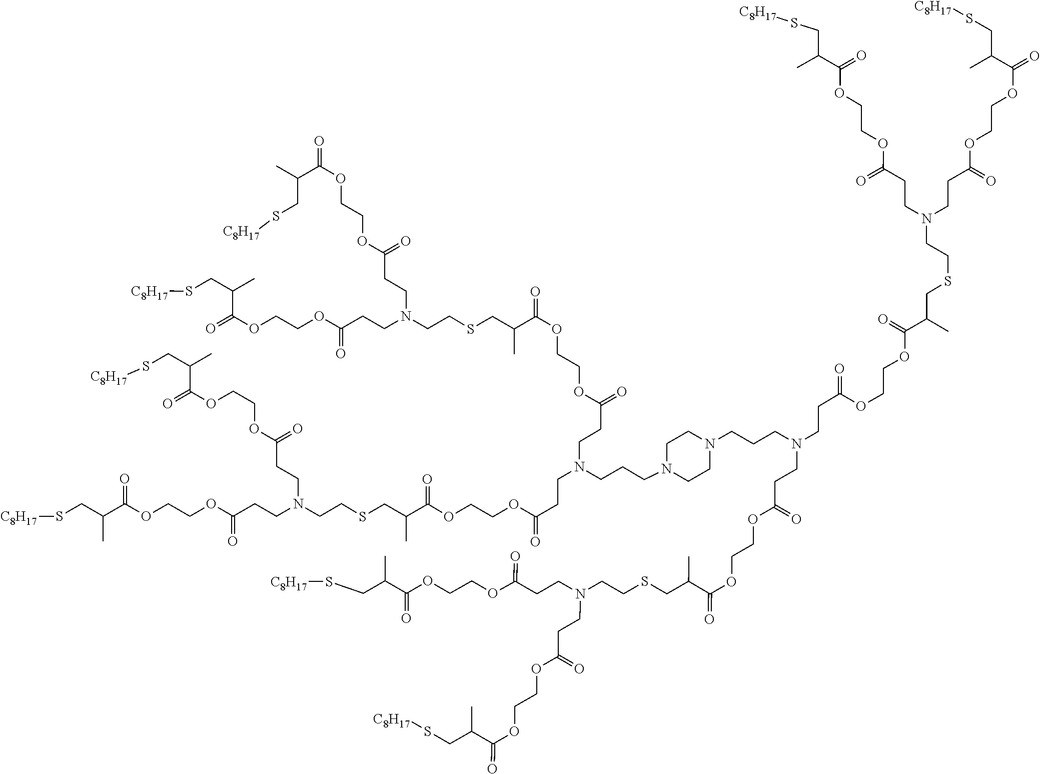 |
| 4A3-g2-SC12 | 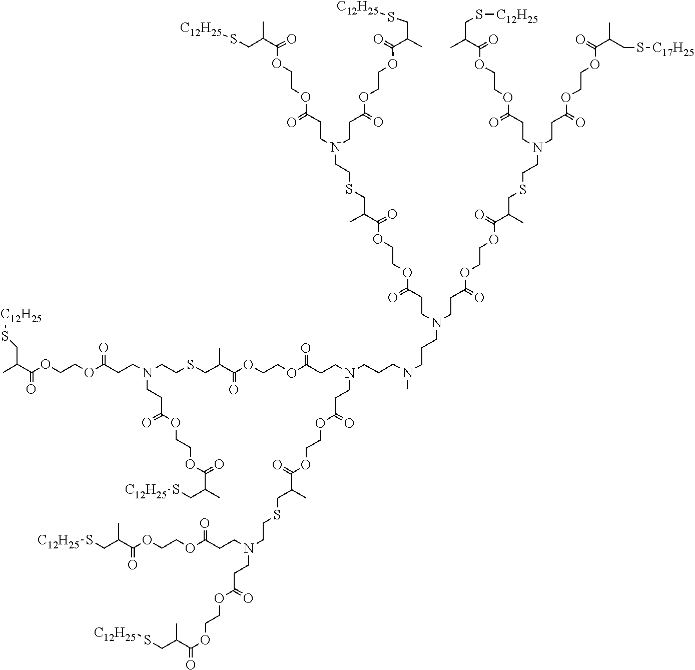 |
| 4A3-g2-SC8 | 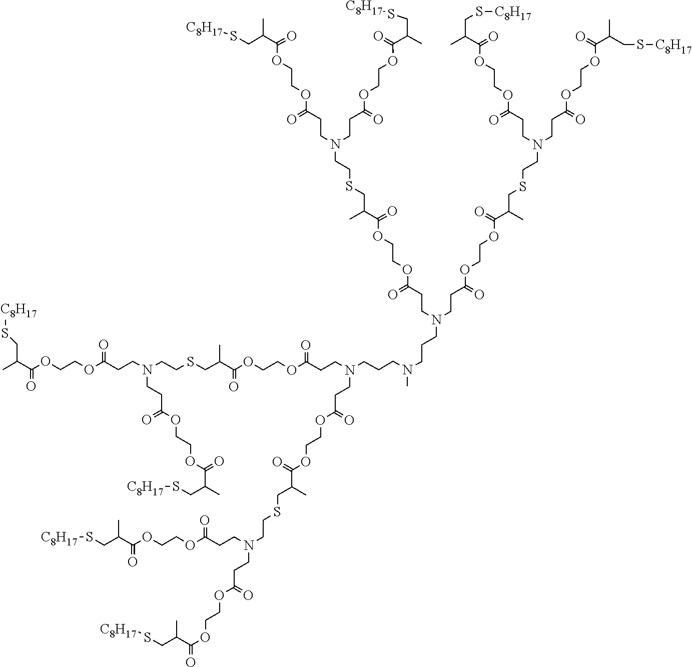 |
| 1A2-g3-SC12 | 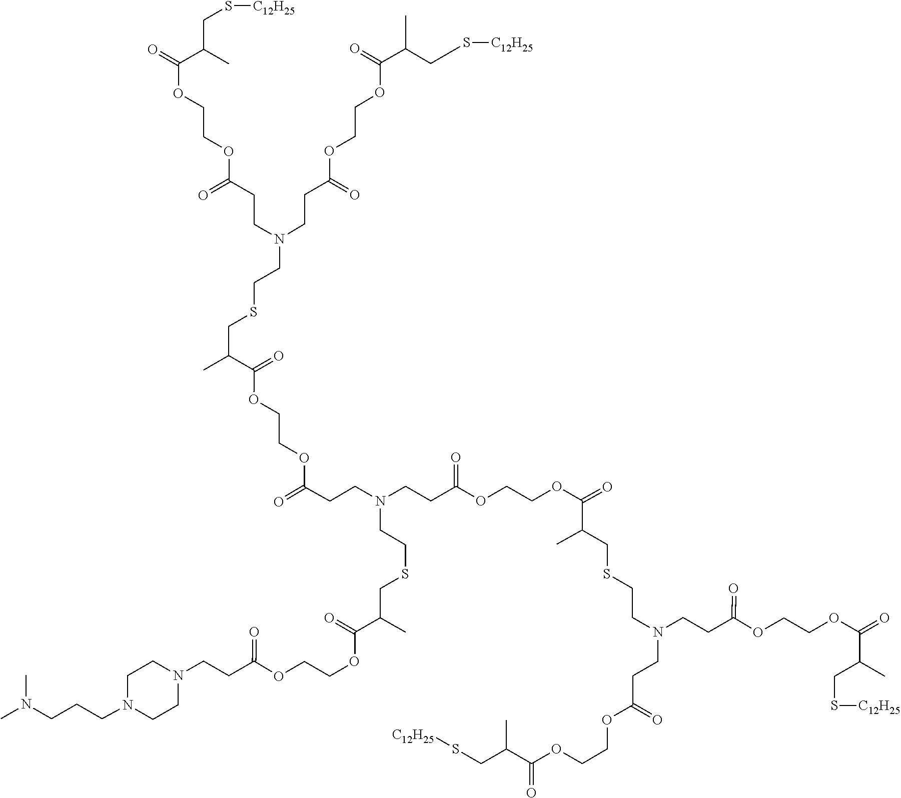 |
| 1A2-g3-SC8 | 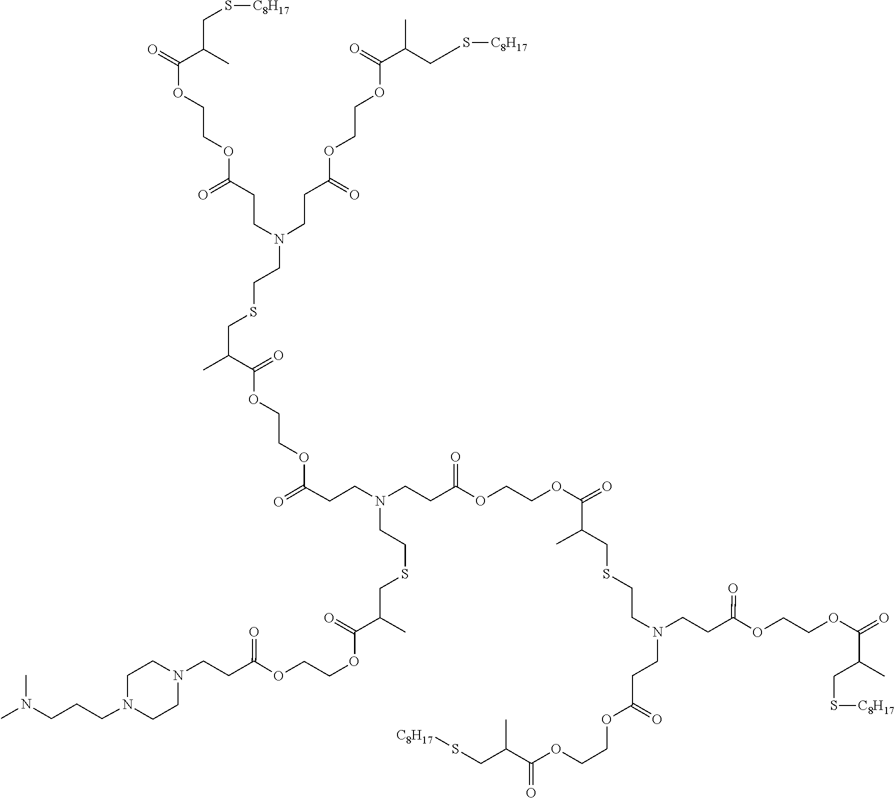 |
| 2A2-g3-SC12 | 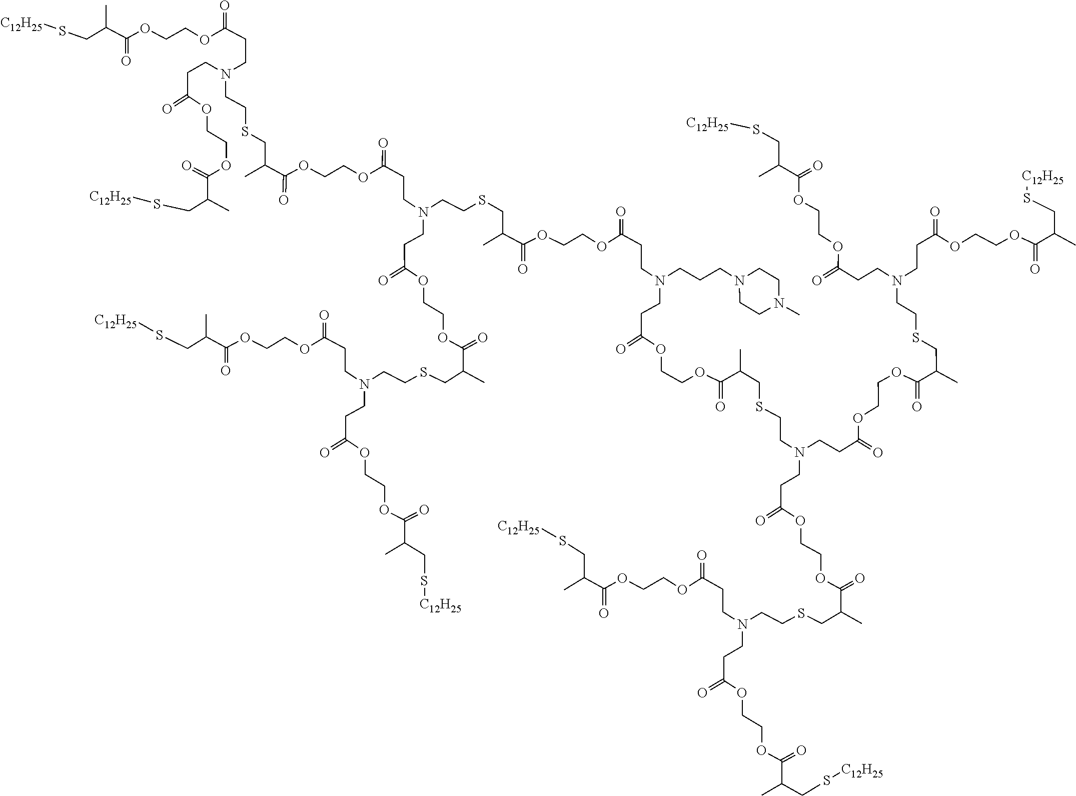 |
| 2A2-g3-SC8 | 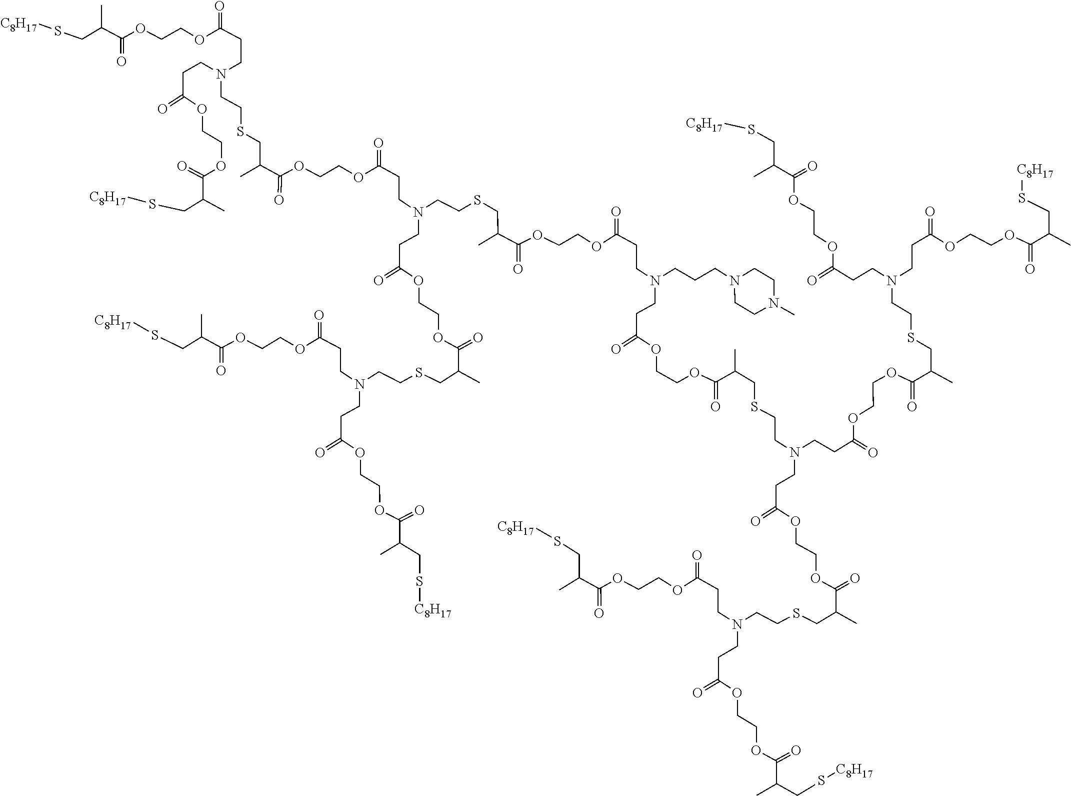 |
| 5A2-4-SC8 (6-arm) | 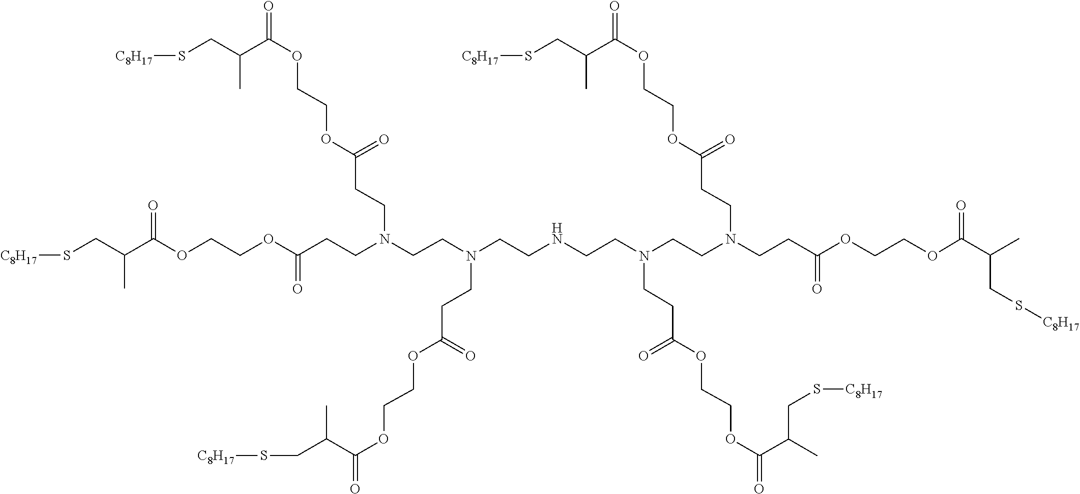 |
| 5A-5-SC8 (6 arm) | 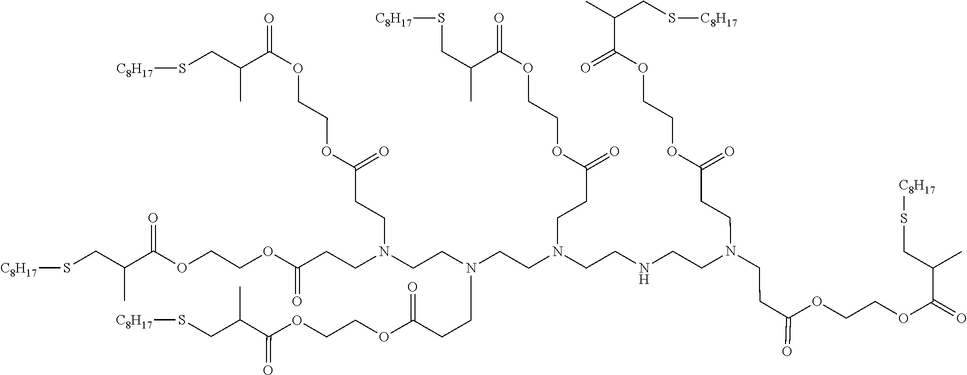 |
| 5A2-6-SC8 (6 arm) | 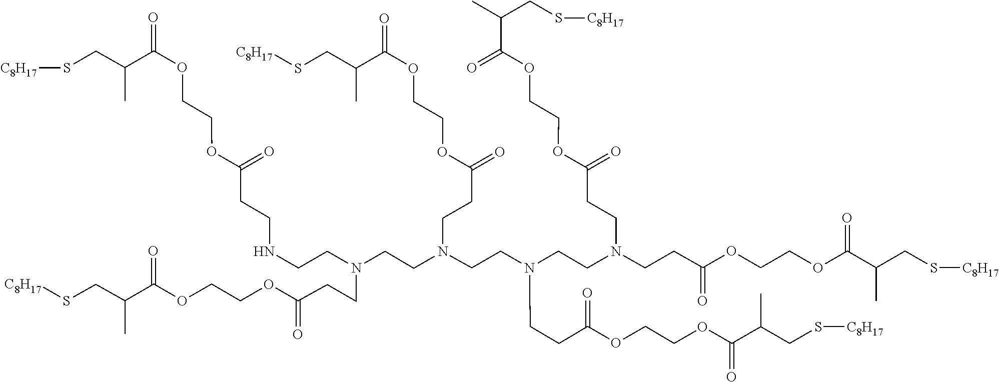 |
| 5A2-1-SC8 (5-arm) | 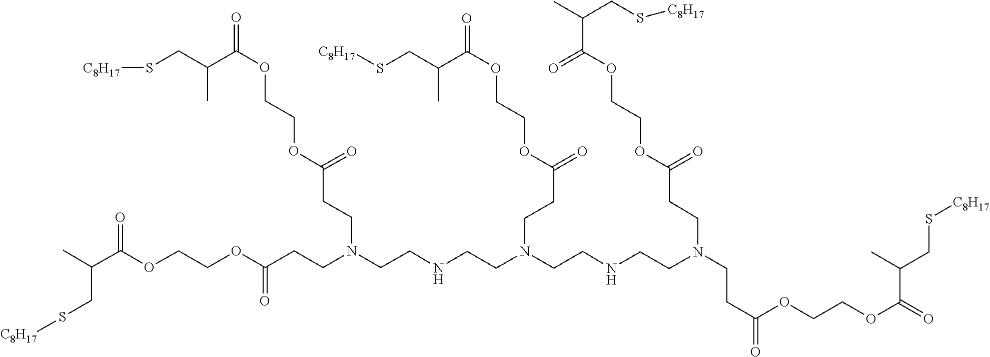 |
| 5A2-2-SC8 | 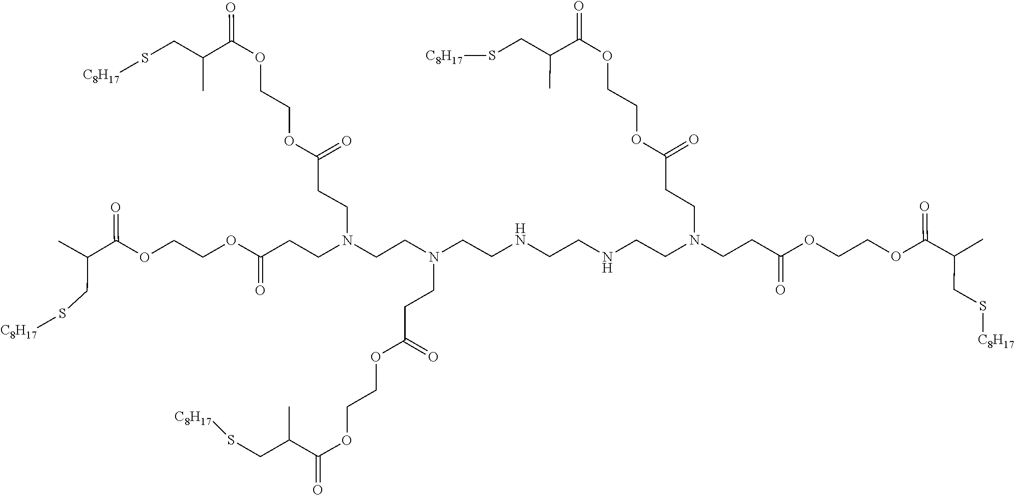 |
| 4A1-SC5 | 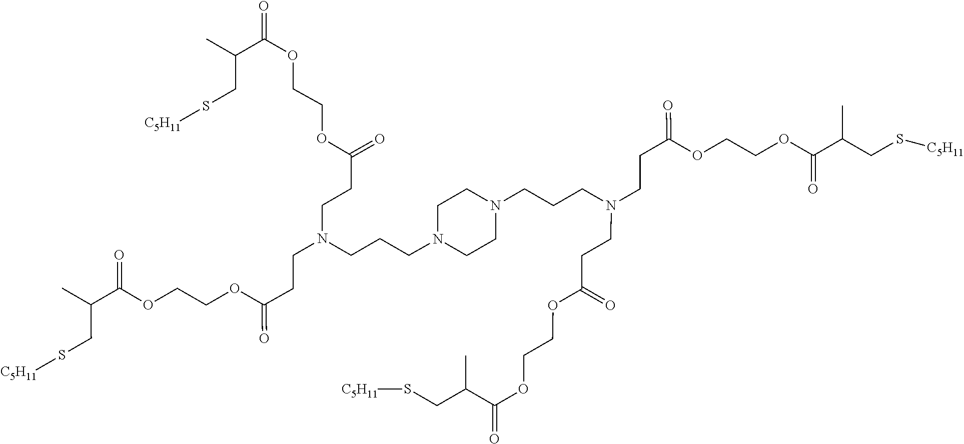 |
| 4A1-SC8 | 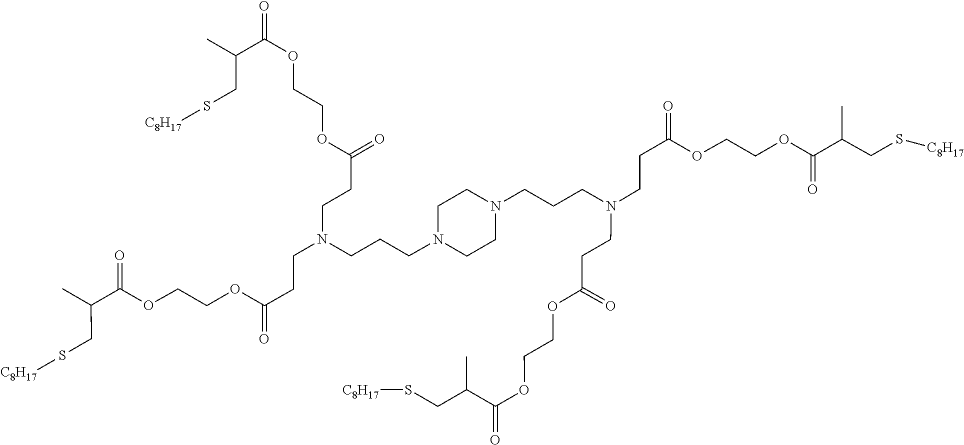 |
| 4A3-SC6 | 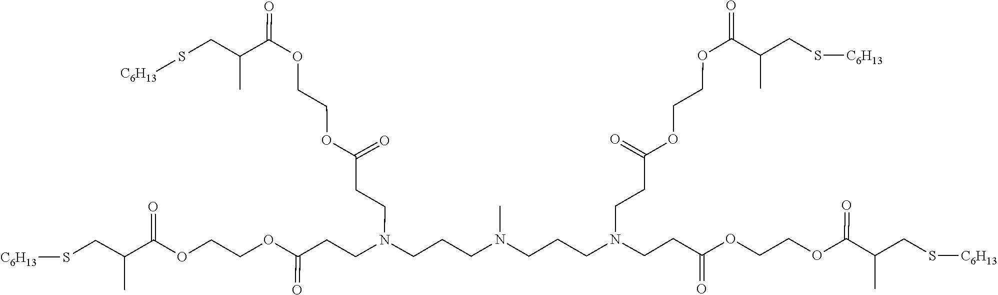 |
| 4A3-SC7 | 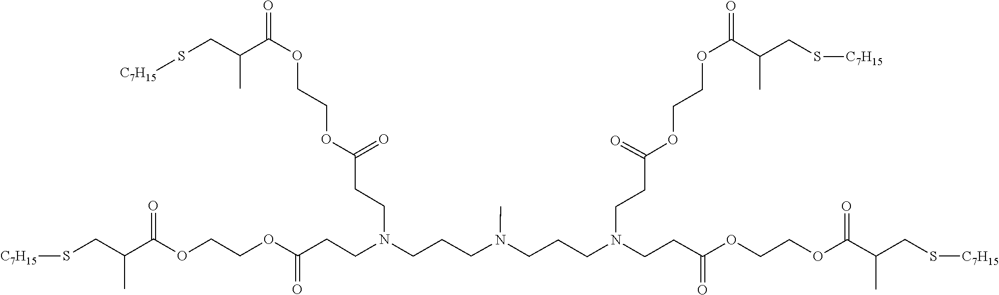 |
| 4A3-SC8 | 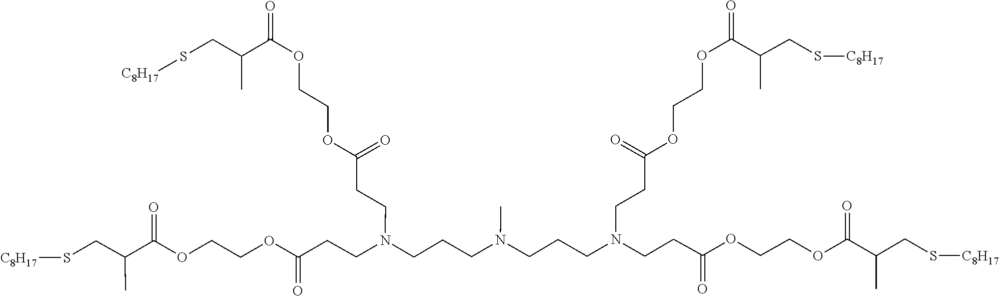 |
| 5A4-2-SC5 (6 arm) | 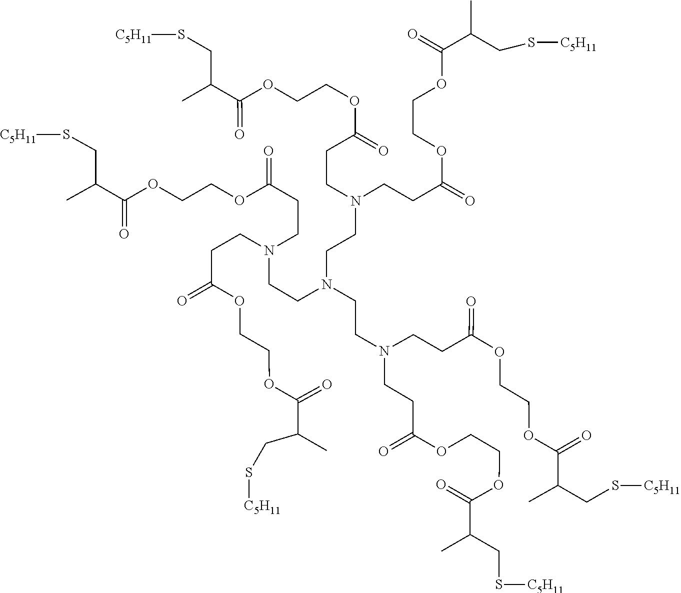 |
| 5A4-2-SC6 (6 arm) | 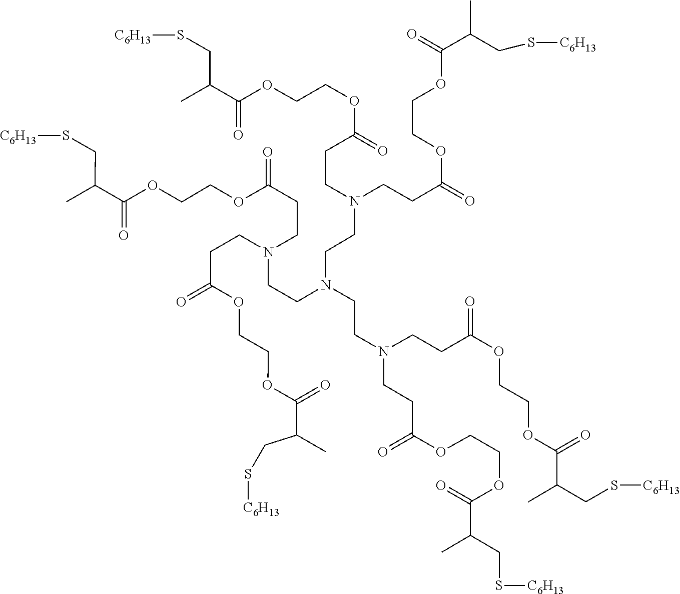 |
| 5A2-4-SC8 (5-arm) | 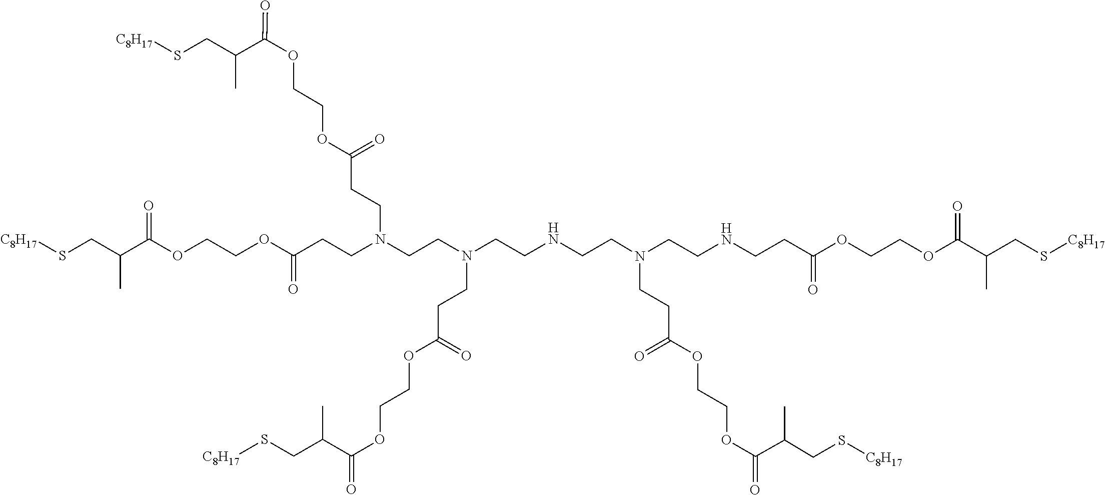 |
| 3A5-g2-SC8 | 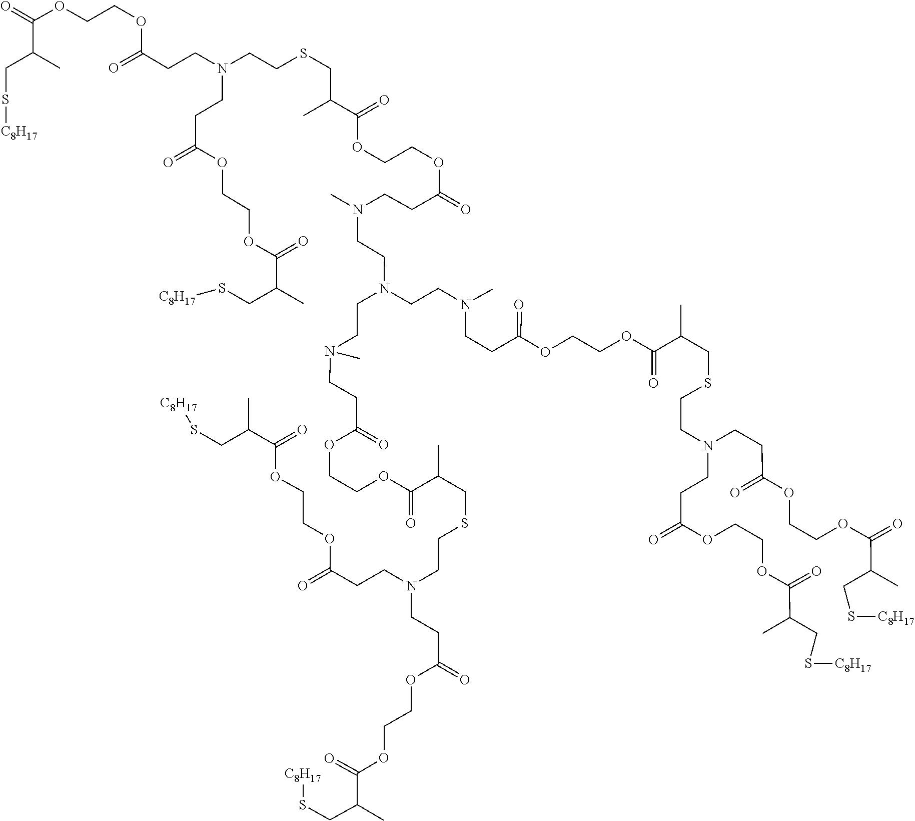 |

### Selective Organ Targeting Compounds

In some embodiments, the lipid (e.g., nanoparticle) compositions described herein are preferentially delivered to a target organ. In some embodiments, the target organ is a lung, a lung tissue or a lung cell. As used herein, the term “preferentially delivered” is used to refer to a composition, upon being delivered, which is delivered to the target organ (e.g., lung), tissue, or cell in at least 25% (e.g., at least 30%, 35%, 40%, 45%, 50%, 55%, 60%, 65%, 70%, or 75%) of the amount administered.

In some embodiments, the lipid compositions disclosed in the present application comprise one or more selective organ targeting (SORT) lipid which leads to the selective delivery of the composition to a particular organ. This SORT compound may be a lipid, a small molecule therapeutic agent, a sugar, a vitamin, a peptide or a protein. The SORT compound may be a lipid. A lipid may be a small molecule with two or more alkyl or alkenyl chains of C₆-C₂₄. A small molecule therapeutic agent is a compound containing less than 100 non-hydrogen atoms and a weight of less than 2,000 Daltons. A sugar is a molecule comprising a molecular formula CₙH₂ₙOₙ, wherein n is from 3 to 7 or a combination of multiple molecules of that formula. A protein is a sequence of amino acids comprising at least 3 amino acid residues. Proteins without a formal tertiary structure may also be referred to as a peptide. The protein may also comprise an intact protein with a tertiary structure. A vitamin is a macronutrient and consists of one or more compounds selected from Vitamin A, Vitamin B₁, Vitamin B₂, Vitamin B₃, Vitamin B₅, Vitamin B₆, Vitamin B₇, Vitamin B₉, Vitamin B12, Vitamin C, Vitamin D, Vitamin E, and Vitamin K.

In some embodiments of the SORT formulations, the SORT lipid comprises permanently positively charged moiety. The permanently positively charged moiety may be positively charged at a physiological pH such that the SORT lipid comprises a positive charge upon delivery of a polynucleotide to a cell. In some embodiments the positively charged moiety is quaternary amine or quaternary ammonium ion. In some embodiments, the SORT lipid comprises, or is otherwise complexed to or interacting with, a counterion.

In some embodiments of the SORT formulations, the SORT lipid is a permanently cationic lipid (i.e., comprising one or more hydrophobic components and a permanently cationic group). The permanently cationic lipid may contain a group which has a positive charge regardless of the pH. One permanently cationic group that may be used in the permanently cationic lipid is a quaternary ammonium group. The permanently cationic lipid may comprise a

structural formula:

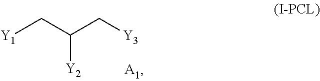

wherein:
Y₁, Y₂, or Y₃ are each independently X₁C(O)R₁ or X₂N⁺R₃R₄R₅;
provided at least one of Y₁, Y₂, and Y₃ is X₂N⁺R₃R₄R₅;
R₁ is C₁-C₂₄ alkyl, C₁-C₂₄ substituted alkyl, C₁-C₂₄ alkenyl, C₁-C₂₄ substituted alkenyl;
X₁ is O or NRₐ, wherein Rₐ is hydrogen, C₁-C₄ alkyl, or C₁-C₄ substituted alkyl;
X₂ is C₁-C₆ alkanediyl or C₁-C₆ substituted alkanediyl;
R₃, R₄, and R₅ are each independently C₁-C₂₄ alkyl, C₁-C₂₄ substituted alkyl, C₁-C₂₄ alkenyl, C₁-C₂₄ substituted alkenyl; and
A₁ is an anion with a charge equal to the number of X₂N⁺R₃R₄R₅ groups in the compound.

In some embodiments of the SORT formulations, the permanently cationic SORT lipid has a structural formula:

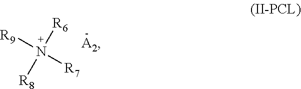

wherein:
R₆—R₉ are each independently C₁-C₂₄ alkyl, C₁-C₂₄ substituted alkyl, C₁-C₂₄ alkenyl, C₁-C₂₄ substituted alkenyl; provided at least one of R₆—R₉ is a group of C₈-C₂₄; and A₂ is a monovalent anion.

In some embodiments of the lipid compositions, the SORT lipid is ionizable cationic lipid (i.e., comprising one or more hydrophobic components and an ionizable cationic group). The ionizable positively charged moiety may be positively charged at a physiological pH. One ionizable cationic group that may be used in the ionizable cationic lipid is a tertiary ammine group. In some embodiments of the lipid compositions, the SORT lipid has a structural formula:

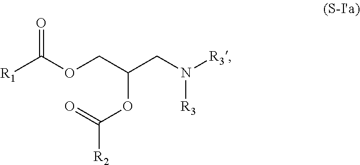

wherein:
R₁ and R₂ are each independently alkyl_{(C8-c24)}, alkenyl_{(C8-c24)}, or a substituted version of either group; and
R₃ and R₃′ are each independently alkyl_{(C≤6)} or substituted alkyl_{(C≤6)}.

In some embodiments of the SORT formulations, the SORT lipid comprises ahead group of a particular structure. In some embodiments, the SORT lipid comprises a headgroup having a structural formula:

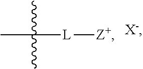

wherein L is a linker; Z⁺is positively charged moiety and X⁻is a counterion. In some embodiment, the linker is a biodegradable linker. The biodegradable linker may be degradable under physiological pH and temperature. The biodegradable linker may be degraded by proteins or enzymes from a subject. In some embodiments, the positively charged moiety is a quaternary ammonium ion or quaternary amine.

In some embodiments of the SORT formulations, the SORT lipid has a structural formula:

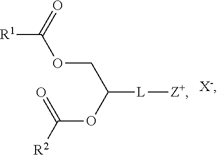

wherein R₁ and R₂ are each independently an optionally substituted C₆-C₂₄ alkyl, or an optionally substituted C₆-C₂₄ alkenyl.

In some embodiments of the SORT formulations, the SORT lipid has a structural formula:

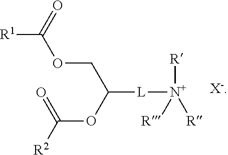

In some embodiments of the SORT formulations, the SORT lipid comprises a Linker (L). In some embodiments, L is

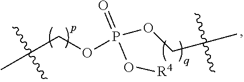

wherein:
p and q are each independently 1, 2, or 3; and
R⁴ is an optionally substituted C₁-C₆ alkyl

In some embodiments of the SORT formulations, the SORT lipid has a structural formula:

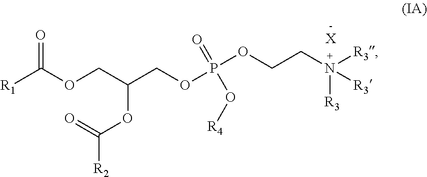

wherein:
R₁ and R₂ are each independently alkyl_{(C8-C24)}, alkenyl_{(C8-c24)}, or a substituted version of either group;
R₃, R₃′, and R₃″ are each independently alkyl_{(C≤6)} or substituted alkyl_{(C≤6)};
R₄ is alkyl_{(C≤6)} or substituted alkyl_{(C≤6)}; and
X⁻is a monovalent anion.

In some embodiments of the SORT formulations, the SORT lipid is a phosphatidylcholine (e.g., 14:0 EPC). In some embodiments, the phosphatidylcholine compound is further defined as:

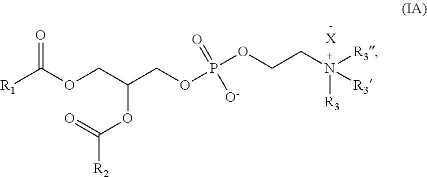

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and X is a monovalent anion.

In some embodiments, the SORT lipid is a phosphocholine lipid. In some embodiments, the SORT lipid is an ethylphosphocholine. The ethylphosphocholine may be, by way of example, without being limited to, 1,2-dimyristoleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1-palmitoyl-2-oleoyl-sn-glycero-3-ethylphosphocholine.

In some embodiments of the SORT formulations, the SORT lipid has a structural formula:

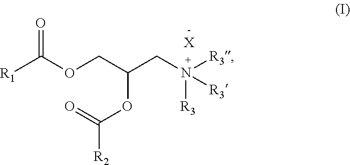

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
X⁻is a monovalent anion.

By way of example, and without being limited thereto, a SORT lipid of the structural formula of the immediately preceding paragraph is 1,2-dioleoyl-3-trimethylammonium-propane (18:1 DOTAP) (e.g., chloride salt).

In some embodiments of the SORT formulations, the SORT lipid has a structural formula:

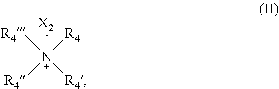

wherein
R₄ and R₄′ are each independently alkyl_{(C6-C24)}, alkenyl_{(C6-C24)}, or a substituted version of either group;
R₄″ is alkyl_{(C≤24)}, alkenyl_{(C≤24)}, or a substituted version of either group;
R₄′″ is alkyl_{(C1-C8)}, alkenyl_{(C2-C8)}, or a substituted version of either group; and
X₂⁻ is a monovalent anion.

By way of example, and without being limited thereto, a SORT lipid of the structural formula of the immediately preceding paragraph is dimethyldioctadecylammonium (DDAB) (e.g., bromide salt).

In some embodiments of the lipid compositions, the SORT lipid has a structural formula:

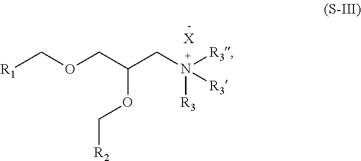

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻is a monovalent anion.

By way of example, and without being limited thereto, a SORT lipid of the structural formula of the immediately preceding paragraph is N-[1-(2, 3-dioleyloxy)propyl]-N,N,N-trimethylammonium chloride (DOTMA).

In some embodiments of the lipid compositions, the SORT lipid is an anionic lipid. In some embodiments of the lipid compositions, the SORT lipid has a structural formula:

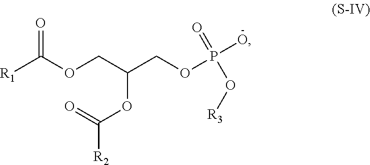

wherein:
R₁ and R₂ are each independently alkyl_{(C8-C24)}, alkenyl_{(C8-C24)}, or a substituted version of either group;
R₃ is hydrogen, alkyl_{(C≤6)}, or substituted alkyl_{(C≤6)}, or —Y₁—R₄, wherein:
Y₁ is alkanediyl_{(C≤6)} or substituted alkanediyl_{(C≤6)}; and
R₄ is acyloxy_{(C≤8-24)} or substituted acyloxy_{(C≤8-24)}.

In some embodiments of the SORT formulations, the SORT lipid comprises one or more selected from the lipids set forth in Table 7.

TABLE 7
Example SORT lipids

| Lipid Name | Structure |
|---|---|
| 1,2-Dioleoyl-3- dimethylammonium- propane (18:1 DODAP) | 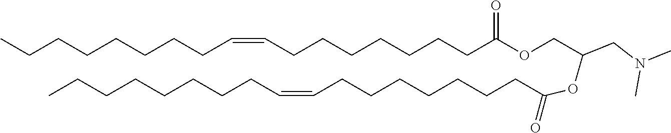 [SMILES: CCCCCCCC/C=C\CCCCCCCC(=O)OCC(CN(C)C)OC(=O)CCCCCCC/C=C\CCCCCCCC] |
| 1,2-dimyristoyl-3- trimethylammonium- propane (14:0 TAP) (e.g., chloride salt) | 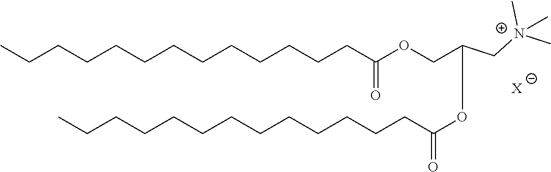 |
| 1,2-dipalmitoyl-3- trimethylammonium- propane (16:0 TAP) (e.g., chloride salt) | 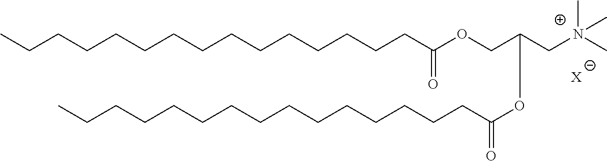 |
| 1,2-stearoyl-3- trimethylammonium- propane (18:0 TAP) (e.g., chloride salt) | 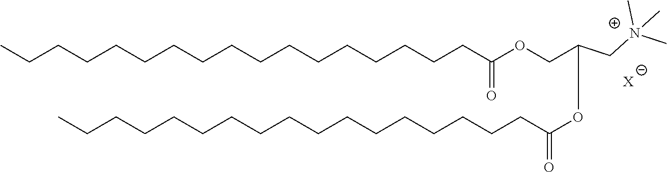 |
| 1,2-Dioleoyl-3- trimethylammonium- propane (18:1 DOTAP) (e.g., chloride salt) | 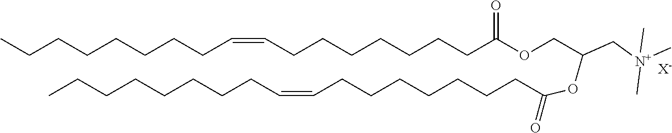 |
| 1,2-Di-O- octadecenyl-3- trimethylammonium propane (DOTMA) (e.g., chloride salt) | 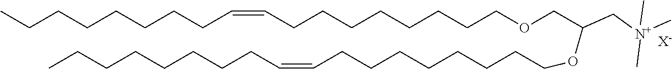 |
| Dimethyldiocta- decylammonium (DDAB) (e.g., bromide salt) | 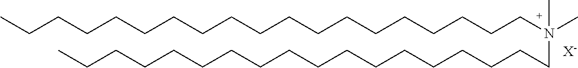 |
| 1,2-dilauroyl-sn- glycero-3- ethylphosphocholine (12:0 EPC) (e.g., chloride salt) | 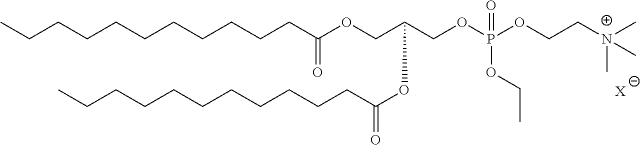 |
| 1,2-Dioleoyl-sn- glycero-3- ethylphosphocholine (14:0 EPC) (e.g., chloride salt) | 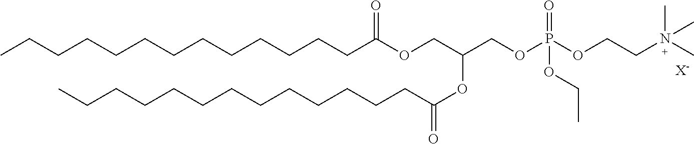 |
| 1,2-dimyristoleoyl- sn-glycero-3- ethylphosphocholine (14:1 EPC) (e.g., triflate salt) | 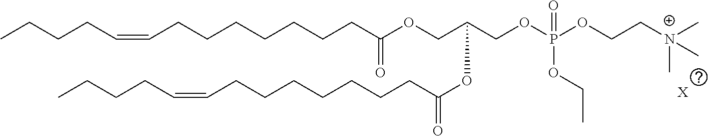 |
| 1,2-dipalmitoyl- sn-glycero- 3-ethylphosphocholine (16:0 EPC) (e.g., chloride salt) | 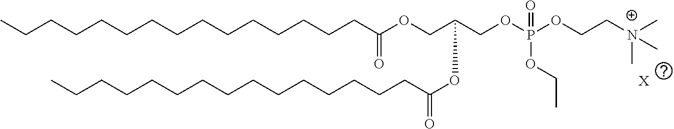 |
| 1,2-distearoyl-sn- glycero-3- ethylphosphocholine (18:0 EPC) (e.g., chloride salt) | 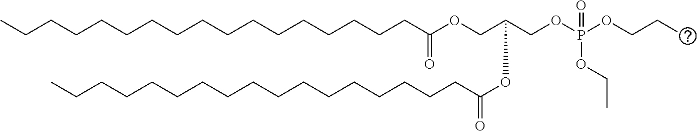 [SMILES: CCCCCCCCCCCCCCCCCC(=O)OC[C@@H](COP(=O)(OCC)OCCC)OC(=O)CCCCCCCCCCCCCCCCC] |
| 1,2-dioleoyl-sn- glycero-3- ethylphosphocholine (18:1 EPC) (e.g., chloride salt) | 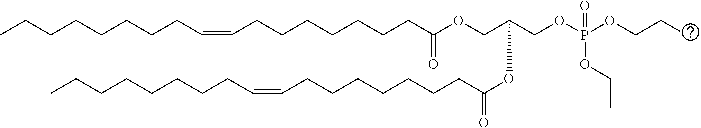 [SMILES: CCCCCCCC/C=C\CCCCCCCC(=O)OC[C@@H](COP(=O)(OCC)OCCC)OC(=O)CCCCCCC/C=C\CCCCCCCC] |
| 1-palmitoyl-2-oleoyl- sn-glycero-3- ethylphosphocholine (16:0-18:1 EPC) (e.g., chloride salt) | 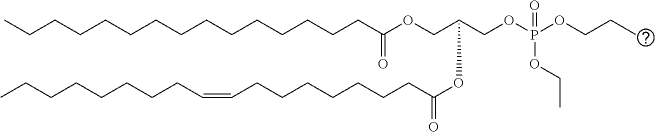 [SMILES: CCCCCCCC/C=C\CCCCCCCC(=O)O[C@@H](COC(=O)CCCCCCCCCCCCCCC)COP(=O)(OCC)OCCC] |
| 1,2-di-O- octadecenyl-3- trimethylammonium propane (18:1 DOTMA) (e.g., chloride salt) | 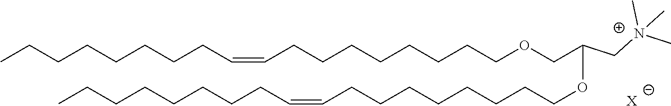 |
| 1,2-dioleoyl-sn- glycero-3- phosphate (18.1 PA) | 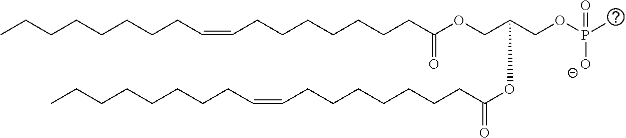 [SMILES: CCCCCCCC/C=C\CCCCCCCC(=O)OC[C@@H](COP(C)(=O)[O-])OC(=O)CCCCCCC/C=C\CCCCCCCC] |

X⁻ is a counterion (e.g., Cl⁻, Br⁻, etc.)

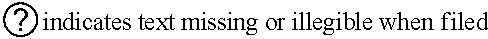

In some embodiments of the SORT formulations, the phospholipid is not an ethylphosphocholine.

In some embodiments of the SORT formulations, the selective organ targeting (SORT) compound is present in the composition in a molar ratio from about 2%, 4%, 5%, 10%, 15%, 20%, 22%, 24%, 26%, 28%, 30%, 32%, 34%, 36%, 38%, 40%, 45%, 50%, 55%, 60%, 65%, to about 70%, or any range derivable therein. In some embodiments, the SORT compound may be present in an amount from about 5% to about 40%, from about 10% to about 40%, from about 20% to about 35%, from about 25% to about 35%, or from about 28% to about 34%.

In some embodiments, the components of the (e.g., pharmaceutical) composition or the lipid composition are present at a particular molar percentage or range of molar percentages. In some embodiments, a component of the lipid composition is present at a molar percentage of at least 1%, 5%, 10%, 15, 20%, 25%, 30%, 35%, 40%, 45%, 50%, 55%, 60%, 65%, 70%, 75%, 80%, 85%, 90%, 95%, or more. In some embodiments, a component of the lipid composition is present at a molar percentage of at no more than 1%, 5%, 10%, 15, 20%, 25%, 30%, 35%, 40%, 45%, 50%, 55%, 60%, 65%, 70%, 75%, 80%, 85%, 90%, 95%, or less. In some embodiments, the lipid composition comprises the SORT lipid at a molar percentage from about 20% to about 65%. In some embodiments, the lipid composition comprises the ionizable cationic lipid at a molar percentage from about 5% to about 30%. In some embodiments, the lipid composition comprises a phospholipid at a molar percentage from about 8% to about 23%.

In some embodiments, the lipid composition comprises a steroid or steroid derivative. In some embodiments, the steroid or steroid derivative is at a molar percentage of about 15%. In some embodiments, the steroid or steroid derivative is at a molar percentage from about 15% to about 46%. In some embodiments, the steroid or steroid derivative is at a molar percentage of 15% or greater. In some embodiments, the steroid or steroid derivative is at a molar percentage of 46% or less. In some embodiments, the lipid composition further comprises a polymer-conjugated lipid. In some embodiments, the polymer-conjugated lipid is a poly(ethylene glycol) (PEG)-conjugated lipid). In some embodiments, the polymer-conjugated lipid is at a molar percentage of about 0.5%. In some embodiments, the polymer-conjugated lipid is at a molar percentage of about 10%. In some embodiments, the polymer-conjugated lipid is at a molar percentage from about 0.5% to 10%. In some embodiments, the polymer-conjugated lipid is at a molar percentage of 0.5% or greater. In some embodiments, the polymer-conjugated lipid is at a molar percentage of 10% or less.

Provided herein are (e.g., pharmaceutical) composition s that comprise components that allow for an improved efficacy or outcome based on the delivery of the polynucleotide. The compositions described elsewhere herein may be more effective at delivery to a particular cell, cell type, organ, or bodily region as compared to a reference composition or compound. The compositions described elsewhere herein may be more effective at generating increase expression of a corresponding polypeptide of a delivered polynucleotide. The compositions described elsewhere herein may be more effective at generating a larger number of cells that express a corresponding polypeptide of a delivered polynucleotide. The compositions described elsewhere herein may result in an increase uptake of the polynucleotide as compared to a reference polynucleotide. The increased uptake may be result of improved stability of polynucleotide or an improved targeting of the composition to a particular cell type or organ. In some embodiments, the SORT lipid is present in an amount in the lipid composition to effect a greater expression or activity of the polynucleotide (or corresponding polypeptide of the polynucleotide) in a cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect at least a 1.1 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 2-fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 5 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 10-fold greater expression or activity of the polynucleotide (or corresponding polypeptide of the polynucleotide) in a cell compared to that achieved with a reference lipid composition comprising LF92, a phospholipid, cholesterol, and a PEG-lipid.

In some embodiments, the SORT lipid is present in an amount in the lipid composition to effect an expression or activity of the polynucleotide (or corresponding polypeptide of the polynucleotide) in a greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 1.1-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 2-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 5-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 10-fold greater plurality of cells compared to that achieved with a reference lipid composition comprising LF92, a phospholipid, cholesterol, and a PEG-lipid.

In some embodiments, the SORT lipid is present in an amount in the lipid composition to effect an uptake of the polynucleotide in a greater plurality of cells compared to that achieved with a reference lipid composition comprising LF92, a phospholipid, cholesterol, and a PEG-lipid. In some embodiments, the SORT lipid is present in an amount in the lipid composition to effect an uptake of the polynucleotide in a greater amount to a cell compared to that achieved with a reference lipid composition comprising LF92, a phospholipid, cholesterol, and a PEG-lipid.

### Phospholipids or Other Zwitterionic Lipids

In various embodiments described herein in the “lipid formulations” section, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

### Steroids or Steroid Derivatives

In various embodiments described herein in the “lipid formulations” section, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

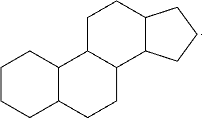 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

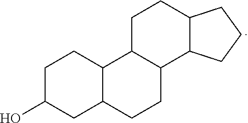 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

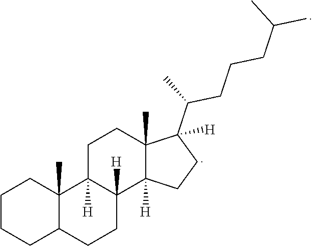

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

### Polymer-Conjugated Lipids

In various embodiments described herein in the “lipid formulations” section, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

In various embodiments of the lipid formulations, the PEG lipid has a structural formula:

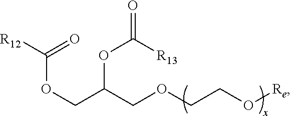

wherein: R₁₂ and R₁₃ are each independently alkyl_{(C≤24)}, alkenyl_{(C≤24)}, or a substituted version of either of these groups; Re is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In various embodiments, the PEG lipid has a structural formula:

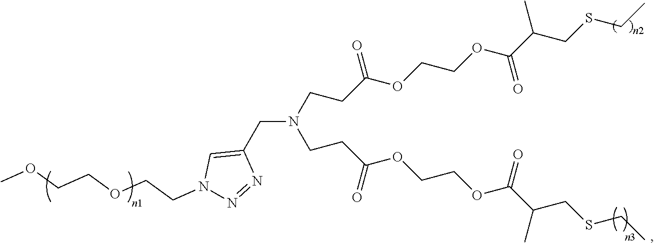

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

### Pharmaceutical Compositions

Some embodiments of the (e.g., pharmaceutical) composition disclosed herein comprise a particular molar ratio of the components or atoms. In some embodiments, the (e.g., pharmaceutical) composition comprises a particular molar ratio of nitrogen in the lipid composition to the phosphate in the polynucleotide (N/P ratio). In some embodiments, the molar ratio of nitrogen in the lipid composition to phosphate in the polynucleotide (N/P ratio) is no more than about 20:1. In some embodiments, the N/P ratio is from about 5:1 to about 20:1. In some embodiments, the N/P ratio is no more than 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, or less. In some embodiments, the N/P ratio is at least 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, or more. In some embodiments, the N/P ratio is of any one of the following values or within a range of any two of the following values: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1.

In some embodiments, composition comprises a particular molar ratio of the polynucleotide to total lipids of the lipid composition. In some embodiments, the molar ratio of the polynucleotide to total lipids of the lipid composition is no more than about 1:1, 1:10, 1:50, or 1:100. In some embodiments, the molar ratio of the polynucleotide to total lipids of the lipid composition is no more than about 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:75, or 1:100 or less. In some embodiments, the molar ratio of the polynucleotide to total lipids of the lipid composition is at least about 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:75, or 1:100 or more. In some embodiments, the molar ratio of the polynucleotide to total lipids of the lipid composition is of any one of the following values or within a range of any two of the following values: 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:75, and 1:100.

In some embodiments, the lipid composition comprises a plurality of particles. The plurality of particles may be characterized by a particular size. For example, the plurality of particles may have an average size. In some embodiments the lipid composition comprises a plurality of particles characterized by a size (e.g., average size) of 100 nanometers (nm) or less. The plurality of particles may be characterized by a size of no more than 10 nm, 20 nm, 30 nm, 40 nm, 50 nm, 60 nm, 70 nm, 80 nm, 90 nm, 100 nm or less. The plurality of particles may be characterized by a size of at least 10 nm, 20 nm, 30 nm, 40 nm, 50 nm, 60 nm, 70 nm, 80 nm, 90 nm, 100 nm or more. The plurality of particles may be characterized by a size of any one of the following values or within a range of any two of the following values: 10 nm, 20 nm, 30 nm, 40 nm, 50 nm, 60 nm, 70 nm, 80 nm, 90 nm, and 100 nm. The (e.g., average) size may be determined by spectroscopic method(s) or image-based method(s), for example, dynamic light scattering, static light scattering, multi-angle light scattering, laser light scattering, or dynamic image analysis, or a combination thereof.

In some embodiments, the plurality of particles may be characterized by a particular polydispersity index (PDI) In some embodiments, the lipid composition comprises a plurality of particles characterized by a polydispersity index (PDI) of no more than about 0.2.

In some embodiments, the plurality of particles may be characterized by a particular zeta potential. In some embodiments, the lipid composition comprises a plurality of particles characterized by a negative zeta potential of −5, −4, or −3 millivolts (mV) or a smaller negative value. For example, the plurality of particles may be characterized by a negative zeta potential of −2 mV. In some embodiments, the lipid composition comprises a plurality of particles with a negative zeta potential of −5 millivolts (mV) or less. In some embodiments, the lipid composition comprises a plurality of particles with a negative zeta potential of −10 millivolts (mV) or less. In some embodiments, the lipid composition comprises a plurality of particles with a negative zeta potential of −15 millivolts (mV) or less. In some embodiments, the lipid composition comprises a plurality of particles with a negative zeta potential of −20 millivolts (mV) or less. In some embodiments, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments, the lipid composition comprises a plurality of particles with a negative zeta potential of 15 millivolts (mV) or less. In some embodiments, the lipid composition comprises a plurality of particles with a negative zeta potential of 20 millivolts (mV) or less.

In some embodiments, the lipid composition comprises a plurality of particles with a negative zeta potential of −5 millivolts (mV) or more. In some embodiments, the lipid composition comprises a plurality of particles with a negative zeta potential of −10 millivolts (mV) or more. In some embodiments, the lipid composition comprises a plurality of particles with a negative zeta potential of −15 millivolts (mV) or more. In some embodiments, the lipid composition comprises a plurality of particles with a negative zeta potential of −20 millivolts (mV) or more. In some embodiments, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments, the lipid composition comprises a plurality of particles with a negative zeta potential of 15 millivolts (mV) or more. In some embodiments, the lipid composition comprises a plurality of particles with a negative zeta potential of 20 millivolts (mV) or more.

The particles of the lipid composition may encapsulate other components of the (e.g., pharmaceutical) composition. In some embodiments, the polynucleotide is encapsulated in particles of the lipid composition. In some embodiments, at least about 85% of the polynucleotide is encapsulated in particles of the lipid compositions. In some embodiments, at least about 75% of the polynucleotide is encapsulated in particles of the lipid compositions. In some embodiments, at least about 65% of the polynucleotide is encapsulated in particles of the lipid compositions.

In some embodiments (especially of the SORT formulations), the lipid composition (with or without polynucleotide(s) assembled therewith) comprises particular physical characteristic(s). For example, the lipid composition may comprise an apparent ionization constant (pKa). In some embodiments, the lipid composition has an (pKa) is of about 8 or higher. In some embodiments, the lipid composition has an (pKa) is within a range of 8 to 13. In some embodiments, the lipid composition has an (pKa) is of 13 or less.

In some embodiments, the (e.g., pharmaceutical) composition comprises one or more pharmaceutically acceptable excipients.

In some embodiments, the (e.g., pharmaceutical) composition is formulated for inhalation. In some embodiments, the (e.g., pharmaceutical) composition is able to be aerosolized, nebulized, or in an (e.g., inhalable) aerosol composition. In some embodiments, the present disclosure provides an aerosol composition comprising a (e.g., pharmaceutical) composition as described elsewhere herein.

In some embodiments, the (e.g., pharmaceutical) composition may be a dry powder. The dry powder may comprise a polynucleotide (as described anywhere herein) assembled with a lipid composition (as described anywhere herein). The dry powder may be administered to a subject in the dry powder form. The dry powder may be generated by spray drying. The dry powder formulation may maintain an encapsulation or interaction of the polynucleotide with the lipid composition (e.g., nanoparticle or nanocapsule). In some cases, the (e.g., pharmaceutical) composition may be a dry powder for delivery via inhalation.

In some embodiments, the aerosol composition is generated by a nebulizer. The nebulizer may comprise a nebulization rate from 0.2 milliliter (mL) per minute (mL/min) to 1 mL/min. The nebulization rate may allow a therapeutically effective dose to be administered to the subject. In some embodiments, the aerosol composition is generated by a nebulizer at a nebulization rate of no more than 70 mL/minute. In some embodiments, the aerosol composition is generated by a nebulizer at a nebulization rate of no more than 50 mL/minute. In some embodiments, the aerosol composition is generated by a nebulizer at a nebulization rate of no more than 30 mL/minute.

In some embodiments, the aerosol composition has an average or median droplet size. For example, the average or median droplet size may be from 1 micron (μm) to 10 μm. The average or median droplet size may allow a therapeutically effective dose to be administered to the subject. In some embodiments, the aerosol composition has an average droplet size from about to about 0.5 micron (μm) to about 10 μm. In some embodiments, the aerosol composition has an average droplet size from about to about 0.5 micron (μm) to about 10 μm. In some embodiments, the aerosol composition has an average droplet size from about to about 1 micron (μm) to about 10 μm. In some embodiments, the aerosol composition has an average droplet size from about to about 0.5 micron (μm) to about 5 μm. In some embodiments, the aerosol droplets are generated by a nebulizer at a nebulization rate of no more than 70 mL/minute. In some embodiments, the aerosol droplets have a mass median aerodynamic diameter (MMAD) from about 0.5 micron (μm) to about 10 μm. In some embodiments, the droplet size varies less than about 50% for a duration of about 24 hours under a storage condition. In some embodiments, droplets of said aerosol composition are characterized by a geometric standard deviation (GSD) of no more than about 3.

In some embodiments, the dose is administered intradermally, subcutaneously, orally, intravenously, intravitreally (or otherwise injected into the eye), intra-arterially, intra-abdominally, intraperitoneally, intrathecally, or intramuscularly. In some embodiments, the (e.g., pharmaceutical) composition is administered using a device implanted into the eye or other body part. In some embodiments, the subject is selected from the group consisting of mouse, rat, monkey, and human.

The (e.g., pharmaceutical) composition can be administered for therapy by any suitable route including oral, rectal, nasal, topical (including transdermal, aerosol, buccal and sublingual), vaginal, parenteral (including subcutaneous, subcutaneous by infusion pump, intramuscular, intravenous and intradermal), intravitreal, and pulmonary. The (e.g., pharmaceutical) composition can take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition, the (e.g., pharmaceutical) composition can also contain other pharmaceutically active compounds or a plurality of compounds of the invention.

In some embodiments, the (e.g., pharmaceutical) composition can be administered subcutaneously, orally, intramuscularly, or intravenously. In one embodiment, the (e.g., pharmaceutical) composition is administered at a therapeutically effective dose. The (e.g., pharmaceutical) composition may be administered via inhalation. For example, the composition may be aerosolizable or inhalable.

In some cases, the administration of a dose may be performed over a duration, e.g., a short period of time. The duration may be no more than 10 minutes (e.g., from about 5 to 8 min). In some embodiments, the administration of a dose of the therapeutic agent may be repeated.

The administration of the compositions may result in a therapeutic effect in the subject or subject's cells, e.g., comparable to normal controls. For example, the cilia of the lungs may recover or improve in their function. A beat frequency and or synchronized (e.g., wave-like) motion of cilia may be recovered or improved in the subject after administration of the compositions described throughout this application. The administration may have minimal off-target or negative byproducts. For example, the administration of the compositions may retain cellular viability throughout the subject.

### Kits

Provided herein, in some embodiments, is a kit comprising a (e.g., pharmaceutical) composition described herein, a container, and a label or package insert on or associated with the container.

### Methods

Provided herein includes a method for enhancing an expression or activity of dynein axonemal intermediate chain 1 (DNAI1) protein in a (e.g., lung) cell, the method comprising: contacting said (e.g., lung) cell with a composition comprising a synthetic polynucleotide assembled with a lipid composition, wherein said synthetic polynucleotide encodes a DNAI1 protein, wherein said lipid composition comprises an ionizable cationic lipid and a selective organ targeting (SORT) lipid separate from said ionizable cationic lipid, thereby providing a(n) (e.g., therapeutically) effective amount or activity of a functional variant (e.g., wild-type form) of DNAI1 protein in said (e.g., lung) cell.

In some embodiments, the method provides a(n) (e.g., therapeutically) effective amount or activity of said functional variant (e.g., wild-type form) of DNAI1 protein in said (e.g., lung) cell at least about 6 hours after said contacting. The contacting may be in vivo. The contacting may be ex vivo. The contacting may be in vitro.

In some embodiments, the method provides a(n) (e.g., therapeutically) effective amount or activity of said functional variant (e.g., wild-type form) of DNAI1 protein in at least about 5%, 10%, 15%, or 20% lung epithelial cells comprising said (e.g., lung) cell. In some embodiments, the method provides a(n) (e.g., therapeutically) effective amount or activity of said functional variant (e.g., wild-type form) of DNAI1 protein in at least about 2%, 5%, or 10% lung ciliated cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung secretory cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung club cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung goblet cells comprising said (e.g., lung) cell. In some embodiments, the method provides a(n) (e.g., therapeutically) effective amount or activity of said functional variant (e.g., wild-type form) of DNAI1 protein in at least about 5%, 10%, 15%, or 20% lung basal cells comprising said (e.g., lung) cell.

In some embodiments, the (e.g., lung) cell is in a ciliary axoneme. In some embodiments, the (e.g., lung) cell is an airway epithelial cell (e.g., a bronchial epithelial cell). In some embodiments, the (e.g., lung) cell is a ciliated cell, a basal cell, a goblet cell, or a club cell. In some embodiments, the (e.g., lung) cell is a ciliated cell, a basal cell, or a club cell. In some embodiments, the (e.g., lung) cell exhibits a mutation in DNAI1 gene or transcript.

In some embodiments, the contacting comprises contacting a plurality of (e.g., lung) cells that comprises said (e.g., lung) cell. In some embodiments, the plurality of (e.g., lung) cells comprises ciliated cell(s), basal cell(s), goblet cell(s), club cell(s), or a combination thereof. In some embodiments, the plurality of (e.g., lung) cells comprises ciliated cell(s), basal cell(s), club cell(s), or a combination thereof. In some embodiments, mucus is present in said contacting.

In some embodiments, the contacting is repeated (e.g., at least about 2, 4, 6, 8, or 10 times). In some embodiments, the repeated contacting is at least once a week, at least twice a week, or at least three times a week. In some embodiments, at least one contacting steps of said repeated contacting is followed by a treatment holiday. In some embodiments, the repeated contacting is characterized by a duration of at least 1, 2, 3, 4, or 5 week(s). In some embodiments, mucus is present in one or more contacting steps of said repeated contacting.

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, or 7 days) after said contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The contacting may be ex vivo or in vitro.

Ciliary function may be measured by any method known in the art. In some embodiments, ciliary activity is measured by comparing a measured ciliary beat frequency (CBF) to a normal value (e.g., 7-16 Hz). In some embodiments, CBF may be determined by imaging and counting ciliary beating cycles. In some embodiments, the imaging technique may comprise microscopy, tomography, videography, or any combination thereof. In some embodiments, ciliary function may be measured by measurement of ciliary shaft structure. In some embodiments, the measurement may comprise imaging comprising microscopy, tomography, videography, or any combination thereof. In some embodiments, ciliary function may be measured by expression of ciliary proteins. Expression of ciliary proteins may be determined by any technique known in the art (e.g., proteomics, immunofluorescence, western blotting).

In some embodiments, the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, 7, 8, 9, 10, 11, 12, 13, or 14 days) after a contacting of said repeated contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof. The repeated contacting(s) may be (e.g., partially) ex vivo or in vitro.

Provided herein includes a method for treating a subject having or suspected of having primary ciliary dyskinesia (PCD), comprising administering to the subject a (e.g., pharmaceutical) composition as provided hereinabove or elsewhere herein. The (e.g., pharmaceutical) compositions as described hereinabove or elsewhere herein may be effective at treating a subject having PCD. The (e.g., pharmaceutical) compositions as described hereinabove or elsewhere herein may be effective at treating a subject suspected of having PCD. The (e.g., pharmaceutical) compositions may alleviate or eliminate symptoms of PCD in the subject (e.g., regardless whether the subject has been determined to have PCD).

Provided herein, in some aspects, is a method for treating a subject having or suspected of having primary ciliary dyskinesia (PCD), comprising administering to the subject a (e.g., pharmaceutical) composition comprising a heterologous polynucleotide assembled with a lipid composition, which heterologous polynucleotide encodes a dynein axonemal intermediate chain 1 (DNAI1) protein, thereby resulting in a heterologous expression of the DNAI1 protein within cells of the subject.

Provided herein method of treating a having or suspected of having primary ciliary dyskinesia (PCD) comprising administrating the (e.g., pharmaceutical) compositions that comprise component, thereby generating an improved efficacy or outcome of treatment. The methods for treating a subject having or suspected of having primary ciliary dyskinesia (PCD) may be more effective at delivery to a particular cell, cell type, organ, or bodily region as compared to a treating with a reference composition or compound. The methods for treating a subject having or suspected of having primary ciliary dyskinesia (PCD) described elsewhere herein may be more effective at generating increase expression of a corresponding polypeptide of a delivered polynucleotide. The methods for treating a subject having or suspected of having primary ciliary dyskinesia (PCD) described elsewhere herein may be more effective at generating a larger number of cells that express a corresponding polypeptide of a delivered polynucleotide. The methods for treating a subject having or suspected of having primary ciliary dyskinesia (PCD) described elsewhere herein may result in an increase uptake of the polynucleotide as compared to a reference polynucleotide. The increased uptake may be result of improved stability of polynucleotide or an improved targeting of the composition to a particular cell type or organ. In some embodiments, the methods for treating a subject having or suspected of having primary ciliary dyskinesia (PCD) comprise administering to the subject a composition comprising a SORT lipid present in an amount in the lipid composition to effect a greater expression or activity of the polynucleotide (or corresponding polypeptide of the polynucleotide) in a cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect at least a 1.1 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 2-fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 5 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 10-fold greater expression or activity of the polynucleotide (or corresponding polypeptide of the polynucleotide) in a cell compared to that achieved with a reference lipid composition comprising LF92, a phospholipid, cholesterol, and a PEG-lipid.

In some embodiments, the methods for treating a subject having or suspected of having primary ciliary dyskinesia (PCD) comprise administering to the subject a composition comprising a SORT lipid present in an amount in the lipid composition to effect an expression or activity of the polynucleotide (or corresponding polypeptide of the polynucleotide) in a greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 1.1-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 2-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 5-fold greater plurality of cells compared to that achieved with a reference lipid composition comprising LF92, a phospholipid, cholesterol, and a PEG-lipid. In some embodiments, the SORT lipid is present in an amount in the lipid composition to effect an expression or activity of the polynucleotide (or corresponding polypeptide of the polynucleotide) in at least a 10-fold greater plurality of cells compared to that achieved with a reference lipid composition comprising LF92, a phospholipid, cholesterol, and a PEG-lipid.

In some embodiments, the methods for treating a subject having or suspected of having primary ciliary dyskinesia (PCD) comprise administering to the subject a composition comprising a SORT lipid present in an amount in the lipid composition to effect an uptake of the polynucleotide in a greater plurality of cells compared to that achieved with a reference lipid composition comprising LF92, a phospholipid, cholesterol, and a PEG-lipid. In some embodiments, the SORT lipid is present in an amount in the lipid composition to effect an uptake of the polynucleotide in a greater amount to a cell compared to that achieved with a reference lipid composition comprising LF92, a phospholipid, cholesterol, and a PEG-lipid

In some embodiments, the lipid composition is described hereinabove in the “SORT formulations” section. The lipid composition may comprise (i) an ionizable cationic lipid (such as one described hereinabove in the “SORT formulations” section), (ii) a phospholipid (such as one described hereinabove in the “SORT formulations” section), and (iii) a selective organ targeting (SORT) lipid separate from the ionizable cationic lipid and the phospholipid. The SORT lipid may be one described hereinabove in the “Selective Organ Targeting Compounds” section. The ionizable cationic lipid may be one described hereinabove in the “Dendrimers or dendrons of Formula (I)” or “Dendrimers or dendrons of Formula (X)” section.

In some other embodiments, the lipid composition is described hereinabove in the “LF92 formulations” section. For example, the lipid composition comprises a cationic lipid having a structural formula (I′):

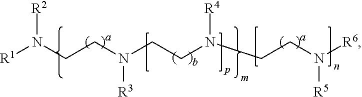

wherein:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R₇, wherein R⁷ is independently selected from C₃-C₁₈ alkyl or C₃-C₁₈ alkenyl having one C═C double bond; and wherein one or more of the nitrogen atoms indicated in formula (I′) may be protonated to provide a cationic lipid. In some embodiments, the cationic lipid is 13,16,20-tris(2-hydroxydodecyl)-13,16,20,23-tetraazapentatricontane-11,25-diol. In some embodiments, the cationic lipid is (11R,25R)-13,16,20-tris((R)-2-hydroxydodecyl)-13,16,20,23-tetraazapentatricontane-11,25-diol.

In various embodiments of the method described herein in this section, the heterologous polynucleotide may be one described hereinabove in the “Nucleic Acids” section. For example, the polynucleotide may comprise a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15.

The methods for treating the subject may comprise administering using a variety of administration methods as described elsewhere herein. The administration may be performed in such a way to target or come in contact with an organ of interest. In some embodiments, the administering comprises administering to a lung by nebulization. The methods as described herein may comprises treating or administering a composition to the subject. In some cases, the subject may be determined to have PCD. The subject may be observed or determined to have a genetic or expression profile that is aberrant from a health individual. An aberrant genetic profile or expression profile may be indicative of a particular disease or disorder. The subject may be determined to exhibit aberrant expression or activity of the DNAI1 gene or protein. The subject may have a pathogenic mutation in the DNAI1 gene or protein. The aberrant expression or activity may be an excess or increased activity of a protein or gene that results in a disease state. The aberrant expression or activity may be a decrease or loss of activity of a protein or gene that results in a disease state. The aberrant expression may be a loss of activity such that a particular function of a protein is lost. The aberrant expression may be alleviated by the introduction of a composition that increases the expression of a protein and allows a regain of protein function in a cell or organ. The subject may have a decrease functionality of their lungs. The subject may have a decreased lung capacity or ability to expel air from the lungs. For example, subject may have a lower forced expiratory volume in one second (FEV1) as compared to a healthy or baseline individual. The subject may have a FEV1 value of 30% to 90% or 40% to 90%.

The cells comprising aberrant expression and/or the cells wherein the composition are administered to may be a particular type of cell or located in a particular area of the body of the subject. The cells may be lung cells. The cells may be located in the lung of the subject. The cells may be undifferentiated or differentiated. In some embodiments, the cells comprise ciliated cell(s), club cell(s), or basal cell(s), or any combination thereof. In some embodiments, the cells comprise lung epithelial cell(s). In some embodiments, the cells comprise or are ciliated cells. In some embodiments, the ciliated cells are ciliated epithelial cells. For example, the ciliated cells may be ciliated airway epithelial cells. In some embodiments, the epithelial cells are undifferentiated. In some embodiments, the epithelial cells are differentiated. The cells may be located in the trachea, bronchi, bronchioles, or other parts of the lung or associated areas.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A pharmaceutical composition comprising a polynucleotide assembled with a lipid composition, wherein: said polynucleotide encodes a dynein axonemal intermediate chain 1 (DNAI1) protein; and said lipid composition comprises (i) an ionizable cationic lipid, and (ii) a selective organ targeting (SORT) lipid separate from said ionizable cationic lipid; optionally, wherein said SORT lipid is selected from those set forth in Table 7, or pharmaceutically acceptable salts thereof, or a subset of the lipids and the pharmaceutically acceptable salts thereof.

Embodiment 2. The pharmaceutical composition of Embodiment 1, wherein said lipid composition further comprises (iii) a phospholipid.

Embodiment 3. The pharmaceutical composition of Embodiment 1 or 2, wherein said polynucleotide comprises a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15.

Embodiment 4. The pharmaceutical composition of Embodiment 1 or 3, wherein said nucleic acid sequence has at least about 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence over at least 1,000 bases (e.g., nucleotide residues 1 to 1,000) of SEQ ID NO: 15.

Embodiment 5. The pharmaceutical composition of Embodiment 4, wherein said nucleic acid sequence has 100% sequence identity to a sequence over at least 1,000 bases (e.g., nucleotide residues 1 to 1,000) of SEQ ID NO: 15.

Embodiment 6. The pharmaceutical composition of any one of Embodiments 1-5, wherein at least 90%, 95%, or 97% nucleotides replacing uridine within said polynucleotide are nucleotide analogues.

Embodiment 7. The pharmaceutical composition of any one of Embodiments 1-6, wherein fewer than 15% of nucleotides within said polynucleotide are nucleotide analogues.

Embodiment 8. The pharmaceutical composition of any one of Embodiments 1-7, wherein said polynucleotide comprises 1-methylpseudouridine.

Embodiment 9. The pharmaceutical composition of any one of Embodiments 1-8, wherein said nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CAG, AGG, CGG, CGA, CGT, CGC, TCG, TCA, TCT, TCC, ACG, ACT, GTA, GTT, GTC, and TAT, as compared to a corresponding wild-type sequence selected from SEQ ID NO:16.

Embodiment 10. The pharmaceutical composition of any one of Embodiments 1-9, wherein said nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence selected from SEQ ID NO: 16

Embodiment 11. The pharmaceutical composition of any one of Embodiments 1-10, wherein said nucleic acid sequence comprises fewer codon types encoding an amino acid as compared to a corresponding wild-type sequence selected from SEQ ID NO: 16.

Embodiment 12. The pharmaceutical composition of any one of Embodiments 1-11, wherein at least one type of an isoleucine-encoding codons in said corresponding wild-type sequence is substituted with a synonymous codon type in said nucleic acid sequence.

Embodiment 13. The pharmaceutical composition of any one of Embodiments 1-12, wherein at least one type of a valine-encoding codons in said corresponding wild-type sequence is substituted with a synonymous codon type in said nucleic acid sequence.

Embodiment 14. The pharmaceutical composition of any one of Embodiments 1-13, wherein at least one type of an alanine-encoding codons in said corresponding wild-type sequence is substituted with a synonymous codon type in said nucleic acid sequence.

Embodiment 15. The pharmaceutical composition of any one of Embodiments 1-14, wherein at least one type of a glycine-encoding codons in said corresponding wild-type sequence is substituted with a synonymous codon type in said nucleic acid sequence.

Embodiment 16. The pharmaceutical composition of any one of Embodiments 1-15, wherein at least one type of a proline-encoding codons in said corresponding wild-type sequence is substituted with a synonymous codon type in said nucleic acid sequence.

Embodiment 17. The pharmaceutical composition of any one of Embodiments 1-16, wherein at least one type of a threonine-encoding codons in said corresponding wild-type sequence is substituted with a synonymous codon type in said nucleic acid sequence.

Embodiment 18. The pharmaceutical composition of any one of Embodiments 1-17, wherein at least one type of a leucine-encoding codons in said corresponding wild-type sequence is substituted with a synonymous codon type in said nucleic acid sequence.

Embodiment 19. The pharmaceutical composition of any one of Embodiments 1-18, wherein at least one type of an arginine-encoding codons in said corresponding wild-type sequence is substituted with a synonymous codon type in said nucleic acid sequence.

Embodiment 20. The pharmaceutical composition of any one of Embodiments 1-19, wherein at least one type of a serine-encoding codons in said corresponding wild-type sequence is substituted with a synonymous codon type in said nucleic acid sequence.

Embodiment 21. The pharmaceutical composition of any one of Embodiments 1-20, wherein said pharmaceutical composition comprises an excipient.

Embodiment 22. The pharmaceutical composition of any one of Embodiments 1-21, wherein said polynucleotide is present in said pharmaceutical composition at a concentration of no more than about 5, no more than about 4, no more than about 3, or no more than about 2 mg/mL.

Embodiment 23. The pharmaceutical composition of any one of Embodiments 1-22, wherein said polynucleotide is present in said pharmaceutical composition at a concentration of no more than 1 mg/mL.

Embodiment 24. The pharmaceutical composition of any one of Embodiments 1-23, wherein a molar ratio of nitrogen in said lipid composition to phosphate in said polynucleotide (N/P ratio) is no more than about 20:1.

Embodiment 25. The pharmaceutical composition of Embodiment 24, wherein said N/P ratio is from about 5:1 to about 20:1.

Embodiment 26. The pharmaceutical composition of any one of Embodiments 1-25, wherein a molar ratio of said polynucleotide to total lipids of said lipid composition is no more than about 1:1, 1:10, 1:50, or 1:100.

Embodiment 27. The pharmaceutical composition of any one of Embodiments 1-26, wherein at least about 85% of said polynucleotide is encapsulated in particles of said lipid compositions.

Embodiment 28. The pharmaceutical composition of any one of Embodiments 1-27, wherein said lipid composition comprises a plurality of particles characterized by a (e.g., average) size of 100 nanometers (nm) or less.

Embodiment 29. The pharmaceutical composition of any one of Embodiments 1-28, wherein said lipid composition comprises a particles characterized by a polydispersity index (PDI) of no more than about 0.2.

Embodiment 30. The pharmaceutical composition of any one of Embodiments 1-29, wherein said lipid composition comprises a plurality of particles characterized by a negative zeta potential of −5, −4, or −3 millivolts (mV) or a lower negative number.

Embodiment 31. The pharmaceutical composition of any one of Embodiments 1-30, wherein said SORT lipid is present in an amount in said lipid composition to effect a (e.g., 1.1-or 10-fold) greater expression or activity of said polynucleotide in a (e.g., lung) cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid.

Embodiment 32. The pharmaceutical composition of any one of Embodiments 1-31, wherein said SORT lipid is present in an amount in said lipid composition to effect a (e.g., 1.1-or 10-fold) greater expression or activity of said polynucleotide in a (e.g., lung) cell compared to that achieved with a corresponding reference lipid composition that does not comprise said SORT lipid.

Embodiment 33. The pharmaceutical composition of Embodiment 31 or 32, wherein said cell is a ciliated cell.

Embodiment 34. The pharmaceutical composition of any one of Embodiments 1-33, wherein said SORT lipid is present in an amount in said lipid composition to effect an expression or activity of said polynucleotide in a (e.g., 1.1- or 10-fold) greater plurality of (e.g., lung) cells compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid.

Embodiment 35. The pharmaceutical composition of any one of Embodiments 1-34, wherein said SORT lipid is present in an amount in said lipid composition to effect an expression or activity of said polynucleotide in a (e.g., 1.1- or 10-fold) greater plurality of (e.g., lung) cells (e.g., ciliated lung cells) compared to that achieved with a corresponding reference lipid composition that does not comprises said SORT lipid.

Embodiment 36. The pharmaceutical composition of Embodiment 34, wherein said plurality of cells are ciliated cells.

Embodiment 37. The pharmaceutical composition of any one of Embodiments 1-36, wherein said lipid composition comprises said SORT lipid at a molar percentage from about 20% to about 65%.

Embodiment 38. The pharmaceutical composition of any one of Embodiments 1-37, wherein said lipid composition comprises said ionizable cationic lipid at a molar percentage from about 5% to about 30%.

Embodiment 39. The pharmaceutical composition of any one of Embodiments 1-38, wherein said lipid composition comprises said phospholipid at a molar percentage from about 8% to about 23%.

Embodiment 40. The pharmaceutical composition of any one of Embodiments 1-39, wherein said phospholipid is not an ethylphosphocholine.

Embodiment 41. The pharmaceutical composition of any one of Embodiments 1-40, wherein said lipid composition further comprises a steroid or steroid derivative (e.g., at a molar percentage from about 15% to about 46%).

Embodiment 42. The pharmaceutical composition of any one of Embodiments 1-41, wherein said lipid composition further comprises a polymer-conjugated lipid (e.g., poly(ethylene glycol) (PEG)-conjugated lipid) (e.g., at a molar percentage from about 0.5% to about 10%, from about T % to about 10%, or from about 2% to about 10%).

Embodiment 43. The pharmaceutical composition of any one of Embodiments 1-42, wherein said lipid composition has an apparent ionization constant (pKa) is of about 8 or higher (e.g., about 8 to about 13).

Embodiment 44. The pharmaceutical composition of any one of Embodiments 1-43, wherein said SORT lipid comprises a permanently positively charged moiety (e.g., a quaternary ammonium ion).

Embodiment 45. The pharmaceutical composition of Embodiment 44, wherein said SORT lipid comprises a counterion.

Embodiment 46. The pharmaceutical composition of any one of Embodiments 1-45, wherein said SORT lipid is a phosphocholine lipid.

Embodiment 47. The pharmaceutical composition of Embodiment 46, wherein said SORT lipid is an ethylphosphocholine, optionally selected from 1,2-dimyristoleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, and 1-palmitoyl-2-oleoyl-sn-glycero-3-ethylphosphocholine.

Embodiment 48. The pharmaceutical composition of any one of Embodiments 1-43, wherein said SORT lipid comprises a headgroup having a structural formula:

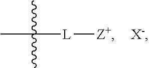

wherein L is a (e.g., biodegradable) linker; Z⁺ is positively charged moiety (e.g., a quaternary ammonium ion); and X⁻ is a counterion.

Embodiment 49. The pharmaceutical composition of any one of Embodiments 1-43, wherein said SORT lipid has a structural formula:

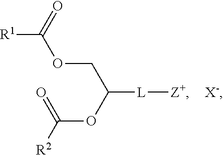

wherein R₁ and R₂ are each independently an optionally substituted C₆-C₂₄ alkyl, or an optionally substituted C₆-C₂₄ alkenyl.

Embodiment 50. The pharmaceutical composition of any one of Embodiments 1-43, wherein said SORT lipid has a structural formula:

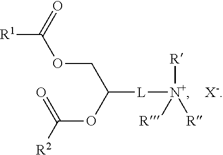

Embodiment 51. The pharmaceutical composition of Embodiment 50, wherein L is

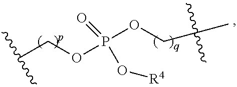

wherein: p and q are each independently 1, 2, or 3; and R⁴ is an optionally substituted C₁-C₆ alkyl.

Embodiment 52. The pharmaceutical composition of any one of Embodiments 1-43, wherein said SORT lipid has a structural formula:

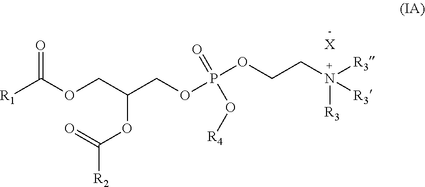

wherein: R₁ and R₂ are each independently alkyl_{(C8-C24)}, alkenyl_{(C8-C24)}, or a substituted version of either group; R₃, R₃′, and R₃′ are each independently alkyl_{(C≤6)} or substituted alkyl_{(C≤6)}; R₄ is alkyl_{(C≤6)} or substituted alkyl_{(C≤6)}; and X is a monovalent anion.

Embodiment 53. The pharmaceutical composition of any one of Embodiments 1-43, wherein said SORT lipid has a structural formula:

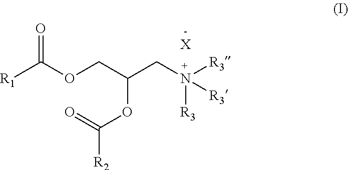

wherein:
R₁ and R₂ are each independently alkyl_{(C8-C24)}, alkenyl_{(C8-C24)}, or a substituted version of either group;
R₃, R₃′, and R₃′ are each independently alkyl_{(C≤6)} or substituted alkyl_{(C≤6)};
X⁻ is a monovalent anion.

Embodiment 54. The pharmaceutical composition of any one of Embodiments 1-43, wherein said SORT lipid has a structural formula:

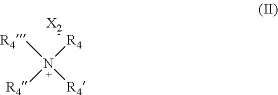

wherein:
R₄ and R₄′ are each independently alkyl_{(C6-C24)}, alkenyl_{(C6-C24)}, or a substituted version of either group;
R₄″ is alkyl_{(C≤24)}, alkenyl_{(C≤24)}, or a substituted version of either group;
R₄′″ is alkyl_{(C1-C8)}, alkenyl_{(C2-C8)}, or a substituted version of either group; and
X₂⁻is a monovalent anion.

Embodiment 55. The pharmaceutical composition of any one of Embodiments 1-43, wherein said SORT lipid has a structural formula:

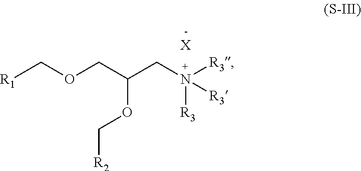

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

Embodiment 56. The pharmaceutical composition of any one of Embodiments 1-43, wherein said SORT lipid has a structural formula:

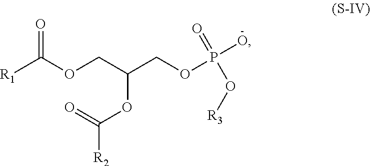

wherein:
R₁ and R₂ are each independently alkyl_{(C8-C24)}, alkenyl_{(C8-C24)}, or a substituted version of either group;
R₃ is hydrogen, alkyl_{(C≤6)}, or substituted alkyl_{(C≤6)}, or —Y₁—R₄, wherein:
Y₁ is alkanediyl_{(C≤6)} or substituted alkanediyl_{(C≤6)}; and
R₄ is acyloxy_{(C≤8-24)} or substituted acyloxy_{(C≤8-24)}.

Embodiment 57. The pharmaceutical composition of any one of Embodiments 1-56, wherein said ionizable cationic lipid is a dendrimer or dendron having the formula:

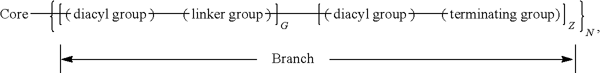

or a pharmaceutically acceptable salt thereof, wherein:
(a) the core comprises a structural formula (X_{Core}):
wherein:

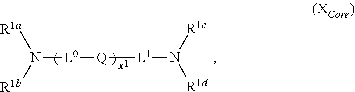

Q is independently at each occurrence a covalent bond, —O—, —S—, —NR²—, or —CR³aR^{3b}—;
R² is independently at each occurrence R^{1g} or —L²—NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}):

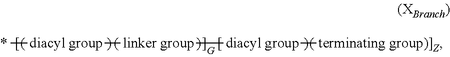

wherein:
* indicates a point of attachment of the branch to the core;
g is 1, 2, 3, or 4;
Z=2^{(g-1)};
G=0, when g=1; or G=Σᵢ₌₀^{i=g-2} 2ⁱ, when g≠1;
(c) each diacyl group independently comprises a structural formula

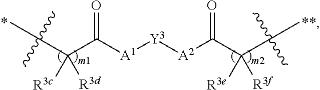

wherein:
* indicates a point of attachment of the diacyl group at the proximal end thereof;
** indicates a point of attachment of the diacyl group at the distal end thereof;
Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR4—wherein:
R₄ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula

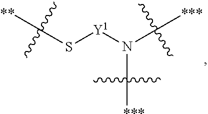

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

Embodiment 58. The pharmaceutical composition of Embodiment 57, wherein x¹ is 0, 1, 2, or 3.

Embodiment 59. The pharmaceutical composition of Embodiment 57 or 58, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl

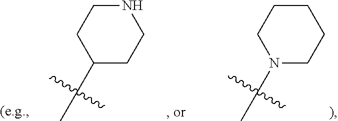 [SMILES: *C1CCNCC1 ; *N1CCCCC1]

N—(C₁-C₃ alkyl)-piperidinyl

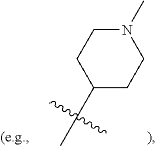 [SMILES: *C1CCN(C)CC1]

piperazinyl

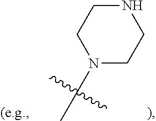 [SMILES: *N1CCNCC1]

N—(C₁-C₃ alkyl)-piperadizinyl

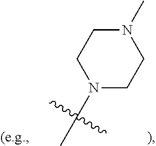 [SMILES: *N1CCN(C)CC1]

morpholinyl

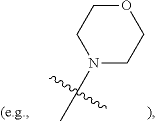 [SMILES: *N1CCOCC1]

### N-pyrrolidinyl

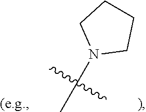 [SMILES: *N1CCCC1]

pyrrolidinyl

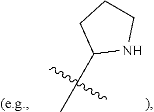 [SMILES: *C1CCCN1]

or N—(C₁-C₃ alkyl)-pyrrolidinyl

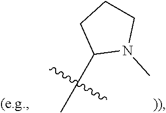 [SMILES: *C1CCCN1C]

and C₃-C₅ heteroaryl (e.g., imidazolyl

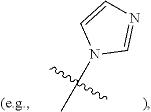 [SMILES: *n1ccnc1]

or pyridinyl

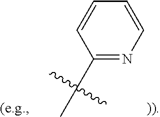 [SMILES: *c1ccccn1]

Embodiment 60. The pharmaceutical composition of Embodiment 59, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

Embodiment 61. The pharmaceutical composition of Embodiment 60, wherein R^{3a} and R^{3b} are each independently at each occurrence hydrogen.

Embodiment 62. The pharmaceutical composition of any of one of Embodiments 57-61, wherein the plurality (N) of branches comprises at least 3 (e.g., at least 4, or at least 5) branches.

Embodiment 63. The pharmaceutical composition of any of one of Embodiments 57-62, wherein g=1; G=0; and Z=1.

Embodiment 64. The pharmaceutical composition of Embodiment 63, wherein each branch of the plurality of branches comprises a structural formula *diacyl group terminating group).

Embodiment 65. The pharmaceutical composition of any of one of Embodiments 57-62, wherein g=2; G=1; and Z=2.

Embodiment 66. The pharmaceutical composition of Embodiment 65, wherein each branch of the plurality of branches comprises a structural formula

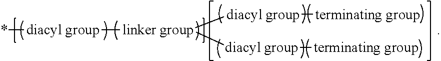

Embodiment 67. The pharmaceutical composition of any of one of Embodiments 57-66, wherein the core comprises a structural formula:

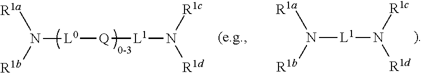

Embodiment 68. The pharmaceutical composition of any of one of Embodiments 57-66, wherein the core comprises a structural formula:

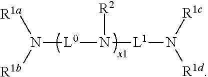

Embodiment 69. The pharmaceutical composition of Embodiment 68, wherein the core comprises a structural formula:

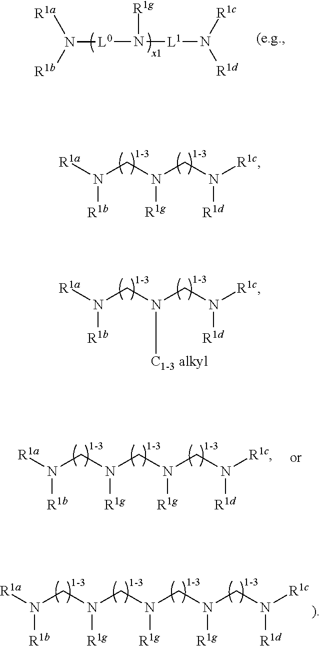

Embodiment 70. The pharmaceutical composition of Embodiment 68, wherein the core comprises a structural formula:

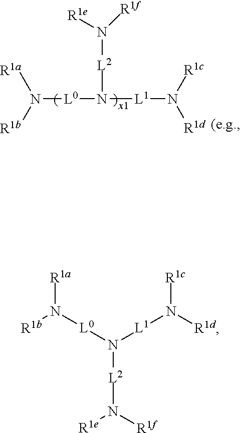

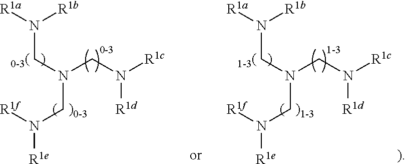

Embodiment 71. The pharmaceutical composition of any of one of Embodiments 57-66, wherein the core comprises a structural formula:

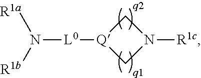

wherein Q′ is —NR²— or —CR^{3a}R^{3b}—; q¹ and q² are each independently 1 or 2.

Embodiment 72. The pharmaceutical composition of Embodiment 71, wherein the core comprises a structural formula:

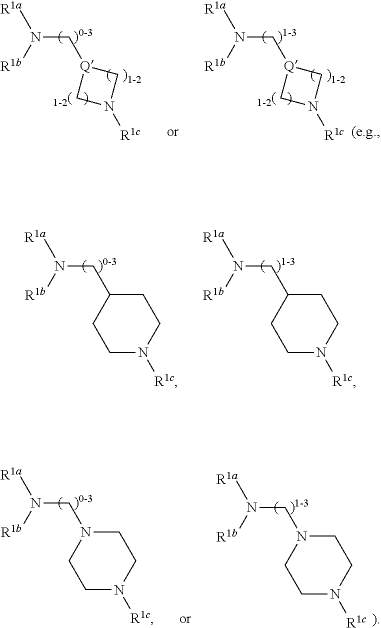

Embodiment 73. The pharmaceutical composition of any of one of Embodiments 57-66, wherein the core comprises a structural formula

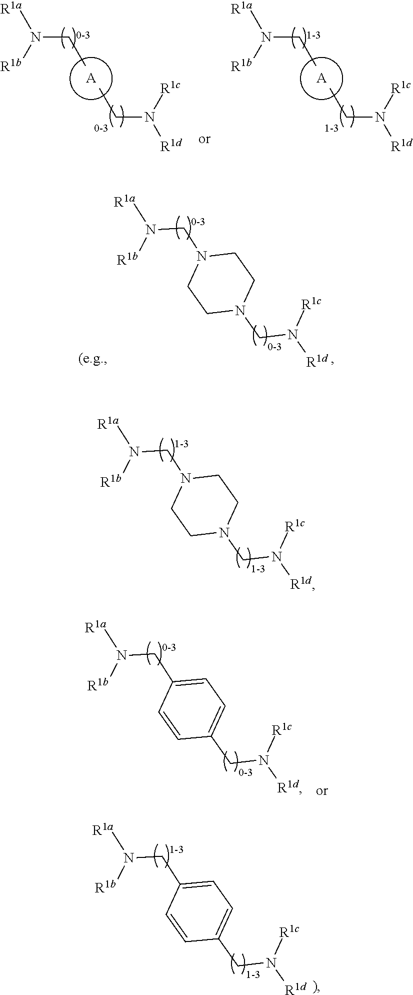

wherein ring A is an optionally substituted aryl or an optionally substituted (e.g., C₃-C₁₂, such as C₃-C₅) heteroaryl.

Embodiment 74. The pharmaceutical composition of any of one of Embodiments 57-66, wherein the core comprises has a structural formula

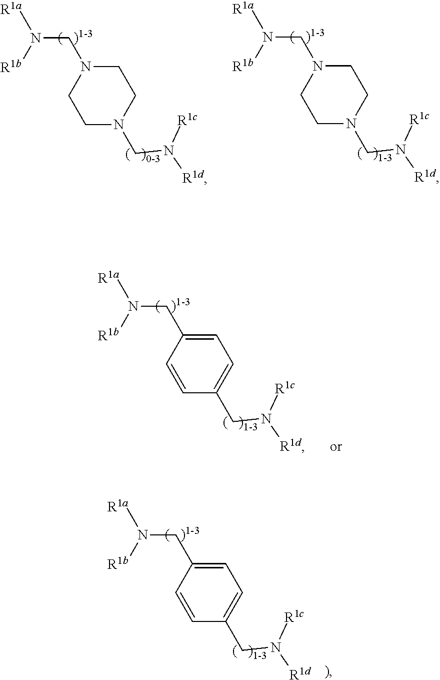

Embodiment 75. The pharmaceutical composition of any of one of Embodiments 57-66, wherein the core is selected from those set forth in Table 3 or a subset thereof; or wherein the core comprises a structural formula selected from the group consisting of:

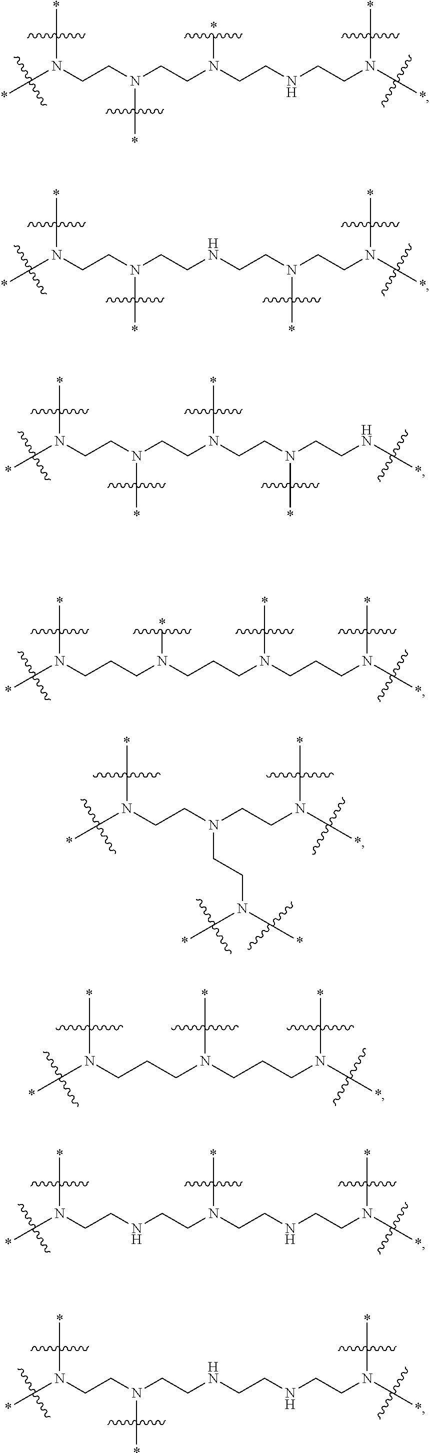

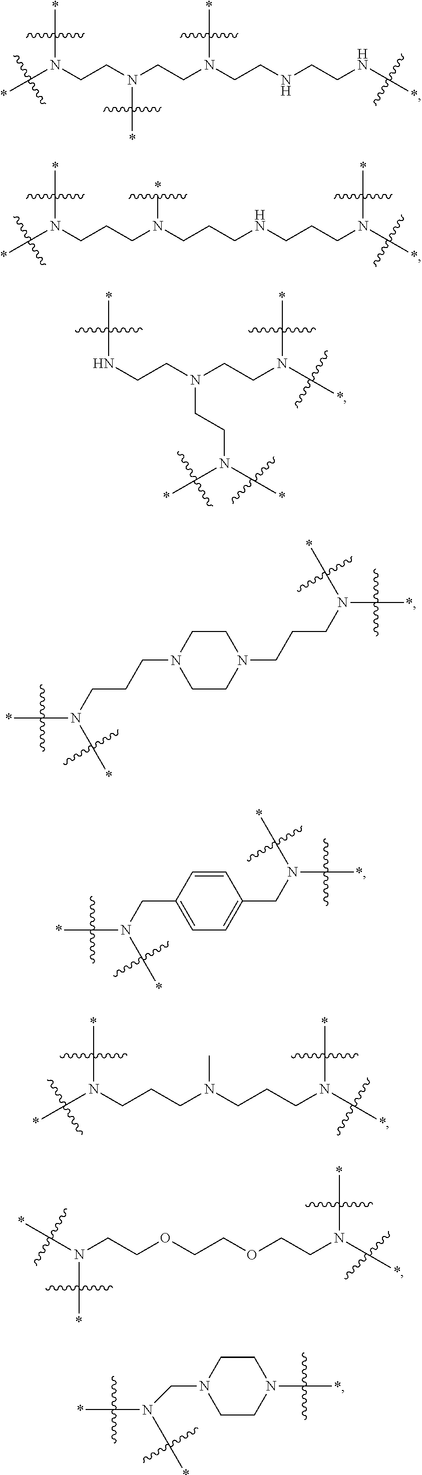

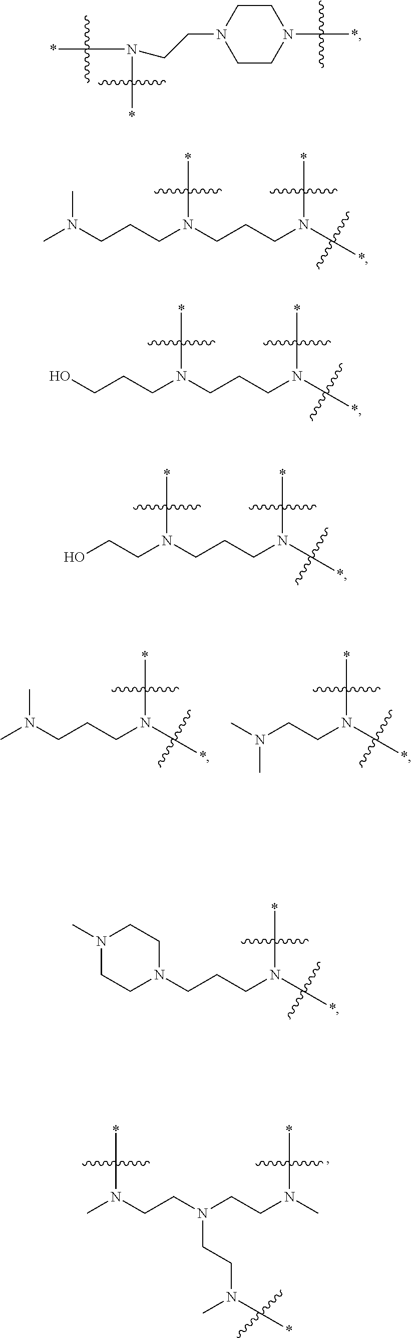

and pharmaceutically acceptable salts thereof, wherein * indicates a point of attachment of the core to a branch of the plurality of branches.

Embodiment 76. The pharmaceutical composition of any of one of Embodiments 57-66, wherein the core comprises a structural formula selected from the group consisting of:

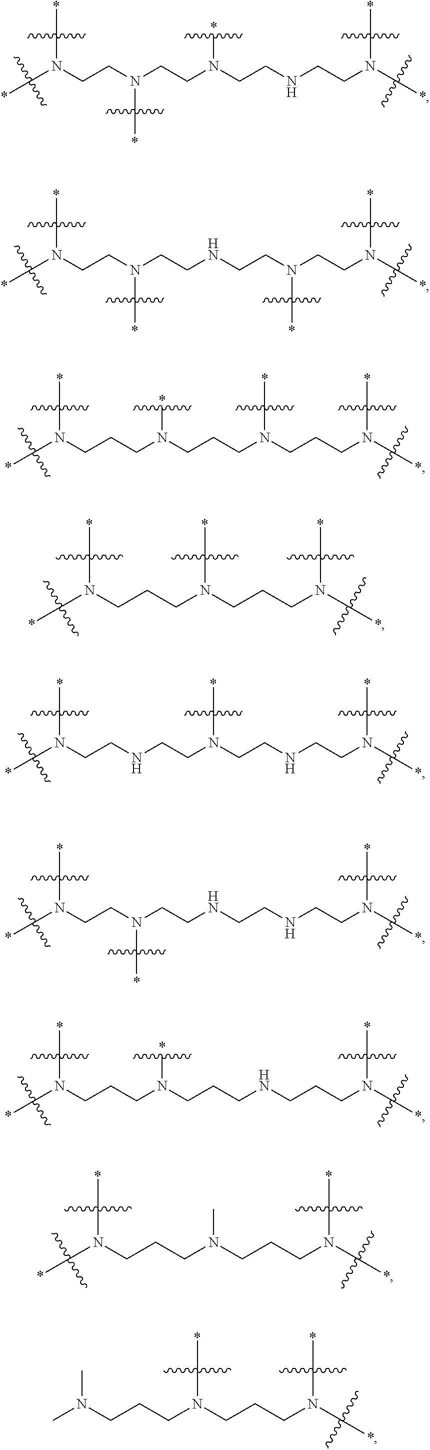

and pharmaceutically acceptable salts thereof, wherein * indicates a point of attachment of the core to a branch of the plurality of branches.

Embodiment 77. The pharmaceutical composition of any of one of Embodiments 57-66, wherein the core comprises a structural formula selected from the group consisting of:

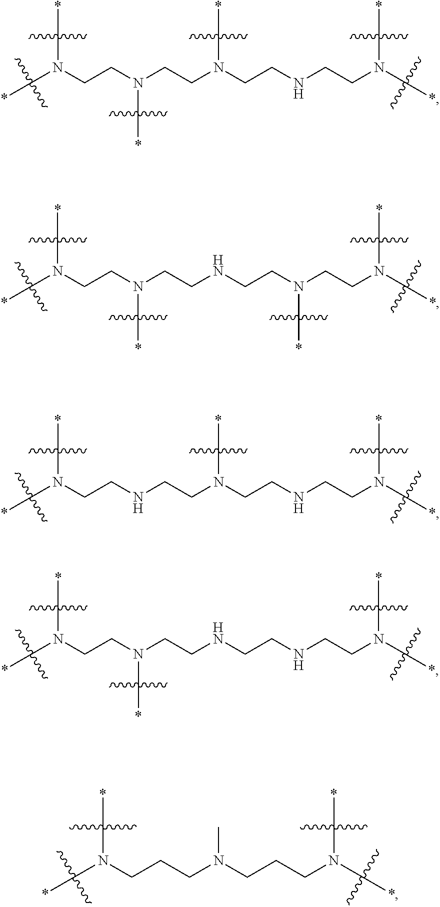

and pharmaceutically acceptable salts thereof, wherein * indicates a point of attachment of the core to a branch of the plurality of branches.

Embodiment 78. The pharmaceutical composition of any of one of Embodiments 57-66, wherein the core has the structure

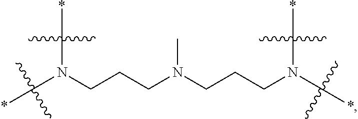

wherein * indicates a point of attachment of the core to a branch of the plurality of branches or H, wherein at least 2 (e.g., at least 3, or at least 4) branches are attached to the core.

Embodiment 79. The pharmaceutical composition of any of one of Embodiments 57-66, wherein the core has the structure

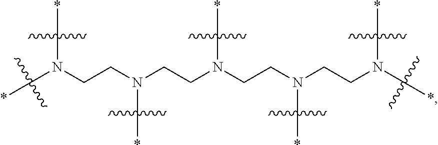

wherein * indicates a point of attachment of the core to a branch of the plurality of branches or H, wherein at least 4 (e.g., at least 5, or at least 6) branches are attached to the core.

Embodiment 80. The pharmaceutical composition of any of one of Embodiments 57-79, wherein A¹ is —O— or —NH—.

Embodiment 81. The pharmaceutical composition of any of one of Embodiments 80, wherein A¹ is —O—.

Embodiment 82. The pharmaceutical composition of any of one of Embodiments 57-81, wherein A² is —O— or —NH—.

Embodiment 83. The pharmaceutical composition of Embodiment 82, wherein A² is -0-.

Embodiment 84. The pharmaceutical composition of any of one of Embodiments 57-83, wherein Y³ is C₁-C₁₂ (e.g., C₁-C₆, such as C₁-C₃) alkylene.

Embodiment 85. The pharmaceutical composition of any of one of Embodiments 57-83, wherein the diacyl group independently at each occurrence comprises a structural formula

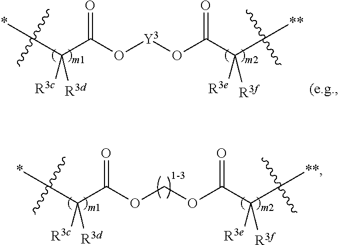

such as

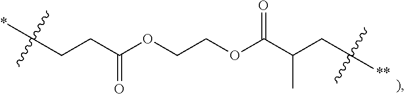

optionally wherein R^{3c}, R^{3d}, R^{3e}, and R^{3f} are each independently at each occurrence hydrogen or C₁-C₃ alkyl.

Embodiment 86. The pharmaceutical composition of any of one of Embodiments 51-85, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

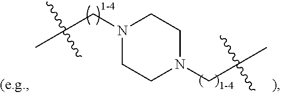

and [(C₁-C₄) alkylene]-phenylene-[(C₁-C₄) alkylene]

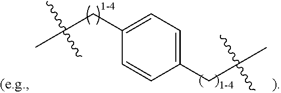

Embodiment 87. The pharmaceutical composition of Embodiment 86, wherein L⁰, L¹, and L² are each independently at each occurrence selected from C₁-C₆ alkylene (e.g., C₁-C₃ alkylene), —(C₁-C₃ alkylene-0)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

Embodiment, 88. The pharmaceutical composition of Embodiment 86, wherein L⁰, L¹, and L² are each independently at each occurrence C₁-C₆ alkylene (e.g., C₁-C₃ alkylene).

Embodiment 89. The pharmaceutical composition of Embodiment 86, wherein L⁰, L¹, and L² are each independently at each occurrence C₂-C₁₂ (e.g., C₂-C₈) alkyleneoxide (e.g., —(C₁-C₃ alkylene-0)₁-₄-(C₁-C₃ alkylene)).

Embodiment 90. The pharmaceutical composition of Embodiment 86, wherein L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

Embodiment 91. The pharmaceutical composition of any one of Embodiments 57-90, wherein each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

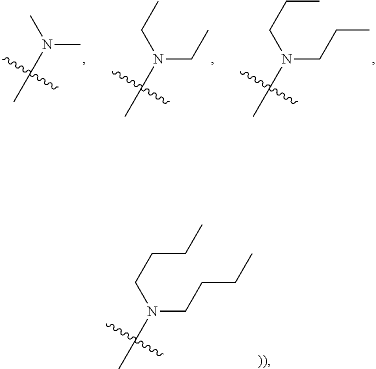

C₄-C₆ N-heterocycloalkyl (e.g., N-pyrrolidinyl

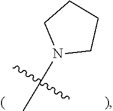 [SMILES: *N1CCCC1]

(N)-pipenidinyl

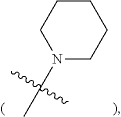 [SMILES: *N1CCCCC1]

### N-azepanyl

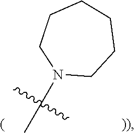 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

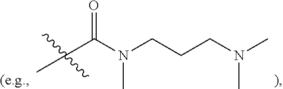 [SMILES: *C(=O)N(C)CCCN(C)C]

—C(O)N(C₁-C₃ alkyl)-(C₁-C₆ alkylene)-(C₄-C₆ N-heterocycloalkyl)

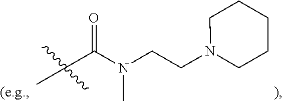 [SMILES: *C(=O)N(C)CCN1CCCCC1]

(e.g., |), —C(O)—(C₁-C₁₂ alkylamino (e.g., mono- or di-alkylamino)), and —C(O)—(C₄-C₆ N-heterocycloalkyl)

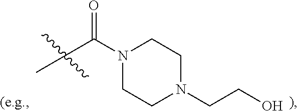 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 92. The pharmaceutical composition of Embodiment 91, wherein each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

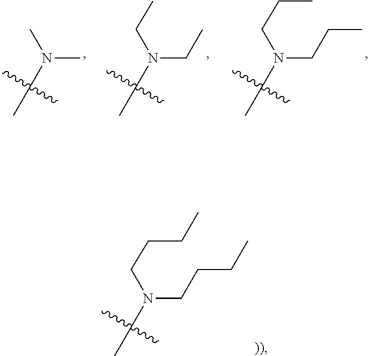

C₄-C₆ N-heterocycloalkyl (e.g., N-pyrrolidinyl

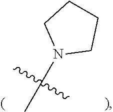 [SMILES: *N1CCCC1]

### N-pipedinyl

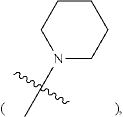 [SMILES: *N1CCCCC1]

### N-azepanyl

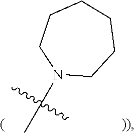 [SMILES: *N1CCCCCC1]

—OH, —C(O)OH, —C(O)N(C₁-C₃ alkyl)-(C₁-C₆ alkylene)-(C₁-C₁₂ alkyl

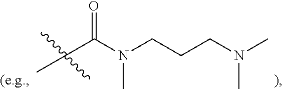 [SMILES: *C(=O)N(C)CCCN(C)C]

—C(O)N(C₁-C₃ alkyl)-(C₁-C₆ alkylene)—(C₄-C₆ N-heterocycloalkyl)

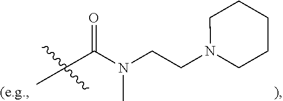 [SMILES: *C(=O)N(C)CCN1CCCCC1]

and —C(O)—(C₄-C₆ N-heterocycloalkyl

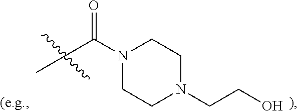 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 93. The pharmaceutical composition of Embodiment 92, wherein each terminating group is independently C₁-C₁₈ (e.g., C₄-C₁₈) alkylthiol, wherein the alkyl moiety is optionally substituted with one substituent —OH.

Embodiment 94. The pharmaceutical composition of Embodiment 92, wherein each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

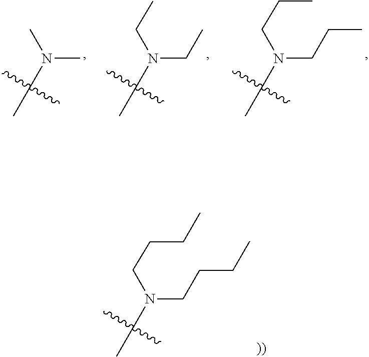

and C₄-C₆ N-heterocycloalkyl (e.g., N-pyrrolidinyl

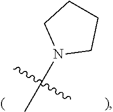 [SMILES: *N1CCCC1]

### N-piperidinyl

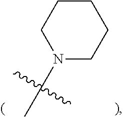 [SMILES: *N1CCCCC1]

### N-azepanyl

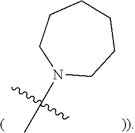 [SMILES: *N1CCCCCC1]

Embodiment 95. The pharmaceutical composition of Embodiment 91, wherein each terminating group is independently C₁-C₁₈ (e.g., C₄-C₁₈) alkenylthiol or C₁-C₁₈ (e.g., C₄-C₁₈) alkylthiol.

Embodiment 96. The pharmaceutical composition of Embodiment 92 or 95, wherein each terminating group is independently C₁-C₁₈ (e.g., C₄-C₁₈) alkylthiol.

Embodiment 97. The pharmaceutical composition of any one of Embodiments 57-90, wherein each terminating group is independently selected from those set forth in Table 5 or a subset thereof; or wherein each terminating group is independently selected from the group

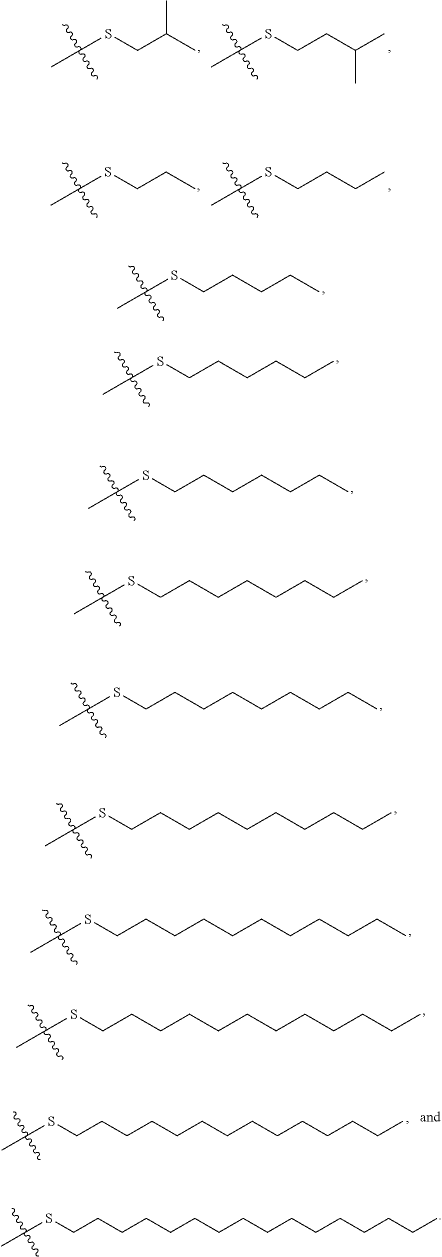

Embodiment 98. The pharmaceutical composition of Embodiment 57, wherein the ionizable cationic lipid is selected from those set forth in Table 6, or pharmaceutically acceptable salts thereof, or a subset of the lipids and the pharmaceutically acceptable salts thereof.

Embodiment 99. The pharmaceutical composition of any one of Embodiments 1-98, wherein said pharmaceutical formulation is formulated for inhalation.

Embodiment 100. The pharmaceutical composition of any one of Embodiments 1-99, wherein said pharmaceutical composition is an (e.g., inhalable) aerosol composition.

Embodiment 101. An aerosol composition comprising a pharmaceutical composition of any one of Embodiments 1-99.

Embodiment 102. The pharmaceutical composition of Embodiment 100 or the aerosol composition of Embodiment 101, wherein said aerosol composition is generated by a nebulizer.

Embodiment 103. The pharmaceutical composition of Embodiment 100 or the aerosol composition of Embodiment 101 or 102, wherein said aerosol composition has a (e.g., median, or average) droplet size from 1 micron (μm) to 10 μm.

Embodiment 104. The aerosol composition of any one of Embodiments 100-103, wherein said aerosol droplets are generated by a nebulizer at a nebulization rate of no more than 70 mL/minute.

Embodiment 105. The aerosol composition of any one of Embodiments 100-104, wherein said aerosol droplets have a mass median aerodynamic diameter (MMAD) from about 0.5 micron (μm) to about 10 μm.

Embodiment 106. The aerosol composition of any one of Embodiments 100-105, wherein said droplet size varies less than about 50% for a duration of about 24 hours under a storage condition.

Embodiment 107. The aerosol composition of any one of Embodiments 100-106, wherein droplets of said aerosol composition are characterized by a geometric standard deviation (GSD) of no more than about 3.

Embodiment 108. A method for treating a subject having or suspected of having primary ciliary dyskinesia (PCD), comprising administering to said subject a pharmaceutical composition of any one of Embodiments 1-100 and 102-103.

Embodiment 109. A method for treating a subject having or suspected of having primary ciliary dyskinesia (PCD), comprising administering to said subject a pharmaceutical composition comprising a heterologous polynucleotide assembled with a lipid composition, which heterologous polynucleotide encodes a dynein axonemal intermediate chain 1 (DNAI1) protein, thereby resulting in a heterologous expression of said DNAI1 protein within cells of said subject, wherein said lipid composition comprises (i) an ionizable cationic lipid, and (ii) a selective organ targeting (SORT) lipid separate from said ionizable cationic lipid.

Embodiment 110. The method of Embodiment 109, wherein said lipid composition further comprises (iii) a phospholipid.

Embodiment 111. The method of Embodiment 108-110, wherein said administering comprises administering to a lung by nebulization.

Embodiment 112. The method of any one of Embodiments 108-111, wherein said subject is determined to exhibit an aberrant expression or activity of DNAI1 gene or protein.

Embodiment 113. The method of any one of Embodiments 108-112, wherein said subject is a human.

Embodiment 114. The method of any one of Embodiments 108-113, wherein said (e.g., ciliated) cells are in a lung of said subject.

Embodiment 115. The method of Embodiment 114, wherein said cells comprises ciliated cell(s), basal cell(s), club cell(s), or a combination thereof.

Embodiment 116. The method of Embodiment 114, wherein said cells comprises ciliated cells.

Embodiment 117. The method of Embodiment 114, wherein said cells are undifferentiated.

Embodiment 118. The method of Embodiment 114, wherein said cells are differentiated.

Embodiment 119. The method of Embodiment 115, wherein said ciliated cells are ciliated epithelial cells (e.g., ciliated airway epithelial cells).

Embodiment 120. The method of Embodiment 119, wherein said ciliated epithelial cells are undifferentiated.

Embodiment 121. The method of Embodiment 119, wherein said ciliated epithelial cells are differentiated.

Embodiment 122. A method for enhancing an expression or activity of dynein axonemal intermediate chain 1 (DNAI1) protein in a (e.g., lung) cell, the method comprising: contacting said (e.g., lung) cell with a composition comprising a synthetic polynucleotide assembled with a lipid composition, wherein said synthetic polynucleotide encodes a DNAI1 protein, wherein said lipid composition comprises an ionizable cationic lipid and a selective organ targeting (SORT) lipid separate from said ionizable cationic lipid, thereby providing a(n) (e.g., therapeutically) effective amount or activity of a functional variant (e.g., wild-type form) of DNAI1 protein in said (e.g., lung) cell.

Embodiment 123. The method of Embodiment 122, wherein the method provides a(n) (e.g., therapeutically) effective amount or activity of said functional variant (e.g., wild-type form) of DNAI1 protein in said (e.g., lung) cell at least about 6 hours after said contacting.

Embodiment 124. The method of Embodiment 123, wherein said contacting is in vivo.

Embodiment 125. The method of Embodiment 123, wherein said contacting is ex vivo.

Embodiment 126. The method of Embodiment 123, wherein said contacting is in vitro.

Embodiment 127. The method of Embodiment 122 or 123, wherein said (e.g., lung) cell is in a ciliary axoneme.

Embodiment 128. The method of any one of Embodiments 122-127, wherein mucus is present in said contacting

Embodiment 129. The method of any one of Embodiments 122-128, wherein said (e.g., lung) cell is an airway epithelial cell (e.g., a bronchial epithelial cell).

Embodiment 130. The method of Embodiment 128, wherein said (e.g., lung) cell is a ciliated cell, a basal cell, a goblet cell, or a club cell.

Embodiment 131. The method of Embodiment 128, wherein said (e.g., lung) cell is a ciliated cell, a basal cell, or a club cell.

Embodiment 132. The method of any one of Embodiments 122-131, wherein said (e.g., lung) cell exhibits a mutation in DNAI1 gene or transcript.

Embodiment 133. The method of any one of Embodiments 122-132, wherein said contacting comprises contacting a plurality of (e.g., lung) cells that comprises said (e.g., lung) cell.

Embodiment 134. The method of any one of Embodiments 122-133, wherein the method provides a(n) (e.g., therapeutically) effective amount or activity of said functional variant (e.g., wild-type form) of DNAI1 protein in at least about 5%, 10%, 15%, or 20% lung epithelial cells comprising said (e.g., lung) cell.

Embodiment 135. The method of any one of Embodiments 122-134, wherein the method provides a(n) (e.g., therapeutically) effective amount or activity of said functional variant (e.g., wild-type form) of DNAI1 protein in at least about 2%, 5%, or 10% lung ciliated cells comprising said (e.g., lung) cell.

Embodiment 136. The method of any one of Embodiments 122-135, wherein the method provides a(n) (e.g., therapeutically) effective amount or activity of said functional variant (e.g., wild-type form) of DNAI1 protein in at least about 5%, 10%, 15%, or 20% lung secretory cells comprising said (e.g., lung) cell.

Embodiment 137. The method of any one of Embodiments 122-136, wherein the method provides a(n) (e.g., therapeutically) effective amount or activity of said functional variant (e.g., wild-type form) of DNAI1 protein in at least about 5%, 10%, 15%, or 20% lung club cells comprising said (e.g., lung) cell.

Embodiment 138. The method of any one of Embodiments 122-137, wherein the method provides a(n) (e.g., therapeutically) effective amount or activity of said functional variant (e.g., wild-type form) of DNAI1 protein in at least about 5%, 10%, 15%, or 20% lung goblet cells comprising said (e.g., lung) cell.

Embodiment 139. The method of any one of Embodiments 122-138, wherein the method provides a(n) (e.g., therapeutically) effective amount or activity of said functional variant (e.g., wild-type form) of DNAI1 protein in at least about 5%, 10%, 15%, or 20% lung basal cells comprising said (e.g., lung) cell.

Embodiment 140. The method of any one of Embodiments 122-139, wherein said contacting is repeated (e.g., at least about 2, 4, 6, 8, or 10 times).

Embodiment 141. The method of Embodiment 140, wherein said repeated contacting is at least once a week, at least twice a week, or at least three times a week.

Embodiment 142. The method of Embodiment 140 or 141, wherein at least one contacting steps of said repeated contacting is followed by a treatment holiday.

Embodiment 143. The method of any one of Embodiments 140-142, wherein said repeated contacting is characterized by a duration of at least 1, 2, 3, 4, or 5 week(s).

Embodiment 144. The method of any one of Embodiments 140-143, wherein mucus is present in one or more contacting steps of said repeated contacting.

Embodiment 145. The method of any one of Embodiments 122-144, wherein the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, or 7 days) after said contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof.

Embodiment 146. The method of Embodiment 145, wherein said contacting is ex vivo or in vitro.

Embodiment 147. The method of any one of Embodiments 122-144, wherein the method provides a(n) (e.g., therapeutically) effective amount or activity of said functional variant (e.g., wild-type form) of DNAI1 protein in said (e.g., lung) cell at least about 6, 24, 48, or 72 hours (such as at least about 3, 4, 5, 6, 7, 8, 9, 10, 11, 12, 13, or 14 days) after a contacting of said repeated contacting, e.g., as determined by measuring a change or recovery in a ciliary beat activity (e.g., a ciliary beat frequency or synchronization rate) or in an area with the ciliary beat activity at an air-liquid-interface (ALI) comprising said (e.g., lung) cell, said plurality of (e.g., lung) cells, or a derivative thereof.

Embodiment 148. The method of Embodiment 145, wherein said repeated contacting(s) are ex vivo or in vitro.

### EXAMPLES

### Example 1. Preparation of DOTAP or DODAP Modified Lipid Nanoparticles

Lipid nanoparticles (LNPs) are the most efficacious carrier class for in vivo nucleic acid delivery. Historically, effective LNPs are composed of 4 components: an ionizable cationic lipid, zwitterionic phospholipid, cholesterol, and lipid poly(ethylene glycol) (PEG). However, these LNPs result in only general delivery of nucleic acids, rather than organ or tissue targeted delivery. LNPs typically delivery RNAs only to the liver. Therefore, new formulations of LNPs were sought in an effort to provide targeted nucleic acid delivery.

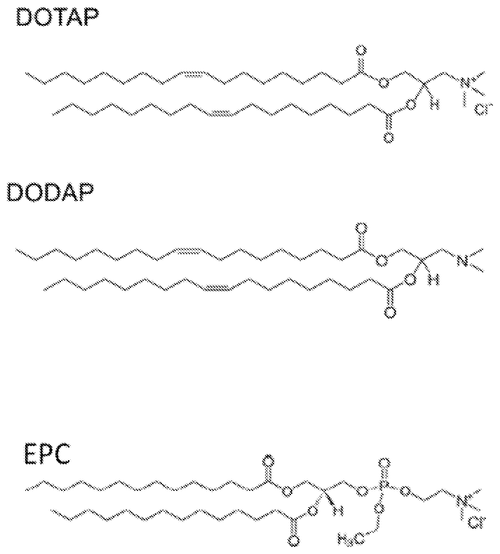

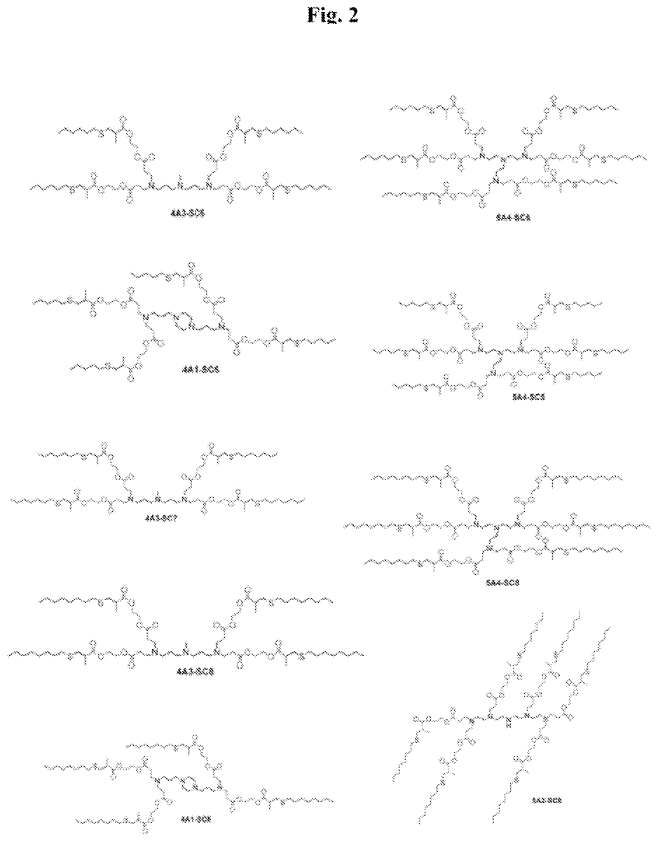

The four canonical types of lipids were mixed in a 15:15:30:3 molar ratio, with or without the addition of a permanently cationic lipid. Briefly, LNPs were prepared by mixing a dendrimer or dendron lipid (ionizable cationic), DOPE (zwitterionic), cholesterol, DMG-PEG, and DOTAP (permanently cationic). Alternatively, DOTAP can be substituted for DODAP to generate a LNP comprising DODAP. The structure of DODAP and DODAP are shown in FIG. 1. Various dendrimer or dendron lipids that may be used are shown in FIG. 2.

For preparation of the LNP formulation, a dendrimer or dendron lipid, DOPE, Cholesterol and DMG-PEG were dissolved in ethanol at desired molar ratios. The mRNA was dissolved in citrate buffer (10 mM, pH 4.0). The mRNA was then diluted into the lipids solution to achieve a weight ratio of 40:1 (total lipids:mRNA) by rapidly mixing the mRNA into the lipids solution at a volume ratio of 3:1 (mRNA:lipids, v/v). This solution was then incubated for 10 min at room temperature. For formation of DOTAP modified LNP formulations, mRNA was dissolved in 1×PBS or citrate buffer (10 mM, pH 4.0), and mixed rapidly into ethanol containing 5A2-SC8, DOPE, Cholesterol, DMG-PEG and DOTAP, fixing the weight ratio of 40:1 (total lipids:mRNA) and volume ratio of 3:1 (mRNA:lipids). Formulations are named X % DOTAP Y (or X % DODAP Y) where X represents the DOTAP (or DODAP) molar percentage in total lipids, and Y represents the type of dendrimer or dendron lipid. Alternatively, formulation may be named Y X % DOTAP or Y X % DODAP where X represents the DOTAP (or DODAP) molar percentage in total lipids, and Y represents the type of dendrimer or dendron lipid.

### Example 2. SORT LNP Stability

LNPs were tested for stability. 5A2-SC8.20% DODAP (“Liver-SORT) and 5A2-SC8 50% DOTAP (“Lung-SORT”) were generated using either a microfluidic mixing method or a cross/tee mixing method. The different LNP formulations were characterized by size, polydispersity index (PDI) and zeta-potential, were examined by dynamic light scattering, 3 separate times for each formulation. The characteristics of the LNPs are show in Table 8.

TABLE 8
SORT LNP characteristics

| | | | | Encapsulation |
|---|---|---|---|---|
| | Size (nm) | PDI | Zeta (mV) | Efficiency (%) |
| Lung-SORT - | 82.3 | 0.10 | 3.0 | 100 |
| microfluidic | | | | |
| Lung-SORT - | 78.1 | 0.09 | 2.2 | 100 |
| cross/tee mixing | | | | |
| Liver-SORT - | 59.1 | 0.10 | −2.3 | 97 |
| microfluidic | | | | |
| Liver-SORT - | 60.0 | 0.11 | −30 | 96 |
| cross/tee mixing | | | | |

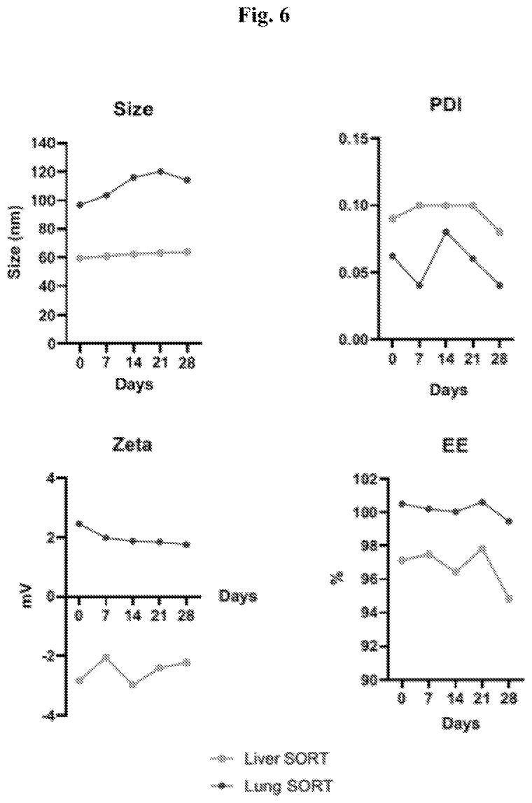

The encapsulation efficiency was tested using a Ribogreen RNA assay (Zhao et al., 2016). Briefly, mRNA was encapsulated with >95% efficiency in LNPs when the mRNA was dissolved in acidic buffer (10 mM citrate, pH 4). The characteristics were observed over 28 days for the two types of LNPs (5A2-SC8 20% DODAP (“Liver-SORT) and 5A2-SC8 50% DOTAP (“Lung-SORT”)). FIG. 6 shows the changes of the characteristics of the LNP over the course of 28 days.

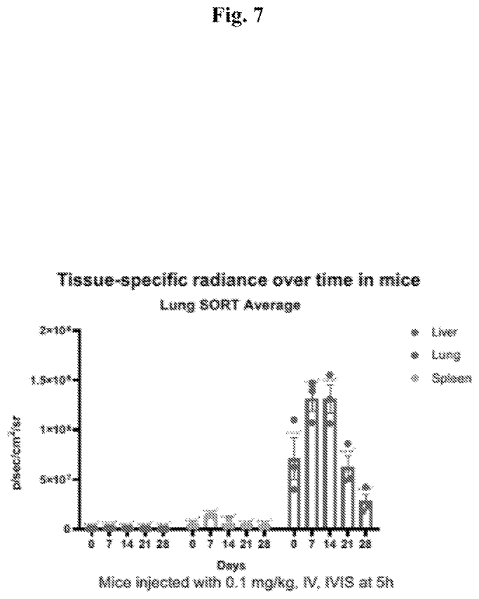

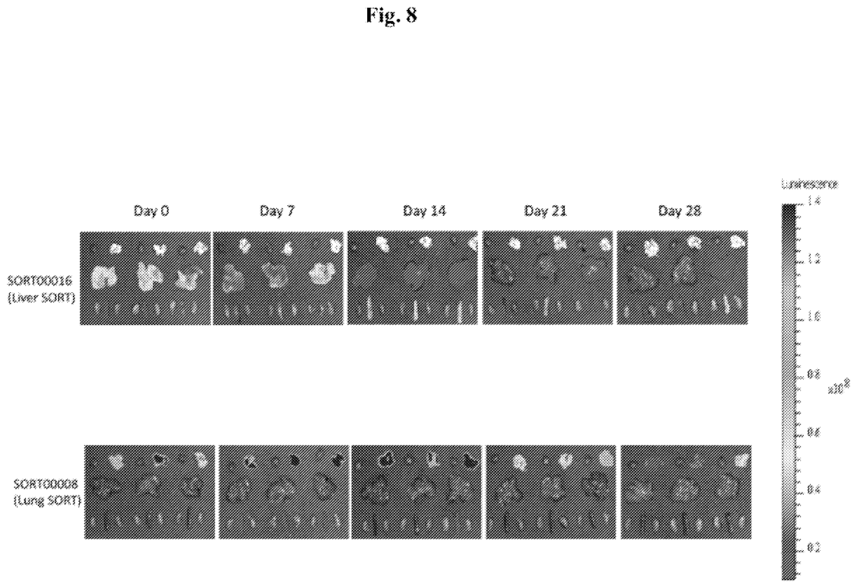

In addition, to the measure of the stability of the LNPs in solution, the stability of the LNPs and resulting mRNA expression was observed in mice. Briefly, mice were injected intravenously with 0.1 mg/kg and observed in vivo. Luciferin was added 5 hrs. after injection and visualized. As shown in FIG. 7, the Lung-SORT LNP generated tissue specific radiance in the lungs which remained high even after 14 day with a slight decay in signal by the 21^{st} and^{28th}day. FIG. 8 shows images of the organs of the mouse at specific times periods after treated with Lung-SORT or Liver-SORT.

### Example 3. Expression of TR mRNA in Different Cell Types

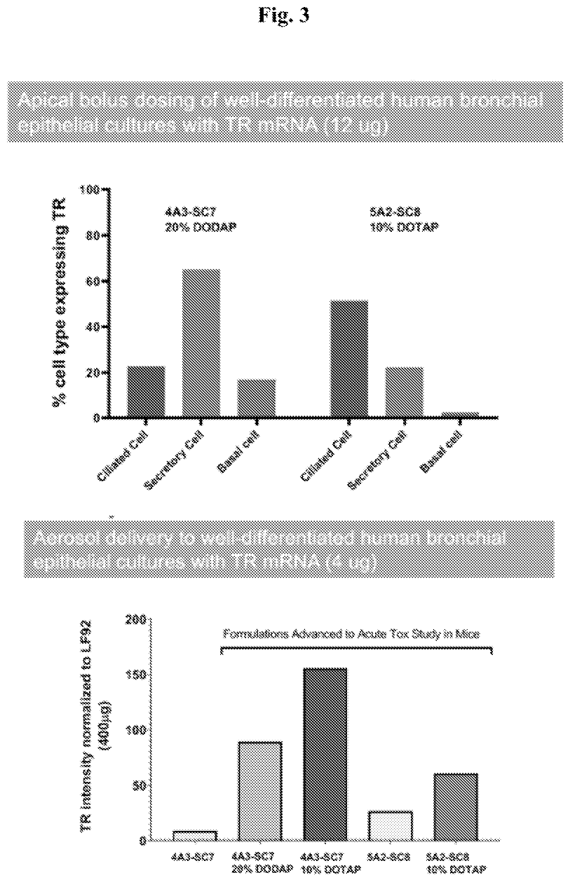

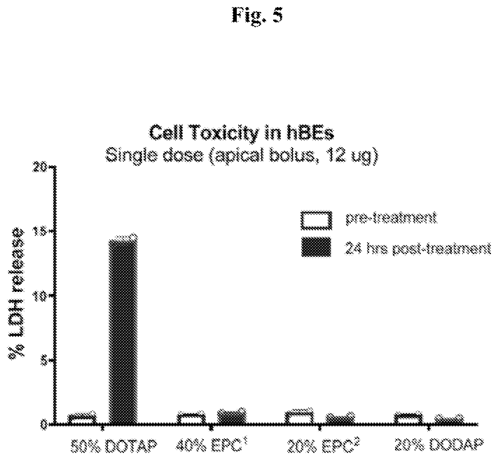

TR mRNA was loaded into either 20% DODAP 4A3-SC7 LNP or 10% DOTAP 5A2-SC8 LNPs and delivered into well-differentiated human bronchial epithelial cultures using apical bolus dosing. Cell expression was observed in various cell type and the percent of the cell type that expressed TR was plotted. As shown in the top panel of FIG. 3, the 20% DODAP 4A3-SC7 LNPs preferentially caused secretory cells to express TR, while 10% DOTAP 5A2-SC8 LNPs cause the ciliated cells to preferentially express TR. This preferential delivery may allow a treatment delivered to the lungs to preferentially affect a specific cell type in the lungs The TR mRNA was also loaded into LNPs without the SORT lipid (e.g., DODAP or DOTAP) to identify how the DODAP or DOTAP affected the potency. As shown in the bottom panel of FIG. 5, the LNPs comprising DOTAP or DODAP showed increase TR expression compared to their corresponding LNP without DOTAP or DODAP.

### Example 4. Luciferase Activity and Histopathology from LNPs Delivered Via Inhaled Aerosol

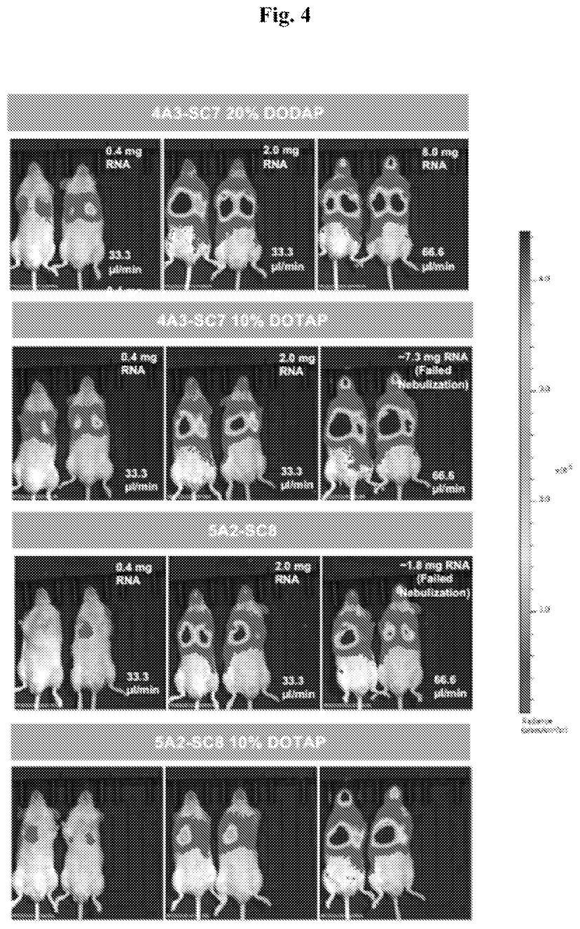

Luc mRNA was loaded into a number of LNPs including LNPs of comprising a SORT lipid and a dendrimer or dendron. LNPs of 4A3-SC7 20% DODAP, 4A3-SC7 10% DODAP, 5A2-SC8, 5A2-SC8 10% DOTAP were generated and loaded with Luc mRNA. 0.4/2/8 mg of LNP-formulatedLuc2 mRNA (1 mg/ml) was delivered into the pie chamber by nebulization (Aerogen solo), with an estimated (not measured) per mouse delivered dose of 0.01, 0.06 or 0.22 mg/kg. The mice were 7-week-old B6 male albino mice. Luciferin was administered to the mice 5 hrs. after delivery of the LNPs. The luciferase activity was detected as a measure of delivery to a target. FIG. 4 shows the distribution and expression of the luciferase in the mice demonstrating the expression was successful and delivery of the LNPs could be performed using inhaled aerosol delivery.

### Example 5. Toxicity of EPC Containing LNPs

LNPs comprising ethylphosphocholine (EPC) in place of DOTAP or DODAP were tested for toxicity by using apical bolus dosing on human bronchial epithelial cells. The % of lactate dehydrogenase (LDH) that was released was used as a metric of cellular death and indicative of the toxicity of the LNP. The release of LDH was detected prior to treatment (pre-treatment) and 24 post treatment. As shown in FIG. 5, the treatment of 50% DOTAP LNP resulted in an ˜15% LDH release whereas EPC didn't show a significant % LDH release. Importantly, DOTAP and EPC have a similar quaternary amine moiety, indicating that the activity for cell targeting may be similar, but that EPC is considerably less toxic.

### Example 6. Production of DNAI1 mRNA

DNA corresponding to the gene of DNAI1 was synthesized at GenScript. pUC57/DNAI1 was digested with HindIII and EcoRI HF restriction enzymes. Moreover, a digested pVAX120 vector and DNAI1 cDNA were gel purified and ligated (the ORF for DNAI1 is codon optimized). Standard in vitro translation procedure was used for RNA production utilizing unmodified nucleotides. Capping reaction was carried out using Vaccinia Virus capping system and cap 2′-O-methyl transferase.

### Example 7. Detection of DNAI1 mRNA Delivery to a Subject

A subject having or suspected of having primary ciliary dyskinesia (PCD) is given a treatment by administering a composition as described elsewhere herein. The subject is monitored at regular intervals for expression of DNAI1 in the lungs. A sample of lung tissue from the subject is taken comprising ciliated cells of the lung. The cells are harvested and prepared for RNA isolation. cDNA is produced from the RNA using a first strand synthesis kit and random hexamer. qPCR reactions are run using a set of forward and reverse primers and a fluorescent probe, specific to DNAI1 and a second set specific to a control or housekeeping gene for expression normalization. Expression of DNAI1 is detected using a fluorescent readout corresponding the DNAI1 probe.

### Example 8. Functional Rescue in hBEs with DNAI1 mRNA

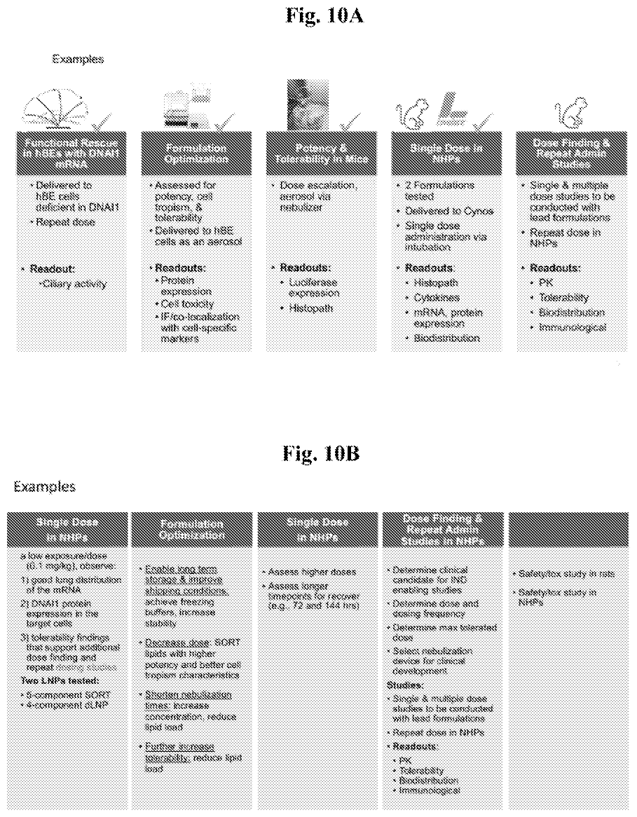

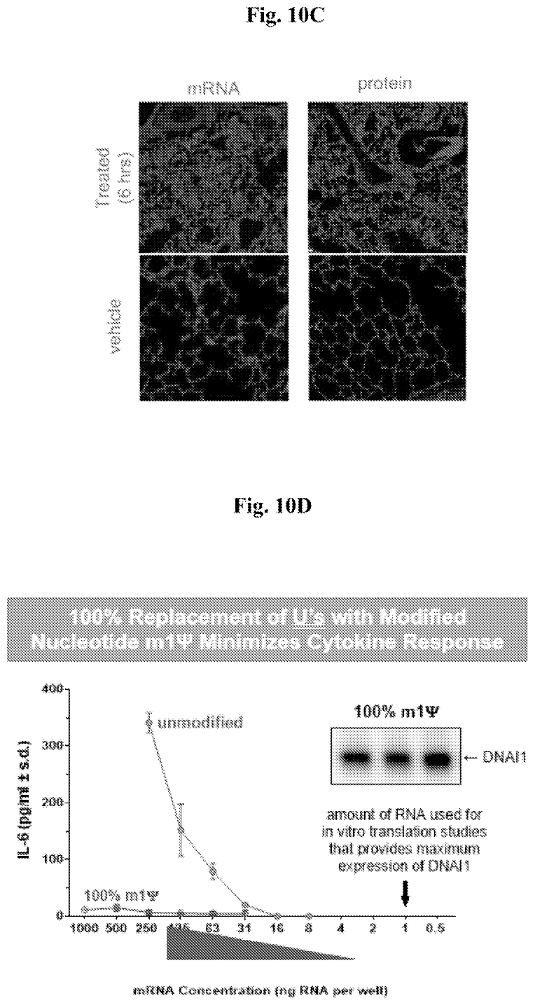

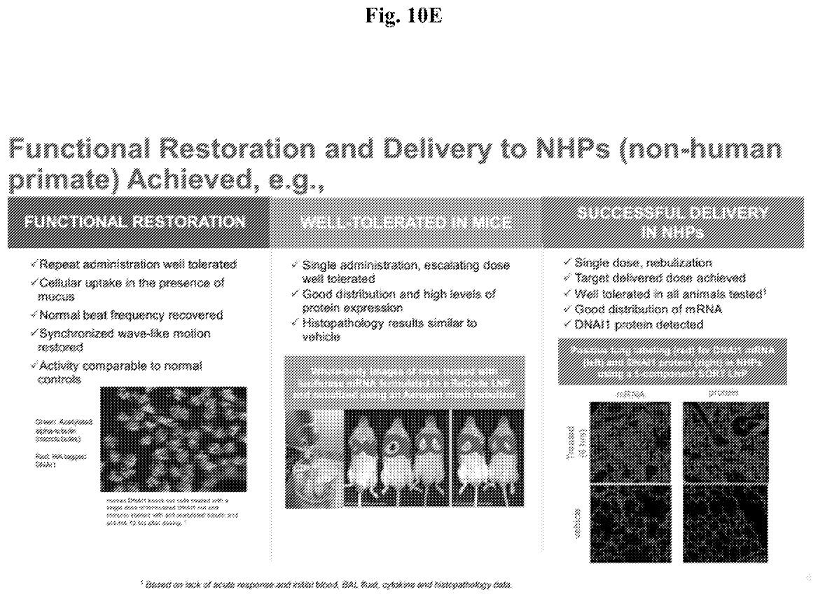

Repeated doses of lipid compositions described herein were delivered to human basal epithelial cells (hBEs) deficient in DNAI1 as described in the first column of FIG. 10A. Results of the study are summarized in the left column of FIG. 10E. Cellular uptake was observed in the presence of mucus. The activity of cilia post treatment was comparable to normal controls. Normal beat frequency and synchronized wave-like motion of cilia was recovered. The first column of FIG. 10B further illustrates targeting of DNAI1-HA mRNA to ciliated cells, Immunofluorescence of DNAI1-HA and acetylated tubulin (biomarker for ciliated cells) shows the expression of DNAI1-HA in hBEs 72 h after dosing.

### Example 9. Potency and Tolerability Study in Mice

Single administration and escalating doses of lipid compositions described herein comprising a luciferase mRNA payload are tested for potency and tolerability in mice. Key features of the study are summarized in the middle column of FIG. 10A. Mice are treated with a lipid composition comprising an ionizable cationic lipid (e.g., 4A3SC7, 5A2SC8) and a SORT lipid (e.g., DODAP, DOTAP) aerosolized via a nebulizer. Luciferase expression is measured to assess potency and histopathology is measured to assess tolerability. Good distribution and high levels of protein expression are observed in whole-body images of mice treated with the lipid composition, such as that depicted in the middle column of FIG. 10E. Histopathology results are comparable to control animals indicating high tolerability. The results from testing a short delivery time (e.g., 5-8 min) and low concentration used (e.g., 0.5 mg/mL) provides support for increasing the dosage.

### Example 10. DNA11 Expression in Lung Tissues of NHPs

Two lipid compositions, RTX0001 (5 components) and RTX0004 (4 components), were evaluated in a non-human primate (NHP, cynomolgus macaques) study to demonstrate DNAI1 expression in lung tissues. RTX0001 comprises 4A3-SC7, DODAP, DOPE, cholesterol, and DMG-PEG at a molar ratio of 19.05:20:19.05:38.09:3.81, respectfully. RTX0004 comprises 5A2-SC8, DOPE, cholesterol, and DMG-PEG at a molar ration of 19.05:23.81:47.62:4.76, respectfully. Key features of the study are summarized in the fourth column from the left of FIG. 10A. Further experimental details are summarized in the middle column of FIG. 10B. Briefly, two formulations, one comprising a SORT LNP formulation (comprising, e.g., DODAP, DOTAP) and another comprising an LNP formulation were delivered to NHPs as a single dose via intubation. Both compositions contained DNAI1-HA mRNA. DNA1-HA mRNA and DNAI1-HA protein expression were detected in lungs of NHPs at 6 and 24 hours at doses of the aerosolized compositions of 0.1 mg/kg or less. The right column of FIG. 10C shows DNAI1-HA mRNA and corresponding protein expression observed 6 hours post treatment in the lung tissues of treated NHPs. The composition comprising a SORT molecule resulted in stronger observed expression of DNAI1-HA and DNAI1-HA mRNA in the lungs of NHPs. No adverse clinical observation or tolerability issues were detected precluding use of the formulations at higher doses or in multi-dose settings. FIG. 10D shows that that by replacing 100% of U's in the mRNA with modified nucleotide m1Ψ minimized cytokine response.

As used herein, “RTX0001” refers to an example lipid composition tested herein. RTX0001 is a 5-component lipid nanoparticle composition comprising about 19.05% 4A3-SC7 (ionizable cationic lipid), about 20% DODAP (SORT lipid), about 19.05% DOPE, about 38.9% cholesterol, and about 3.81% DMG-PEG (PEG conjugated lipid), wherein each lipid component is defined as mol % of the total lipid composition.

As used herein, “RTX0004” refers to an example lipid composition tested herein. RTX0004 is a 4-component lipid nanoparticle composition comprising about 23.81% 5A2-SC8 (ionizable cationic lipid), about 23.81% DOPE, about 47.62% cholesterol, and about 4.76% DMG-PEG (PEG conjugated lipid), wherein each lipid component is defined as mol % of the total lipid composition.

### Example 11. Screening Lipid Formulations

Lipid compositions comprising an ionizable cationic lipid (e.g., 4A3SC7, 5A2SC8) with or without a SORT lipid (e.g., DODAP, DOTAP) were screened to enable longer storage and shipping, decrease required dose, shorten nebulization times (e.g., nebulization flow rate), and increase tolerability, as described in the second column from the left of FIG. 10A. Lipid compositions were screened by changing the ionizable lipid, the SORT lipid, buffer identity (e.g., PBS) and concentration, salt identity (e.g., NaCl) and concentration, cryopreservative identity (e.g., sucrose, trehalose, mannitol, xylitol, lactose) and concentration, N/P ratio, and PEG content. For screening N/P ratios: the FA, psd, % free, and yield were recorded, the potency and tolerability/toxicity (e.g., ciliary activity, LDH, cytokines) are assessed, and the nebulization time were evaluated (e.g., single dose, mice model). Various formulations were evaluated on the basis of particle size, polydispersity index (PDI), encapsulation efficiency (percent free mRNA). The lipid compositions were then screened for potency, targeting efficiency/specificity, stability, and tolerability/toxicity (e.g., ciliary activity, LDH, cytokines, blood chemistry markers) in human basal epithelial (hBE) cell cultures, mouse models, and mouse models for NHPs.

Experimental details and readouts are summarized in the second column from the left of FIG. 10A. Three different formulations with varying N/P ratios are summarized in Table 9.

TABLE 9
Lipid formulations tested for potency, stability, and tolerability

| | Formulation A | Formulation B | | Formulation C | |
|---|---|---|---|---|---|
| Concentration/potency | 0.5 mg/mL | 1 | mg/mL | 1-2 | mg/mL |
| Human dose | Est ≥0.5 mg/kg | 0.1-0.5 | mg/kg | 0.1-0.3 | mg/kg |

| | | | |
|---|---|---|---|
| Administration | TBD | Once or twice per | Once or twice per |
| Frequency | | week | two weeks |
| Tolerability | TBD | Adequate to support | Adequate to support |
| | | administration | administration |
| | | frequency | frequency |
| Sterilization | Sterile filtration | Sterile filtration | Sterile filtration |
| Stability/ | 2-4 weeks at 2-8° C. | ≥1 year at −80° C.; | ≥2 year at −20° C.; |
| shelf life | (cannot be frozen) | after thaw at 5° C. ≥1 | after thaw 5° C. 1 |
| | | week | month |
| | | | Alternatively, |
| | | | lyophilized, stored at |
| | | | 2-8° C. ≥2 years |
| Nebulization: | >0.2 mL/min at 0.5 | 0.4 mL/min at 0.5 | >0.4 mL/min at 0.5 |
| Flow rate | mg/mL | mg/mL | mg/mL |
| Device | Aerogen solo | Aerogen Solo or Pari | Breath-actuated |
| Nebulization time | TBD | 30-60 min | 30 min or less |

Moreover, aerosol compositions without the presence of lipid compositions (e.g., only salt(s), buffer(s), cryopreservative(s)) are tested to determine effect of each component and its concentration on the nebulization flow rate.

The aerosolized lipid compositions may provide synergy with the additional administration of the lipid composition through IV.

### Example 12. Clinical Study of mRNA Treatment for Primary Ciliary Dyskinesia

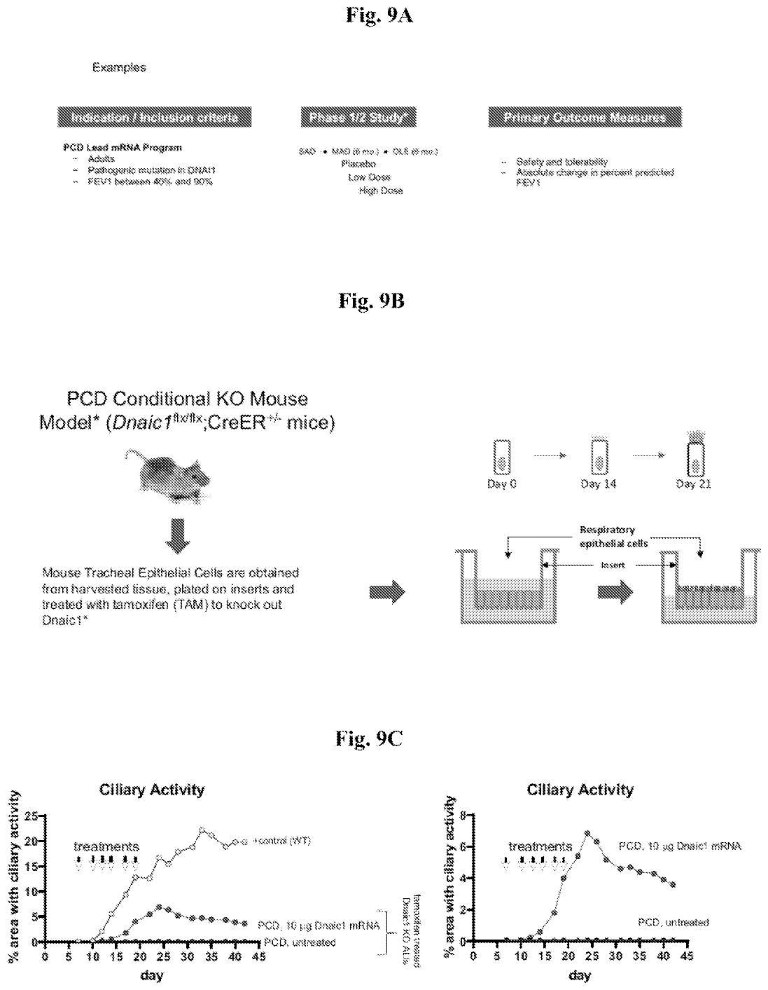

Adult subjects having or suspected of having primary ciliary dyskinesia (PCD) are given a treatment by administering a composition as described elsewhere herein. Subjects may be selected on the basis of a pathogenic mutation in DNAI1 and/or FEV1 between 40% and 90%. The study is single ascending dose (SAD), multiple ascending dose (MAD), or open-label extension (OLE) study. Subjects are sorted into placebo, low dose, and high dose treatment groups and receive a corresponding amount of formulation (or placebo). Subjects are observed for safety and tolerability and absolute change in percent predicted FEV1. Subjects treated with the formulation show signs of high tolerability and increase in percent predicted FEV1. FIG. 9A summarizes the main components of such a study. FIG. 9B illustrates an ex vivo model of ciliated epithelial cells (mouse tracheal epithelial cells or MTECs cultured at an air-liquid Interface (ALI) for testing the efficacy of rescue by the DNAI1 mRNA treatment described herein. MTECs were obtained from PCD conditional KO mouse model (Dnaic1 KO) and cultured at an air-liquid interface. The cells (ciliated, goblet, or basal) formed tight junctions and produced mucus, thus remodeling and restoring properties similar the native epithelium. FIG. 9C illustrates that ciliary activity in KO mouse cells was rescued by the DNAI1 mRNA treatment, and the treatment effect remained stable for weeks after dosing was stopped. Dnaic1 KO mouse cells were treated basally three times a week starting on day 7 during differentiation. The last dose was Armentieres on day 19. Ciliary activity in treated Dnaic1 KO culture was first detected 5 days after dosing was initiated. Activity in treated Dnaic1 KO cells reached 36% of normal (vs PCD/no TAM controls) cells by day 24. Ciliary activity in treated Dnaic1 KO cells remained above 20% of normal (more than 50% of max) cells 23 days after the last treatment (the last timepoint assessed). No ciliary activity was detected in untreated Dnaic1 KO cultures throughout the duration of the study.

### Example 13. Dose Finding and Repeat Administration Studies in NHPs

Lipid compositions as described herein are used in dose finding and repeated administration studies in non-human primates (NHPs). An overview of such a study is described in the rightmost column of FIG. 10A. Lipid compositions are tested to determine clinical candidates for an investigational new drug study, determine appropriate dose and dosing frequency, determine the maximum tolerated dose, and select a nebulization device for clinical development. The three rightmost columns FIG. 10B summarize these and other goals of the study. Experimental readouts are pharmacokinetics (PK), tolerability, biodistribution, and immunological response as measured by techniques described above.

### Example 14. Detection of DNAI1-HA mRNA Expression in Cells

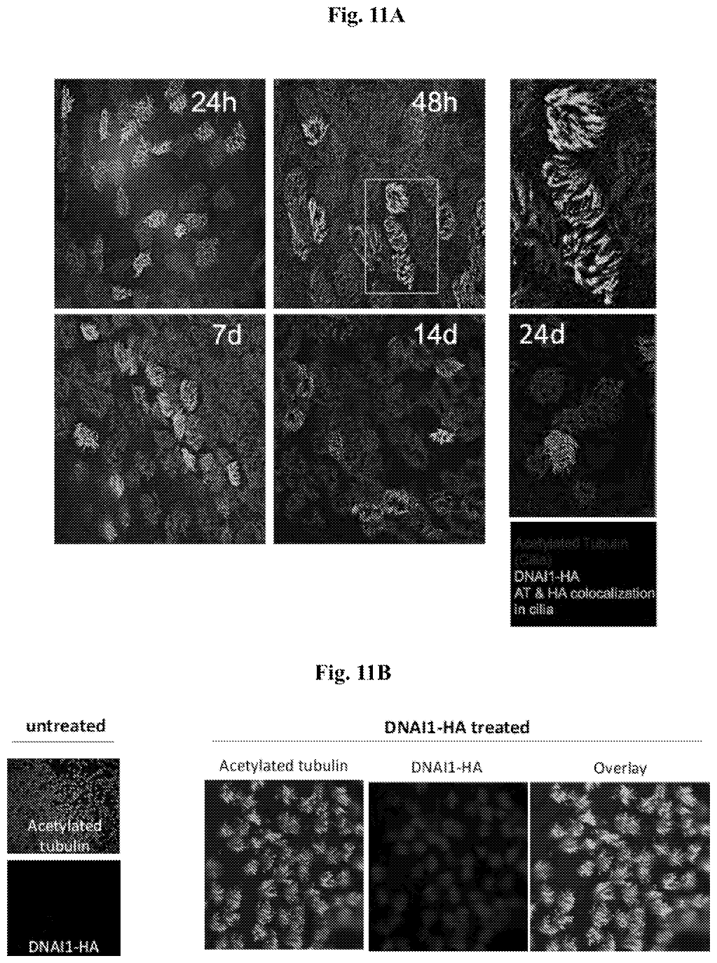

Human DNAI1 knock-down cells were cultured at an air-liquid interface (ALI). The cultured cells were treated with a single dose of LNPs containing DNAI1-HA mRNA (10 μg/mL of media). Cells were immunostained with anti-acetylated tubulin (ciliated cell marker) and anti-HA antibodies 24 hours, 48 hours, 7 days, and 14 days after dosing. FIG. 11A shows immunofluorescence imaging of these cells at the indicated timepoints, demonstrating targeted cells successfully expressed the DNAI1-HA mRNA. Integration of DNAI1-HA into axoneme of cilia is seen to peak between 48-72 hours after treatment. Well-differentiated human DNAI1 knock-down cells were treated with a single dose of a formulation of DNAI1-HA mRNA described herein and immunostained with anti-acetylated tubulin and anti-HA. Integration of newly-expressed DNAI1-HA into axoneme of cilia peaked between 48 to 72 hours after treatment. DNA11-HA was detected in ciliary axoneme for more than 24 days after single administration. Repeated administration resulted in rescue of ciliary activity that remained for weeks after the dosing was stopped. FIG. 11B illustrates that newly-made HA-tagged DNAI1 was rapidly incorporated into the cilia of human bronchial epithelial cells (hBEs). Well-differentiated human DNAI1 knock-down cells were treated (basal administration) with a single dose of LNP formulated DNAI1-HA (10 μg in 2 ml of media). Cells were immunostained with anti-acetylated tubulin and anti-HA 72 hours after dosing. More than 90% of ciliated cells was positive for DNAI1-HA.

### Example 15. Biomarkers and Multiplex Immunofluorescence Panel for Epithelial Cell Types

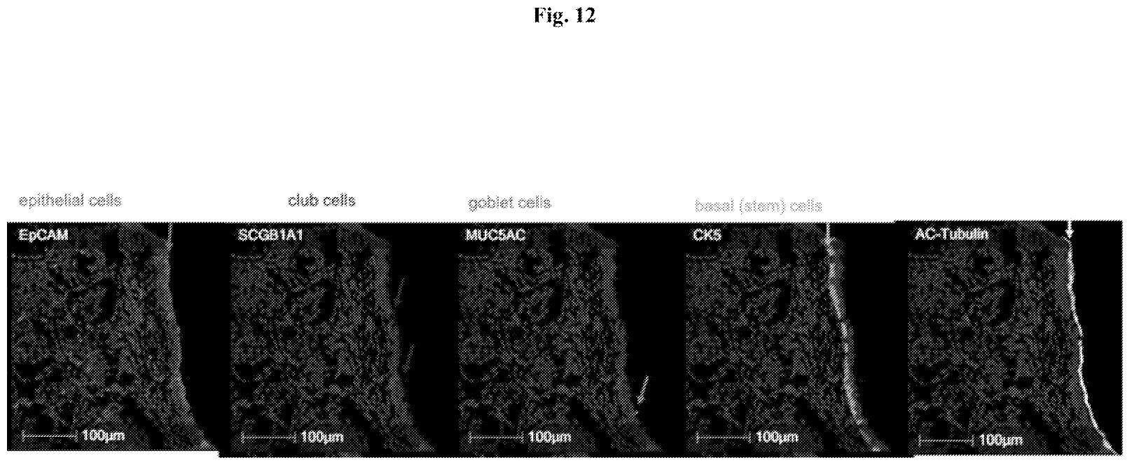

A multiplexed immuncofluroescence panel was developed to distinguish certain epithelial cell types on the basis of certain biomarkers. The particular biomarkers and corresponding cell types targeted in the panel are summarized in Table 10. FIG. 12 shows the results of the panel. In each panel, the corresponding cell type is identified via immunofluorescence by the presence of a biomarker or biomarkers.

TABLE 10
Multiplex IF Panel cell types and corresponding markers

| Cell Type | Example Marker |
|---|---|
| Epithelium | EPCAM |
| Ciliated | acetylated-tubulin (AC-Tubulin) |
| Club | Secretoglobin Family 1A Member 1 (SCGB1A1) |
| Goblet | Mucin 5AC (MUC5AC) |
| Basal (stem) | Cytokeratin 5 (CK5) |

### Example 16. Observation of Specific Cell Tropism Signatures in LNP Formulations

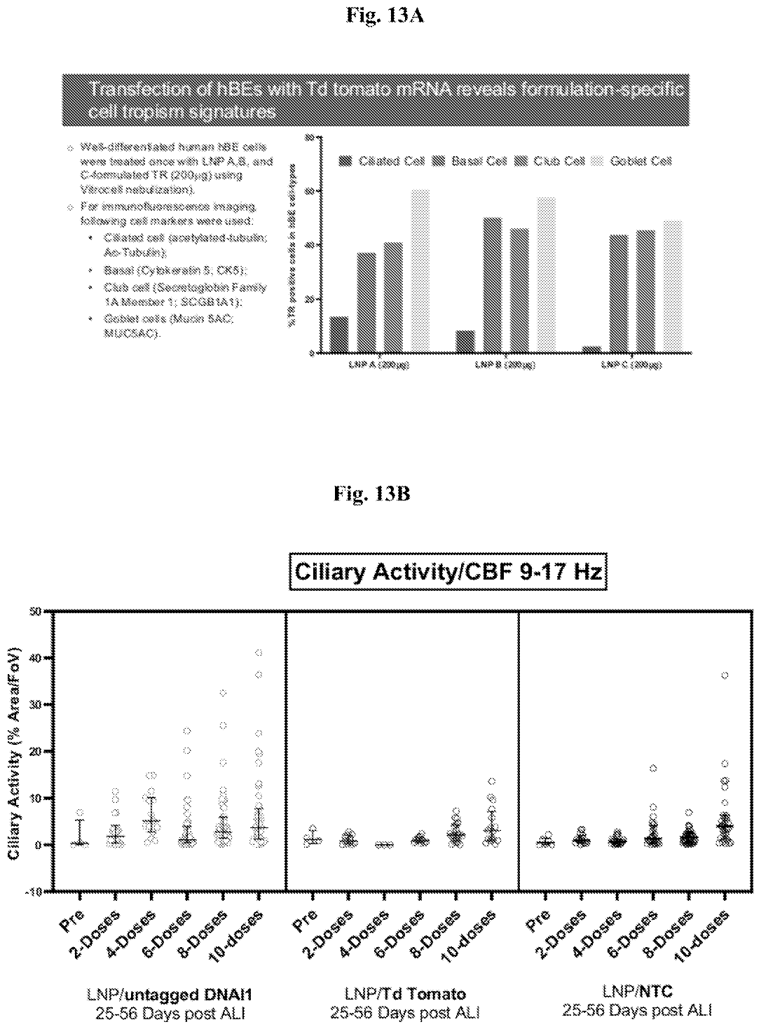

Well-differentiated human bronchial epithelial (hBE) cells were treated once with either LNP A, B, or D (200 μg) using Vitrocell nebulization. Ciliated cells, basal cells, club cells, and goblet cells were distinguished on the basis of cell markers as detailed in Table 11. FIG. 13A shows the percentage of each cell type successfully transfected with tdTomato mRNA as measured by percentage of each cell type expressing tdTomato (% TR positive) and demonstrates specific cell tropism signatures for each LNP formulation. FIG. 13B shows illustrates aerosol administration of formulated DNAI1 mRNA rescued ciliary activity in knock-down primary hBE ALI cultures. Well-differentiated human DNAI1-knock-down cells (hBEs) were treated 2 times per week with LNP-formulated DNAi1 (300 μg per Vitrocell nebulization) starting on day 25 post ALI (culture age). Last dose was administered on day 50 post ALI. Increased ciliary activity in treated DNAI1 knock-down cultures was first detected seven days after dosing was initiated. Rescued ciliary activity had normal beat frequency (9-17 Hz) and appeared synchronized.

TABLE 11
Cell markers for distinguishing cell types

| | |
|---|---|
| Ciliated cell | acylated-tubulin (Ac-Tubulin) |
| Basal cell | Cytokeratin 5 (CK5) |
| Club cell | Secretoglobin Family 1A |
| | Member 1 (SCGB1A1) |
| Goblet cell | Mucin 5AC (MUC5AC) |

### Example 17. Expression of Tomato Red (TR) in Basal an Secretory Cells

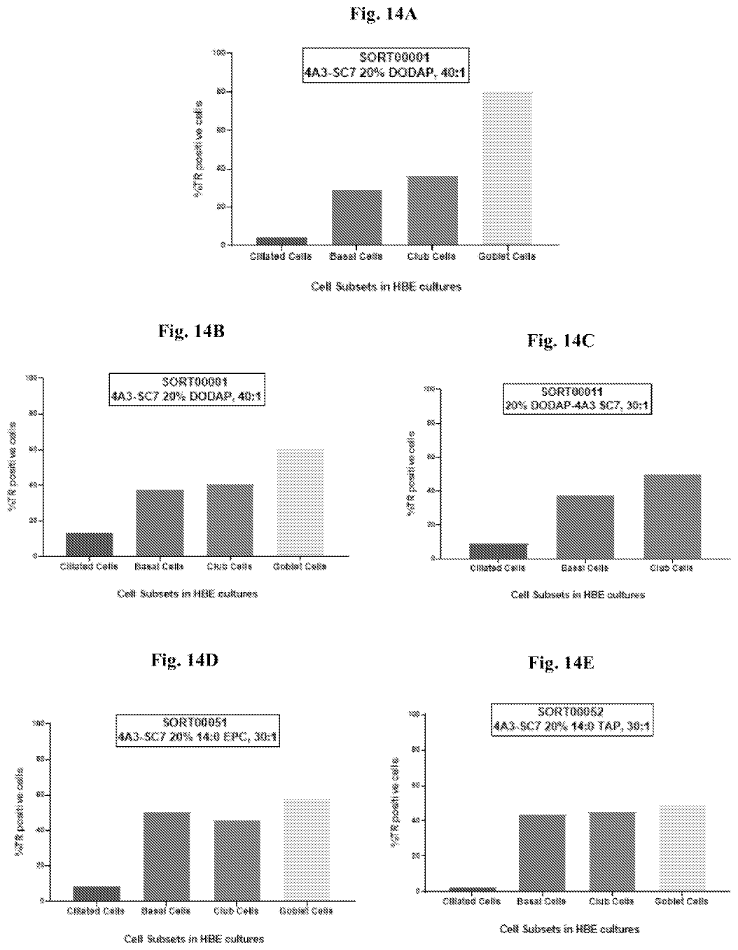

Expression of TR (Tomato Red) mRNA in different cell types in HBE cultures (human bronchial epithelial cultures) was analyzed. TR mRNA was loaded into one of 20% DODAP-4A3 SC7 40:1/PBS (FIG. 14A,), 4A3-SC7-20% DODAP 40:1/Buffer 27/frozen (FIG. 14B), 4A3-SC7-20% DODAP 30:1/Buffer 27/frozen (FIG. 14C), 4A3-SC7 20% 14:0 EPC, 30:1/Buffer 27/frozen (FIG. 14D), 4A3-SC7 20% 14:0 TAP, 30:1/Buffer 27, frozen (FIG. 14E) and delivered into well-differentiated human bronchial epithelial cultures by aerosol delivery. TR protein expression in various cell-types was observed and the percent positive TR cells in different cell-types was plotted. Cell types observed and corresponding cell markers are as described in Example 16. As shown in each panel of FIG. 14, TR was seen primarily in basal and secretory cells in treated cultures. Note that HBE cultures have high numbers of goblet cells.

### Example 18. DNA11-HA is Expressed in Cells of the Respiratory Epithelium from NHP Lung Samples

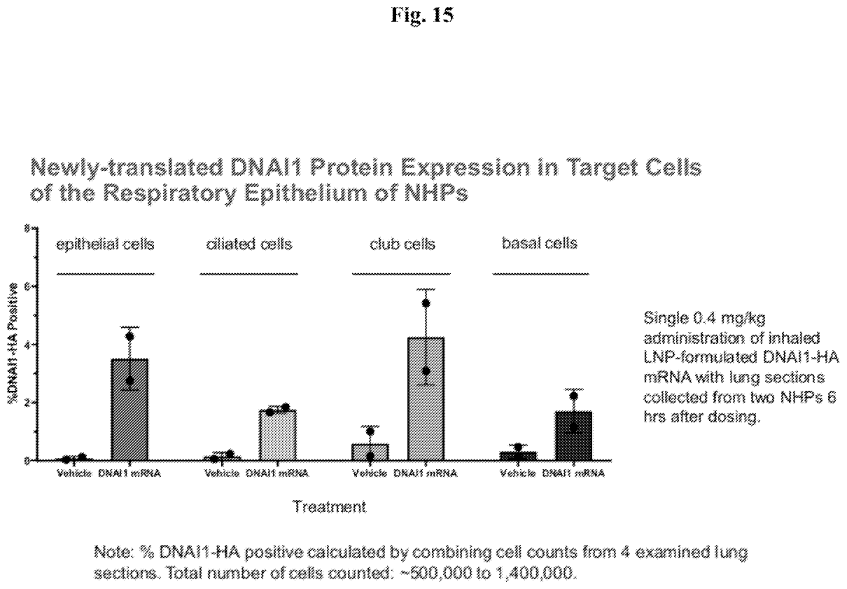

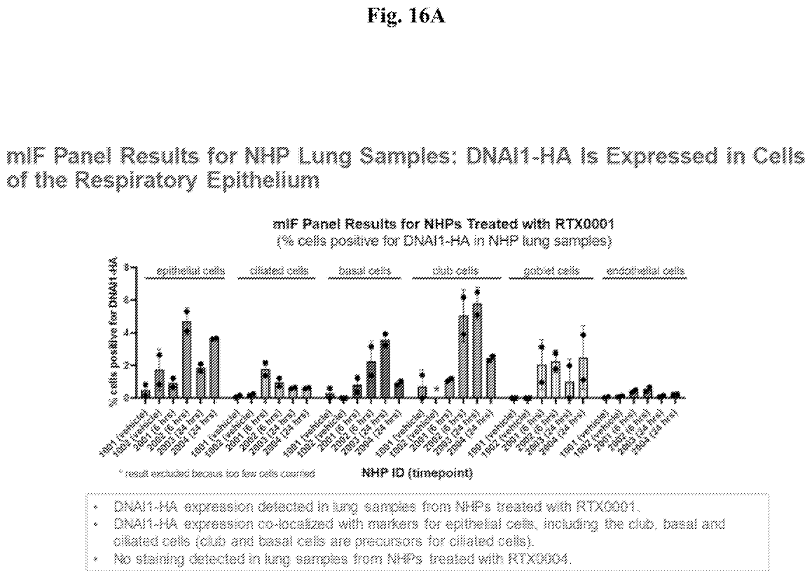

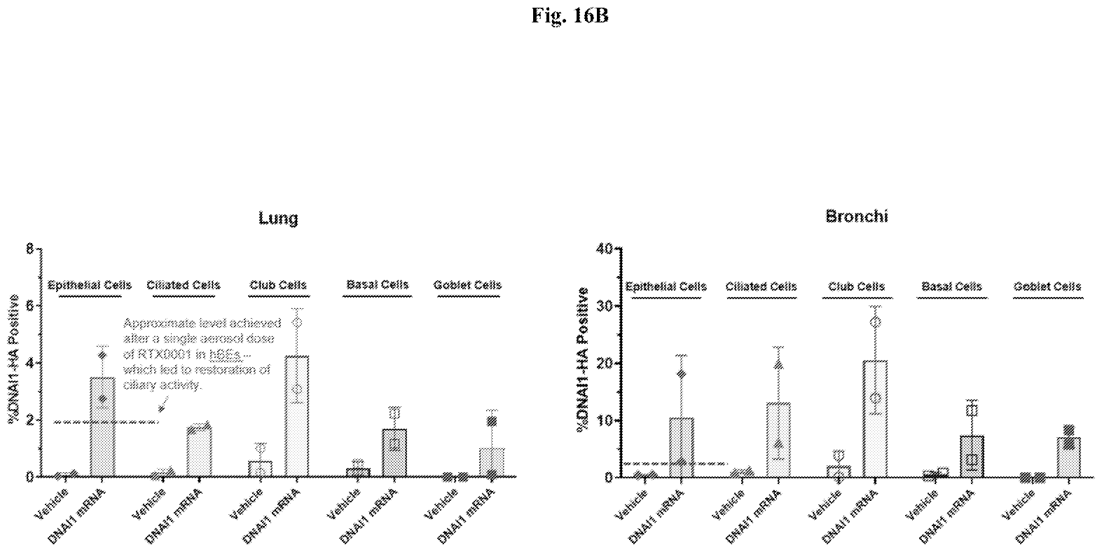

Non-human primates (NHPs) were treated by aerosol delivery with a low dose of DNAI1-HA mRNA contained in lipid compositions comprising a SORT lipid as described herein. Multiplex immunofluorescence was used to quantify DNAI1-HA expression in lung tissue blocks from treated animals. Lung tissue blocks were analyzed 6 h after treatment (6 hrs) or 24 h after treatment (24 hrs) with lipid compositions containing buffer as a control (vehicle). Cell markers for each cell type are as detailed in Example 6. As shown in FIG. 15 and FIGS. 16A-B, DNAI1-HA expression was detected in lung samples from NHPs treated with the lipid composition containing DNAI1-HA mRNA. Further, DNA11-HA expression co-localized with markers for epithelial cells, including club, basal, and ciliated cells, indicating the lipid composition preferentially targeted the respiratory epithelium. Single 0.4 mg/kg administration of inhaled LNP-formulated DNAI1-HA mRNA was introduced to two NHPs (one male and one female). Lung and bronchial sections were collected six hours after dosing. Percentage of DNAI1-HA positive was calculated by combining cell counts from 4 lung sections (˜500,000 to 1,400,0000 cells counted) and 1 bronchial section (˜16,000 to 65,000 cells counted) from each animal.

### Example 19. Aerosol Administration of Formulated DNAI1 mRNA Rescues Ciliary Activity in Knock-Down Primary Bronchial Human ALI Cultures

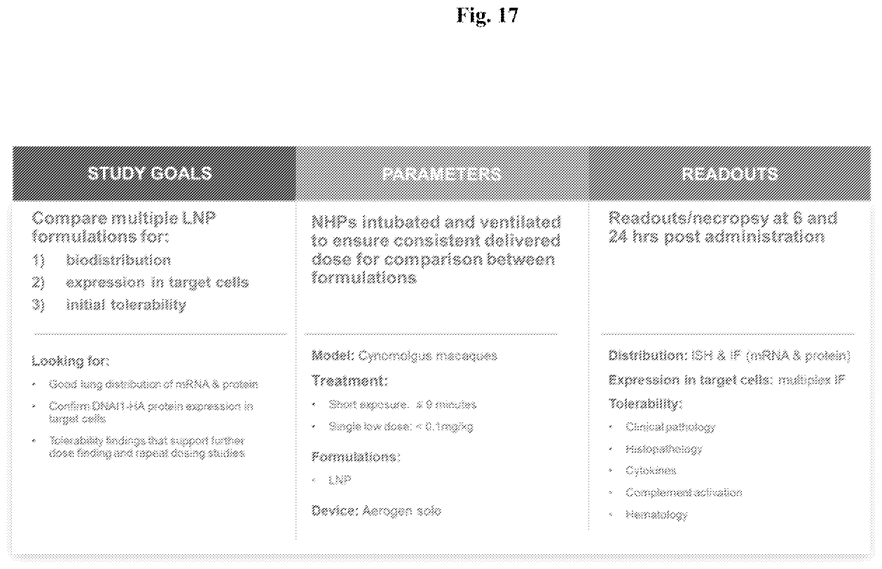

Human primary bronchial epithelial DNAI1 knock-down cells were cultured at an ALI. Well-differentiated cells were treated 2×/week (T, F) with LNP C (300 μg/d using Vitrocell nebulization) starting on day 25 post ALI culture age. The last does was administered on day 50 post ALI culture. Ciliary activity was measured by cross-sectional area (CSA) and beat frequency following certain doses. FIG. 17 shows increased ciliary activity in treated DNAI1 knock-down cultures was first detected 7 days after dosing was initiated.

### Example 20. Prolonged Rescue of Ciliary Activity in KO-Primary Tracheal Mouse ALI Cultures

Mouse tracheal epithelial cells (MTEC) are harvested from Dnaic1 mice. The cells are cultured as described in Example 8 and grown until differentiating. The differentiating Dnaic1 knock-out (KO) mouse cells are treated with a low dose of a lipid composition comprising a SORT compound disclosed herein and carrying a DNAI1 mRNA. Ciliary activity as measured by ciliary cross-sectional area (CSA) and ciliary beat frequency (CSF) is determined at certain timepoints. Wild type (WT) and PCD/no TAM cells are used as positive controls and untreated Dnaic1 KO cells as negative controls. Ciliary activity in treated Dnaic1 KO cells may be higher than untreated Dnaic1 KO cells.

### Example 21. Additional SORT Molecules

SORT lipids were screened for strong lung or spleen specificity and tolerability in mouse IV studies. The second from the left column of FIG. 10B discusses the cell tropism achieved through the SORT lipids screened. Five SORT molecules were evaluated and resulted in 2-7 times higher potency in hBEs cell cultures compared to RTX0001.

### Example 22. Stability and Efficacy Study

Buffers and cryopreservatives for freezing, concentrations, osmolality and ionic strength were screened. Certain buffers provided stability across different SORT lipids and lipid compositions. The screened buffers resulted in some formulations to increase in particle size after a freeze/thaw cycle. The final particle size after a freeze/thaw cycle for all formulation resulted in acceptable ranges for particle size (<130 nm).

Potency and tolerability of formulations were tested after being stored in frozen conditions (freeze/thaw) in in vitro and in vivo nebulization experiments. Lipid compositions with SORT lipids in the screened buffer under a freeze/thaw cycle and without freeze/thaw was nebulized in hBEs. Some formulations had small change in potency (either increase or decrease), but all formulations maintained higher potency than RTX0001. The study showed that high potency was remained in the screened buffer for lipid compositions.

### Example 23. Additional Screening Studies

Lipid compositions comprising SORT lipids were evaluated with a reduced 25% and 50% total lipid/mRNA ratio (N/P ratio). The 50% mRNA reduction resulted in small decrease in potency in both hBE and mouse nebulization for some lipid compositions, while other tested lipid compositions retained their potency. Two lipid compositions comprising SORT lipids were observed to have a small increase in particle size when tested with a 50% reduction in total lipid compared to RTX0001.

The lipid compositions were screened with changes in PEG lipid content and different N/P ratios. A small increase in particle size was observed with decrease in PEG lipid amount. A screened range of PEG concentration provided 120 nm particle size.

The results provide support that a change (e.g., increase) in % PEG lipid in the lipid composition may result in a change in potency (e.g., increase). In a hBE nebulization study, a decrease in PEG lipid amount resulted in an increased potency and an increase in PEG lipid amount resulted in a decrease in potency.

### Example 24. SORT NHP Study

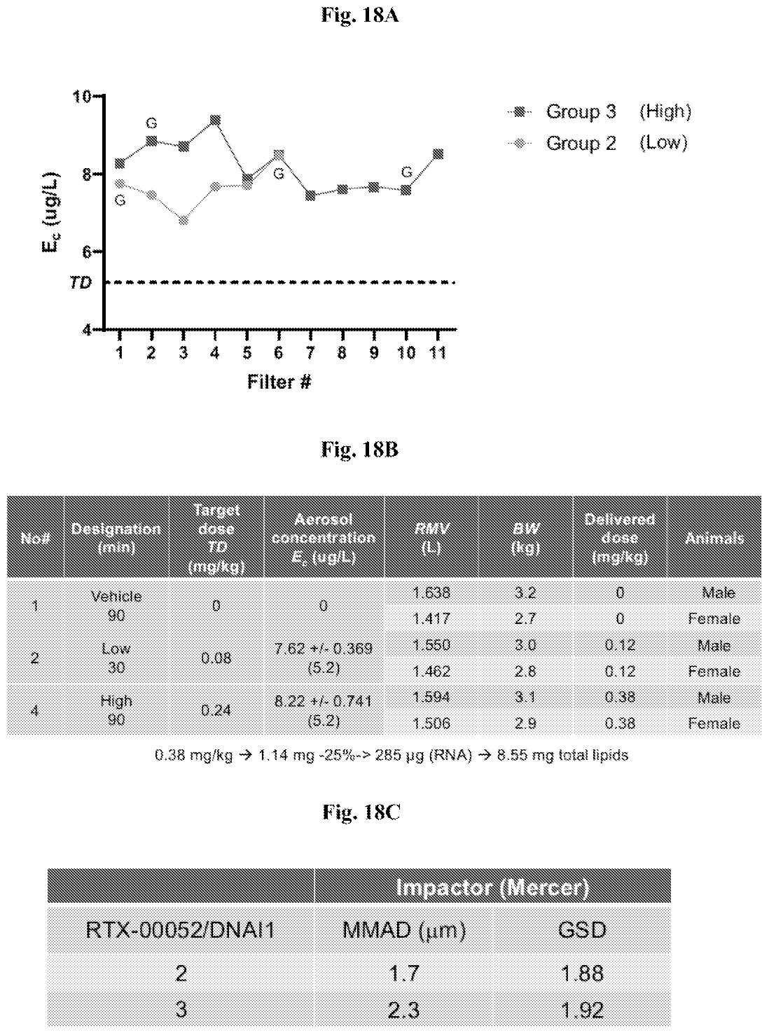

NHPs (Cynomolgus monkey, Macaca fascicularis, Mauritius origin, 2.5 to 3 years old, male: 2.7-3.3 kg/female: 2.5-3.0 kg; N=18 total, N=8 per dose group (4 male/4 female), N=2 vehicle control (1 male/1 female) were examined for the efficacy of aerosol delivery by inhalation using oronasal face mask. The delivery doses were 0.12 mg/kg or 0.24 mg/kg. Expression of DNAI1 was examined at six hours, 24 hours, 72 hours, or seven days after administration of RTX0052 (a lipid composition described herein. Readout for determining the efficacy of the was determined in NHPs administered with vehicle (Group 1); low dose (Group 2 with a target dose of 0.08 mg/kg; target aerosol concentration Eₑ of 0.0052 mg/L for 30 minutes); and high dose (Group 3 with a target dose of 0.24 mg/kg; target aerosol concentration E, of 0.0052 mg/L for 90 minutes). FIG. 18A illustrates the aerosol concentration administered to the NHPs, and FIG. 18B illustrates exemplary measurements of the doses delivered to the NHPs. FIG. 18C illustrates characterization of the aerosol composition droplet (MMAD: mass median aerodynamic diameter; GSDL: geometric standard deviation). The droplet characterization results were within recommended range of the Organization for Economic Co-operation and Development (OECD) guidance 433 for inhalation toxicity studies with an MMAD <4 μm and a GSD between 1.0 and 3.0.

As used herein, “RTX0052” refers to an example lipid composition tested herein. RTX0052 is a 5-component lipid nanoparticle composition comprising about 19.05% 4A3-SC7 (ionizable cationic lipid), about 20% 14:0 TAP (SORT lipid), about 19.05% DOPE, about 38.9% cholesterol, and about 3.81% DMG-PEG (PEG conjugated lipid), wherein each lipid component is defined as mol % of the total lipid composition.

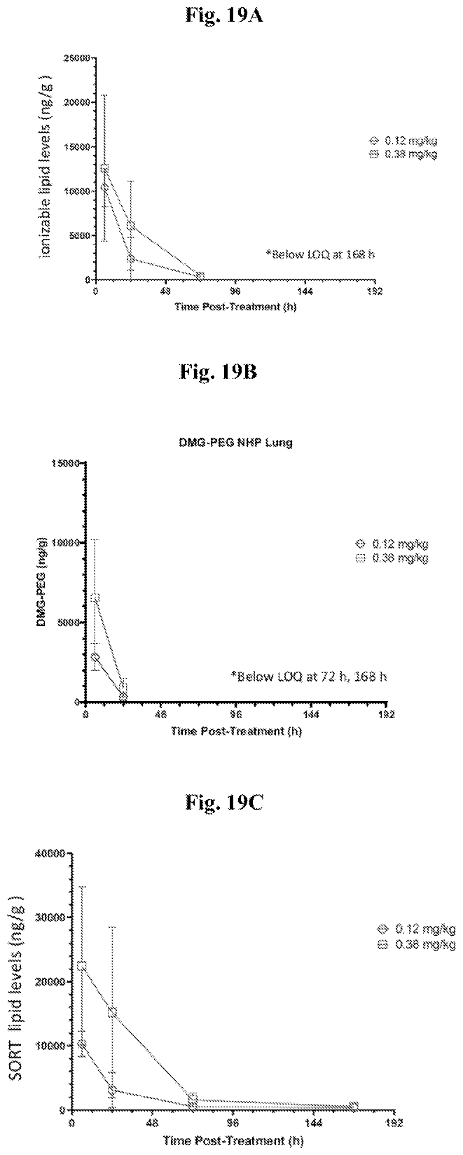

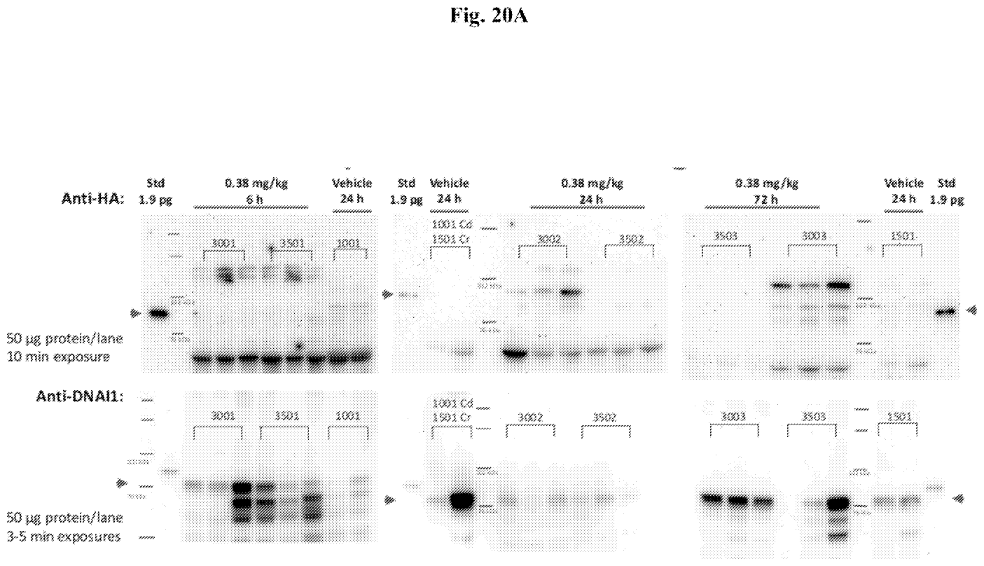

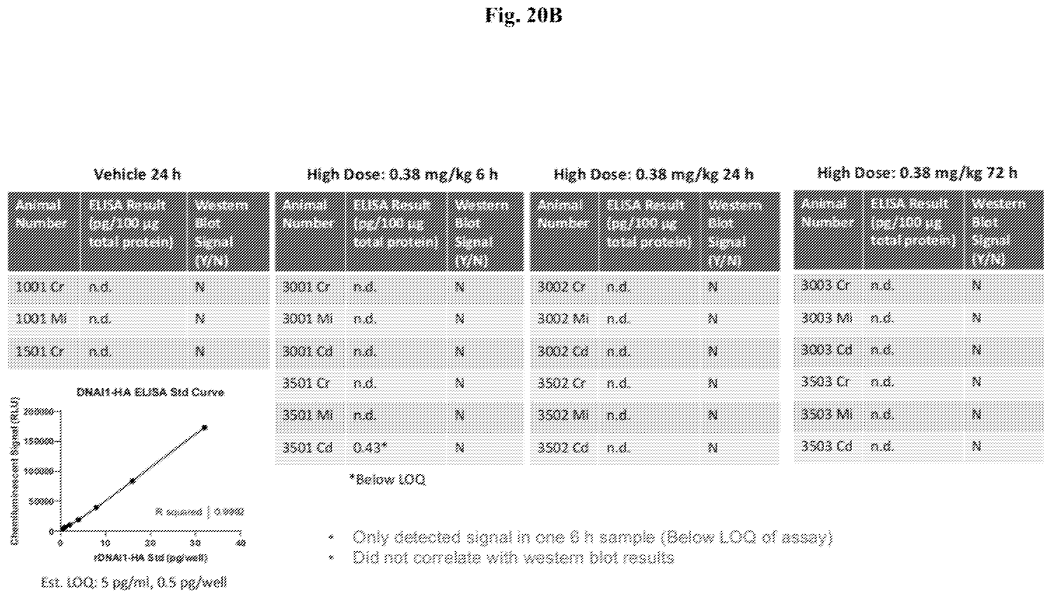

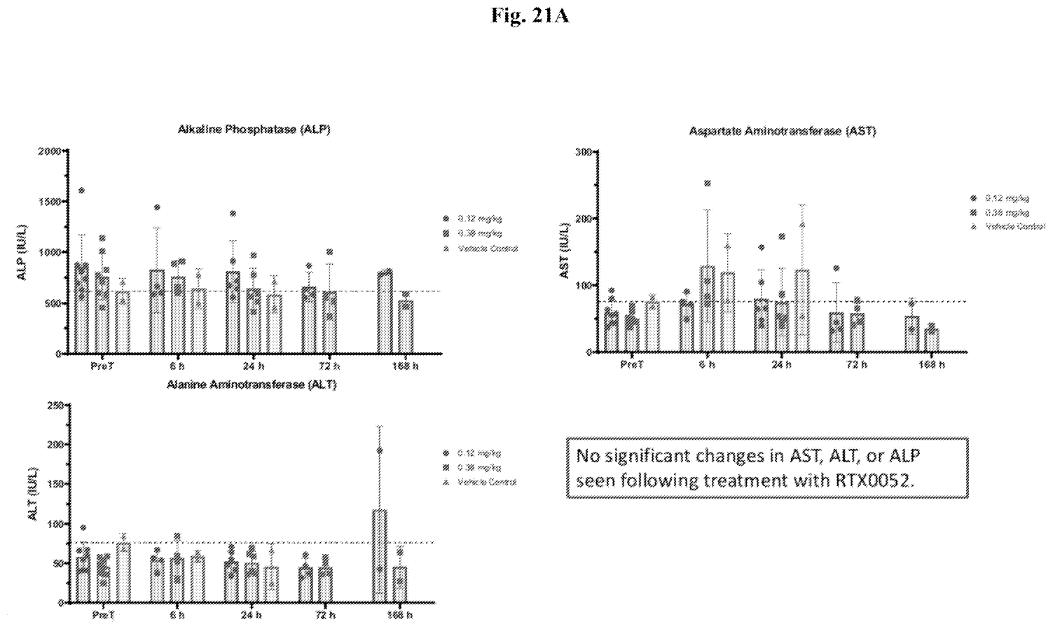

Measurement of the droplet (lipid) in NHP blood, lung, liver, and spleen tissue was determined by liquid chromatography and mass spectrometry (LC/MS-MS). Sample Matrices: Blood (plasma and blood cell fractions), Lung, Liver, Spleen. Limit of quantification (LOQ) of ionizable lipids in plasma was 4 ng/ml. LOQ of ionizable lipid in blood cell fraction was 4 ng/ml. LOQ of ionizable lipid in lung tissue cell fraction was 10 ng/ml. LOQ of PEGylation of myristoyl diglyceride (DMG-PEG) in plasma was 20 ng/ml. LOQ of DMG-PEG in blood cell fraction was 40 ng/ml. LOQ of DMG-PEG in lung tissue was 20 ng/ml. LOQ SORT lipid in lung tissue cell fraction was 10 ng/ml. LOQ of SORT lipid in plasma was 1 ng/ml. LOQ of PEGylation of SORT lipid in blood cell fraction was 1 ng/ml. LOQ of SORT lipid in lung tissue was 2 ng/ml. FIGS. 19A-C illustrate measurement of LNP lipid (stemmed from aerosol droplet) in lung in both low dose and high dose NHP group (FIG. 19A: ionizable lipid in lung; FIG. 19B: DMG-PEG in lung; and FIG. 19C: SORT lipid). Table 12 illustrates detection of DNAI1-HA in the processed sample of the NHP. For Table 12, 2 sets of lung samples per animal (6 total) processed and assayed for: Western blot: anti-HA and anti-DNAI1; and ELISA: DNA11-HA (Capture Ab: Anti-HA, Detection Ab: anti-DNAI1). FIG. 20A illustrates DNAI1-HA protein expression in the NHP lung by Western blotting. FIG. 20B illustrates DNAI1-HA protein expression in the NHP lung by ELISA.

TABLE 12
DNAI1-HA detection in NHP sample

| Animal | Group | Necropsy | | Lung Samples |
|---|---|---|---|---|
| 3001 | High Dose 0.38 | 6 | h | N = 3 (1 ea. Caudal, Cranial, |
| | mg/kg | | | Middle Lobe) |
| 3501 | High Dose 0.38 | 6 | h | N = 3 (1 ea. Caudal, Cranial, |
| | mg/kg | | | Middle Lobe) |
| 3002 | High Dose 0.38 | 24 | h | N = 3 (1 ea. Caudal, Cranial, |
| | mg/kg | | | Middle Lobe) |
| 3502 | High Dose 0.38 | 24 | h | N = 3 (1 ea. Caudal, Cranial, |
| | mg/kg | | | Middle Lobe) |
| 3003 | High Dose 0.38 | 72 | h | N = 3 (1 ea. Caudal, Cranial, |
| | mg/kg | | | Middle Lobe) |
| 3503 | High Dose 0.38 | 72 | h | N = 3 (1 ea. Caudal, Cranial, |
| | mg/kg | | | Middle Lobe) |
| 1001 | Vehicle | 24 | h | N = 3 (1 ea. Caudal, Cranial, |
| | | | | Middle Lobe) |
| 1501 | Vehicle | 24 | h | N = 3 (1 ea. Caudal, Cranial, |
| | | | | Middle Lobe) |

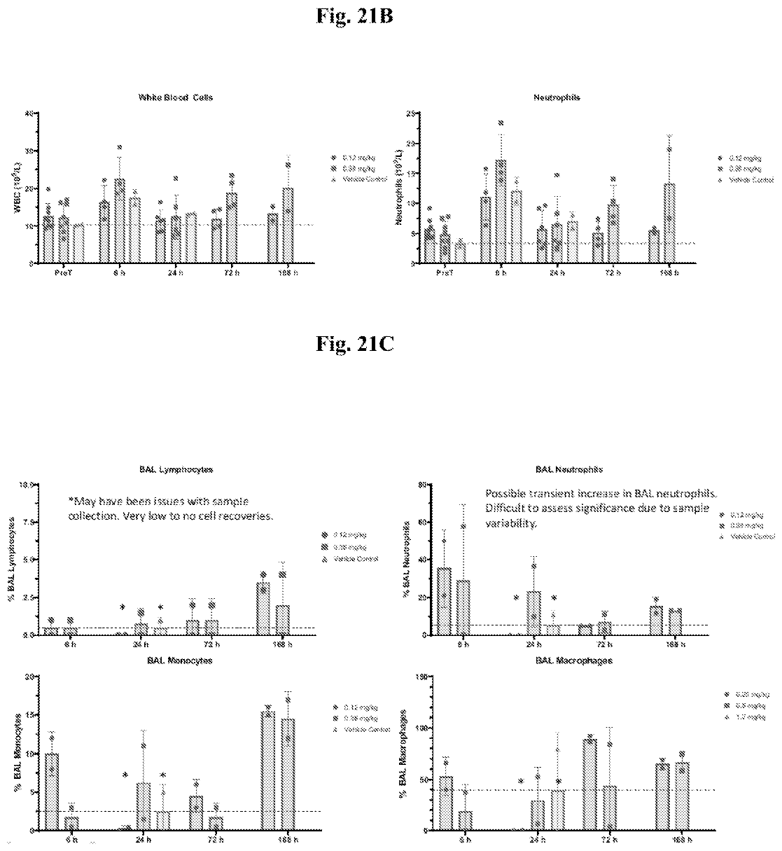

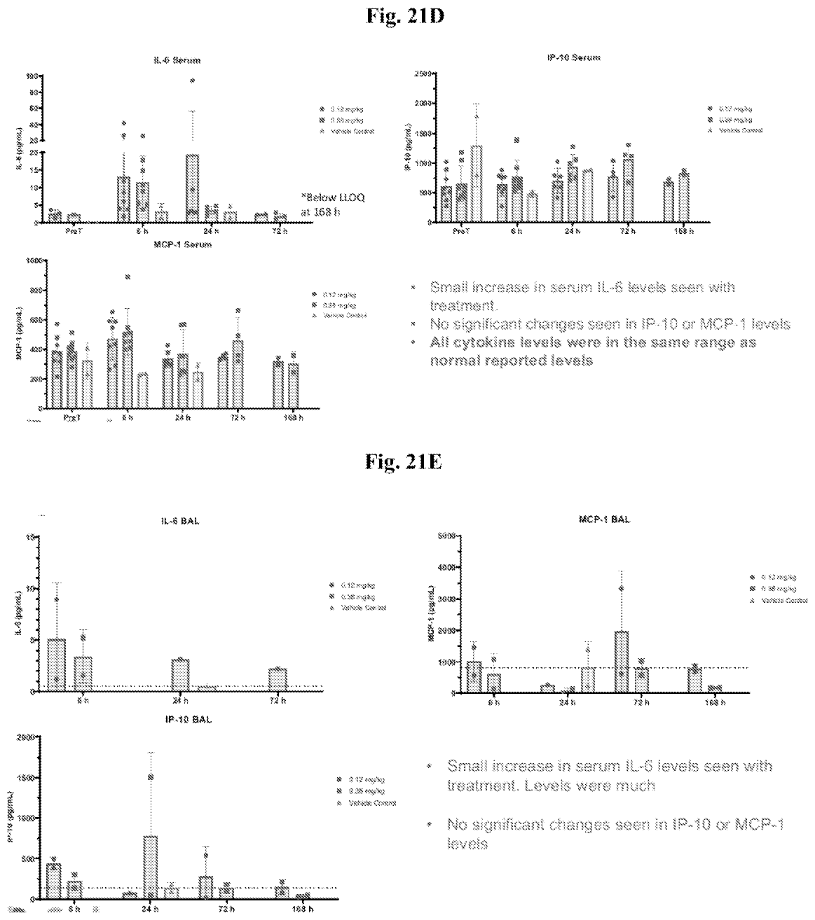

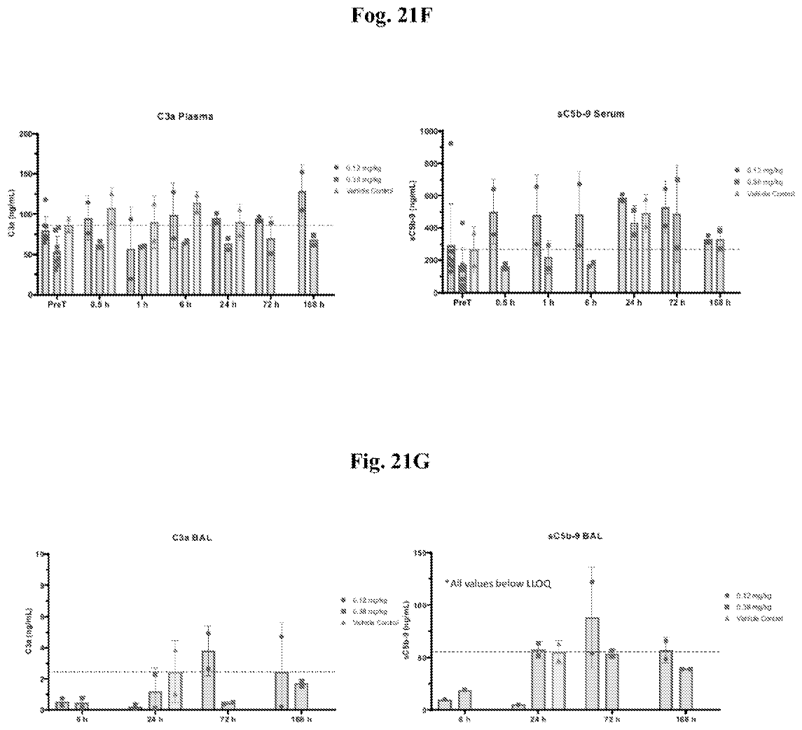

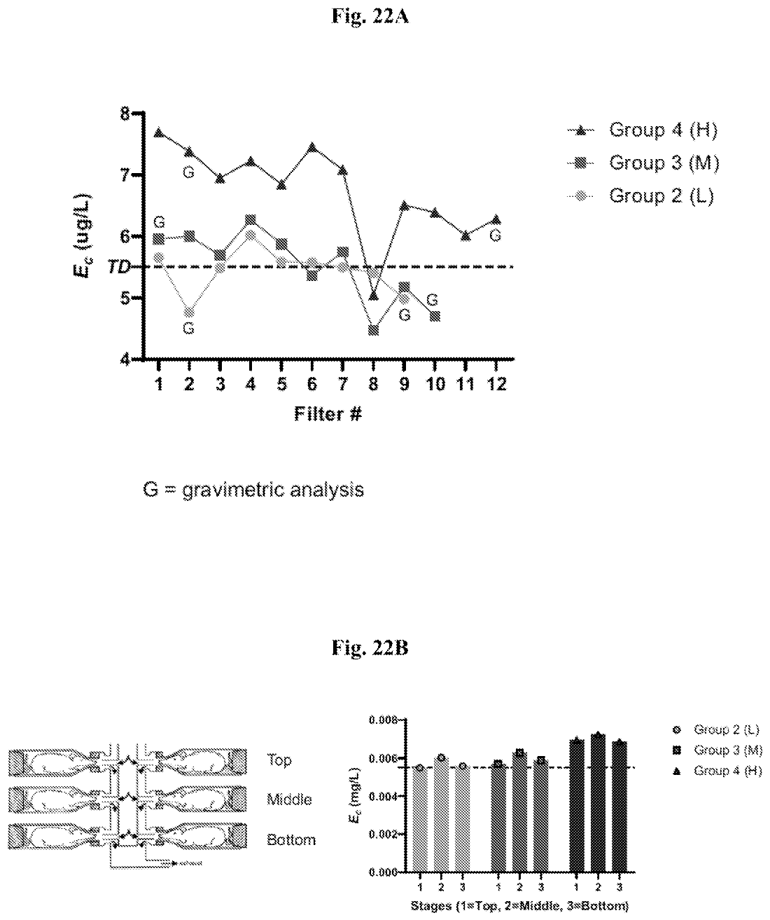

Tolerability of the RTX0052 was determined based on clinical observations; body and organ weights; clinical chemistry and hematology; bronchoalveolar lavage (BAL) cell differentials; cytokine and complement levels in serum and BAL; and histopathology. There were no adverse clinical signs observed that were considered related to treatment with RTX0052. No significant changes in body weight were observed between the treatment groups. There were also no organ weight changes (absolute and relative to body weight) that were clearly related to RTX0052. All changes observed in other tissues/organs, with/without statistical significance, in males and females at all dose levels were independent of dose and/or sex or were minor in magnitude or within ITR background ranges, thus, considered to be incidental or procedure/stress-related. FIG. 21A illustrates clinical chemistry measurements for AST, ALT, and ALP. No significant changes of AST, ALT, or ALP were observed following treatment with RTX0052. For hematology and coagulation, there were no RTX0052-DNAI1-related changes in hematology parameters measured in monkeys at 6-hour, 24-hour and 72-hour post end of exposure, and 7 days of observation after inhalation exposure. Some female monkeys had relatively higher white blood cells and neutrophils counts in blood at 6 hours, 72 hours and 7 days post end of exposure but were not considered adverse. There were no RTX0052-DNAI1-related changes in coagulation parameters measured in monkeys at 6-hour, 24-hour and 72-hour post end of exposure, and 7 days of observation after inhalation exposure. FIG. 21B illustrates the hematology counts of white blood cells and neutrophils. Some increase in neutrophils was observed in the post-treatment measurements of both vehicle and RTX0052 groups. FIG. 21C illustrates BAL cell differentials. For cytokine and complement analysis, cytokines levels were measured in NHP serum and BAL. Analytes measured included IFN-α2a, IFN-γ, IL-β, IL-4, IL-6, IL-10, IL-17A, IP-10, MCP-1, and TNFα. All cytokine levels were in the same range as normal reported elves. BAL results were similarly normal as all cytokines were at or below serum lower limit of quantification (LLOQ) (except for IL-6, IL-10, and MCP-1). Table 13 illustrates the serum and BAL LLOQ measurements of analytes. FIG. 21D illustrates exemplary measurements of cytokine in serum. FIG. 21E illustrates exemplary measurements of cytokine in BAL. FIG. 21F illustrates exemplary complement measures of C3a and sC5b-9 measurements in plasma and serum respectively. FIG. 21G illustrates exemplary complement measures of C3a and sC5b-9 measurements in BAL.

TABLE 13
LLOQ Analyte measurement in serum and BAL

| | Serum LLOQ | BAL LLOQ |
|---|---|---|
| Analyte | (pg/mL) | (pg/mL) |
| IFN-α2a | 18.36 | 9.18 |
| IFN-γ | 13.38 | 6.69 |
| IL-1β | 2.2 | 1.1 |
| IL-4 | 0.98 | 0.49 |
| IL-6 | 1 | 0.5 |
| IL-10 | 2.08 | 1.04 |
| IL-17A | 13.52 | 6.76 |
| IP-10 | 4.6 | 2.3 |
| MCP-1 | 2.66 | 1.29 |
| TNFα | 1.8 | 0.9 |

Based on the histopathology analysis performed herein, there was no evidence of test item-related macroscopic findings. All gross observations were considered to be incidental, as they were sporadic and not dose related, of low incidence, or occurred in control and treated animals or lacked the relevant histopathology correlates. Minimal to mild increase in the alveolar mixed cell infiltrates were observed in the lungs of 3/8 animals treated at target total inhaled dose level of 0.24 mg/kg. Due to low incidence and severity, this change was considered to be potentially test item-related, but non-adverse. All other microscopic observations were considered to be incidental, background or agonal changes, as they were of low incidence or severity, or occurred in control and test item-treated animals. Overall treatment was well-tolerated: No changes seen in clinical observations, clinical chemistry, or complement measurements. Slight and transient increased in blood and BAL neutrophils was observed. All cytokine levels were within the normal reported range. Small transient increase in IL-6 levels was observed in both serum and BAL. Histopathology indicated minimal to mild increase in the alveolar mixed cell infiltrates in 3/8 animals.

### Example 25. SORT Rat Study

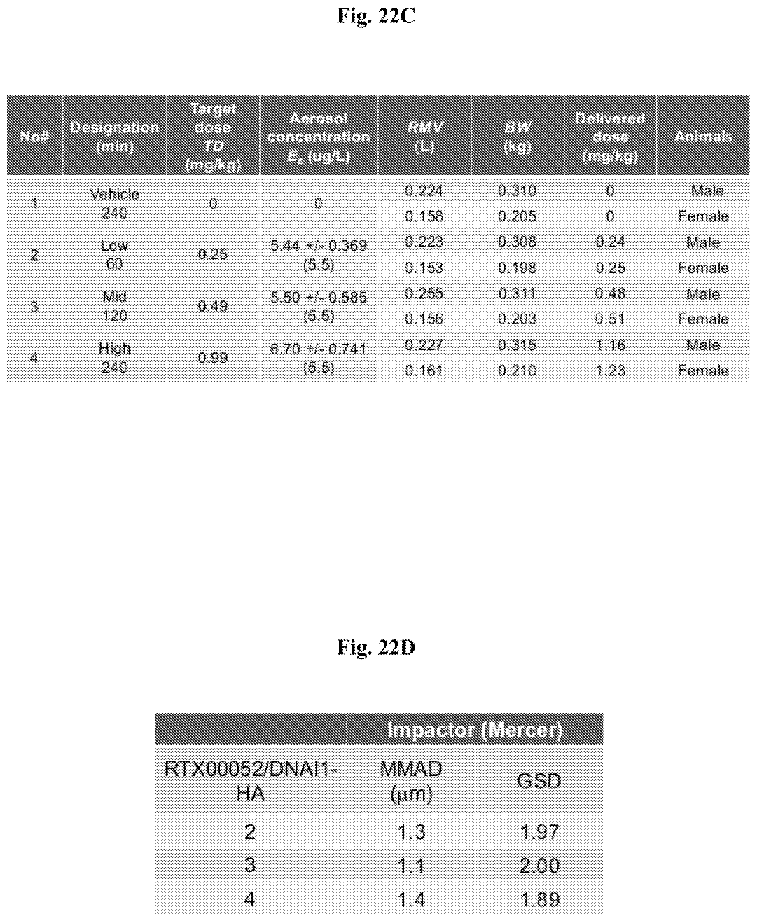

Rats (Sprague-Dawley (SD), 8 to 11 weeks old, male: 300-350 g/female: 175-250 g; N=130 total, N=40 per dose group (20 male/20 female), N=10 vehicle control (5 male/5 female)) were examined for the efficacy of aerosol delivery by inhalation using flow-past exposure system. The delivery dose was low (0.25 mg/kg target dose), mid (0.49 mg/kg target dose), or high (0.99 mg/kg target dose). Expression of DNAI1 was examined at six hours, 24 hours, 72 hours, or seven days after administration of RTX0052. Readout for determining the efficacy of the RTX0052 was determined in rats administered with vehicle (Group 1); low dose (Group 2 with a target dose of 0.25 mg/kg; target aerosol concentration Ec of 0.0055 mg/L for 60 minutes); mid dose (Group 3 with a target dose of 0.49 mg/kg; target aerosol concentration Ec of 0.0055 mg/L for 120 minutes); and high dose (Group 4 with a target dose of 0.99 mg/kg; target aerosol concentration Ec of 0.0055 mg/L for 240 minutes). FIG. 22A illustrates the aerosol concentration administered to the rats. FIG. 22B illustrates exemplary measurements aerosol homogeneity across three stages. FIG. 22C illustrates the amount of doses delivered to the rats. FIG. 22D illustrates characterization of the aerosol composition droplet (MMAD: mass median aerodynamic diameter; GSDL: geometric standard deviation). The droplet characterization results were within recommended range of the Organization for Economic Co-operation and Development (OECD) guidance 433 for inhalation toxicity studies with an MMAD <4 μm and a GSD between 1.0 and 3.0.

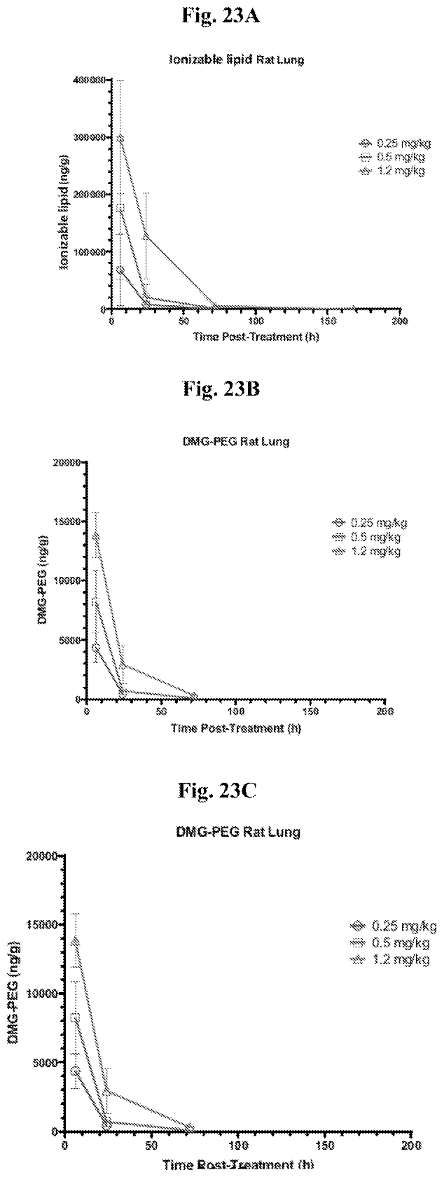

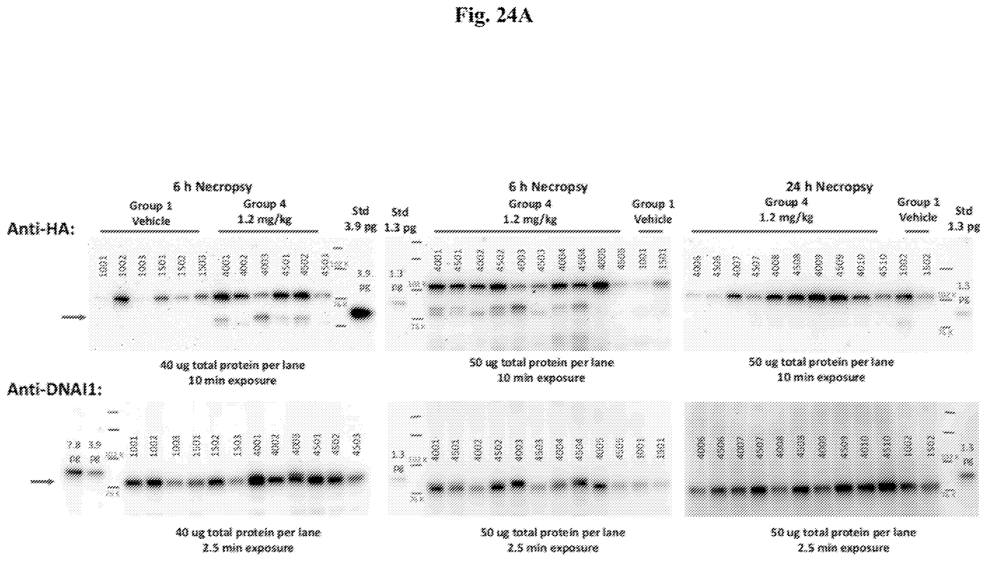

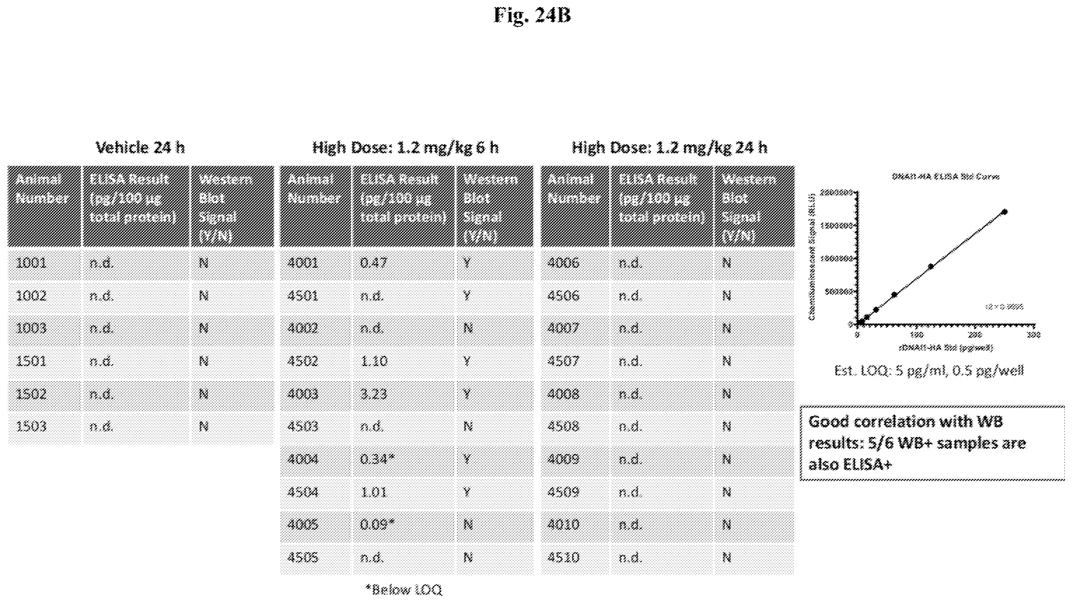

Measurement of the droplet (lipid) in rate blood, lung, liver, and spleen tissue was determined by liquid chromatography and mass spectrometry (LC/MS-MS). Sample Matrices: Blood (plasma and blood cell fractions), Lung, Liver, Spleen. Limit of quantification (LOQ) of ionizable lipids in plasma was 4 ng/ml. LOQ of ionizable lipid in blood cell fraction was 4 ng/ml. LOQ of ionizable lipid in lung tissue cell fraction was 10 ng/ml. LOQ of PEGylation of myristoyl diglyceride (DMG-PEG) in plasma was 20 ng/ml. LOQ of DMG-PEG in blood cell fraction was 40 ng/ml. LOQ of DMG-PEG in lung tissue was 20 ng/ml. LOQ SORT lipid in lung tissue cell fraction was 10 ng/ml. LOQ of SORT lipid in plasma was 1 ng/ml. LOQ of PEGylation of SORT lipid in blood cell fraction was 1 ng/ml. LOQ of SORT lipid in lung tissue was 2 ng/ml. FIGS. 23A-C illustrate measurement of LNP lipid (stemmed from aerosol droplet) in lung in low dose, mid dose, and high dose rat group (FIG. 23A: ionizable lipid in lung; FIG. 23B: DMG-PEG in lung; and FIG. 23C: SORT lipid). FIG. 24A illustrates DNAI1-HA protein expression in the rat lung by Western blotting. Six out of ten lung samples I the 1.2 mg/kg, 6 hour group were positive for DNAI1-HA. FIG. 24B illustrates DNAI1-HA protein expression in the rat lung by ELISA.

Tolerability of the RTX0052 was determined based on clinical observations. There were no clinical signs related to treatment with RTX0052-DNAI1. No significant changes in body weight between the treatment groups was observed. Food consumption was unaffected by treatment with RTX0052-DNAI1. There were no organ weight changes (absolute and relative to body weight) that were clearly related to RTX0052-DNAI1. All changes observed in other tissues/organs, with/without statistical significance, in males and females at all dose levels were independent of dose and/or sex or were minor in magnitude or within ITR background ranges, thus, considered to be incidental or procedure/stress-related.

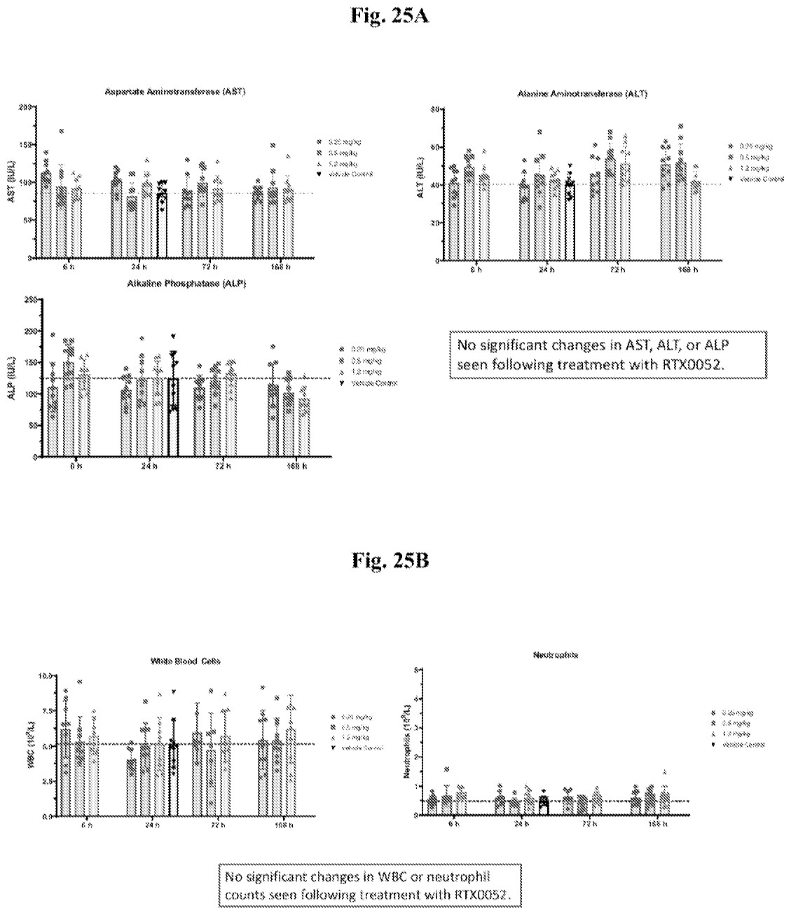

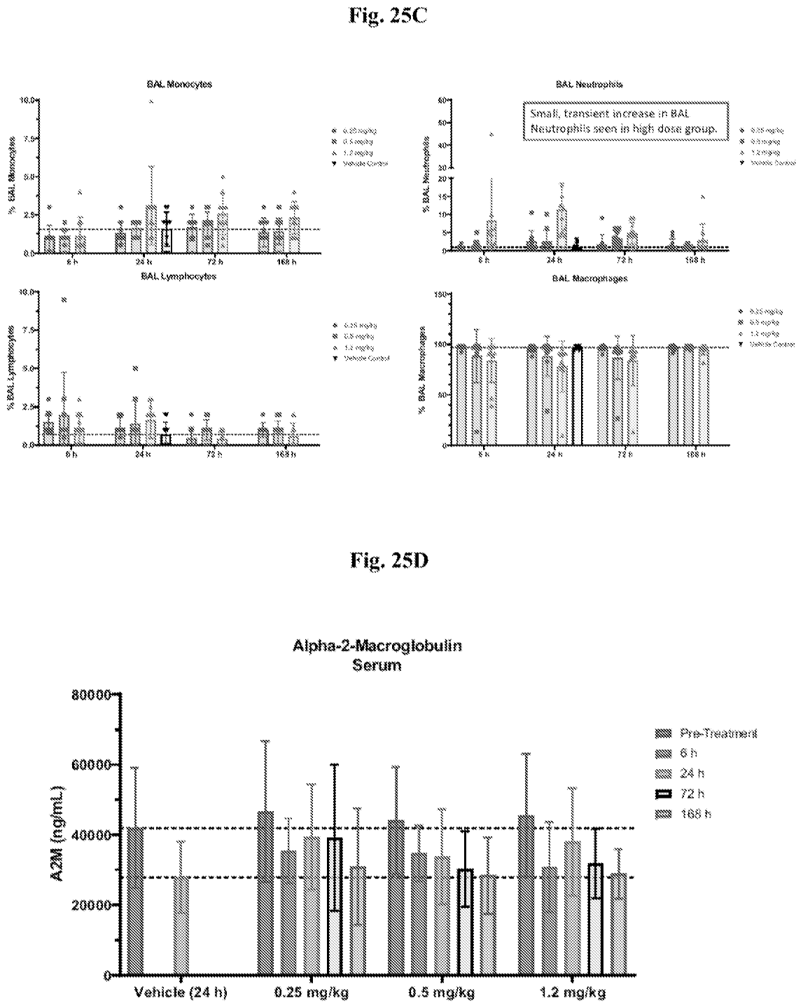

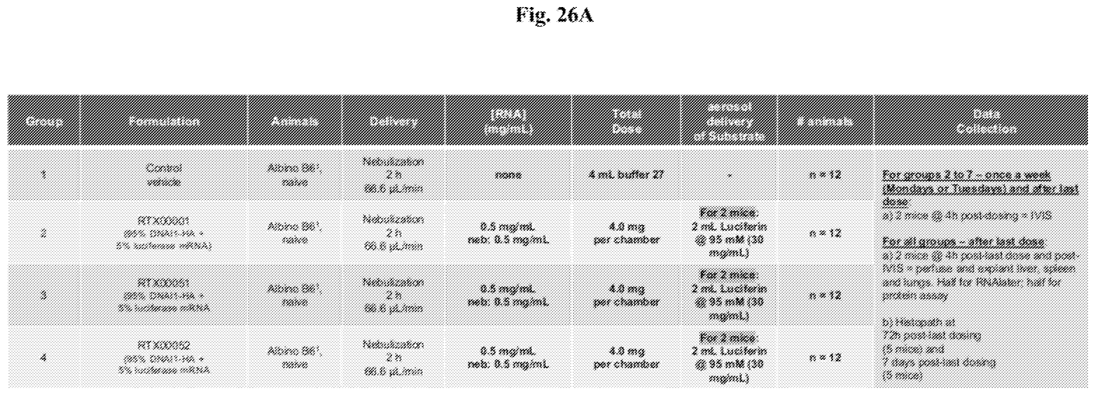

FIG. 25A illustrates clinical chemistry measurements for AST, ALT, and ALP in the treated rats. There were no RTX0052-DNAI1-related changes in clinical parameters measured in rats at 6-hour, 24-hour and 72-hour post end of exposure, and 7 days of observation after inhalation exposure. Some mean values differed from the control values, with and without statistical significance, but the differences were independent of dose and/or sex or were minor in magnitude. Thus, they were considered to have no biological significance. FIG. 25B illustrates the hematology counts of white blood cells and neutrophils in the treated rats. For hematology and coagulation, there were no RTX0052-DNAI1-related changes in hematology parameters measured in rats at 6-hour, 24-hour and 72-hour post end of exposure, and 7 days of observation after inhalation exposure. There were no RTX0052-DNAI1-related changes in coagulation parameters measured in rats at 6-hour, 24-hour and 72-hour post end of exposure, and 7 days of observation after inhalation exposure. Some mean values differed from the control values, with and without statistical significance, but the differences were independent of dose and/or sex or were minor in magnitude. Thus, they were considered to have no biological significance. Some increase in neutrophils was observed in the post-treatment measurements of both vehicle and RTX0052 groups. FIG. 25C illustrates BAL cell differentials in the treated rats. FIG. 25D illustrates exemplary measurements of alpha-2-macroglobulin in the treated rats. A2M is a documented inflammation marker in the rat. Serum levels increased 12 to 48 hours after repeated acute inflammatory stimulations. For safety evaluations, A2M wass the preferred marker for the acute phase response in rats. No significant changes in A2M serum levels were observed following treatment with RTX0052.

Macroscopic finding was that regardless of the time point of termination (at 6-hour, 24-hour and 72-hour post end of exposure, and 7 days post exposure) of the treated rats, all macroscopic finding in the rats was considered incidental or spontaneous and not RTX0052-DNAI1-related. Microscopic finding was that regardless of the time point of termination (at 6-hour, 24-hour and 72-hour post end of exposure, and 7 days post exposure) of the treated rats, there were no microscopic pathology findings in the rats of this study that suggested systemic toxicity or local toxicity (oropharynx, nasopharynx, trachea, larynx, lungs) due to RTX0052-DNAI1. In one terminal Group 2 male rat (2016G), euthanized at 7 days post end of exposure, there was multifocal panlobular hepatic hemorrhagic coagulative necrosis and perilesional acute neutrophilic inflammation of the hepatic caudate lobe that correlated with its macroscopic finding. This microscopic finding was considered a spontaneous change that was not RTX0052-DNAI1- related, but rather associated with spontaneous torsion of the hepatic caudate lobe in rats (1). In another terminal Group 2 female rat (2512E), euthanized at 72-hour post end of exposure, a benign subcutaneous duct cell adenoma was noted in the inguinal skin/subcutis region. This finding was considered a spontaneous change that was not RTX0052-DNAI1- related, since it was not present in any of the Group 3 and Group 4 rats. All other microscopic findings, in the treated rats of this study were considered incidental or spontaneous, and not RTX0052-DNAI1-related. All other microscopic findings in the Control rats were considered incidental and spontaneous. LNP lipid components (ionizable lipid, DMG-PEG, and SORT lipid) were rapidly cleared from lung tissue following treatment. Measured levels for each in blood (plasma and cell fraction) were not detected or below the assay LOQs at each timepoint. Five out of ten lung samples in 1.2 mg/kg, at 6 hour post exposure were positive for DNAI1-HA protein expression by WB (FIG. 24A) and ELISA (FIG. 24B). No significant changes were seen in tolerability endpoints; clinical chemistry, hematology, BAL cell differentials, or A2M. No significant histopathology findings that indicated local or systemic toxicity were observed.

### Example 26. Multi-Dose Histopathology Analysis after Aerosol Delivery of 3 Different LNP

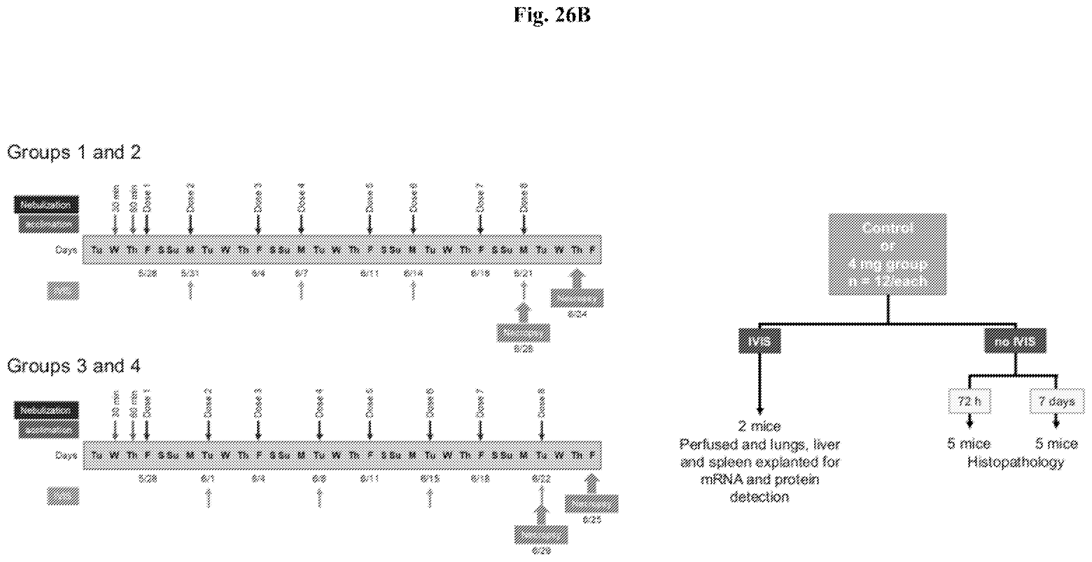

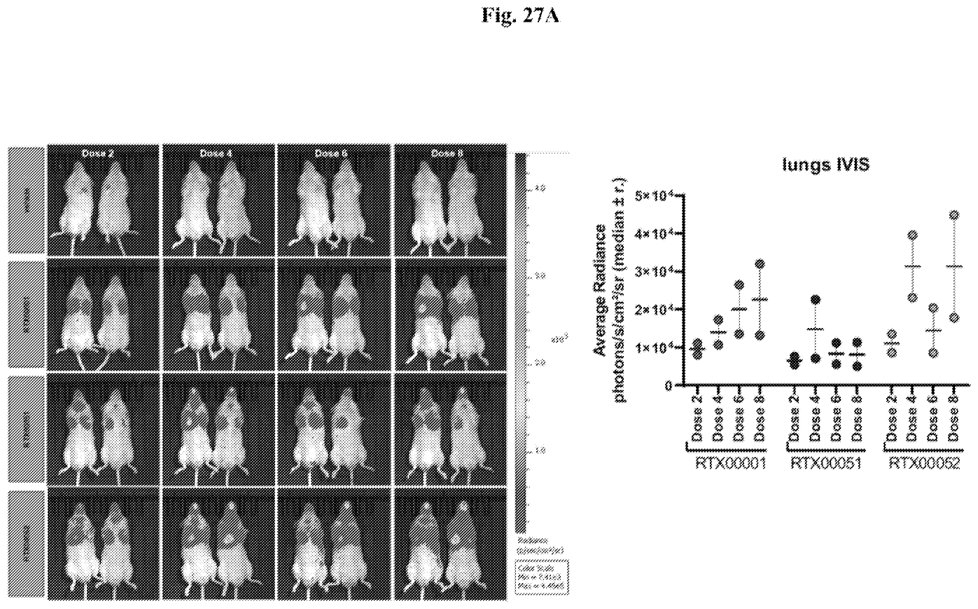

Multidose administration of 4 mg of 95% DNA11+5% Luciferase mRNAs in buffer #27 by nebulization. Three candidate formulations (RTX0001, RTX0051, and RTX0052) were compared. Readout assays included protein detection by ELISA, mRNA levels by qPCR/dPCR at 4our h post-last dose and post-IVIS; and lung histopathology at 72 hour and 7 days post-last dosing. Multi-dose study evaluated toxicity of lead LNP candidates when administered by nebulization to mice. To deliver the LNP formulation, cages were setup for mice to acclimate for 8 days. Four groups of mice were acclimated for this study. FIG. 26A illustrates the information relating to the four groups of mice to be repeatedly treated with nebulization of LNP/DNAI1-HA mRNA. FIG. 26B illustrates the protocol for the dosing, imaging, and necropsy of the repeatedly dosed mice. In vivo imaging was conducted on the treated mice. 2 mL luciferin at 30 mg/mL in PBS were nebulized with constant flow over a period of ca.4 min; 4 h post-dosing. Animals were anesthetized with isoflurane (3% for induction, 2% for maintenance, 1 L/min oxygen flow). Ventral images were captured for 1 min (binning set to 8, f stop at 1). Calibrated units were shown as Average Radiance (photons/s/cm2/sr) representing the flux radiating omni-directionally from a user defined region. Total Flux=the radiance (photons/sec) in each pixel summed or integrated over the ROI area (cm2)×4π. Average Radiance=the sum of the radiance from each pixel inside the ROI/number of pixels or super pixels (photons/see/cm2/sr).

As used herein, “RTX0051” refers to an example lipid composition tested herein. RTX0051 is a 5-component lipid nanoparticle composition comprising about 19.05% 4A3-SC7 (ionizable cationic lipid), about 20% 14:0 EPC (SORT lipid), about 19.05% DOPE, about 38.9% cholesterol, and about 3.81% DMG-PEG (PEG conjugated lipid), wherein each lipid component is defined as mol % of the total lipid composition.

As used herein, “RTX0052” refers to an example lipid composition tested herein. RTX0052 is a 5-component lipid nanoparticle composition comprising about 19.05% 4A3-SC7 (ionizable cationic lipid), about 20% 14:0 TAP (SORT lipid), about 19.05% DOPE, about 38.9% cholesterol, and about 3.81% DMG-PEG (PEG conjugated lipid), wherein each lipid component is defined as mol % of the total lipid composition.

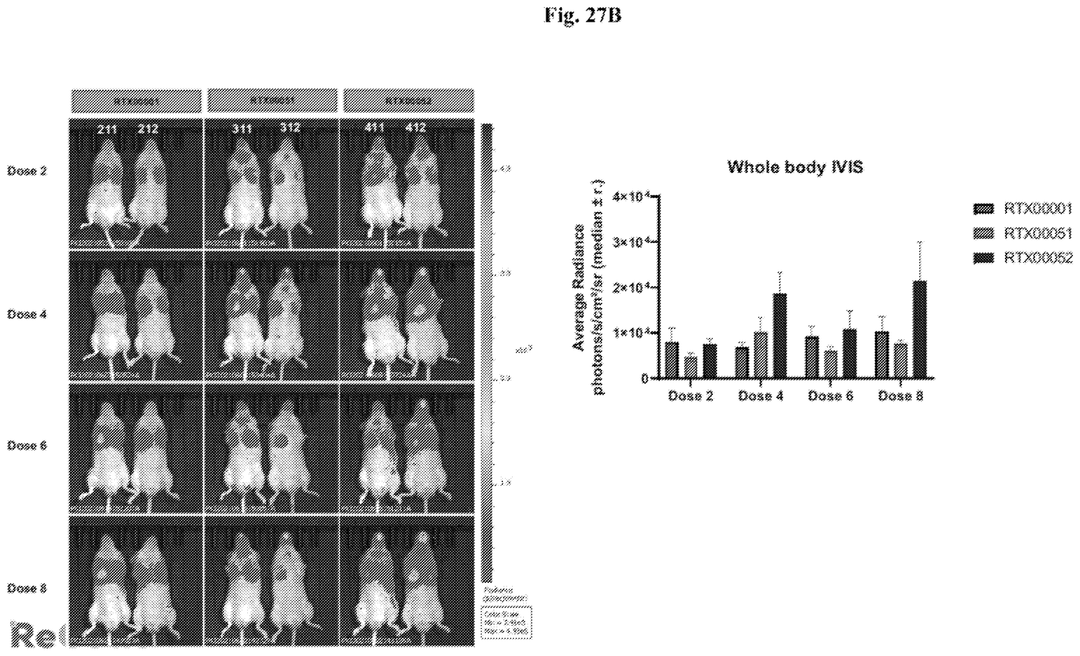

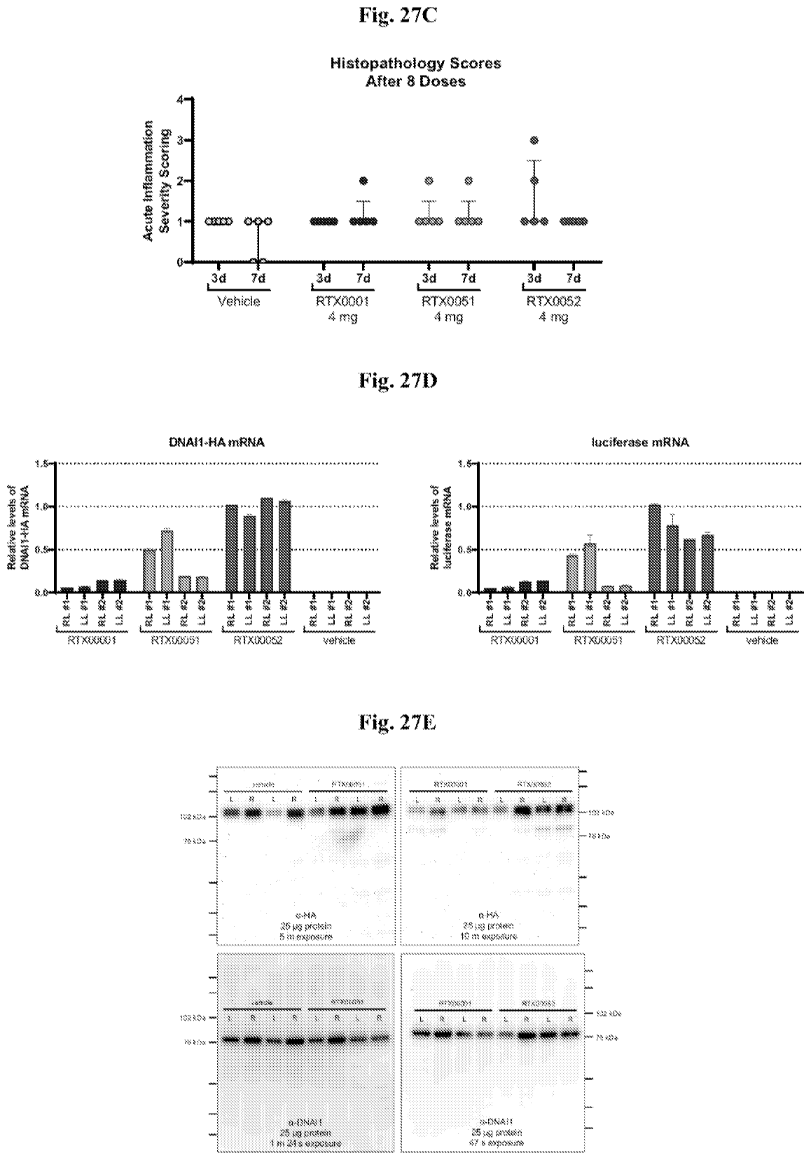

FIG. 27A-B illustrate whole body in vivo imaging (IVIS) of the repeatedly dosed mice. Animals, B6 Albino, male, about 7 weeks of age, naïve, were administered 4.0 mg of LNP-formulated DNAI1-HA/Luciferase by nebulization in 2 hours at 66.6 μL/min with Zero grade dry air flow at 2 L/min. 4 hour post-dosing, two mice were administered 2 mL of luciferin (30 mg/mL) by nebulization and imaged on IVIS within 1-15 min post-luciferin administration. Pseudo coloring was applied on the same scale for all images. Lung signal was plotted in graph of FIG. 27A. Whole body signal is plotted in the graph of FIG. 27B. FIG. 27C illustrates histopathology results of the repeatedly dosed mice. All formulations were well tolerated with most animals displaying minimal to mild inflammation scores. A single RTX0052-treated animal had moderate inflammation at 3 days post-exposure; however, this was resolved by 7 days with all animals showing only minimal inflammation. Tolerability data supports further studies in rats or NHPs. Histopathologic scoring system were as followed: 0 or normal: tissued considered to be normal under the conditions of the study and considering the age, sex, and strain of the animal concerned. Alterations may be present which, under other circumstances, would be considered deviation from normal; 1 or minimal: the amount of change barely exceeded that which was considered to be within normal limits; 3 or moderate: the lesion was prominent but there was significant potential for increased severity; limited tissue or organ dysfunction was possible; and 4 or severe: the degree was either as complete as considered possible or great enough in intensity or extent to expect significant tissue or organ dysfunction. FIG. 27D illustrates qPCR results showing the relative abundance of DNAI1-HA mRNA. After the last imaging of the last dose (dose 8), 2 mice per group were perfused. Spleen, liver and lungs were explanted. Half of each organ was preserved in RNAlater. Tissues were homogenized and total RNA purified with RNeasy Plus Universal Mini kit (Qiagen). Reverser transcription was performed with ProtoScript II First strand cDNA synthesis kit (NEB). Quantitative PCR was performed and analyzed. FIG. 27E illustrates Western blotting showing the protein expression of DNAI1-HA. 25 μg protein were loaded on 4-12% Bis Tris gel. Transferred to 0.45 μm Nitrocellulose and probed with monoclonal rat anti-HA. The blot was stripped and reprobed with rabbit anti-DNAI1.

### Example 27. Single Dose Inhalation Study

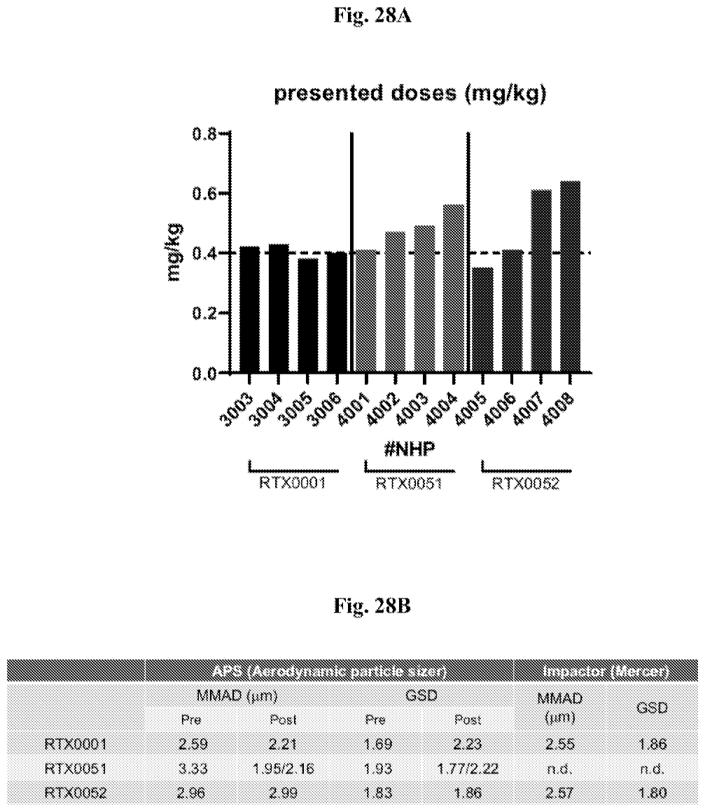

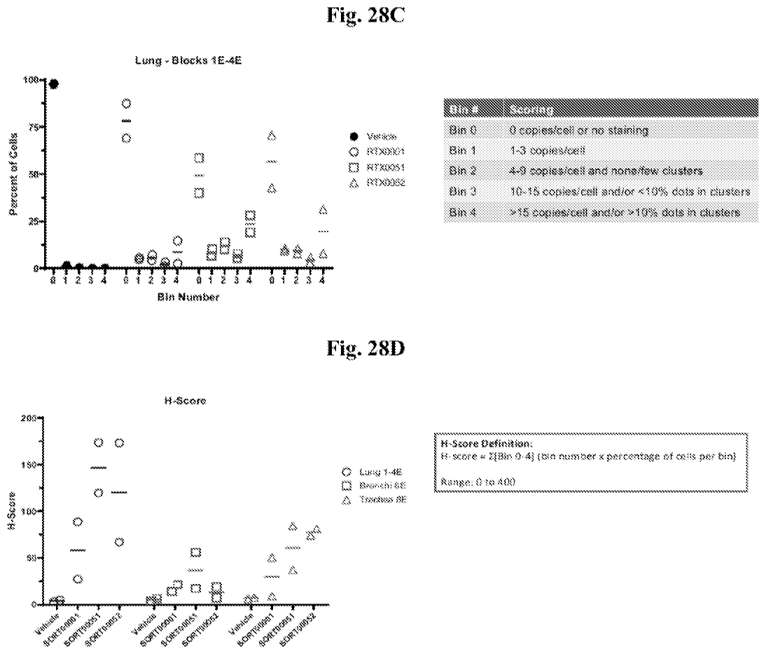

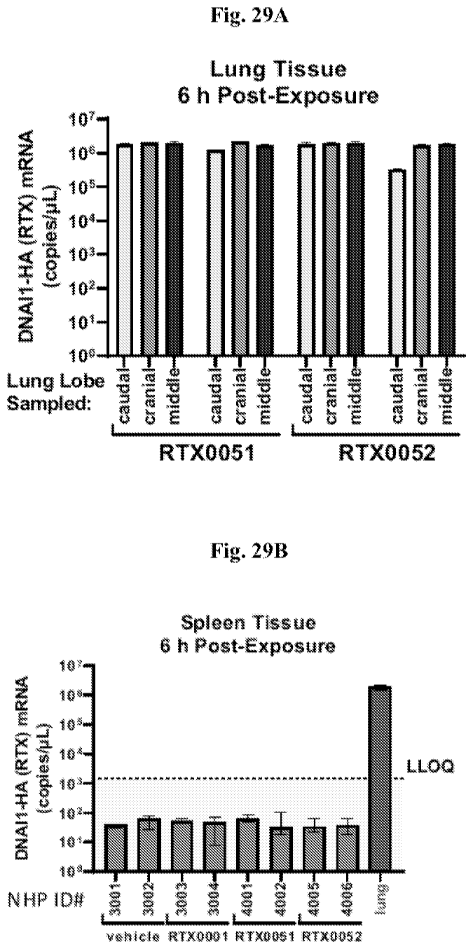

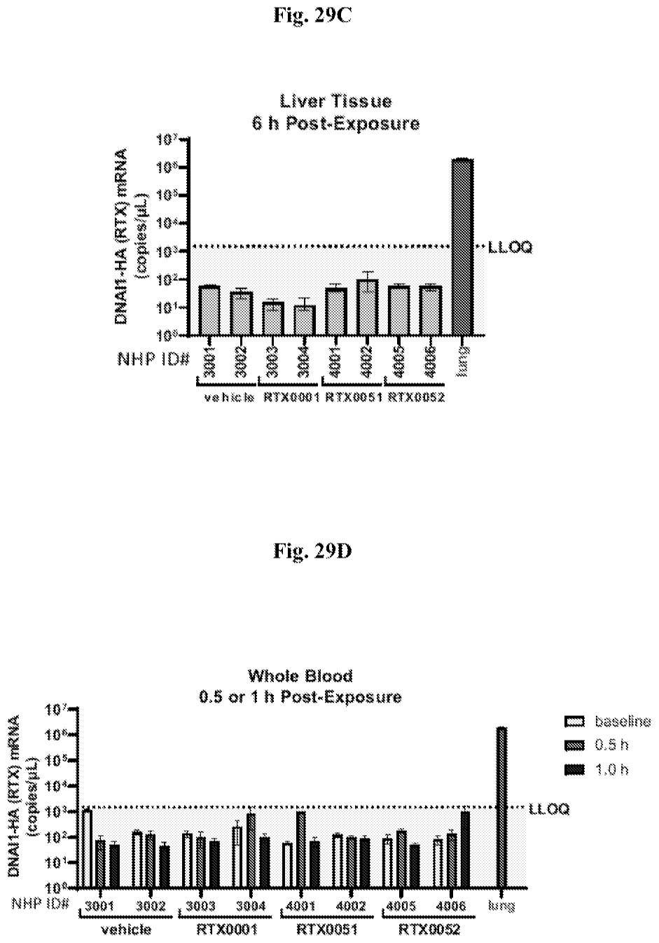

This example illustrates exemplary experimental approaches for selecting a lipid formulation based on tolerability, biodistribution and protein expression profiles in target cells (e.g., ciliated, club, or basal cells) of the lungs. DNAI1 mRNA was selected with three optimized 5-component formulations selected for comparison in NHPs based on studies in Examples 24-26. A dose between 0.4 to 0.6 mg/kg was delivered to achieve deposition in the TB region of 1-2 μg/cm² of DNAI1 mRNA for 5 to 10-fold higher than estimated dose required for efficacy. Necropsy timepoints for clinical assessment were at six hours and at 72 hours. FIG. 28A illustrates delivery of 0.4 mg/kg of LNP-formulated DNAI1 mRNA by inhalation. NHPs were intubated, ventilated, and dosed for fewer than 30 minutes. Targeted doses of 0/4 mg/kg were reached or exceeded for all tested LNP formulation. Presented doses of LNP-formulated DNAI1 were estimated based on gravimetric analysis of glass fiber filters and confirmed by filter elution and direct RNA cargo quantification using Ribogreen fluorescence assay. FIG. 28B illustrates LNP formulation aerosol characteristics. Aerosol particle size ranges for all three formulations were appropriate for deposition in the conducting airways. FIG. 28C illustrates biodistribution of DNAI1-HA mRNA in the targeted cells. High levels of DNAI1-HA mRNA were confirmed by in situ hybridization (ISH) in the lung by qPCR six hours post exposure, while no DNAI1-HA mRNA was detected above background in spleen (at six hours), liver (at six hours), or whole blood (at 30 minutes or at 60 minutes). ISH results demonstrated that up to 30% of lung cells contained more than 15 copies of the DNAI1-HA mRNA per cell after treatment with RTX0051 or RTX0052. FIG. 28D illustrates DNAI1-HA mRNA ISH results by H-Score. ISH results demonstrated high levels of DNAI1-HA mRNA were delivered to lung cells with lower levels in the bronchi and trachea. FIGS. 29A-D illustrate delivery of high levels of DNAI1-HA mRNA to the lung without exposure to liver, spleen, or blood. Digital PCR was used to measure DNAI1-HA mRNA levels in whole blood, lung, liver, and spleen tissue following a single 0.4 mg/kg administration. High levels of DNAI1-HA mRNA were detected in all three lung regions sampled at 6 hours post-exposure with RTX0051 and RTX0052. No DNAI1-HA mRNA was detected above background in spleen (6 hours, FIG. 29B), liver (6 hours, FIG. 29C), or whole blood (30 minutes or 60 minutes, FIG. 29D).

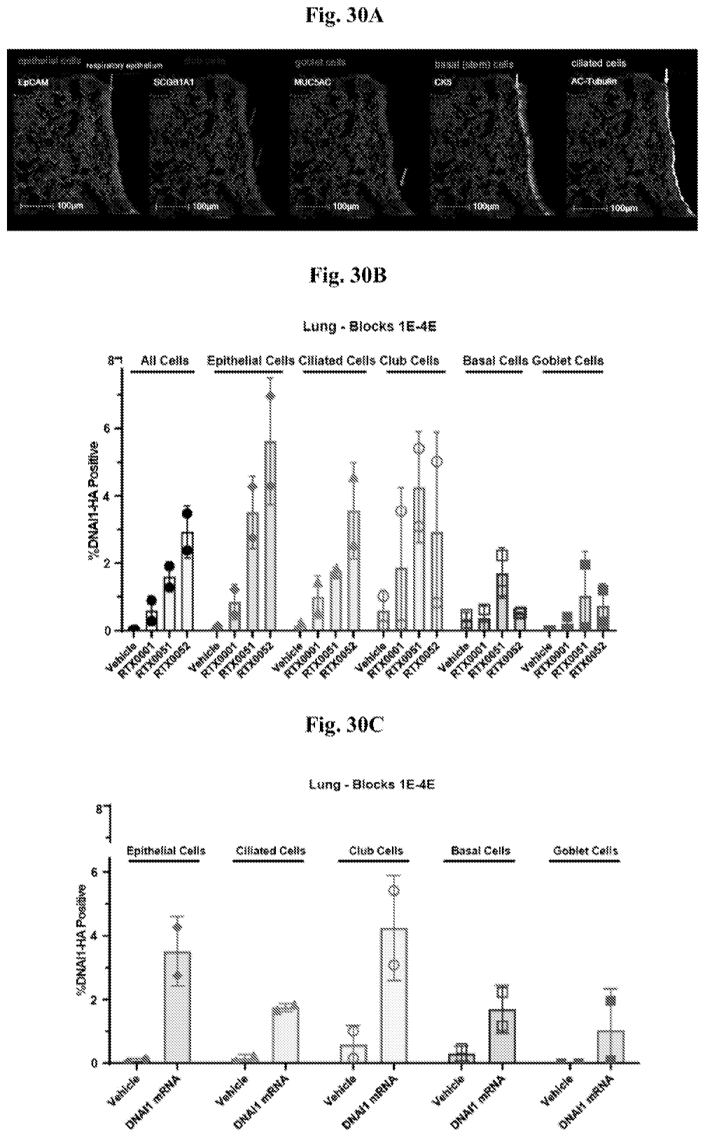

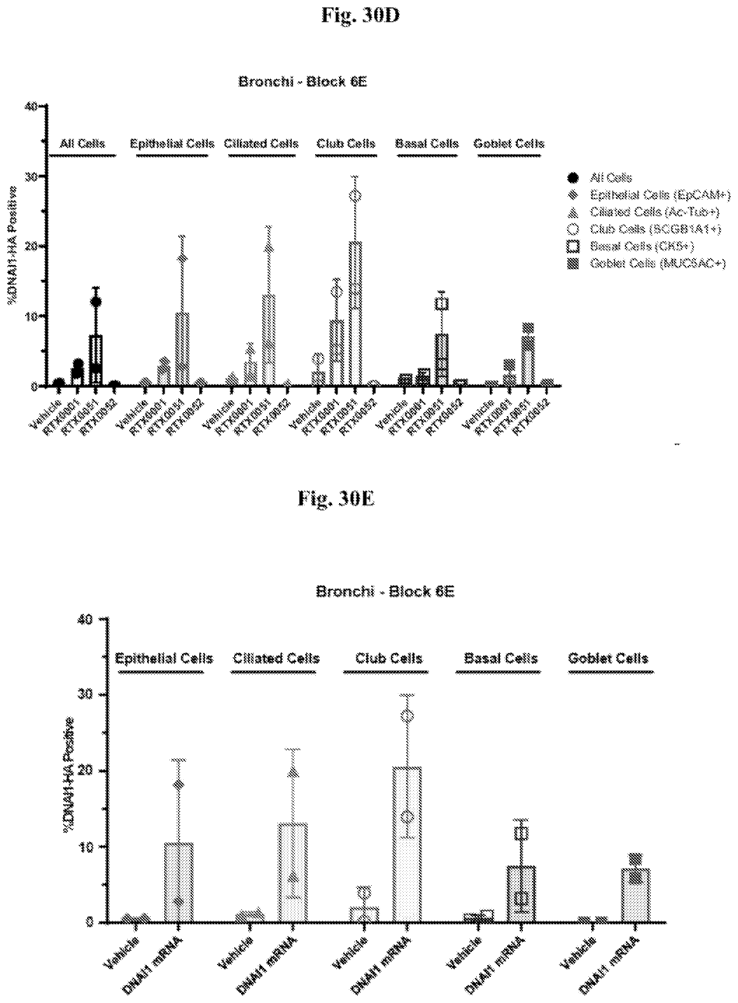

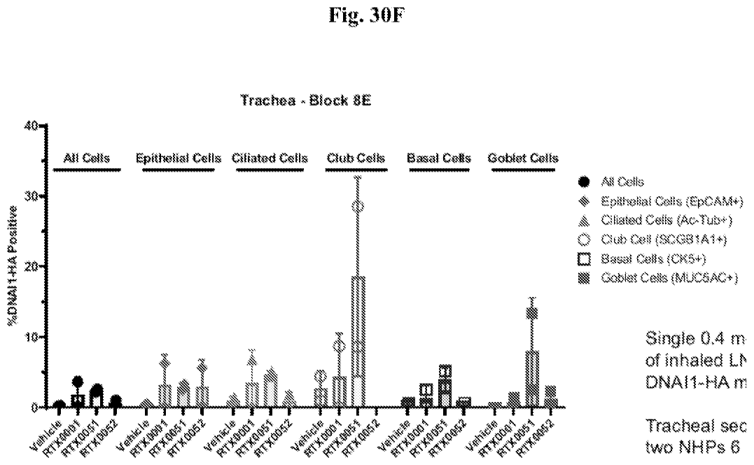

FIG. 30A illustrates multiplex immunofluorescent (IF) images for epithelial cell types. Epithelium cell was marked with EPCAM. Ciliated cell was marked with acetylated-tubulin (AC-tubulin). Club cell was marked with secretoglobin family 1A member 1 (SCGB1A1). Goblet cell was marked with mucin 5AC (MUC5AC). Basal cell (stem cell) was marked with cytokeratin 5 (CK5). FIG. 30B illustrates multiplex IF analysis demonstrating expression of DNAI1-HA protein in target cells in lung. FIG. 30C illustrates multiplex IF analysis demonstrating expression of DNAI1-HA protein in target cells in lung with RTX0051. High sensitivity of mIF enabled detection of protein expression in tissue and cells not detectable by other protein measurement methods. Single dose of 0.4 mg/kg was administered via inhalation of LNP-formulated DNAI1-HA mRNA. Lung sections were collected from two NHPs six hours after dosing. Percentage of DNAI1-HA positive cell was calculated by combining cell counts from all 4 examined lung sections for an individual animal. Total number of cells counted per animal was about 500,000 to 1,400,000 cells. Shown are the individual data points for each treated animal and the mean±std. dev. for each group (N=2).

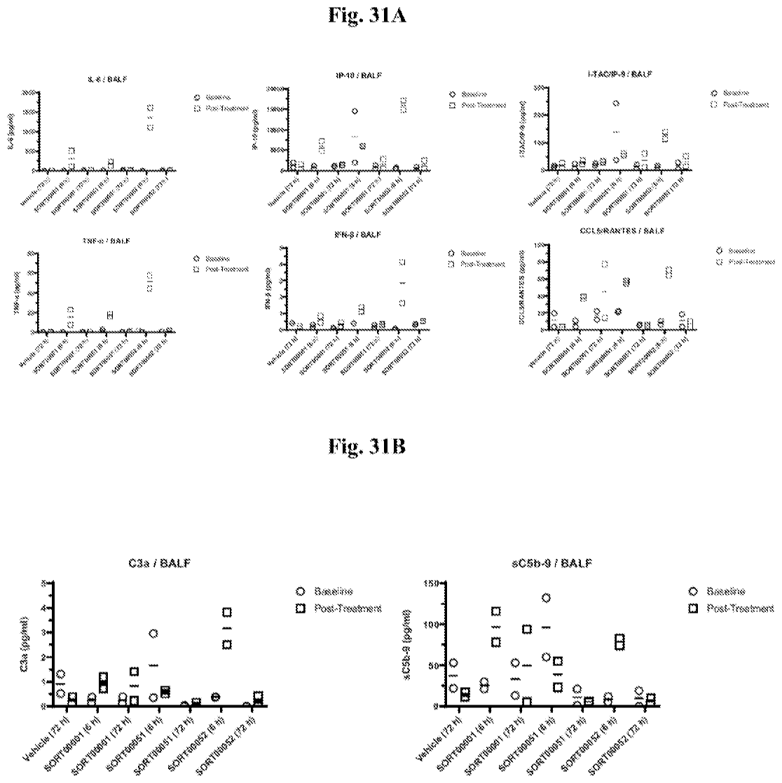

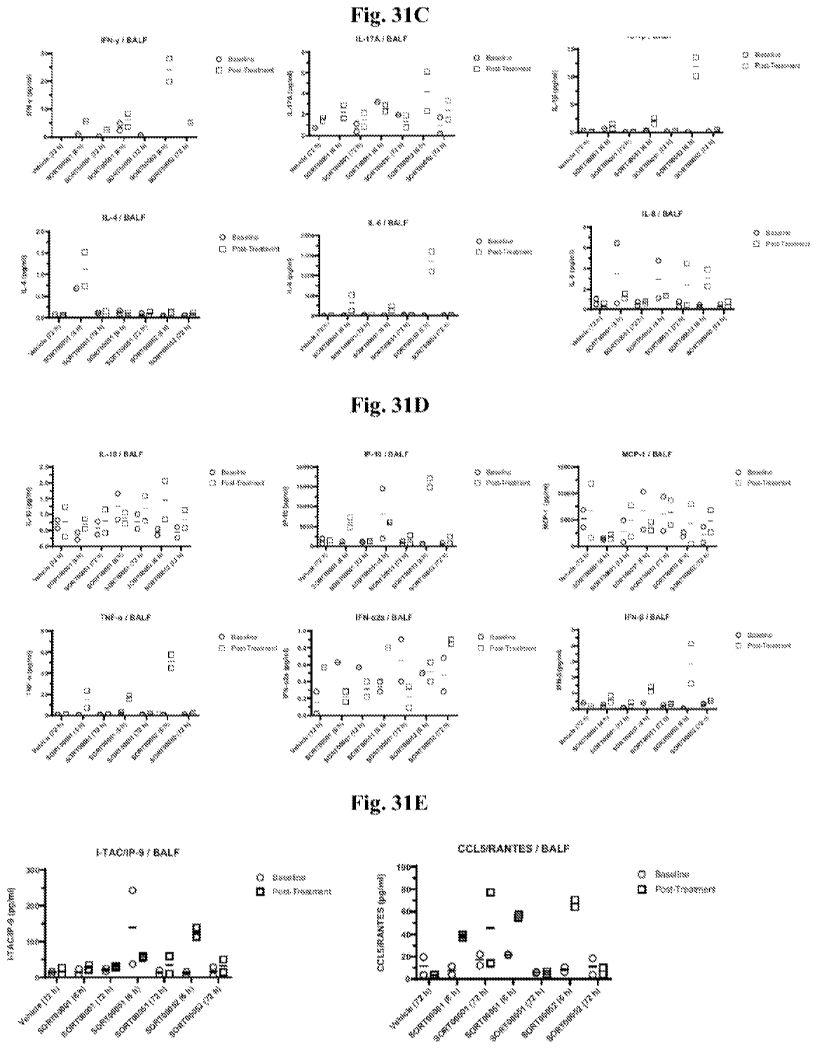

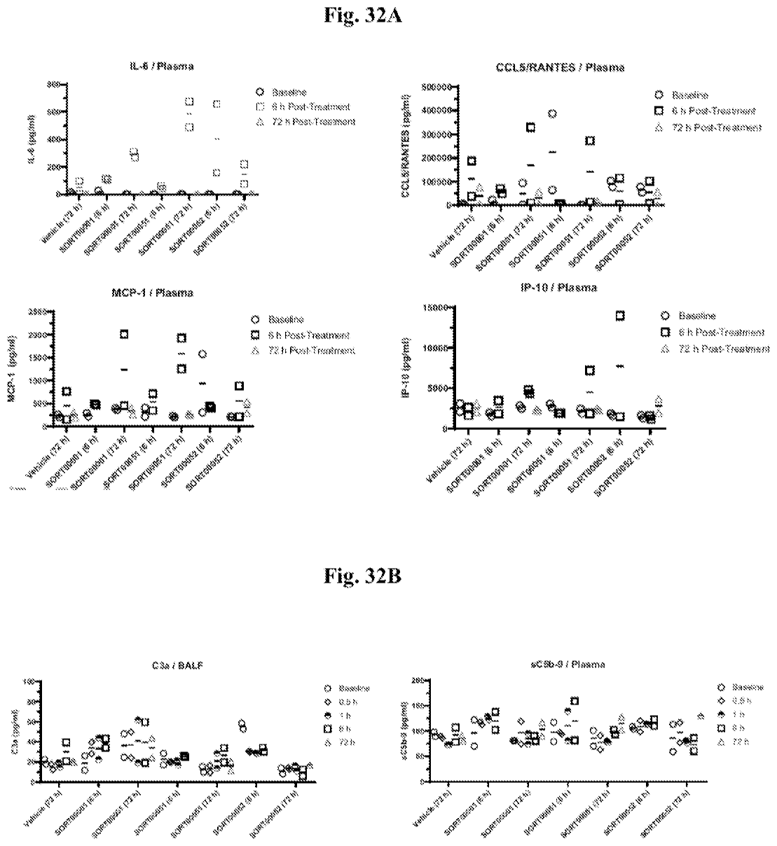

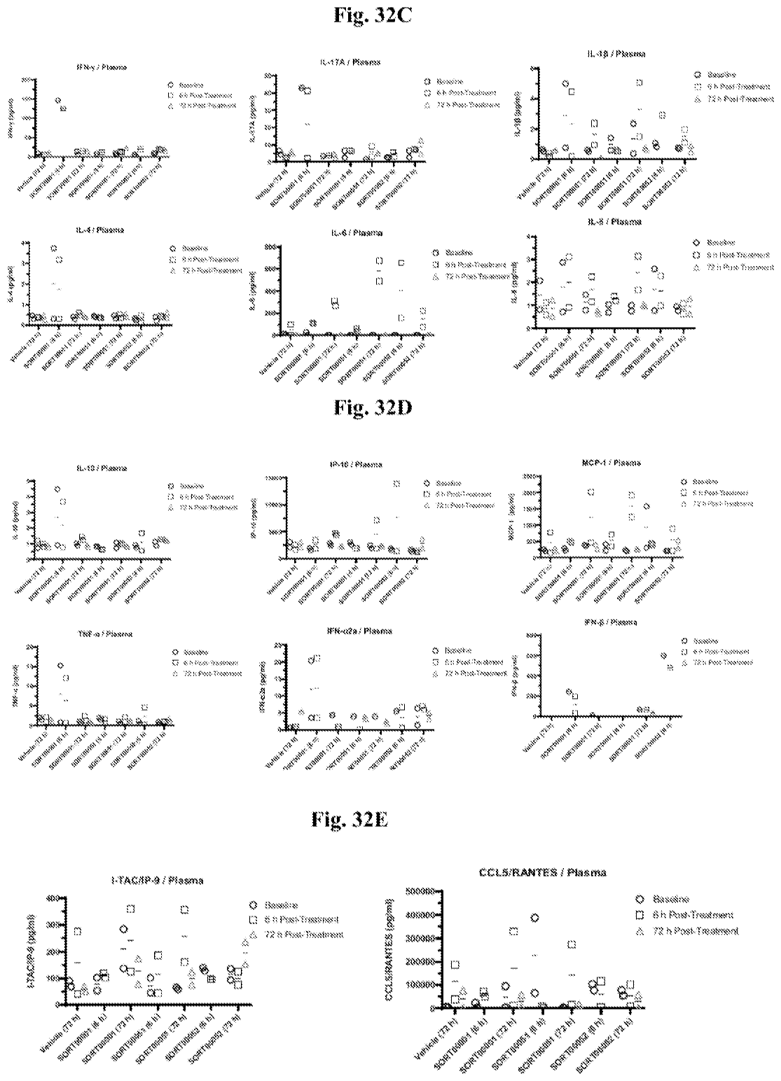

FIG. 30D illustrates multiplex IF analysis demonstrating expression of DNAI1-HA protein in target cells in bronchi. FIG. 30E illustrates multiplex IF analysis demonstrating expression of DNAI1-HA protein in target cells in bronchi with RTX0051. Single dose of 0.4 mg/kg was administered via inhalation of LNP-formulated DNAI1-HA mRNA. Bronchial sections were collected from two NHPs six hours after dosing. Percentage of DNAI1-HA positive cell was calculated from a single stained section per animal. Total number of cells counted was about 16,000 to 65,000 cells. Shown are the individual data points for each treated animal and the mean±std. dev. for each group (N=2). FIG. 30F illustrates multiplex IF analysis demonstrating expression of DNAI1-HA protein in target cells in trachea. Single dose of 0.4 mg/kg was administered via inhalation of LNP-formulated DNAI1-HA mRNA. Tracheal sections were collected from two NHPs six hours after dosing. Percentage of DNAI1-HA positive cell was calculated from a single stained section per animal. Total number of cells counted was about 16,000 to 28,000 cells. Shown are the individual data points for each treated animal and the mean±std. dev. for each group (N=2). FIG. 31A-E illustrate BAL cytokine and complement results. FIGS. 32A-E illustrate plasma cytokine results.

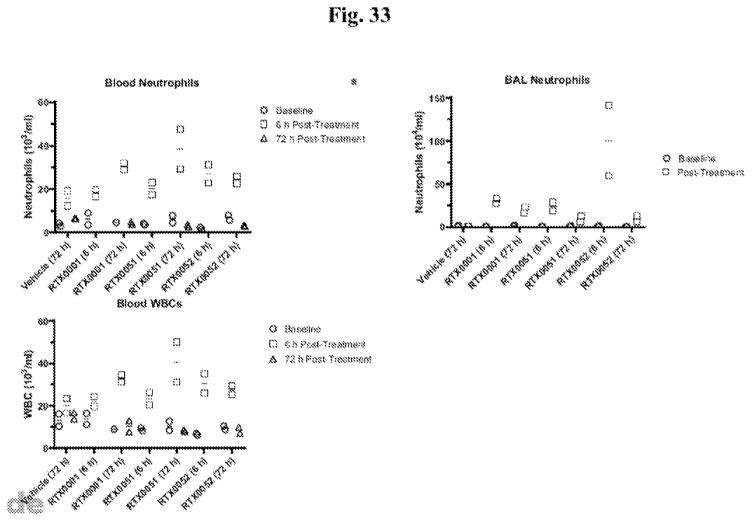

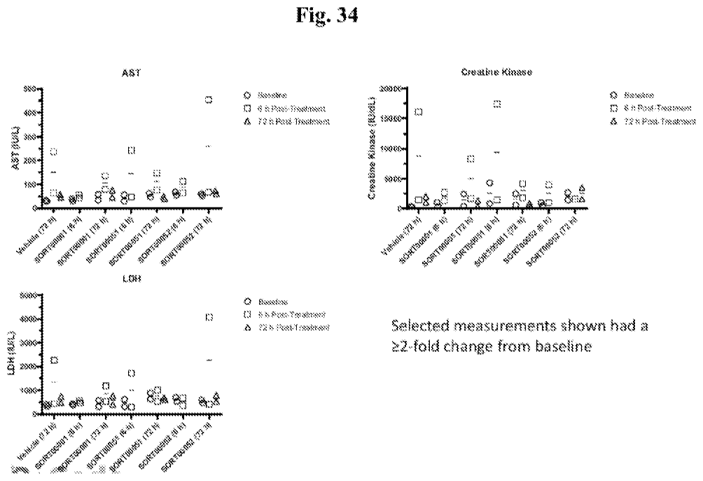

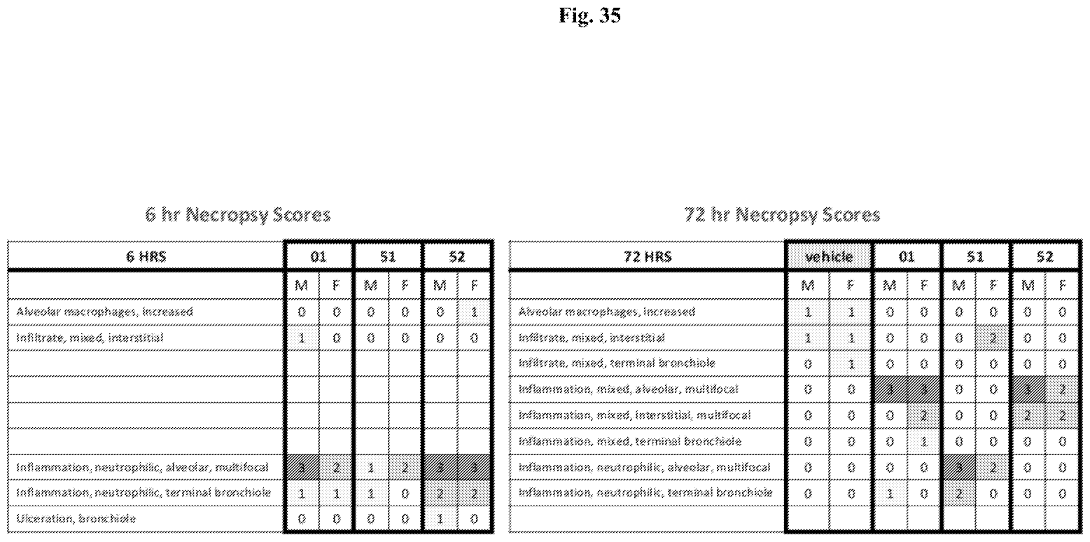

FIG. 33 illustrates transient increase in neutrophils observed in BAL and blood at six hours post-exposure. Transient increases in BAL neutrophils and blood WBCs and neutrophils seen at six hours post-exposure. Levels of neutrophils and WBCs had returned to baseline at 72 hours. No other significant changes in blood or BAL cell populations were observed. FIG. 34 illustrates selected clinical chemistry results. Small increases were observed for AST, LDH, and creatine kinase in individual animals after treatment. These increases were seen in both vehicle and TA treated animals but transient and values returned to baseline by 72 hours. No significant increases were observed for rest of the blood chemistry panel. Coagulation assays showed similar results for vehicle and TA treated animals. FIG. 35 illustrates summary of tolerability as determined by clinical observations and organ weights. Animals were observed during exposure, for up to three hours post exposure and 6 hours thereafter. Cage side clinical observation (e.g., clinical signs, etc.) was performed in the AM and PM on exposure day until euthanasia. Special attention was paid to clinical signs including but not limited to apnea, dyspnea (labored breathing), malaise, marked nasal discharge, lethargy, abnormal heartbeat, cyanosis, discoloration of mucous membrane, bloody stool/urine, excessive body weight loss (>20% from baseline bodyweight). No adverse reactions were reported in any of the animals during and after exposure. No change in body weight was observed during duration of the study. Organ Weights and weights normalized to body weights were not statistically different between treatment groups. Normalized lung weights appeared to be slightly higher in the animals treated with RTX0001 and RTX0052 but no statistical significance due to small sample size. Following a single high dose administration of RTX0001, RTX0051, and RTX0052, inflammation of the lung was observed with all three formulations. The observed severity of Grade 3 (moderate) inflammation is a concern and could impact lung function at these high concentrations. While this degree of inflammation was seen with all formulations, the presentation was different. With RTX0001 and RTX0052, the early observation at 6 hours included Grade 3 multifocal neutrophilic alveolar inflammation, which by 72 hours had progressed to mixed cell inflammation (also Grade 3). At 72 hours, RTX0051 was observed with Grade 3 multifocal neutrophilic alveolar inflammation, similar to that observed at 6 hours for RTX0001 and RTX0052; it is unknown if this would have progressed to mixed cell inflammation similar to RTX0001 and RTX0052 given a later sampling.

### Example 28. Single Dose Inhalation Study

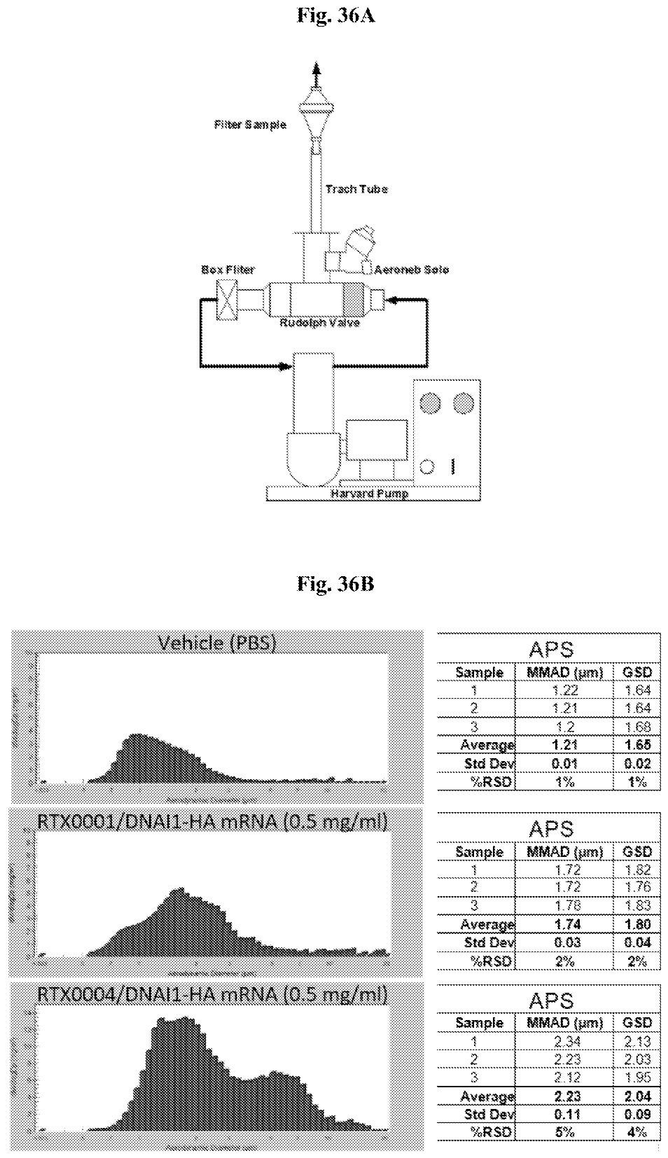

This example compared 4-component (RTX0004) and 5-component (SORT, RTX0001) LNPs for lung distribution of the mRNA and protein expression in target cells. The comparison included LNP formulation assessment; LNP formulation with mRNA as cargo (e.g., mRNA encoding DNAI1-GA), LNP formulation with modified nucleotide as cargo (e.g., mRNA comprising modified nucleotides); and LNP formulation with sequence optimized mRNA for stability and translation efficiency. NHPs (Mauritius cynomolgus macaques, 1-3 yrs. old, female, about 3 kg, N=4 per formulation, 2 per necropsy timepoints) were intubated and ventilated and treated with the LNPs by aerosol delivery. The target delivered dose was 0.1 mg/kg. Necropsy timepoints for assessment were at six hours and at 24 hours. FIG. 36A illustrates a diagram of the aerosol delivery system. The amount of aerosolized drug delivered past the endotracheal tube was estimated using the test setup shown on the left. Pre-weighed glass fiber and MCE filters were attached directly at the exit of the endotracheal tube. Multiple collections were performed before, during and after treatment of the animals. The glass filters were dried and quantified using both gravimetric analyses. The MCE filters were analyzed for amount of mRNA using a RiboGreen assay. FIG. 36B illustrates the results of aerosol particle size measurements. Particle sizes for test article exposure were measured for deposition in the conducting airways (branching generations 0-15 in humans).

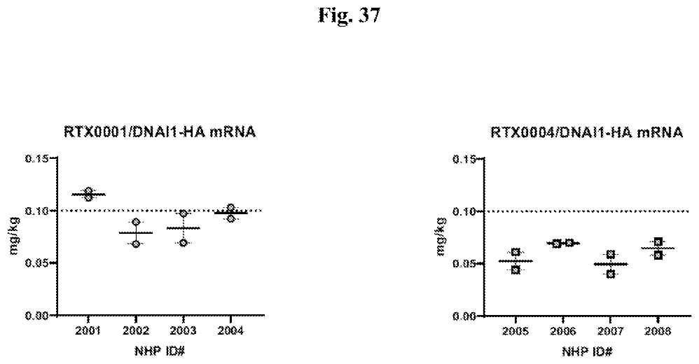

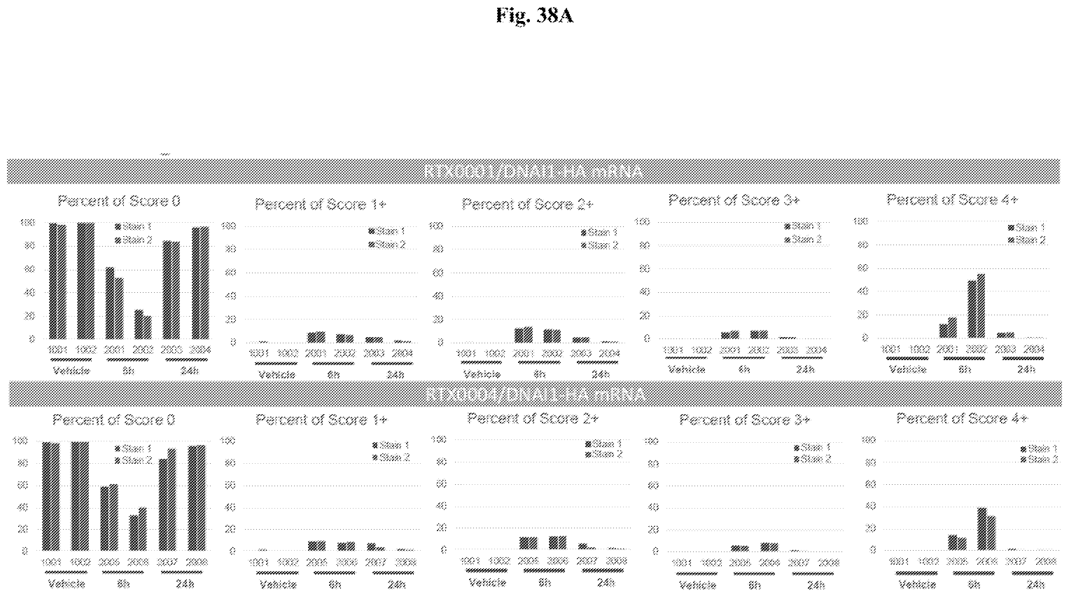

FIG. 37 illustrates DBAI1-HA mRNA dose present in the NHP. A dashed black horizontal line represents the targeted presented dose of 0.1 mg/kg. Open yellow circles show filter collections before and after dosing (GF, n=2) using RTX0001/DNAI1-HA mRNA. Open yellow squares show filter collections before and after dosing (GF, n=2) using RTX0004/DNAI1-HA mRNA. Similar results obtained for mRNA using MCE filters and a RiboGreen assay. FIGS. 36 and 37 collectively show that aerosol particle size ranges appropriate for deposition in the conducting airways (branching generations 0-15 in humans). Target delivered dose of 0.1 mg/kg was achieved for RTX0001/DNAI1-HA mRNA and between 25-50% lower than targeted dose for RTX0004/DNAI1-HA mRNA. Problems with clogging or changes in device performance/flow rates were not observed. Both test articles nebulized well with minimal differences in flow rates. RTX004/DNAI1-HA mRNA had a slightly higher flow rate compared RTX0001/DNAI1-HA mRNA. The faster flow rates and lower exposures for RTX0004/DNAI1-HA mRNA could be due to the larger aerosol droplet sizes observer by APS measurements.

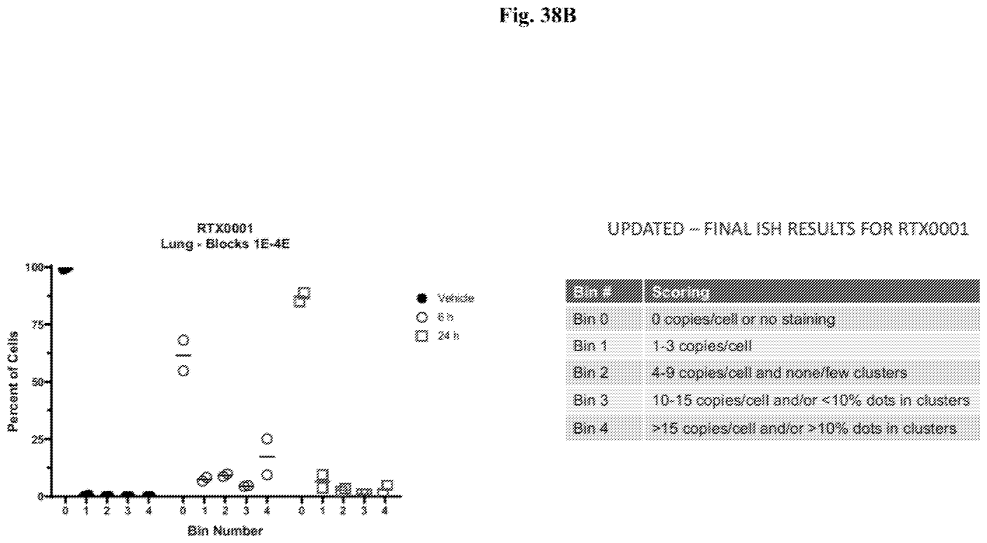

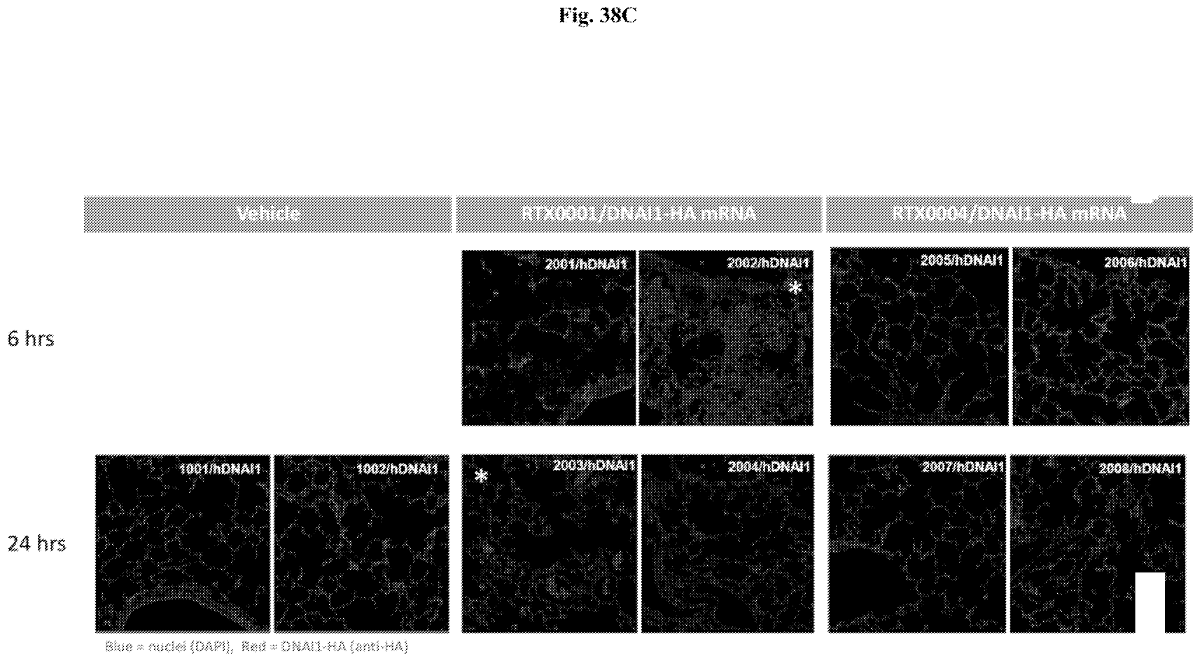

In situ hybridization (ISH) assay was used to detect DNAI1-HA mRNA delivered to the lungs of NHPs using custom designed ISH probe. ISH results were analyzed into bins: 0+: zero minimum copies/cell; 1+: one minimum copy/cell; 2+: 4 minimum copies/cell; 3+: 10 minimum copies/cell; and 4+: 16 minimum copies/cell. FIG. 38A illustrates DNAI1-HA mRNA ISH results for lung tissue. Data from assay qualification: 1 of 4 samples per animal analyzed. DNAI1-HA mRNA detected in all animals. FIG. 38B illustrates that a significant fraction of lung cells contained DNAI1-HA mRNA after treatment with RTX0001 as measured by ISH and the bin scoring. FIG. 38B demonstrates that up to 25% of lung cells contained more than 15 copies of the DNAI1-HA mRNA per cell after treatment with RTX0001. FIG. 38C illustrates the imaging of the lung tissue used for the ISH analysis.

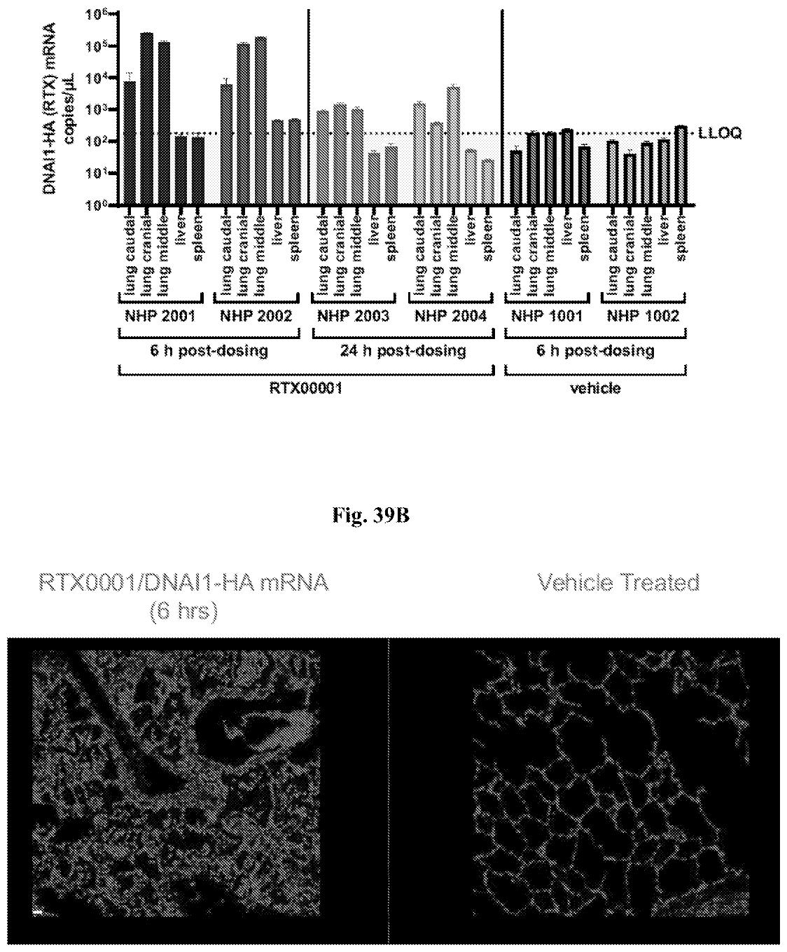

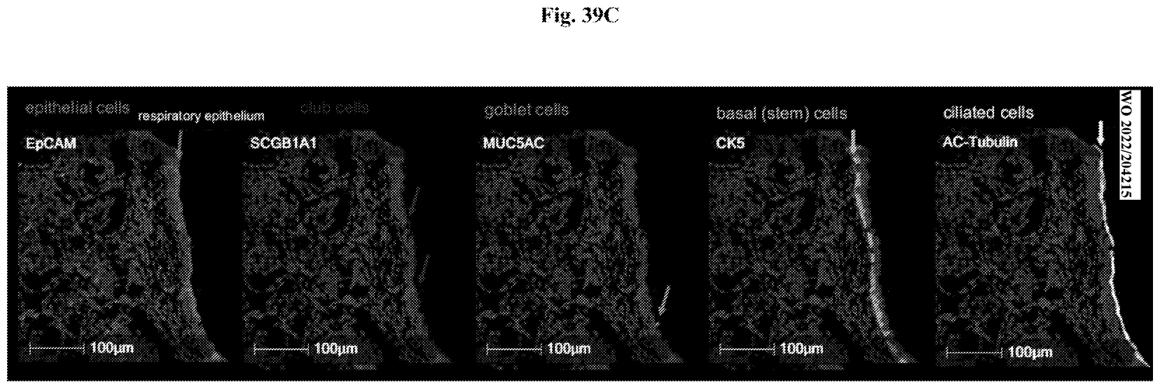

FIG. 39A illustrates that the delivery of high levels of DNAI1-HA to the lung did not lead to similar deliver to liver or spleen. Digital PCR was used to measure DNAI1-HA mRNA levels in whole blood, lung, liver, and spleen tissue following a single 0.1 mg/kg administration. Primers used were specific for the RTX sequence optimized DNAI1-HA sequence. High levels of DNAI1-HA mRNA were detected in all three lung regions sampled at 6 hour post-exposure with RTX0001. In spleen and liver, DNAI1-HA mRNA was only measured at or below the LLOQ of the assay. FIG. 39B illustrates the positive staining of DNAI1-HA tagged protein in NHPs. For RTX0001, DNAI1-HA was detected six hours or 24 hours after administration. Regions with higher mRNA levels correlated with regions showing highest levels of DNAI1-HA protein. DNAI1-HA mRNA was present in all eight treated animals. No signal detected in vehicle treated animals. mRNA levels were highest at six hours and lower at 24 hours. mRNA levels were highest for RTX0001 treated animals compared to RTX0004 (consistent with emitted dose measurements). Using serial sections, regions with higher mRNA levels were correlated with regions showing highest levels of DNAI1-HA protein. DNAI1-HA was detected at six hours and 24 hours in NHPs treated with RTX0001. FIG. 39C illustrates multiplex IF panel for key epithelial cell types. 10 NHP FFPE lung tissue blocks (1 from each animal) were used for mIF assay qualification. Two slides from each block were stained in duplicates. The cell counts of single marker positive cells, double positive cells with DNAI1 expression, and DNAI1 MFI in double positive cells were reported. Epithelium cell was marked with EPCAM. Ciliated cell was marked with acetylated-tubulin (AC-tubulin). Club cell was marked with secretoglobin family TA member 1 (SCGB1A1). Goblet cell was marked with mucin 5AC (MUC5AC). Basal cell (stem cell) was marked with cytokeratin 5 (CK5).

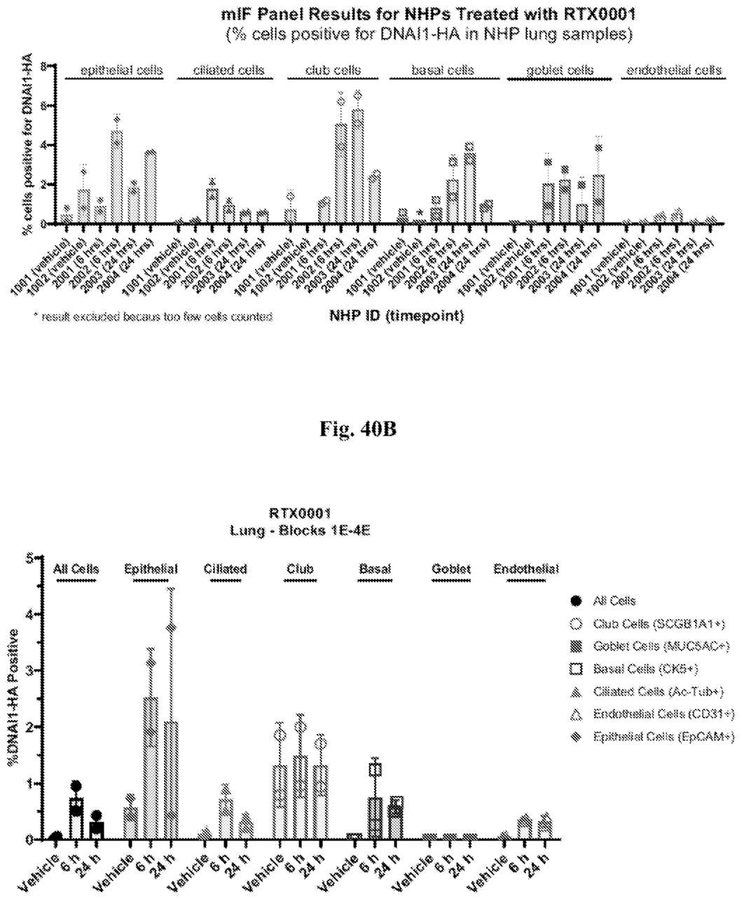

FIG. 40A illustrates multiplex IF panel results for NHP lung samples. DNAI1-HA was expressed in cells of the respiratory epithelium. Percentage of DNAI1-HA positive cell was calculated by combining cell counts from 1 examined lung section per animal. DNAI1-HA expression was detected in lung samples from NHPs treated with RTX0001. DNAI1-HA expression was co-localized with markers for epithelial cells, including the club, basal and ciliated cells (club and basal cells are precursors for ciliated cells). No staining detected was in lung samples from NHPs treated with RTX0004. FIG. 40B illustrates multiplex IF analysis of expression of DNAI1-HA protein in target cell in the lung. Single dose of 0.1 mg/kg of RTX0001/DNAI1-HA mRNA was administered via inhalation. Lung sections were collected from two NHPs at six hours and 24 hours after dosing. Percentage of DNAI1-HA positive cell was calculated by combining cell counts from all 4 examined lung sections for an individual animal. Total number of cells counted per animal was about 690,000 to 1,100,000. Shown are the individual data points for each treated animal and the mean±std. dev. for each group (N=2).

The present study showed that the aerosol particle size was consistent with deposition in the conducting airways for both formulations, and the flow rates were consistent throughout exposures (more than >0.20 mL/min for both formulations). The exposure time for both formulations were short (between four to nine minutes). The biodistribution results indicated good distribution of mRNA, with higher levels for NHPs treated with formulation RTX0001. DNAI1 protein was detected in ciliated, club, and basal cells of animals treated with formulation RTX0001. Little or no DNAI1-HA expression was detected with RTX0004 (note: delivered dose was 25-50% lower compared to RTX0001)

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-108. (canceled)

109. A method for treating a subject having or suspected of having primary ciliary dyskinesia (PCD), comprising administering to said subject a pharmaceutical composition comprising a heterologous polynucleotide assembled with a lipid composition, which heterologous polynucleotide encodes a dynein axonemal intermediate chain 1 (DNAI1) protein, thereby resulting in a heterologous expression of said DNAI1 protein within cells of said subject,
wherein said lipid composition comprises
(i) an ionizable cationic lipid, and
(ii) a selective organ targeting (SORT) lipid separate from said ionizable cationic lipid.

110. The method of claim 109, wherein said lipid composition further comprises (iii) a phospholipid.

111. The method of claim 109, wherein said administering comprises administering to a lung by nebulization.

112. The method of claim 109, wherein said subject is determined to exhibit an aberrant expression or activity of DNAI1 gene or protein.

113. The method of claim 112, wherein said subject is a human.

114. The method of claim 109, wherein said cells are in a lung of said subject.

115. The method of claim 114, wherein said cells comprises ciliated cells, basal cells, club cells, or a combination thereof.

116. (canceled)

117. The method of claim 114, wherein said cells are undifferentiated or are differentiated.

118. (canceled)

119. The method of claim 114, wherein said ciliated cells are ciliated epithelial cells.

120.-121. (canceled)

122. A method for enhancing an expression or activity of dynein axonemal intermediate chain 1 (DNAI1) protein in a cell, the method comprising:
contacting said cell with a composition comprising a synthetic polynucleotide assembled with a lipid composition, wherein said synthetic polynucleotide encodes a DNAI1 protein, wherein said lipid composition comprises an ionizable cationic lipid and a selective organ targeting (SORT) lipid separate from said ionizable cationic lipid,
thereby providing a therapeutically) effective amount or activity of a functional DNAI1 protein in said cell.

123. The method of claim 122, wherein the method provides a therapeutically effective amount or activity of said functional DNAI1 protein in said cell at least about 6 hours after said contacting.

124. The method of claim 122, wherein said contacting is in vivo, ex vivo, or in vitro.

125.-126. (canceled)

127. The method of claim 122, wherein said cell is in a ciliary axoneme.

128. The method of claim 122, wherein mucus is present in said contacting.

129. The method of claim 122, wherein said cell is an airway epithelial cell, a ciliated cell, a basal cell, a goblet cell, or a club cell.

130-131. (canceled)

132. The method of claim 122, wherein said cell exhibits a mutation in DNAI1 gene, transcript, or protein.

133. (canceled)

134. The method of claim 122, wherein the method provides a therapeutically effective amount or activity of said functional DNAI1 protein in at least about 5%, 10%, 15%, or 20% lung epithelial, ciliated, secretory, club, goblet, and/or basal cells.

135.-139. (canceled)

140. The method of claim 122, wherein said contacting is repeated at least once or is repeated about 2, 4, 6, 8, or 10 times).

141. The method of claim 140, wherein said contacting is repeated at least once per week, at least twice per week, or at least three times per week.

142. The method of claim 140, wherein a treatment holiday follows at least one contacting step.
